(19) United States
(12) Patent Application Publication
(10) Pub. No.: US 2022/0062296 A1
(43) Pub. Date: Mar. 3, 2022

(54) COMBINATIONS OF GABAA ALPHA 5 AGONISTS AND SV2A INHIBITORS AND METHODS OF USING IN THE TREATMENT OF COGNITIVE IMPAIRMENT

(71) Applicant: AGENEBIO, INC., Baltimore, MD (US)

(72) Inventors: Michela Gallagher, Baltimore, MD (US); Sharon Rosenzweig-Lipson, East Brunswick, NJ (US)

(21) Appl. No.: 17/372,405

(22) Filed: Jul. 9, 2021

Related U.S. Application Data
(60) Provisional application No. 63/050,730, filed on Jul. 10, 2020.

(51) Int. Cl. A61K 31/5517; A61K 45/06

(52) U.S. Cl. CPC A61K 31/5517; A61K 45/06

(57) ABSTRACT

This disclosure relates to methods, uses, combinations, pharmaceutical compositions, combinations for use, and pharmaceutical compositions for use useful for treating cognitive impairment associated with central nervous system (CNS) disorders. In particular, it relates to the use of inhibitors of synaptic vesicle glycoprotein 2A (SV2A), in combination with GABA_{A} α5 receptor agonists, in treating cognitive impairment associated with central nervous system (CNS) disorders in a subject in need or at risk thereof, including, without limitation, subjects having or at risk for age-related cognitive impairment, mild cognitive impairment (MCI), amnestic MCI (aMCI), age-associated memory impairment (AAMI), age related cognitive decline (ARCD), dementia, Alzheimer's disease (AD), prodromal AD, post traumatic stress disorder (PTSD), schizophrenia, bipolar disorder, amyotrophic lateral sclerosis, cancer-therapy-related cognitive impairment, mental retardation, Parkinson's disease, autism, compulsive behavior, and substance addiction. Further, the disclosure relates to methods, uses, combinations, pharmaceutical compositions, combinations for use, and pharmaceutical compositions for use useful for treating cognitive impairment associated with brain cancer or for treating brain cancer itself in a subject in need thereof. Additionally, the disclosure relates to methods, uses, combinations, pharmaceutical compositions, combinations for use, and pharmaceutical compositions for use useful for treating Parkinson's disease psychosis in a subject in need thereof.

### RELATED APPLICATIONS

This application claims the benefit of and priority from U.S. Provisional Application 63/050,730, filed Jul. 10, 2020, which is incorporated herein by reference in its entirety.

### STATEMENT OF GOVERNMENT SUPPORT

This invention was made with government support under Grant No. UH3NS101856 awarded by the National Institutes of Health (NIH), and in particular, its National Institute on Aging (NIA) division, an agency of the United States Government. The United States Government has certain rights in the invention.

### FIELD OF THE DISCLOSURE

This disclosure relates to methods, uses, combinations, and pharmaceutical compositions useful for treating cognitive impairment and improving cognitive function by using a synaptic vesicle glycoprotein 2A (SV2A) inhibitor in combination with a GABA_{A} α5 receptor agonist. In particular, it relates to the use of a SV2A inhibitor in combination with a GABA_{A} α5 receptor agonist in treating cognitive impairment associated with central nervous system (CNS) disorders in a subject in need thereof or at risk thereof, including, without limitation, subjects having or at risk for having age-related cognitive impairment, mild cognitive impairment (MCI), amnestic MCI (aMCI), age-associated memory impairment (AAMI), age related cognitive decline (ARCD), dementia, Alzheimer's disease (AD), prodromal AD, post-traumatic stress disorder (PTSD), schizophrenia, bipolar disorder, amyotrophic lateral sclerosis (ALS), cancer-therapy-related cognitive impairment, mental retardation, Parkinson's disease (PD), autism, compulsive behavior, and substance addiction. The present disclosure also provides combinations for use and pharmaceutical compositions for use in treating cognitive impairment and improving cognitive function. Further, the disclosure relates to methods, uses, combinations, pharmaceutical compositions, combinations for use, and pharmaceutical compositions for use in treating cognitive impairment associated with a brain cancer or for treating a brain cancer itself in a subject in need thereof. Additionally, the disclosure relates to methods, uses, combinations, pharmaceutical compositions, combinations for use, and pharmaceutical compositions for use in treating Parkinson's disease psychosis in a subject in need thereof.

### Background of the Disclosure

Cognitive ability may decline as a normal consequence of aging or as a consequence of a central nervous system (CNS) disorder or a brain cancer.

For example, a significant population of elderly adults experiences a decline in cognitive ability that exceeds what is typical in normal aging. Such age-related loss of cognitive function is characterized clinically by progressive loss of memory, cognition, reasoning, and judgment. Age-associated memory impairment (AAMI), age-related cognitive decline (ARCD), or similar clinical groupings may be related to such age-related loss of cognitive function. According to some estimates, there are more than 16 million people with AAMI in the U.S. alone (Barker et al., 1995).

Cognitive impairment is also associated with other central nervous system (CNS) disorders, such as mild cognitive impairment (MCI), dementia, Alzheimer's disease (AD), prodromal AD, post-traumatic stress disorder (PTSD), schizophrenia, bipolar disorder (e.g., mania), amyotrophic lateral sclerosis (ALS), cancer-therapy-related cognitive impairment, mental retardation, Parkinson's disease (PD), autism, compulsive behavior, and substance addiction. For example, MCI is estimated to affect 5.5 million to 7 million people in the U.S. over the age of 65 (Plassman et al., 2008).

There is, therefore, a need for effective treatment of these cognitive impairments as well as cognitive impairments associated with other central nervous system (CNS) disorders.

Further, there is a need to treat cognitive impairment associated with a brain cancer or a brain cancer itself in a subject in need thereof. Additionally, there is a need to treat Parkinson's disease psychosis in subject in need thereof.

### BRIEF SUMMARY OF THE DISCLOSURE

The present disclosure provides methods, uses, combinations, and pharmaceutical compositions useful for treating cognitive impairment and improving cognitive function. In particular, it relates to combinations of SV2A inhibitors and GABA_{A} α5 receptor agonists useful for treating cognitive impairment associated with central nervous system (CNS) disorders in a subject in need thereof or at risk thereof, including, without limitation, subjects having or at risk for having age-related cognitive impairment, mild cognitive impairment (MCI), amnestic MCI (aMCI), age-associated memory impairment (AAMI), age related cognitive decline (ARCD), dementia, Alzheimer's disease (AD), prodromal AD, post-traumatic stress disorder (PTSD), schizophrenia, bipolar disorder, amyotrophic lateral sclerosis (ALS), cancer-therapy-related cognitive impairment, mental retardation, Parkinson's disease (PD), autism, compulsive behavior, and substance addiction. The present disclosure also provides combinations for use and pharmaceutical compositions for use in treating cognitive impairment and improving cognitive function. Further, the present disclosure provides methods, uses, combinations, pharmaceutical compositions, combinations for use, and pharmaceutical compositions for use in treating cognitive impairment associated with a brain cancer or for treating a brain cancer itself in a subject in need thereof. Additionally, the present disclosure provides methods, uses, combinations, pharmaceutical compositions, combinations for use, and pharmaceutical compositions for use in treating Parkinson's disease psychosis in a subject in need thereof.

An aspect of the disclosure relates to a pharmaceutical composition comprising:
A) an SV2A inhibitor, or a pharmaceutically acceptable salt, hydrate, solvate, isomer, or polymorph thereof; and
B) a GABA_{A} α5 receptor agonist, or a pharmaceutically acceptable salt, hydrate, solvate, isomer, or polymorph thereof. In some embodiments, the GABA_{A} α5 receptor agonist, or the pharmaceutically acceptable salt, hydrate, solvate, isomer, or polymorph thereof, is selected from the group consisting of:
   i) a compound of formula I:

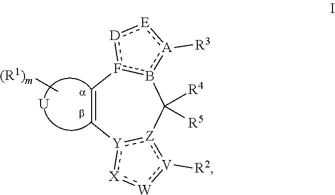

or a pharmaceutically acceptable salt, hydrate, solvate, polymorph, or isomer thereof, wherein:
U and the two carbon atoms designated by α and β together form a 5- or 6-membered aromatic ring having 0-2 nitrogen atoms;
A is C, CR⁶, or N;
B and F are each independently selected from the group consisting of C, CR⁶, and N, wherein B and F cannot both be N;
D is N, NR⁷, O, CR⁶ or C(R⁶)₂;
E is N, NR⁷, CR⁶ or C(R⁶)₂;
W is N, NR⁷, CR⁶ or C(R⁶)₂;
X is N, NR⁷, O, CR⁶ or C(R⁶)₂;
Y and Z are each independently selected from the group consisting of C, CR⁶, and N, wherein Y and Z cannot both be N;
V is C or CR⁶,
or when Z is C or CR⁶, V is C, CR⁶, or N;
wherein when the ring formed by X, Y, Z, V and W is

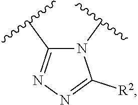

then R² is —OR⁸, —SR⁸, —(CH₂)ₙOR⁸, —(CH₂)ₙO(CH₂)ₙR⁸, —(CH₂)_{P}R⁸ or —(CH₂)ₙN(R″)R¹⁰; and wherein R² is independently substituted with 0-5 R′;
m and n are independently integers selected from 0-4;
p is an integer selected from 2-4;
each occurrence of the bond “ [symbol] ” is independently either a single bond or a double bond;
each occurrence of R¹, R², R⁴, and R⁵ are each independently selected from the group consisting of:
   halogen, —R, —OR, —NO₂, —NCS, —CN, —CF₃, —OCF₂H —OCF₃, —SiR₃, —N(R)₂, —SR, —SOR, —SO₂R, —SO₂N(R)₂, —SO₃R, —(CR₂)₁₋₃R, —(CR₂)₁₋₃—OR, —(CR₂)₁₋₃—O(CR₂)₁₋₃—R, —(CR₂)₀₋₃—C(O)NR(CR₂)₀₋₃R, —(CR₂)₀₋₃—C(O)NR(CR₂)₀₋₃OR, —C(O)R, —C(O)C(O)R, —C(O)CH₂C(O)R, —C(S)R, —C(S)OR, —C(O)OR, —C(O)C(O)OR, —C(O)C(O)N(R)₂, —OC(O)R, —C(O)N(R)₂, —OC(O)N(R)₂, —C(S)N(R)₂, —(CR₂)₀₋₃NHC(O)R, —N(R)N(R)COR, —N(R)N(R)C(O)OR, —N(R)N(R)CON(R)₂, —N(R)SO₂R, —N(R)S O₂N(R)₂, —N(R)C(O)OR, —N(R)C(O)R, —N(R)C(S)R, —N(R)C(O)N(R)₂, —N(R)C(S)N(R)₂, —N(COR)COR, —N(OR)R, —C(═NH)N(R)₂, —C(O)N(OR)R, —C(═NOR)R, —OP(O)(OR)₂, —P(O)(R)₂, —P(O)(OR)₂, —P(O)(H)(OR), —C≡C—R⁸, —CH₂CF₃, and CHF₂;
each occurrence of R⁸ is independently —H, —(C1-C6) alkyl, —(C3-C6) cycloalkyl, —(C1-C6) alkyl-(C3-C6) cycloalkyl, —(C1-C6) alkyl-(C6-C10) aryl, —(C6-C10) aryl, -5-10 membered heteroaryl, or —(C1-C6) alkyl-5-10 membered heteroaryl;
   wherein each R⁸ excluding —H and —(C1-C6) alkyl is independently substituted by 0-5 of -halogen, —(C1-C6) alkyl, —CF₃, —OCF₃, or O—(C1-C6) alkyl;
R³ is absent or is selected from the group consisting of:
   halogen, —R, —OR, —NO₂, —NCS, —CN, —CF₃, —OCF₃, —SiR₃, —N(R)₂, —SR, —SOR,
   —SO₂R, —SO₂N(R)₂, —SO₃R, —(CR₂)₁₋₃R, —(CR₂)₁₋₃—OR, —(CR₂)₀₋₃—C(O)NR(CR₂)₀₋₃R,
   —(CR₂)₀₋₃—C(O)NR(CR₂)₀₋₃OR, —C(O)R, —C(O)C(O)R, —C(O)CH₂C(O)R, —C(S)R,
   —C(S)OR, —C(O)OR, —C(O)C(O)OR, —C(O)C(O)N(R)₂, —OC(O)R, —C(O)N(R)₂,
   —OC(O)N(R)₂, —C(S)N(R)₂, —(CR₂)₀₋₃NHC(O)R, —N(R)N(R)COR, —N(R)N(R)C(O)OR,
   —N(R)N(R)CON(R)₂, —N(R)SO₂R, —N(R)SO₂N(R)₂, —N(R)C(O)OR, —N(R)C(O)R,
   —N(R)C(S)R, —N(R)C(O)N(R)₂, —N(R)C(S)N(R)₂, —N(COR)COR, —N(OR)R, —C(═NH)N(R)₂, —C(O)N(OR)R, —C(═NOR)R, —OP(O)(OR)₂, —P(O)(R)₂, —P(O)(OR)₂, —P(O)(H)(OR), C≡C—R⁹, COOMe, COOEt, —(C1-C6)alkyl-C≡C—R¹⁰, CH₂—OR¹⁰, and CH₂—O—CH₂—R¹⁰;
wherein each of R⁹ is independently selected from the group consisting of —H, —(C1-C6) alkyl, —(C6-C10) aryl, -5-10 membered heteroaryl, —(C1-C6) alkyl-(C6-C10) aryl, —(C1-C6) alkyl-5-10 membered heteroaryl, —(C3-C6) cycloalkyl, —(C1-C6) alkyl-(C3-C6) cycloalkyl, —C(O)—(C6-C10) aryl, —(C3-C6)cycloalkyl-(C6-C10)aryl,

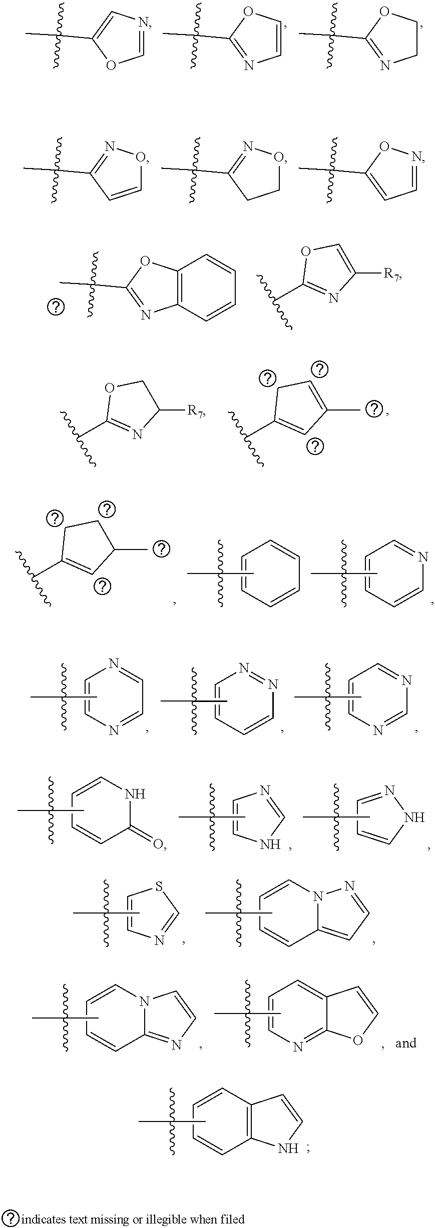

wherein each R⁹ is independently substituted with 0-5 R¹¹;
wherein each occurrence of R¹¹ is independently selected from the group consisting of -halogen, —CF₃, —OH, —OCF₃, OCHF₂, —O—(C1-C6)alkyl, —O—CH₂—(C3-C6)cycloalkyl, —CN, —SCH₃—(C6-C10) aryl, —(C1-C6)alkyl, and -5 to 10 membered heteroaryl,
wherein R¹⁰ is selected from the group consisting of —H, —(C1-C6) alkyl, —(C6-C10) aryl, -5-10 membered heteroaryl, —(C3-C6) cycloalkyl, —CH₂—(C3-C6) cycloalkyl, —CH₂—(C6-C10) aryl, and —CH₂-5-10-membered heteroaryl,
wherein each R¹⁰ is independently substituted with 0-5 R′;
wherein R₇ is selected from the group consisting of —(C1-C6)alkyl, —(C3-C6)cycloalkyl, -5 to 10 membered heteroaryl, —(C6-C10) aryl, —(C6-C10)aryl-(C1-C6)alkyl, and -5 to 10 membered heteroaryl-(C1-C6)alkyl, and -5-10 membered heteroaryl,
wherein each R₇ is independently substituted with 0-5 R′;
each R⁶ is independently —H or —(C1-C6)alkyl;
each R⁷ is independently —H or —(C1-C6)alkyl;
each R⁸ is independently —(C1-C6)alkyl, —(C3-C10)-cycloalkyl, (C6-C10)-aryl, or 5- to 10-membered heteroaryl, wherein each occurrence of R⁸ is independently substituted with 0-5 R′;
each R¹⁰ is independently —(C3-C10)-cycloalkyl, 3- to 10-membered heterocyclyl-, (C6-C10)-aryl, or 5- to 10-membered heteroaryl, wherein each occurrence of R¹⁰ is independently substituted with 0-5 R′;
each R is independently selected from the group consisting of:
   H—,
   (C1-C12)-aliphatic-,
   (C3-C10)-cycloalkyl-,
   (C3-C10)-cycloalkenyl-,
   [(C3-C10)-cycloalkyl]—(C1-C12)-aliphatic-,
   [(C3-C10)-cycloalkenyl]—(C1-C12)-aliphatic-,
   [(C3-C10)-cycloalkyl]-O—(C1-C12)-aliphatic-,
   [(C3-C10)-cycloalkenyl]-O—(C1-C12)-aliphatic-,
   (C6-C10)-aryl-,
   (C6-C10)-aryl-(C1-C12)aliphatic-,
   (C6-C10)-aryl-O—(C1-C12)aliphatic-,
   (C6-C10)-aryl-N(R″)—(C1-C12)aliphatic-,
   3- to 10-membered heterocyclyl-,
   (3- to 10-membered heterocyclyl)-(C1-C12)aliphatic-,
   (3- to 10-membered heterocyclyl)-O—(C1-C12)aliphatic-,
   (3- to 10-membered heterocyclyl)-N(R″)—(C1-C12)aliphatic-,
   5- to 10-membered heteroaryl-,
   (5- to 10-membered heteroaryl)-(C1-C12)-aliphatic-,
   (5- to 10-membered heteroaryl)-O—(C1-C12)-aliphatic-; and
   (5- to 10-membered heteroaryl)-N(R″)—(C1-C12)-aliphatic-;
wherein said heterocyclyl has 1-4 heteroatoms independently selected from the group consisting of N, NH, O, S, SO, and SO₂, and said heteroaryl has 1-4 heteroatoms independently selected from the group consisting of N, NH, O, and S;
wherein each occurrence of R is independently substituted with 0-5 R′;
or when two R groups are bound to the same atom, the two R groups may be taken together with the atom to which they are bound to form a 3- to 10-membered aromatic or non-aromatic ring having 0-4 heteroatoms independently selected from the group consisting of N, NH, O, S, SO, and SO₂, wherein said ring is optionally substituted with 0-5 R′, and wherein said ring is optionally fused to a (C6-C10)aryl, 5- to 10-membered heteroaryl, (C3-C10)cycloalkyl, or a 3- to 10-membered heterocyclyl;
wherein each occurrence of R′ is independently selected from the group consisting of halogen, —R″, —OR″, oxo, —CH₂OR″, —CH₂NR″₂, —C(O)N(R″)₂, —C(O)OR″, —NO₂, —NCS, —CN, —CF₃, —OCF₃ and —N(R″)₂;
wherein each occurrence of R″ is independently selected from the group consisting of H, —(C1-C6)-alkyl, —(C1-C6)-aliphatic, (C3-C6)-cycloalkyl, 3- to 6-membered heterocyclyl, 5- to 10-membered heteroaryl-, (C6-C10)-aryl-, (5- to 10-membered heteroaryl)-(C1-C6)-alkyl-, (C6-C10)-aryl-(C1-C6)-alkyl-, (5- to 10-membered heteroaryl)-O—(C1-C6)-alkyl-, and (C6-C10)-aryl-O—(C1-C6)-alkyl-, wherein each occurrence of R″ is independently substituted with 0-3 substituents selected from the group consisting of: halogen, —R^{o}, —OR^{o}, oxo, —CH₂OR^{o}, —CH₂N(R^{o})₂, —C(O)N(R^{o})₂, —C(O)OR^{o}, —NO₂, —NCS, —CN, —CF₃, —OCF₃ and —N(R^{o})₂, wherein each occurrence of R^{o} is independently selected from the group consisting of: —(C1-C6)-aliphatic, (C3-C6)-cycloalkyl, 3- to 6-membered heterocyclyl 5- to 10-membered heteroaryl-, and (C6-C10)-aryl-;
ii) a compound of formula II:

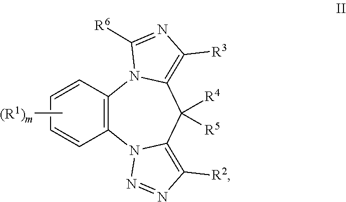

or a pharmaceutically acceptable salt, hydrate, solvate, polymorph, or isomer thereof, wherein:
m is 0-3;
each R¹ is independently selected from the group consisting of: halogen, —H, —(C1-C6)alkyl, —OH, —O((C1-C6)alkyl), —NO₂, —CN, —CF₃, —OCF₃, —OCHF₂, -OMe, —C≡C—R⁸, —CHF₂, —CH₂CF₃, —(C6-C10) aryl, —(C1-C6) alkyl-(C6-C10) aryl, -5-10 membered heteroaryl, —(C1-C6) alkyl-5-10 membered heteroaryl, and —(C3-C6) cycloalkyl; wherein R¹ is independently substituted with 0-5 R′;
R² is selected from the group consisting of:
   —H, halogen, —OH, —(C1-C6)aliphatic, —O((C1-C6)alkyl), —C(O)O((C1-C6)alkyl), —C(O)NR₂, —(CR₂)₁₋₃—OR, —(CR₂)₁₋₃—O(CR₂)₁₋₃—R, —OR⁹, —C(O)R⁸, —CH₂R⁸, —CH₃, —CH₂—OR⁸, (C6-C10)-aryl-,
   (C6-C10)-aryl-(C1-C12)aliphatic-,
      (C6-C10)-aryl-O—(C1-C12)aliphatic-,
   (C6-C10)-aryl-N(R″)—(C1-C12)aliphatic-,
   (5- to 10-membered heteroaryl)-(C1-C12)aliphatic-,
   (5- to 10-membered heteroaryl)-O—(C1-C12)aliphatic-,
   (5- to 10-membered heteroaryl)-N(R″)—(C1-C12)aliphatic-,
   (3- to 10-membered heterocyclyl)-(C1-C12)aliphatic-,
   (3- to 10-membered heterocyclyl)-O—(C1-C12)aliphatic-, and
   (3- to 10-membered heterocyclyl)-N(R″)—(C1-C12)aliphatic-,
   wherein R² is independently substituted with 0-5 R′;
R³ is selected from the group consisting of:
   —(C1-C6)alkyl, —(C2-C6)alkenyl, —C≡CH, —C≡CR⁹, —CN, halogen, —SO₂((C6-C10)-aryl), —SO₂((C1-C6)alkyl), —C(O)N((C1-C6)alkyl)₂, —C(O)NH₂, —C(O)O((C1-C6)alkyl), —C(O)((C1-C6)alkyl), —(C6-C10)aryl, 5- to 10-membered heteroaryl, 5- to 10-membered heterocyclyl, —(C1-C6)alkyl-C≡C—R¹⁰, —CH₂-Q-R¹⁰, —CH₂-Q-CH₂—R¹⁰

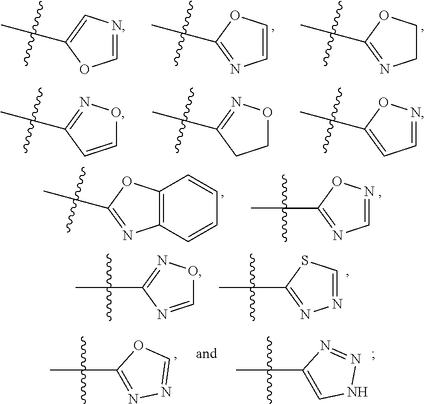

wherein each 5-member heterocycle or heteroaryl is substituted with 0-4 R₇;
wherein R³ is independently substituted with 0-5 R′;
R⁴ and R⁵ are each independently selected from the group consisting of —H, halogen, —(C1-C6)alkyl, or —(C1-C6) alkyl-(C6-C10) aryl; the (C6-C10)aryl being independently substituted with 0-5 halogen;
R⁶ is selected from the group consisting of —H and —(C1-C6)alkyl;
wherein R₇ is selected from the group consisting of —(C1-C6)alkyl, —(C3-C6)cycloalkyl, -5 to 10 membered heteroaryl, —(C6-C10) aryl, (C6-C10)aryl-(C1-C6)alkyl-, -5 to 10 membered heteroaryl-(C1-C6)alkyl, and -5-10 membered heteroaryl; wherein each R₇ is independently substituted with 0-5 R′;
wherein each R⁸ is independently selected from the group consisting of —H, —(C1-C6) alkyl, —(C3-C6) cycloalkyl, —(C1-C6)alkyl-(C3-C6)cycloalkyl, —(C1-C6)alkyl-(C6-C10)aryl, —(C6-C10) aryl, -5-10 membered heteroaryl, and —(C1-C6)alkyl-5-10 membered heteroaryl;
   wherein each R⁸ excluding —H and —(C1-C6) alkyl is independently substituted by 0-5 of -halogen, —(C1-C6) alkyl, —CF₃, —OCF₃, or O—(C1-C6) alkyl;
wherein R⁹ is selected from the group consisting of —H, —(C1-C6) alkyl, —(C6-C10)aryl, -5-10 membered heteroaryl, —(C1-C6)alkyl-(C6-C10) aryl, —(C1-C6) alkyl-5-10 membered heteroaryl, —(C3-C6) cycloalkyl, —(C1-C6) alkyl-(C3-C6) cycloalkyl, —C(O)—(C6-C10)aryl, 5-10 membered heterocycle,

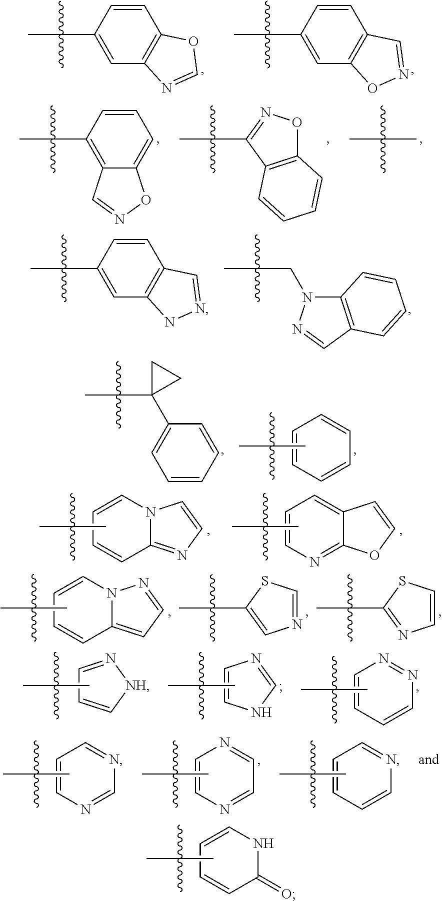

wherein each R⁹ is independently substituted with 0-5 R^{u};
wherein R¹⁰ is selected from the group consisting of —H, halogen, —(C1-C6) alkyl, —(C6-C10) aryl, -5-10 membered heteroaryl, —(C3-C6) cycloalkyl, —CH₂—(C3-C6) cycloalkyl, —CH₂—(C6-C10) aryl, and —CH₂-5-10-membered heteroaryl,
wherein each R¹⁰ is substituted with 0-5 R′;
wherein each occurrence of R¹¹ is independently selected from the group consisting of -halogen, —CN, SCH₃, —CF₃, —OH, —OCF₃, OCHF₂, —O(C1-C6)alkyl, —(C6-C10) aryl, —(C1-C6)alkyl, and -5 to 10 membered heteroaryl;
each R is independently selected from the group consisting of:
   H—,
   (C1-C12)-aliphatic-,
   (C3-C10)-cycloalkyl-,
   (C3-C10)-cycloalkenyl-,
   [(C3-C10)-cycloalkyl]—(C1-C12)-aliphatic-,
   [(C3-C10)-cycloalkenyl]—(C1-C12)-aliphatic-,
   [(C3-C10)-cycloalkyl]-O—(C1-C12)-aliphatic-,
   [(C3-C10)-cycloalkenyl]-O—(C1-C12)-aliphatic-,
   (C6-C10)-aryl-,
   (C6-C10)-aryl-(C1-C12)aliphatic-,
   (C6-C10)-aryl-O—(C1-C12)aliphatic-,
   (C6-C10)-aryl-N(R″)—(C1-C12)aliphatic-,
   3- to 10-membered heterocyclyl-,
   (3- to 10-membered heterocyclyl)-(C1-C12)aliphatic-,
   (3- to 10-membered heterocyclyl)-O—(C1-C12)aliphatic-,
   (3- to 10-membered heterocyclyl)-N(R″)—(C1-C12)aliphatic-,
   5- to 10-membered heteroaryl-,
   (5- to 10-membered heteroaryl)-(C1-C12)-aliphatic-,
   (5- to 10-membered heteroaryl)-O—(C1-C12)-aliphatic-; and
   (5- to 10-membered heteroaryl)-N(R″)—(C1-C12)-aliphatic-;
wherein said heterocyclyl has 1-4 heteroatoms independently selected from the group consisting of N, NH, O, S, SO, and SO₂, and said heteroaryl has 1-4 heteroatoms independently selected from the group consisting of N, NH, O, and S;
wherein each occurrence of R is independently substituted with 0-5 R′;
or when two R groups bound to the same atom, the two R groups may be taken together with the atom to which they are bound to form a 3- to 10-membered aromatic or non-aromatic ring having 0-4 heteroatoms independently selected from the group consisting of N, NH, O, S, SO, and SO₂, wherein said ring is optionally substituted with 0-5 R′, and wherein said ring is optionally fused to a (C6-C10)aryl, 5- to 10-membered heteroaryl, (C3-C10)cycloalkyl, or a 3- to 10-membered heterocyclyl;
wherein each occurrence of R′ is independently selected from the group consisting of halogen, —R″, —OR″, oxo, —CH₂OR″, —CH₂NR″₂, —C(O)N(R″)₂, —C(O)OR″, —NO₂, —NCS, —CN, —CF₃, —OCF₃ and —N(R″)₂;
wherein each occurrence of R″ is independently selected from the group consisting of H, —(C1-C6)-alkyl, —(C1-C6)-aliphatic, (C3-C6)-cycloalkyl, 3- to 6-membered heterocyclyl, 5- to 10-membered heteroaryl-, (C6-C10)-aryl-, (5- to 10-membered heteroaryl)-(C1-C6)-alkyl-, (C6-C10)-aryl-(C1-C6)-alkyl-, (5- to 10-membered heteroaryl)-O—(C1-C6)-alkyl-, (C6-C10)-aryl-O—(C1-C6)-alkyl-, and (C6-C10)-aryl-O—(C1-C6)-alkyl-,
wherein each occurrence of R″ is independently substituted with 0-5 substituents selected from the group consisting of: halogen, —R^{o}, —OR^{o}, oxo, —CH₂OR^{o}, —CH₂N(R^{o})₂, —C(O)N(R^{o})₂, —C(O)OR^{o}, —NO₂, —NCS, —CN, —CF₃, —OCF₃ and —N(R^{o})₂, wherein each occurrence of R^{o} is independently selected from the group consisting of: —(C1-C6)-aliphatic, (C3-C6)-cycloalkyl, 3- to 6-membered heterocyclyl, 5- to 10-membered heteroaryl-, and (C6-C10)-aryl; and
iii) a compound of formula IV:

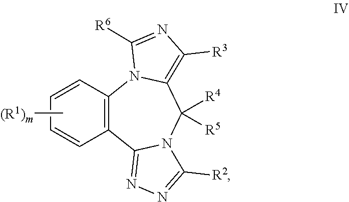

or a pharmaceutically acceptable salt, hydrate, solvate, polymorph, or isomer thereof, wherein:
m is 0-3;
each R¹ is independently selected from the group consisting of: halogen, —H, —(C1-C6)alkyl, —C≡C—R⁹, —OH, —O((C1-C6)alkyl), —NO₂, —CN, —CF₃, —OCF₃, —CHF₂, —CH₂CF₃, —(C6-C10) aryl, —(C1-C6) alkyl-(C6-C10) aryl, -5-10 membered heteroaryl, —(C1-C6) alkyl-5-10 membered heteroaryl, and —(C3-C6) cycloalkyl;
wherein R¹ is independently substituted with 0-5 R′;
R² is selected from the group consisting of —OR⁸, —SR⁸, —(CH₂)ₙOR⁸, —(CH₂)ₙO(CH₂)ₙR⁸, —(CH₂)_{P}R⁸ and —(CH₂)ₙN(R″)R¹⁰, wherein n is an integer selected from 0-4; p is an integer selected from 2-4;
wherein R² is independently substituted with 0-5 R′;
each R³ is independently selected from the group consisting of: —H, —CN, halogen, —(C1-C6)aliphatic, —CH═CR⁹, —C≡CR⁹, —SO₂((C1-C6)alkyl), —C(O)N((C1-C6)alkyl)₂), —C(O)NH((C1-C6)aliphatic), (C6-C10)-aryl-(C1-C12)aliphatic-, —C(O)((C1-C6)alkyl), —C(O)O((C1-C6)alkyl), 5- or 6-membered heterocyclyl, 5- or 6-membered heteroaryl, —CH₂—O—R¹⁰, —CH₂—O—CH₂—R¹⁰

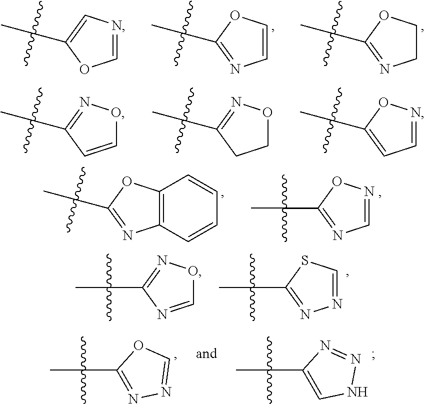

wherein each 5-10-membered heterocycle or heteroaryl are substituted with 0-3 R₇; wherein R³ is independently substituted with 0-5 R′;
R⁴ and R⁵ are each independently selected from the group consisting of —H, halogen and —(C1-C6)alkyl;
R⁶ is selected from the group consisting of —H and —(C1-C6)alkyl;
R₇ is selected from the group consisting of —(C1-C6)alkyl, —(C3-C6)cycloalkyl, -5 to 10 membered heteroaryl, —(C6-C10) aryl, —(C6-C10)aryl-(C1-C6)alkyl, and -5 to 10 membered heteroaryl-(C1-C6)alkyl, and -5-10 membered heteroaryl;
wherein each R₇ is independently substituted with 0-5 R′;
R⁸ is independently selected from the group consisting of —H, —(C1-C6)alkyl, —(C3-C10)-cycloalkyl, (C6-C10)-aryl, or 5- to 10-membered heteroaryl, 5-10 membered heteroaryl-(C1-C6) alkyl-, —(C1-C6) alkyl-(C6-C10) aryl, and —(C1-C6) alkyl-(C3-C6) cycloalkyl;
wherein each occurrence of R⁸ is independently substituted with 0-5 R′;
wherein R⁹ is selected from the group consisting of —H, —(C1-C6) alkyl, —(C3-C6) cycloalkyl, —(C1-C6) alkyl-(C3-C6) cycloalkyl, —(C1-C6)alkyl-(C6-C10) aryl, —(C6-C10)aryl, -5-10 membered heteroaryl, —(C1-C6)alkyl-5-10 membered heteroaryl, 5-10 membered heterocycle, —C(O)—(C6-C10) aryl,

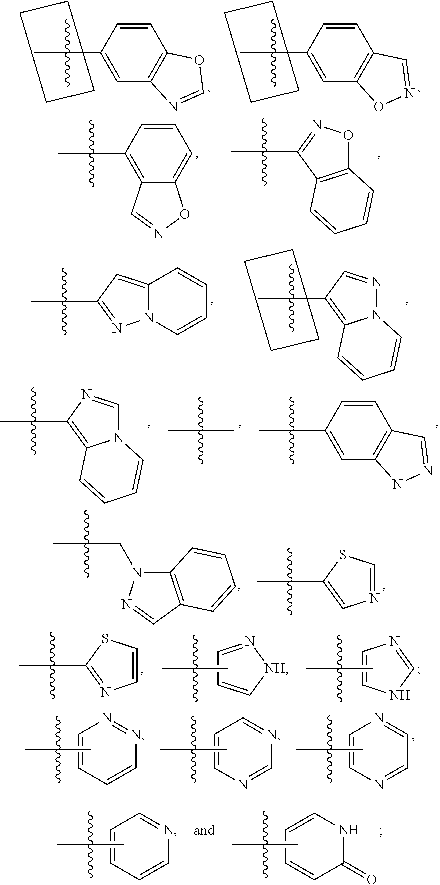

wherein each wherein each R⁹ is independently substituted with 0-5 R′;
R¹⁰ is selected from the group consisting of —H, —(C1-C6) alkyl, —(C3-C10)-cycloalkyl, 3- to 10-membered heterocyclyl-, (C6-C10)-aryl, 5- to 10-membered heteroaryl, —CH₂—(C3-C6) cycloalkyl, —CH₂—(C6-C10) aryl, and —CH₂-5-10-membered heteroaryl, wherein each occurrence of R¹⁰ is independently substituted with 0-5 R′;
wherein each occurrence of R¹¹ is independently selected from the group consisting of -halogen, —CF₃, —OCF₃, OCF₂H, —O—(C1-C6)alkyl, —(C6-C10) aryl, —(C1-C6)alkyl, —O—CH₂—(C3-C6)cycloalkyl, and -5 to 10 membered heteroaryl;
wherein each occurrence of R′ is independently selected from the group consisting of halogen, —R″, —OR″, oxo, —CH₂OR″, —CH₂NR″₂, —C(O)N(R″)₂, —C(O)OR″, —NO₂, —NCS, —CN, —CF₃, —OCF₃ and —N(R″)₂;
wherein each occurrence of R″ is independently selected from the group consisting of H, —(C1-C6)-aliphatic, —(C1-C6)-alkyl, (C3-C6)-cycloalkyl, 3- to 6-membered heterocyclyl, 5- to 10-membered heteroaryl-, (C6-C10)-aryl-, (5- to 10-membered heteroaryl)-(C1-C6)-alkyl-, (C6-C10)-aryl-(C1-C6)-alkyl-, (5- to 10-membered heteroaryl)-O—(C1-C6)-alkyl-, and (C6-C10)-aryl-O—(C1-C6)-alkyl-;
wherein each occurrence of R″ is independently substituted with 0-5 R^{t} independently selected from the group consisting of: halogen, —R^{o}, —OR^{o}, oxo, —CH₂OR^{o}, —CH₂N(R^{o})₂, —C(O)N(R^{o})₂, —C(O)OR^{o}, —NO₂, —NCS, —CN, —CF₃, —OCF₃ and —N(R^{o})₂, wherein each occurrence of R^{o} is independently selected from: —(C1-C6)-aliphatic, (C3-C6)-cycloalkyl, 3- to 6-membered heterocyclyl, 5- to 10-membered heteroaryl-, and (C6-C10)-aryl-.

In some embodiments, the specific compounds that may be useful for the present disclosure are compounds 1-471 as disclosed in published patent application WO2018130868 and WO2018130869. In other embodiments, the specific compounds are compounds 180-730 as disclosed in published patent application WO2019246300. In other embodiments, the specific compounds are compounds 731-740 as disclosed in published patent application WO2021127543. Each of these published documents is incorporated by reference herein in its entirety and in particular in the context of the recited compounds.

In some embodiments, the SV2A inhibitor, or the pharmaceutically acceptable salt, hydrate, solvate, polymorph, or isomer thereof, is levetiracetam, seletracetam, brivaracetam, or a pharmaceutically acceptable salt, hydrate, solvate, polymorph, or isomer of any of the foregoing. In some embodiments, the SV2A inhibitor, or the pharmaceutically acceptable salt, hydrate, solvate, polymorph, or isomer thereof, is levetiracetam, or a pharmaceutically acceptable salt, hydrate, solvate, polymorph, or isomer thereof. In some embodiments, the SV2A inhibitor, or the pharmaceutically acceptable salt, hydrate, solvate, polymorph, or isomer thereof, is seletracetam, or a pharmaceutically acceptable salt, hydrate, solvate, polymorph, or isomer thereof. In some embodiments, the SV2A inhibitor, or the pharmaceutically acceptable salt, hydrate, solvate, polymorph, or isomer thereof, is brivaracetam, or a pharmaceutically acceptable salt, hydrate, solvate, polymorph, or isomer thereof.

In some embodiments, the GABA_{A} α5 receptor agonist, or the pharmaceutically acceptable salt, hydrate, solvate, polymorph, or isomer thereof, is a compound of Formula I, or a pharmaceutically acceptable salt, hydrate, solvate, polymorph, or isomer thereof. In some embodiments, the GABA_{A} α5 receptor agonist, or the pharmaceutically acceptable salt, hydrate, solvate, polymorph, or isomer thereof, is a compound of Formula II, or a pharmaceutically acceptable salt, hydrate, solvate, polymorph, or isomer thereof. In some embodiments, the GABA_{A} α5 receptor agonist, or the pharmaceutically acceptable salt, hydrate, solvate, polymorph, or isomer thereof, is a compound of Formula IV, or a pharmaceutically acceptable salt, hydrate, solvate, polymorph, or isomer thereof. In some embodiments, the GABA_{A} α5 receptor agonist, or the pharmaceutically acceptable salt, hydrate, solvate, polymorph, or isomer thereof, is a compound of Formula I, or a pharmaceutically acceptable salt, hydrate, solvate, polymorph, or isomer thereof, selected from the group consisting of:

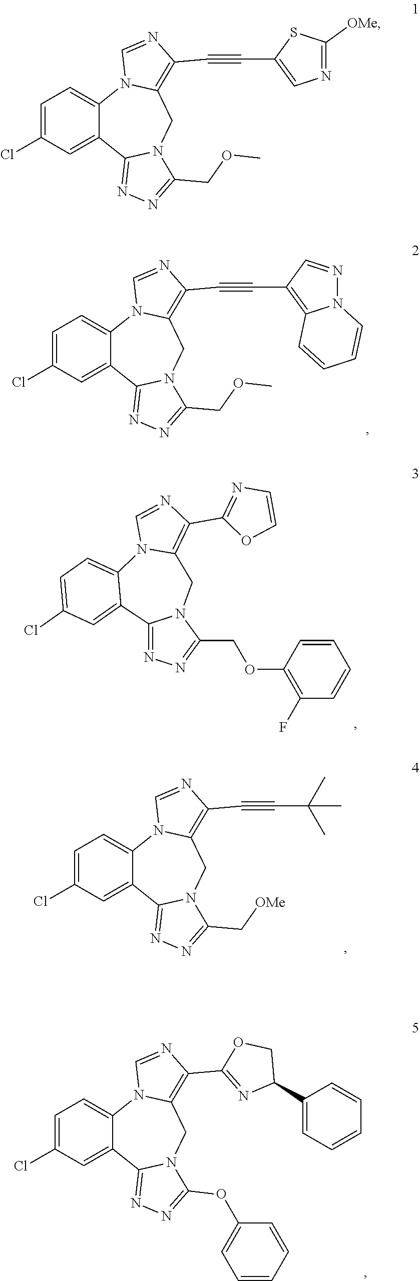 [SMILES: COCc1nnc2n1Cc1c(C#Cc3cnc(OC)s3)ncn1-c1ccc(Cl)cc1-2 ; COCc1nnc2n1Cc1c(C#Cc3cnn4ccccc34)ncn1-c1ccc(Cl)cc1-2 ; Fc1ccccc1OCc1nnc2n1Cc1c(-c3ncco3)ncn1-c1ccc(Cl)cc1-2 ; COCc1nnc2n1Cc1c(C#CC(C)(C)C)ncn1-c1ccc(Cl)cc1-2 ; Clc1ccc2c(c1)-c1nnc(Oc3ccccc3)n1Cc1c(C3=N[C@H](c4ccccc4)CO3)ncn1-2]

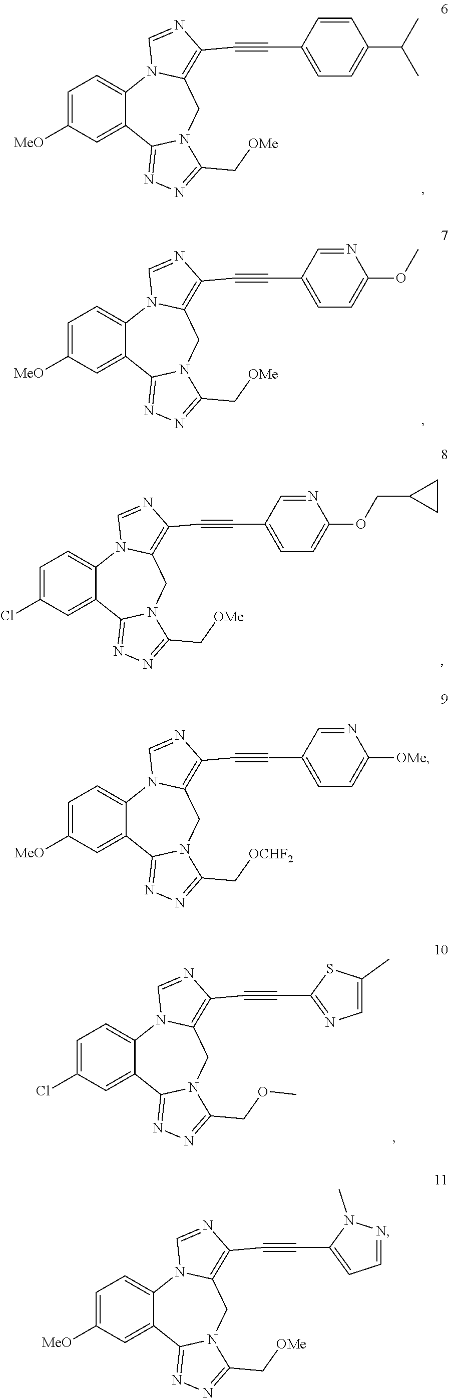 [SMILES: COCc1nnc2n1Cc1c(C#Cc3ccc(C(C)C)cc3)ncn1-c1ccc(OC)cc1-2 ; COCc1nnc2n1Cc1c(C#Cc3ccc(OC)nc3)ncn1-c1ccc(OC)cc1-2 ; COCc1nnc2n1Cc1c(C#Cc3ccc(OCC4CC4)nc3)ncn1-c1ccc(Cl)cc1-2 ; COc1ccc2c(c1)-c1nnc(COC(F)F)n1Cc1c(C#Cc3ccc(OC)nc3)ncn1-2 ; COCc1nnc2n1Cc1c(C#Cc3ncc(C)s3)ncn1-c1ccc(Cl)cc1-2 ; COCc1nnc2n1Cc1c(C#Cc3ccnn3C)ncn1-c1ccc(OC)cc1-2]

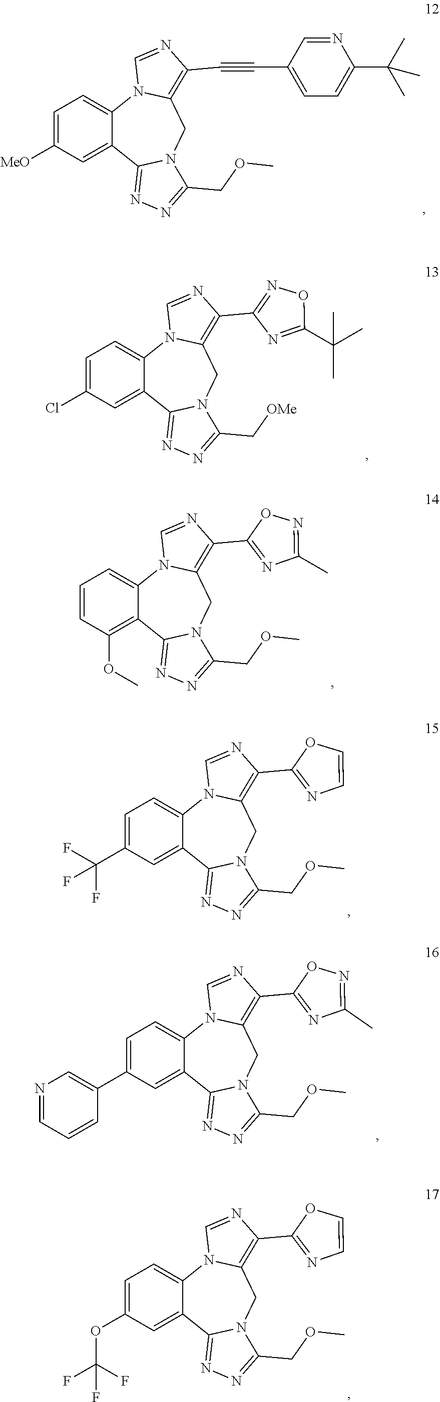 [SMILES: COCc1nnc2n1Cc1c(C#Cc3ccc(C(C)(C)C)nc3)ncn1-c1ccc(OC)cc1-2 ; COCc1nnc2n1Cc1c(-c3noc(C(C)(C)C)n3)ncn1-c1ccc(Cl)cc1-2 ; COCc1nnc2n1Cc1c(-c3nc(C)no3)ncn1-c1cccc(OC)c1-2 ; COCc1nnc2n1Cc1c(-c3ncco3)ncn1-c1ccc(C(F)(F)F)cc1-2 ; COCc1nnc2n1Cc1c(-c3nc(C)no3)ncn1-c1ccc(-c3cccnc3)cc1-2 ; COCc1nnc2n1Cc1c(-c3ncco3)ncn1-c1ccc(OC(F)(F)F)cc1-2]

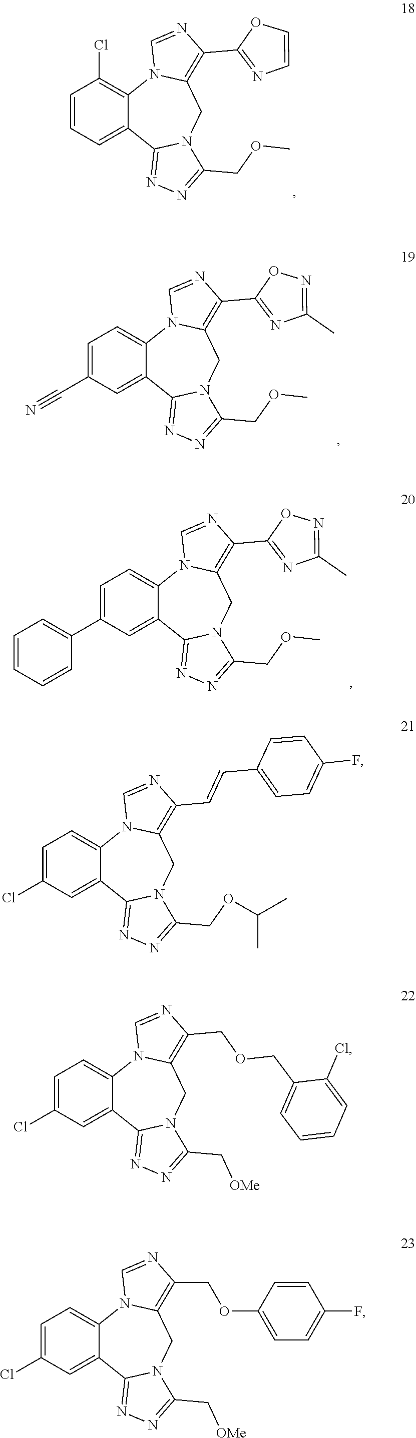 [SMILES: COCc1nnc2n1Cc1c(-c3ncco3)ncn1-c1c(Cl)cccc1-2 ; COCc1nnc2n1Cc1c(-c3nc(C)no3)ncn1-c1ccc(C#N)cc1-2 ; COCc1nnc2n1Cc1c(-c3nc(C)no3)ncn1-c1ccc(-c3ccccc3)cc1-2 ; CC(C)OCc1nnc2n1Cc1c(/C=C/c3ccc(F)cc3)ncn1-c1ccc(Cl)cc1-2 ; COCc1nnc2n1Cc1c(COCc3ccccc3Cl)ncn1-c1ccc(Cl)cc1-2 ; COCc1nnc2n1Cc1c(COc3ccc(F)cc3)ncn1-c1ccc(Cl)cc1-2]

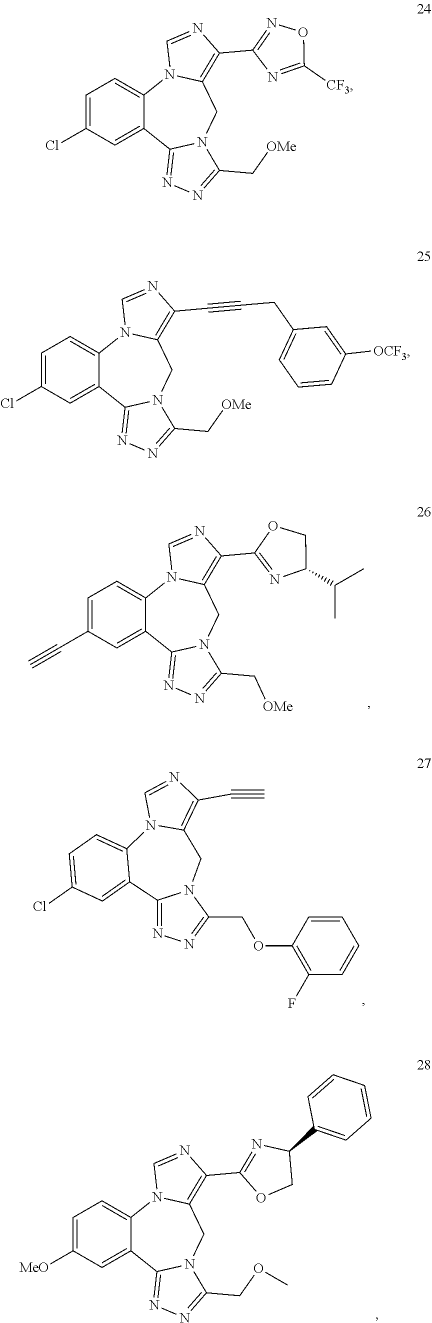 [SMILES: COCc1nnc2n1Cc1c(-c3noc(C(F)(F)F)n3)ncn1-c1ccc(Cl)cc1-2 ; COCc1nnc2n1Cc1c(C#CCc3cccc(OC(F)(F)F)c3)ncn1-c1ccc(Cl)cc1-2 ; C#Cc1ccc2c(c1)-c1nnc(COC)n1Cc1c(C3=N[C@@H](C(C)C)CO3)ncn1-2 ; C#Cc1ncn2c1Cn1c(COc3ccccc3F)nnc1-c1cc(Cl)ccc1-2 ; COCc1nnc2n1Cc1c(C3=N[C@@H](c4ccccc4)CO3)ncn1-c1ccc(OC)cc1-2]

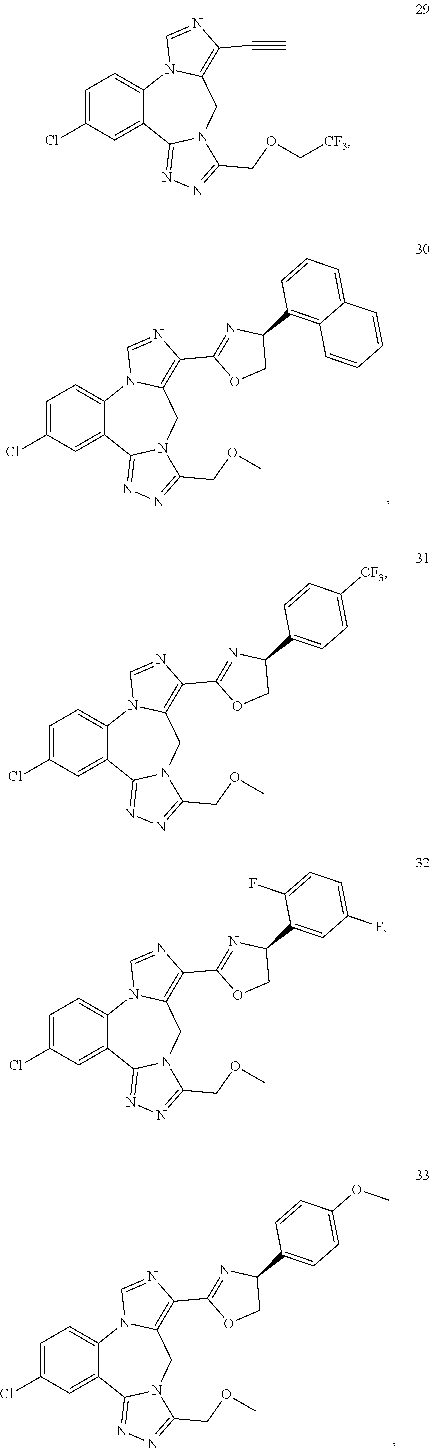 [SMILES: C#Cc1ncn2c1Cn1c(COCC(F)(F)F)nnc1-c1cc(Cl)ccc1-2 ; COCc1nnc2n1Cc1c(C3=N[C@@H](c4cccc5ccccc45)CO3)ncn1-c1ccc(Cl)cc1-2 ; COCc1nnc2n1Cc1c(C3=N[C@@H](c4ccc(C(F)(F)F)cc4)CO3)ncn1-c1ccc(Cl)cc1-2 ; COCc1nnc2n1Cc1c(C3=N[C@@H](c4cc(F)ccc4F)CO3)ncn1-c1ccc(Cl)cc1-2 ; COCc1nnc2n1Cc1c(C3=N[C@@H](c4ccc(OC)cc4)CO3)ncn1-c1ccc(Cl)cc1-2]

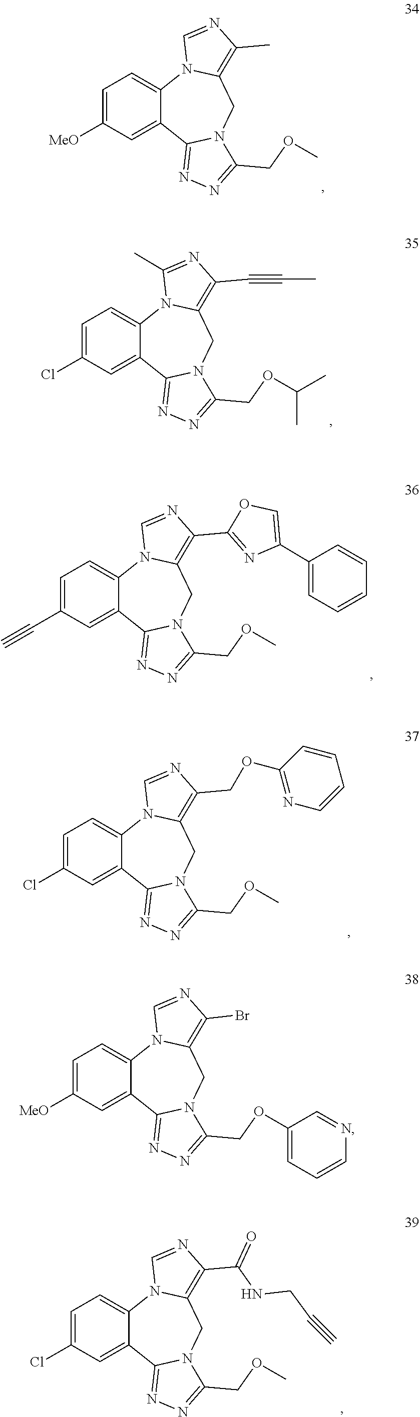

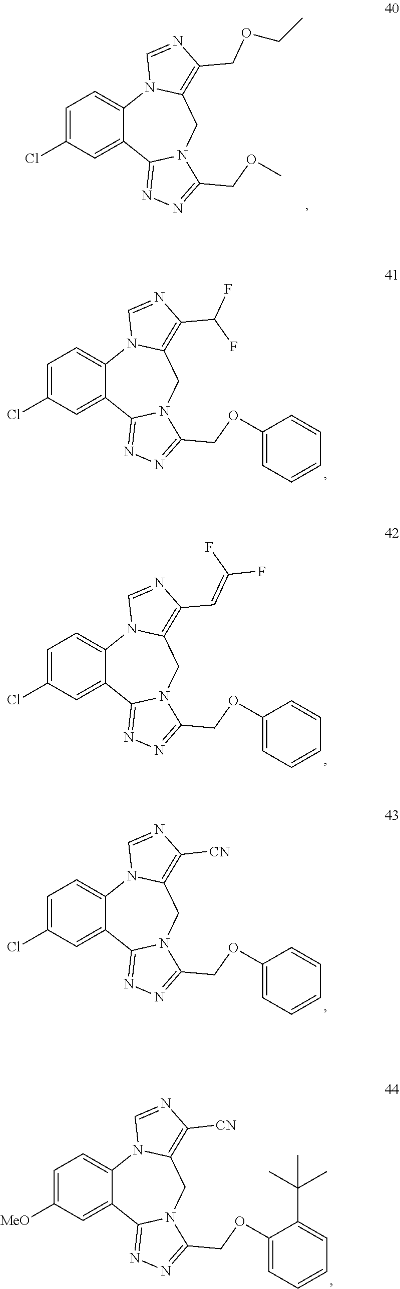 [SMILES: CCOCc1ncn2c1Cn1c(COC)nnc1-c1cc(Cl)ccc1-2 ; FC(F)c1ncn2c1Cn1c(COc3ccccc3)nnc1-c1cc(Cl)ccc1-2 ; FC(F)=Cc1ncn2c1Cn1c(COc3ccccc3)nnc1-c1cc(Cl)ccc1-2 ; N#Cc1ncn2c1Cn1c(COc3ccccc3)nnc1-c1cc(Cl)ccc1-2 ; COc1ccc2c(c1)-c1nnc(COc3ccccc3C(C)(C)C)n1Cc1c(C#N)ncn1-2]

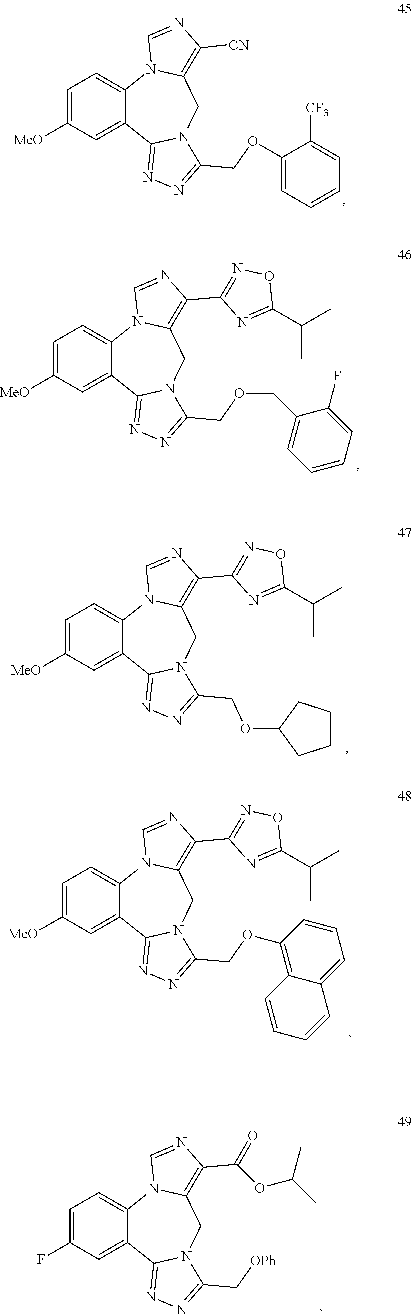 [SMILES: COc1ccc2c(c1)-c1nnc(COc3ccccc3C(F)(F)F)n1Cc1c(C#N)ncn1-2 ; COc1ccc2c(c1)-c1nnc(COCc3ccccc3F)n1Cc1c(-c3noc(C(C)C)n3)ncn1-2 ; COc1ccc2c(c1)-c1nnc(COC3CCCC3)n1Cc1c(-c3noc(C(C)C)n3)ncn1-2 ; COc1ccc2c(c1)-c1nnc(COc3cccc4ccccc34)n1Cc1c(-c3noc(C(C)C)n3)ncn1-2 ; CC(C)OC(=O)c1ncn2c1Cn1c(COc3ccccc3)nnc1-c1cc(F)ccc1-2]

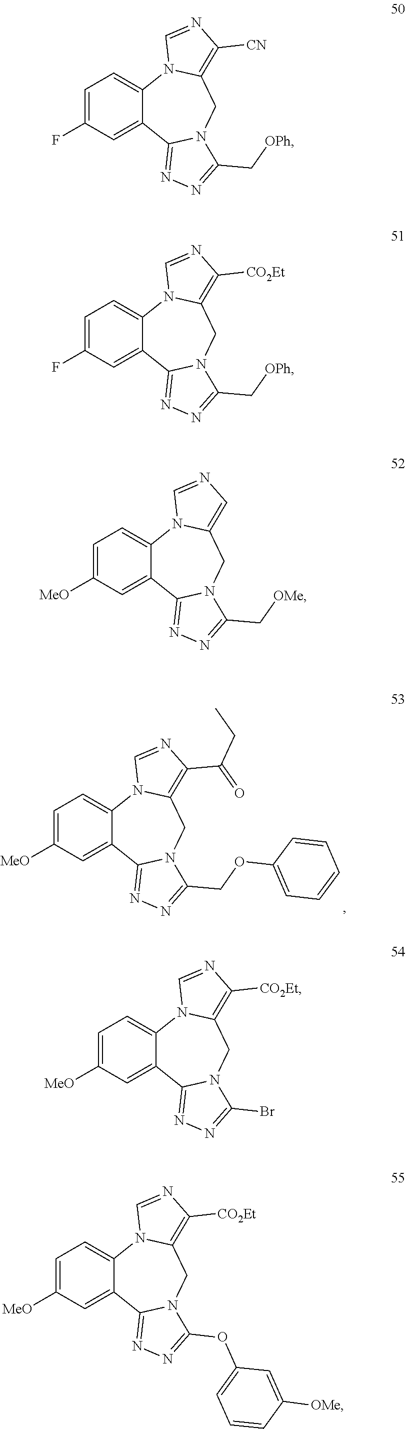 [SMILES: N#Cc1ncn2c1Cn1c(COc3ccccc3)nnc1-c1cc(F)ccc1-2 ; CCOC(=O)c1ncn2c1Cn1c(COc3ccccc3)nnc1-c1cc(F)ccc1-2 ; COCc1nnc2n1Cc1cncn1-c1ccc(OC)cc1-2 ; CCC(=O)c1ncn2c1Cn1c(COc3ccccc3)nnc1-c1cc(OC)ccc1-2 ; CCOC(=O)c1ncn2c1Cn1c(Br)nnc1-c1cc(OC)ccc1-2 ; CCOC(=O)c1ncn2c1Cn1c(Oc3cccc(OC)c3)nnc1-c1cc(OC)ccc1-2]

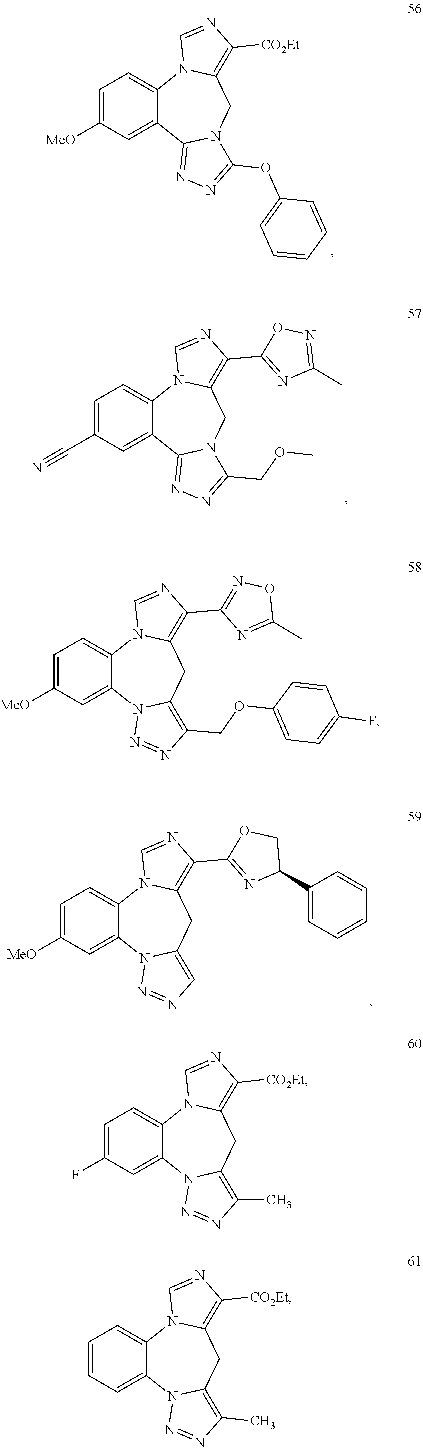 [SMILES: CCOC(=O)c1ncn2c1Cn1c(Oc3ccccc3)nnc1-c1cc(OC)ccc1-2 ; COCc1nnc2n1Cc1c(-c3nc(C)no3)ncn1-c1ccc(C#N)cc1-2 ; COc1ccc2c(c1)-n1nnc(COc3ccc(F)cc3)c1Cc1c(-c3noc(C)n3)ncn1-2 ; COc1ccc2c(c1)-n1nncc1Cc1c(C3=N[C@H](c4ccccc4)CO3)ncn1-2 ; CCOC(=O)c1ncn2c1Cc1c(C)nnn1-c1cc(F)ccc1-2 ; CCOC(=O)c1ncn2c1Cc1c(C)nnn1-c1ccccc1-2]

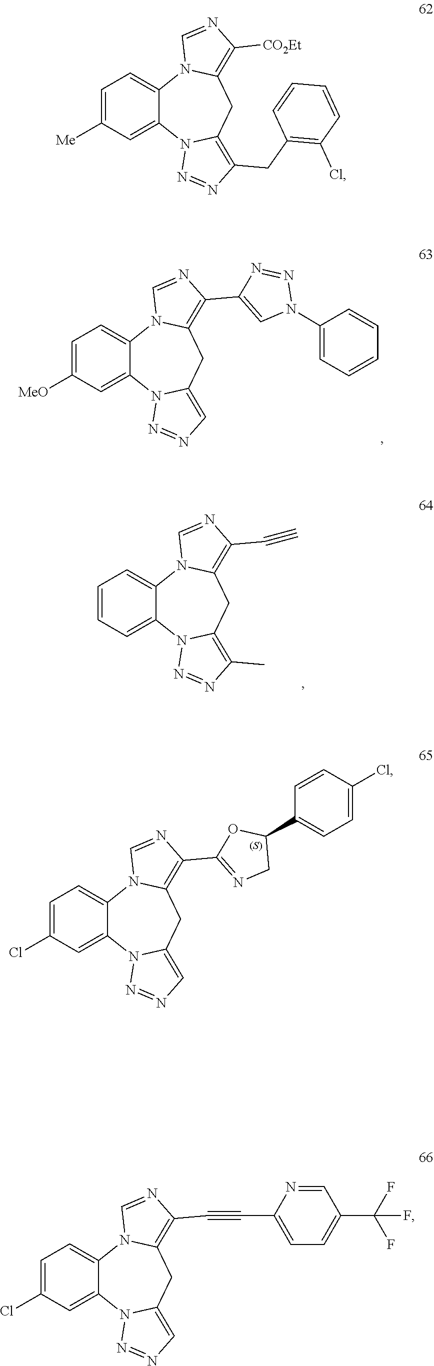

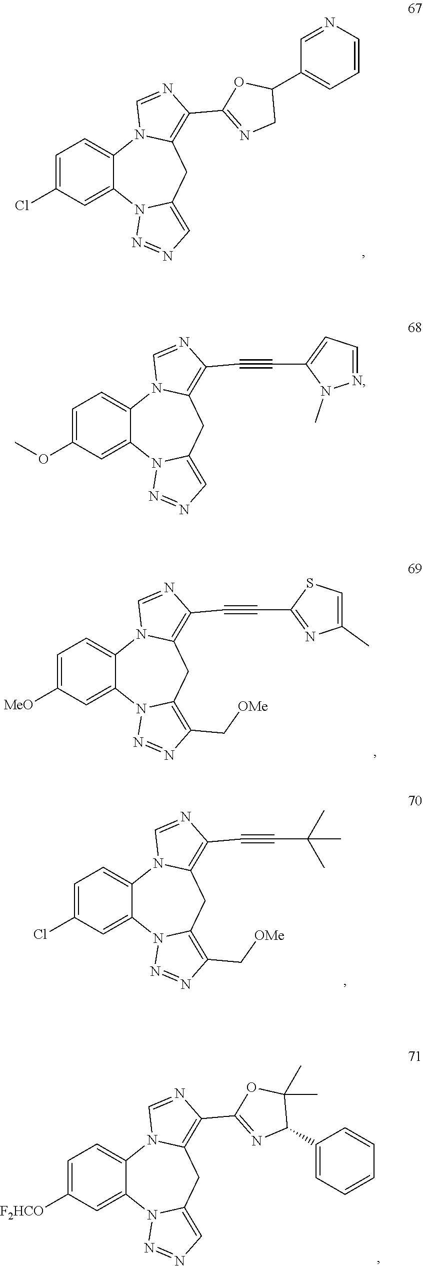 [SMILES: Clc1ccc2c(c1)-n1nncc1Cc1c(C3=NCC(c4cccnc4)O3)ncn1-2 ; COc1ccc2c(c1)-n1nncc1Cc1c(C#Cc3ccnn3C)ncn1-2 ; COCc1nnn2c1Cc1c(C#Cc3nc(C)cs3)ncn1-c1ccc(OC)cc1-2 ; COCc1nnn2c1Cc1c(C#CC(C)(C)C)ncn1-c1ccc(Cl)cc1-2 ; CC1(C)OC(c2ncn3c2Cc2cnnn2-c2cc(OC(F)F)ccc2-3)=N[C@H]1c1ccccc1]

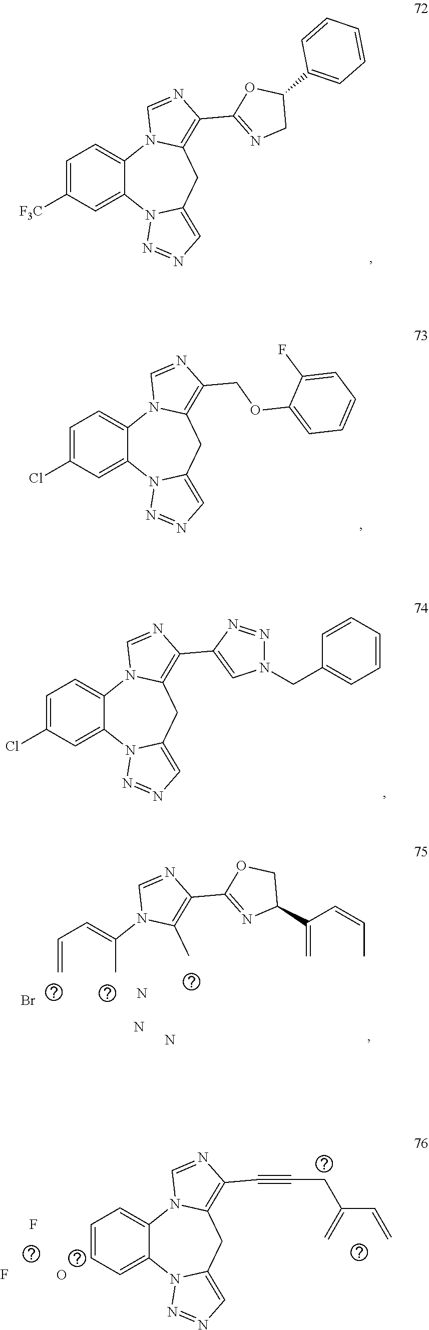

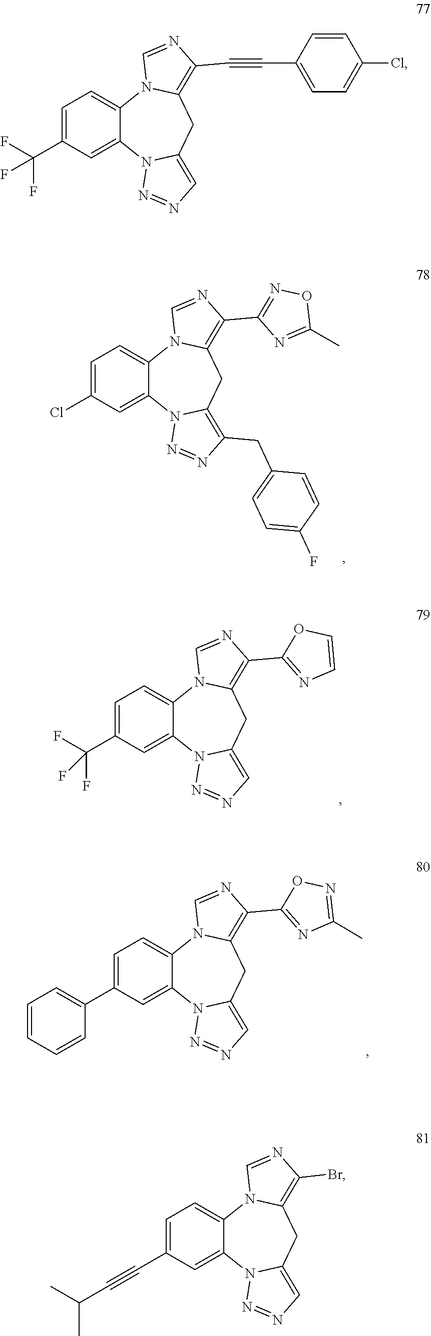

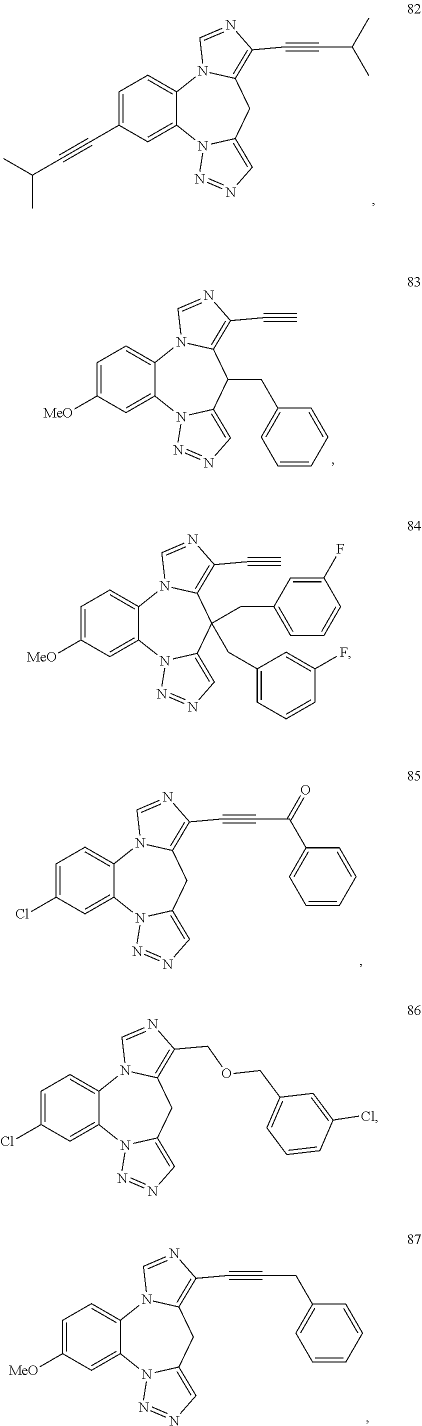 [SMILES: CC(C)C#Cc1ccc2c(c1)-n1nncc1Cc1c(C#CC(C)C)ncn1-2 ; C#Cc1ncn2c1C(Cc1ccccc1)c1cnnn1-c1cc(OC)ccc1-2 ; C#Cc1ncn2c1C(Cc1cccc(F)c1)(Cc1cccc(F)c1)c1cnnn1-c1cc(OC)ccc1-2 ; O=C(C#Cc1ncn2c1Cc1cnnn1-c1cc(Cl)ccc1-2)c1ccccc1 ; Clc1cccc(COCc2ncn3c2Cc2cnnn2-c2cc(Cl)ccc2-3)c1 ; COc1ccc2c(c1)-n1nncc1Cc1c(C#CCc3ccccc3)ncn1-2]

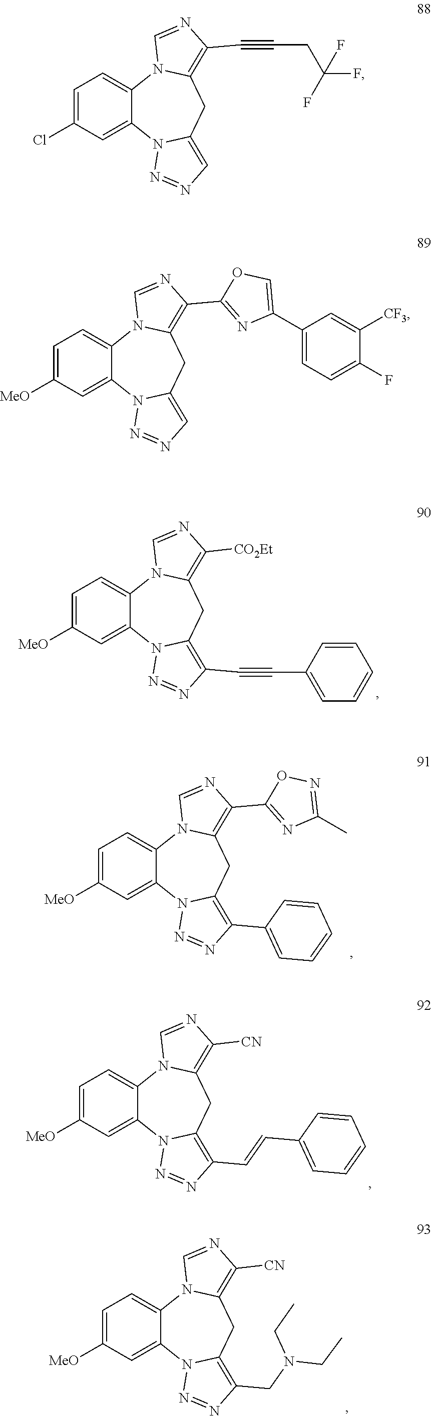 [SMILES: FC(F)(F)CC#Cc1ncn2c1Cc1cnnn1-c1cc(Cl)ccc1-2 ; COc1ccc2c(c1)-n1nncc1Cc1c(-c3nc(-c4ccc(F)c(C(F)(F)F)c4)co3)ncn1-2 ; CCOC(=O)c1ncn2c1Cc1c(C#Cc3ccccc3)nnn1-c1cc(OC)ccc1-2 ; COc1ccc2c(c1)-n1nnc(-c3ccccc3)c1Cc1c(-c3nc(C)no3)ncn1-2 ; COc1ccc2c(c1)-n1nnc(/C=C/c3ccccc3)c1Cc1c(C#N)ncn1-2 ; CCN(CC)Cc1nnn2c1Cc1c(C#N)ncn1-c1ccc(OC)cc1-2]

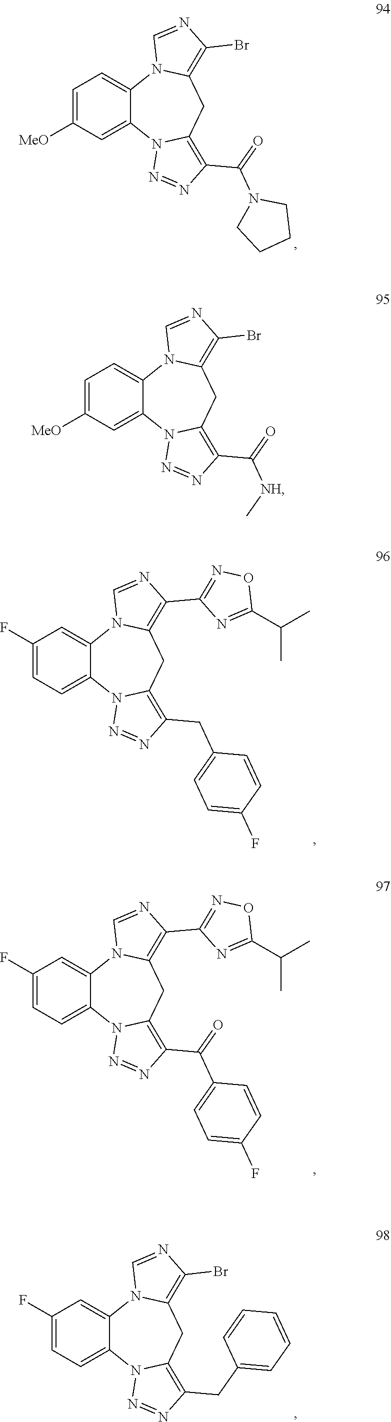 [SMILES: COc1ccc2c(c1)-n1nnc(C(=O)N3CCCC3)c1Cc1c(Br)ncn1-2 ; CNC(=O)c1nnn2c1Cc1c(Br)ncn1-c1ccc(OC)cc1-2 ; CC(C)c1nc(-c2ncn3c2Cc2c(Cc4ccc(F)cc4)nnn2-c2ccc(F)cc2-3)no1 ; CC(C)c1nc(-c2ncn3c2Cc2c(C(=O)c4ccc(F)cc4)nnn2-c2ccc(F)cc2-3)no1 ; Fc1ccc2c(c1)-n1cnc(Br)c1Cc1c(Cc3ccccc3)nnn1-2]

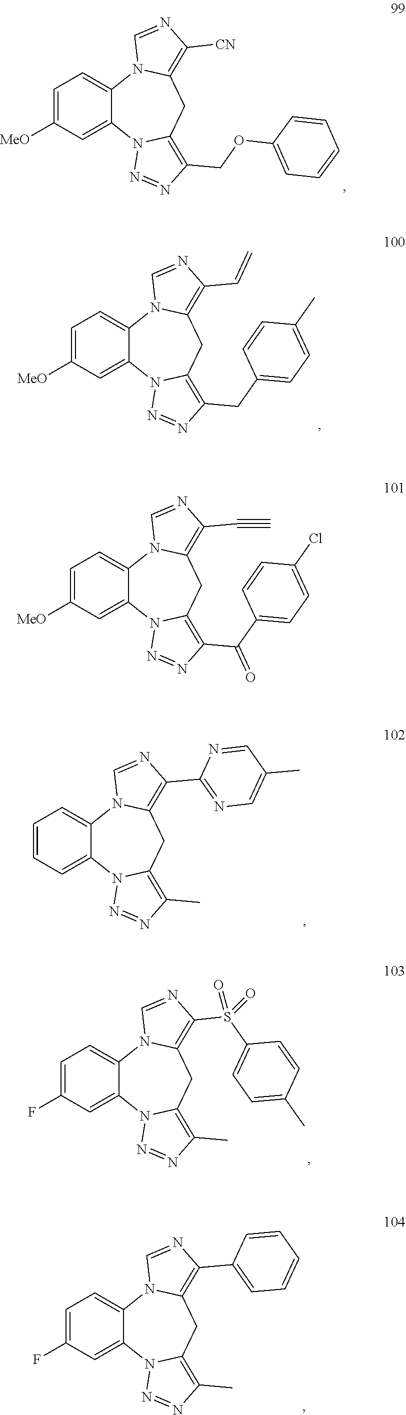 [SMILES: COc1ccc2c(c1)-n1nnc(COc3ccccc3)c1Cc1c(C#N)ncn1-2 ; C=Cc1ncn2c1Cc1c(Cc3ccc(C)cc3)nnn1-c1cc(OC)ccc1-2 ; C#Cc1ncn2c1Cc1c(C(=O)c3ccc(Cl)cc3)nnn1-c1cc(OC)ccc1-2 ; Cc1cnc(-c2ncn3c2Cc2c(C)nnn2-c2ccccc2-3)nc1 ; Cc1ccc(S(=O)(=O)c2ncn3c2Cc2c(C)nnn2-c2cc(F)ccc2-3)cc1 ; Cc1nnn2c1Cc1c(-c3ccccc3)ncn1-c1ccc(F)cc1-2]

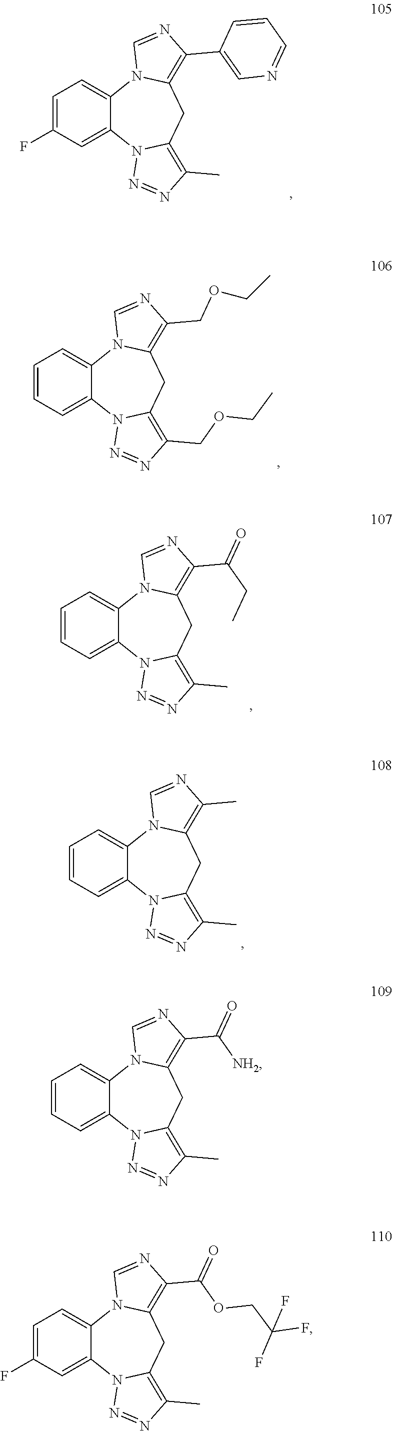 [SMILES: Cc1nnn2c1Cc1c(-c3cccnc3)ncn1-c1ccc(F)cc1-2 ; CCOCc1ncn2c1Cc1c(COCC)nnn1-c1ccccc1-2 ; CCC(=O)c1ncn2c1Cc1c(C)nnn1-c1ccccc1-2 ; Cc1ncn2c1Cc1c(C)nnn1-c1ccccc1-2 ; Cc1nnn2c1Cc1c(C(N)=O)ncn1-c1ccccc1-2 ; Cc1nnn2c1Cc1c(C(=O)OCC(F)(F)F)ncn1-c1ccc(F)cc1-2]

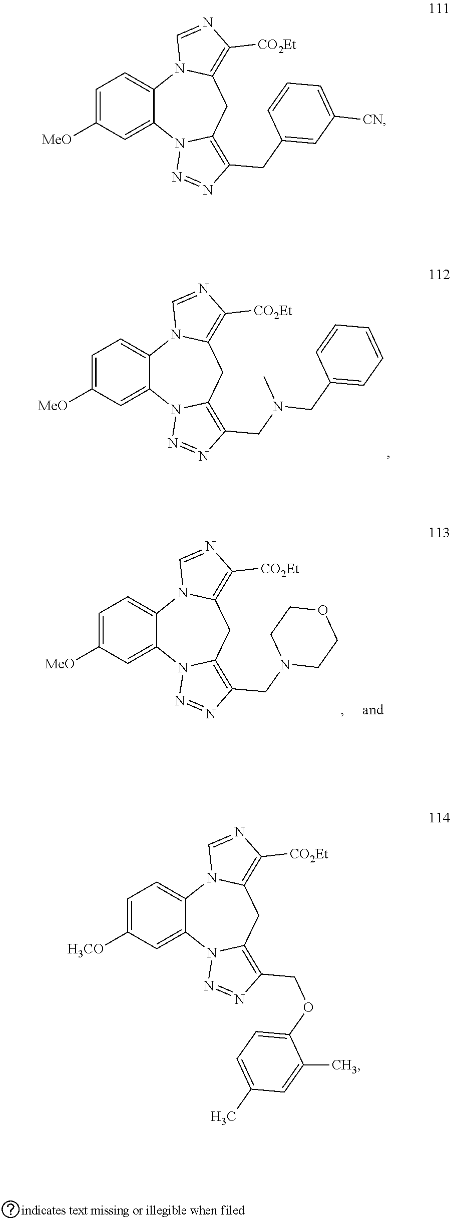 [SMILES: CCOC(=O)c1ncn2c1Cc1c(Cc3cccc(C#N)c3)nnn1-c1cc(OC)ccc1-2 ; CCOC(=O)c1ncn2c1Cc1c(CN(C)Cc3ccccc3)nnn1-c1cc(OC)ccc1-2 ; CCOC(=O)c1ncn2c1Cc1c(CN3CCOCC3)nnn1-c1cc(OC)ccc1-2 ; CCOC(=O)c1ncn2c1Cc1c(COc3ccc(C)cc3C)nnn1-c1cc(OC)ccc1-2]

or a pharmaceutically acceptable salt, hydrate, solvate, polymorph, or isomer of any of the foregoing.

In some embodiments, the compound of Formula I, or the pharmaceutically acceptable salt, hydrate, solvate, polymorph, or isomer thereof, is Compound 1, or a pharmaceutically acceptable salt, hydrate, solvate, polymorph, or isomer thereof. In some embodiments, the compound of Formula I, or the pharmaceutically acceptable salt, hydrate, solvate, polymorph, or isomer thereof, is a crystalline form of Compound 1, wherein the polymorph crystalline form is Form A, Form B, Form C, Form E, or Form F. In some embodiments, the pharmaceutical composition comprises one or more crystalline forms of Compound 1, wherein the one or more crystalline forms are selected from the group consisting of Form A, Form B, Form C, Form E, and Form F. In some embodiments of the pharmaceutical compositions disclosed herein, the GABA_{A} α5 receptor agonist, or the pharmaceutically acceptable salt, hydrate, solvate, isomer, or polymorph thereof, is present in an amount between 5 mg and 1000 mg. In some embodiments of the pharmaceutical compositions disclosed herein, the SV2A inhibitor, or the pharmaceutically acceptable salt, hydrate, solvate, isomer, or polymorph thereof, is present in an amount between 0.07 mg to 350 mg. In some embodiments of the pharmaceutical compositions disclosed herein, the pharmaceutical composition further comprises a pharmaceutically acceptable carrier. In some embodiments of the pharmaceutical compositions disclosed herein, the pharmaceutical composition is formulated as a tablet, capsule, pill, lozenge, powder, granule, solution, or suspension. In some embodiments of the pharmaceutical compositions disclosed herein, the SV2A inhibitor, or the pharmaceutically acceptable salt, hydrate, solvate, isomer, or polymorph thereof, is in an extended release form, a non-extended release form, or an immediate release form. In some embodiments of the pharmaceutical compositions disclosed herein, the SV2A inhibitor, or the pharmaceutically acceptable salt, hydrate, solvate, isomer, or polymorph thereof, is in an extended release form. In some embodiments of the pharmaceutical compositions disclosed herein, the GABA_{A} α5 receptor agonist, or the pharmaceutically acceptable salt, hydrate, solvate, isomer, or polymorph thereof, is in an extended release form, a non-extended release form, or an immediate release form. In some embodiments of the pharmaceutical compositions disclosed herein, the GABA_{A} α5 receptor agonist, or the pharmaceutically acceptable salt, hydrate, solvate, isomer, or polymorph thereof, is in an extended release form. In some embodiments of the pharmaceutical compositions disclosed herein, the GABA_{A} α5 receptor agonist, or the pharmaceutically acceptable salt, hydrate, solvate, isomer, or polymorph thereof, is in a non-extended release form.

Another aspect of the disclosure relates to a combination comprising: Component A: a SV2A inhibitor, or a pharmaceutically acceptable salt, hydrate, solvate, isomer, or polymorph thereof; or a first pharmaceutical composition comprising a SV2A inhibitor, or a pharmaceutically acceptable salt, hydrate, solvate, polymorph or isomer thereof; and Component B: a GABA_{A} α5 receptor agonist, or a pharmaceutically acceptable salt, hydrate, solvate, isomer, or polymorph thereof; or a second pharmaceutical composition comprising a GABA_{A} α5 receptor agonist, or a pharmaceutically acceptable salt, hydrate, solvate, polymorph or isomer thereof.

In some embodiments, the GABA_{A} α5 receptor agonist, or the pharmaceutically acceptable salt, hydrate, solvate, polymorph, or isomer thereof, is selected from the group consisting of: a compound of Formula I, or a pharmaceutically acceptable salt, hydrate, solvate, polymorph, or isomer thereof; a compound of Formula II, or a pharmaceutically acceptable salt, hydrate, solvate, polymorph, or isomer thereof; and a compound of Formula IV, or a pharmaceutically acceptable salt, hydrate, solvate, polymorph, or isomer thereof. In some embodiments, the GABA_{A} α5 receptor agonist, or the pharmaceutically acceptable salt, hydrate, solvate, polymorph, or isomer thereof, is a compound of Formula I, or a pharmaceutically acceptable salt, hydrate, solvate, polymorph, or isomer thereof. In some embodiments, the GABA_{A} α5 receptor agonist, or the pharmaceutically acceptable salt, hydrate, solvate, polymorph, or isomer thereof, is a compound of Formula II, or a pharmaceutically acceptable salt, hydrate, solvate, polymorph, or isomer thereof. In some embodiments, the GABA_{A} α5 receptor agonist, or the pharmaceutically acceptable salt, hydrate, solvate, polymorph, or isomer thereof, is a compound of Formula IV, or a pharmaceutically acceptable salt, hydrate, solvate, polymorph, or isomer thereof. In some embodiments, the compound of Formula I, or the pharmaceutically acceptable salt, hydrate, solvate, polymorph, or isomer thereof, is selected from the group consisting of Compounds 1-114, or a pharmaceutically acceptable salt, hydrate, solvate, polymorph, or isomer of any of the foregoing. In some embodiments, the compound of Formula I, or the pharmaceutically acceptable salt, hydrate, solvate, polymorph, or isomer thereof, is Compound 1, or a pharmaceutically acceptable salt, hydrate, solvate, polymorph, or isomer thereof. In some embodiments, the compound of Formula I, or the pharmaceutically acceptable salt, hydrate, solvate, polymorph, or isomer thereof, is a crystalline form of Compound 1, wherein the polymorph crystalline form is Form A, Form B, Form C, Form E, or Form F. In some embodiments, the combination comprises one or more crystalline forms of Compound 1, wherein the one or more crystalline forms are selected from the group consisting of Form A, Form B, Form C, Form E, and Form F.

In some embodiments, the SV2A inhibitor, or the pharmaceutically acceptable salt, hydrate, solvate, polymorph, or isomer thereof, is levetiracetam, seletracetam, brivaracetam, or a pharmaceutically acceptable salt, hydrate, solvate, polymorph, or isomer of any of the foregoing. In some embodiments, the SV2A inhibitor, or the pharmaceutically acceptable salt, hydrate, solvate, polymorph, or isomer thereof, is levetiracetam, or a pharmaceutically acceptable salt, hydrate, solvate, polymorph, or isomer thereof. In some embodiments, the SV2A inhibitor, or the pharmaceutically acceptable salt, hydrate, solvate, polymorph, or isomer thereof, is seletracetam, or a pharmaceutically acceptable salt, hydrate, solvate, polymorph, or isomer thereof. In some embodiments, the SV2A inhibitor, or the pharmaceutically acceptable salt, hydrate, solvate, polymorph, or isomer thereof, is brivaracetam, or a pharmaceutically acceptable salt, hydrate, solvate, polymorph, or isomer thereof.

In some embodiments of the combinations disclosed herein, the GABA_{A} α5 receptor agonist, or the pharmaceutically acceptable salt, hydrate, solvate, isomer, or polymorph thereof, is present in an amount between 5 mg and 1000 mg. In some embodiments of the combinations disclosed herein, the SV2A inhibitor, or the pharmaceutically acceptable salt, hydrate, solvate, isomer, or polymorph thereof, is present in an amount between 0.07 mg to 350 mg. In some embodiments of the combinations disclosed herein, the GABA_{A} α5 receptor agonist, or a pharmaceutically acceptable salt, hydrate, solvate, isomer, or polymorph thereof, and the SV2A inhibitor, or a pharmaceutically acceptable salt, hydrate, solvate, isomer, or polymorph thereof, are formulated as a tablet, capsule, pill, lozenge, powder, granule, solution, or suspension. In some embodiments of the combinations disclosed herein, the GABA_{A} α5 receptor agonist, or a pharmaceutically acceptable salt, hydrate, solvate, isomer, or polymorph thereof, and the SV2A inhibitor, or a pharmaceutically acceptable salt, hydrate, solvate, isomer, or polymorph thereof, are formulated in a single pharmaceutical composition or separately. In some embodiments, the combination comprises Component A: a first pharmaceutical composition comprising the SV2A inhibitor, or the pharmaceutically acceptable salt, hydrate, solvate, polymorph or isomer thereof; and Component B: a second pharmaceutical composition comprising a GABA_{A} α5 receptor agonist selected from the group consisting of a compound of Formula I, a compound of Formula II, and a compound of Formula IV, or a pharmaceutically acceptable salt, hydrate, solvate, polymorph or isomer of any of the foregoing. In some embodiments of the combinations disclosed herein, the first pharmaceutical composition and the second pharmaceutical composition comprise a pharmaceutically acceptable carrier. In some embodiments of the combinations disclosed herein, the first pharmaceutical composition and the second pharmaceutical composition are formulated as a tablet, capsule, pill, lozenge, powder, granule, solution, or suspension. In some embodiments of the combinations disclosed herein, the first pharmaceutical composition and the second pharmaceutical composition are formulated in a single pharmaceutical composition or separately. In some embodiments of the combinations disclosed herein, the SV2A inhibitor, or the pharmaceutically acceptable salt, hydrate, solvate, isomer, or polymorph thereof, is in an extended release form, a non-extended release form, or an immediate release form. In some embodiments of the combinations disclosed herein, the SV2A inhibitor, or the pharmaceutically acceptable salt, hydrate, solvate, isomer, or polymorph thereof, is in an extended release form. In some embodiments of the combinations disclosed herein, the GABA_{A} α5 receptor agonist, or the pharmaceutically acceptable salt, hydrate, solvate, isomer, or polymorph thereof, is in an extended release form, a non-extended release form, or an immediate release form. In some embodiments of the combinations disclosed herein, the GABA_{A} α5 receptor agonist, or the pharmaceutically acceptable salt, hydrate, solvate, isomer, or polymorph thereof, is in an extended release form. In some embodiments of the combinations disclosed herein, the GABA_{A} α5 receptor agonist, or the pharmaceutically acceptable salt, hydrate, solvate, isomer, or polymorph thereof, is in a non-extended release form.

One aspect of the disclosure relates to a method of treating cognitive impairment associated with a central nervous system (CNS) disorder in a subject in need thereof or at risk thereof, the method comprising administering to the subject a pharmaceutical composition or combination of the disclosure.

Another aspect of the disclosure relates to a method of treating cognitive impairment associated with a brain cancer in a subject in need thereof, the method comprising administering to the subject a pharmaceutical composition or combination of the disclosure.

One aspect of the disclosure relates to a method of treating a brain cancer in a subject in need thereof, the method comprising administering to the subject a pharmaceutical composition or combination of the disclosure.

Another aspect of the disclosure relates to a method of treating Parkinson's disease psychosis in a subject in need thereof, the method comprising administering to the subject a pharmaceutical composition or combination of the disclosure.

One aspect of the disclosure relates to use of a pharmaceutical composition or combination of the disclosure for treating cognitive impairment associated with a central nervous system (CNS) disorder in a subject in need thereof or at risk thereof.

Another aspect of the disclosure relates to use of a pharmaceutical composition or combination of the disclosure for treating cognitive impairment associated with a brain cancer in a subject in need thereof.

One aspect of the disclosure relates to use of a pharmaceutical composition or combination of the disclosure for treating a brain cancer in a subject in need thereof.

Another aspect of the disclosure relates to use of a pharmaceutical composition or combination of the disclosure for treating Parkinson's disease psychosis in a subject in need thereof.

One aspect of the disclosure relates to use of a pharmaceutical composition or combination of the disclosure in the manufacture of a medicament.

Another aspect of the disclosure relates to use of a pharmaceutical composition or combination of the disclosure in the manufacture of a medicament for treating cognitive impairment associated with a central nervous system (CNS) disorder in a subject in need thereof or at risk thereof.

One aspect of the disclosure relates to use of a pharmaceutical composition or combination of the disclosure in the manufacture of a medicament for treating cognitive impairment associated with a brain cancer in a subject in need thereof.

Another aspect of the disclosure relates to use of a pharmaceutical composition or combination of the disclosure in the manufacture of a medicament for treating a brain cancer in a subject in need thereof.

One aspect of the disclosure relates to use of a pharmaceutical composition or combination of the disclosure in the manufacture of a medicament for treating Parkinson's disease psychosis in a subject in need thereof.

Another aspect of the disclosure relates to a pharmaceutical composition or combination of the disclosure for use in treating cognitive impairment associated with a central nervous system (CNS) disorder in a subject in need thereof or at risk thereof.

One aspect of the disclosure relates to a pharmaceutical composition or combination of the disclosure for use in treating cognitive impairment associated with a brain cancer in a subject in need thereof.

Another aspect of the disclosure relates to a pharmaceutical composition or combination of the disclosure for use in treating a brain cancer in a subject in need thereof.

One aspect of the disclosure relates to a pharmaceutical composition or combination of the disclosure for use in treating Parkinson's disease psychosis in a subject in need thereof.

Another aspect of the disclosure relates to a method of increasing the therapeutic index of an SV2A inhibitor, or a pharmaceutically acceptable salt, hydrate, solvate, isomer, or polymorph thereof, in a method of treating cognitive impairment associated with a central nervous system (CNS) disorder in a subject in need thereof or at risk thereof, the method comprising administering to the subject a GABA_{A} α5 agonist, or a pharmaceutically acceptable salt, hydrate, solvate, polymorph, or prodrug thereof containing pharmaceutical composition or combination of the disclosure. In some embodiments, the therapeutic index of the SV2A inhibitor, or the pharmaceutically acceptable salt, hydrate, solvate, polymorph, or isomer thereof, is greater than the therapeutic index of the SV2A inhibitor, or pharmaceutically acceptable salt, hydrate, solvate, polymorph, or isomer thereof, when administered in the absence of the GABA_{A} α5 agonist, or the pharmaceutically acceptable salt, hydrate, solvate, polymorph, or prodrug thereof, by at least about 1.5×, or about 2.0×, or about 2.5×, or about 3.0×, or about 3.5×, or about 4.0×, or about 4.5×, or about 5.0×, or about 5.5×, or about 6.0×, or about 6.5×, or about 7.0×, or about 7.5×, or about 8.0×, or about 8.5×, or about 9.0×, or about 9.5×, or about 10×, or greater than about 10×.

One aspect of the disclosure relates to a method of increasing the therapeutic index of a GABA_{A} α5 receptor agonist, or a pharmaceutically acceptable salt, hydrate, solvate, isomer, or polymorph thereof, in a method of treating cognitive impairment associated with a central nervous system (CNS) disorder in a subject in need thereof or at risk thereof, the method comprising administering to the subject an SV2A inhibitor, or a pharmaceutically acceptable salt, hydrate, solvate, polymorph, or isomer thereof containing pharmaceutical composition or combination of the disclosure. In some embodiments, the therapeutic index of the GABA_{A} α5 receptor agonist, or the pharmaceutically acceptable salt, hydrate, solvate, polymorph, or isomer thereof, is greater than the therapeutic index of the GABA_{A} α5 receptor agonist, or the pharmaceutically acceptable salt, hydrate, solvate, polymorph, or isomer thereof, when administered in the absence of the SV2A inhibitor, or the pharmaceutically acceptable salt, hydrate, solvate, polymorph, or isomer thereof, by at least about 1.5×, or about 2.0×, or about 2.5×, or about 3.0×, or about 3.5×, or about 4.0×, or about 4.5×, or about 5.0×, or about 5.5×, or about 6.0×, or about 6.5×, or about 7.0×, or about 7.5×, or about 8.0×, or about 8.5×, or about 9.0×, or about 9.5×, or about 10×, or greater than about 10×.

In some embodiments of the methods, uses, combinations for use, or compositions for use, the CNS disorder is age-related cognitive impairment. In some embodiments of the methods, uses, combinations for use, or compositions for use, the CNS disorder is mild cognitive impairment (MCI). In some embodiments of the methods, uses, combinations for use, or compositions for use, the CNS disorder is amnestic mild cognitive impairment (aMCI). In some embodiments of the methods, uses, combinations for use, or compositions for use, the CNS disorder is dementia. In some embodiments of the methods, uses, combinations for use, or compositions for use, the CNS disorder is Alzheimer's disease. In some embodiments of the methods, uses, combinations for use, or compositions for use, the CNS disorder is schizophrenia, amyotrophic lateral sclerosis (ALS), posttraumatic stress disorder (PTSD), mental retardation, Parkinson's disease (PD), autism, compulsive behavior, substance addiction, bipolar disorder, or cancer-therapy-related cognitive impairment. In some embodiments of the methods, uses, combinations for use, or compositions for use, the pharmaceutical composition or combination is administered subcutaneously, intravenously, orally, sublingually, buccally, transdermally, arterially, intradermally, intramuscularly, intraperitoneally, ocularly, intranasally, intraspinally or intracerebrally. In some embodiments of the methods, uses, combinations for use, or compositions for use, the pharmaceutical composition or combination is administered orally. In some embodiments of the methods, uses, combinations for use, or compositions for use, the subject is a human. In some embodiments of the methods, uses, combinations for use, or compositions for use, the pharmaceutical composition or combination is administered once daily. In some embodiments of the methods, uses, combinations for use, or compositions for use, the pharmaceutical composition or combination is administered twice daily. In some embodiments of the methods, uses, or combinations for use, Component A and Component B of the combination are administered simultaneously. In some embodiments of the methods, uses, or combinations for use, Component A and Component B of the combination are administered sequentially.

In some embodiments of the methods, uses, combinations for use, or compositions for use, the treatment has a longer therapeutic effect in the subject than is attained by administering the GABA_{A} α5 receptor agonist, or the pharmaceutically acceptable salt, hydrate, solvate, isomer, or polymorph thereof, in the absence of the SV2A inhibitor, or the pharmaceutically acceptable salt, hydrate, solvate, polymorph, or isomer thereof, by at least 1.5×, or at least 2.0×, or at least 2.5×, or at least 3.0×, or at least 3.5×, or at least 4.0×, or at least 4.5×, or at least 5.0×, or at least 5.5×, or at least 6.0×, or at least 6.5×, or at least 7.0×, or at least 7.5×, or at least 8.0×, or at least 8.5×, or at least 9.0×, or at least 9.5×, or at least 10×, or greater than 10×. In some embodiments of the methods, uses, combinations for use, or compositions for use, the treatment has a longer therapeutic effect in the subject than is attained by administering the SV2A inhibitor, or the pharmaceutically acceptable salt, hydrate, solvate, polymorph, or isomer thereof, in the absence of the GABA_{A} α5 receptor agonist, or the pharmaceutically acceptable salt, hydrate, solvate, isomer, or polymorph thereof, by at least 1.5×, or at least 2.0×, or at least 2.5×, or at least 3.0×, or at least 3.5×, or at least 4.0×, or at least 4.5×, or at least 5.0×, or at least 5.5×, or at least 6.0×, or at least 6.5×, or at least 7.0×, or at least 7.5×, or at least 8.0×, or at least 8.5×, or at least 9.0×, or at least 9.5×, or at least 10×, or greater than 10×.

### BRIEF DESCRIPTION OF THE DRAWINGS

FIG. 1 depicts the effective plasma level ranges based on aged-impaired rat studies and phase II study in aMCI patients. The acceptable range goal is established based on the effective plasma level range in aged-impaired rats and in aMCI patients, i.e., between 1.9 and 4.4 μg/ml. The preferred range goal is established based on the effective plasma level range in aMCI patients, i.e., between 2.9 and 4.4 μg/ml.

FIG. 2 depicts the effects of administering levetiracetam on the spatial memory retention of six aged-impaired rats (AI) in a Morris Water Maze (MWM) test. Three treatment conditions were employed: vehicle control, levetiracetam (5 mg/kg/day) and levetiracetam (10 mg/kg/day). The AI rats were trained for two consecutive days, with a one-time treatment prior to the training trials per day. 24 hours later, the AI rats were tested. The time the AI rats, 24 hours after treatment with the different conditions and two days of training, spent swimming in the target quadrant or the target annulus in a memory retention trial is used as a measure of spatial memory retention. The target quadrant refers to the quadrant of the maze (which is a circular pool) where the escape platform is placed during the training trials. The target annulus refers to the exact location of the escape platform during the training trials.

FIG. 3 depicts the effects of administering levetiracetam on the spatial memory retention of ten aged-impaired rats (AI) in an eight-arm Radial Arm Maze (RAM) test. Six treatment conditions were employed: vehicle control, levetiracetam (1.25 mg/kg), levetiracetam (2.5 mg/kg), levetiracetam (5 mg/kg), levetiracetam (10 mg/kg) and levetiracetam (20 mg/kg). In the RAM task used, there was a one-hour delay between presentation of a subset of arms (5 arms available and 3 arms blocked) and completion of the eight-arm win-shift task (eight arms available). Rats were pre-treated 30-40 minutes before daily trials with a one-time drug/control treatment. The number of errors made by the rats after the delay was used as a measure of spatial memory retention. Errors were defined as instances when rats entered an arm from which food had already been retrieved in the pre-delay component of the trial or when rats re-visited an arm in the post-delay session that had already been visited. Paired t-tests were used to compare the number of errors between different doses of levetiracetam and vehicle control.

FIG. 4 depicts the experimental design of the human trials for levetiracetam treatment.

FIG. 5A depicts the average activity in the left CA3 of aMCI subjects with placebo treatment and age-matched control subjects with placebo treatment during the presentation of lure stimuli that the subject correctly identified as “similar.”

FIG. 5B depicts the average activity in the left CA3 of aMCI subjects with placebo treatment or levetiracetam treatment (125 mg twice a day for two weeks) during the presentation of lure stimuli that the subject correctly identified as “similar.”

FIG. 5C is a table of the data represented in FIGS. 5A and 5B.

FIG. 6A depicts the average activity in the left entorhinal cortex of age-matched control subjects with placebo treatment and aMCI subjects with placebo treatment during the presentation of lure stimuli that the subject correctly identified as “similar.”

FIG. 6B depicts the average activity in the left entorhinal cortex of the same aMCI subjects with placebo treatment or levetiracetam treatment (125 mg twice a day for two weeks) during the presentation of lure stimuli that the subject correctly identified as “similar.”

FIG. 6C is a table of the data represented in FIGS. 6A and 6B.

FIG. 7A depicts an example of the sequence of images shown to subjects in the explicit 3-alternative forced choice task described in Example 2.

FIG. 7B shows sample pairs of similar (“lure”) images.

FIG. 8 shows the difference between the aMCI (placebo) subjects and age-matched control (placebo) subjects in their performance of the explicit 3-alternative forced choice task described in Example 2. Each bar represents the proportion of the subject responses (old, similar, or new) when presented with a lure image.

FIG. 9 shows the difference between the same aMCI subjects with placebo treatment or with levetiracetam treatment (125 mg twice a day for two weeks) in their performance of the explicit 3-alternative forced choice task described in Example 1. Each bar represents the proportion of the subjects' responses (old, similar, or new) when presented with a lure image.

FIG. 10 is a table of the data represented in FIGS. 8 and 9.

FIG. 11A shows the difference between the age-matched control (placebo) subjects and the aMCI subjects treated with placebo or levetiracetam (125 mg twice a day for two weeks) in their performance of the Buschke Selective reminding Test-Delayed Recall.

FIG. 11B is a table of the data represented in FIG. 11A.

FIG. 12A shows the difference between the control (placebo) subjects and the aMCI subjects treated with placebo or with levetiracetam (125 mg twice a day for two weeks) in their performance of the Benton Visual Retention Test.

FIG. 12B is a table of the data represented in FIG. 12A.

FIG. 13A shows the difference between the control (placebo) subjects and the aMCI subjects treated with placebo or with levetiracetam (125 mg twice a day for two weeks) in their performance of the Verbal Paired Associates Testμ-Recognition.

FIG. 13B is a table of the data represented in FIG. 13A.

FIG. 14A shows the difference between the control (placebo) subjects and the aMCI subjects treated with placebo or with levetiracetam (125 mg twice a day for two weeks) in their performance of the Verbal Paired Associates Test—Delayed Recall.

FIG. 14B is a table of the data represented in FIG. 14A.

FIG. 15A is a table showing the subject selection process for the human levetiracetam trial described in Example 1.

FIG. 15B is a table showing the characteristics of the subjects selected for the human levetiracetam trial described in Example 1.

FIG. 16 depicts the effects of administering brivaracetam on the memory performance of nine aged-impaired rats in an eight-arm Radial Arm Maze task. Doses of brivaracetam administered to the AI rats include 0.0625 mg/kg, 0.125 mg/kg, 0.25 mg/kg, 0.5 mg/kg, 1 mg/kg, 2 mg/kg and 4 mg/kg. Means and SEMs for the number of errors are shown as the y-axis.

FIG. 17 depicts the effects of administering seletracetam on the memory performance of nine aged-impaired rats in an eight-arm Radial Arm Maze test. Doses of seletracetam administered to the AI rats include 0.0625 mg/kg, 0.125 mg/kg, 0.25 mg/kg, 0.5 mg/kg, 1 mg/kg, 2 mg/kg and 4 mg/kg. Means and SEMs for the number of errors are shown as the y-axis.

FIG. 18A and FIG. 18B depict the performance of aged-impaired rats (n=3/group) treated with brivaracetam at a dose of 2 mg/kg/day after 14 days in the water maze task. Rats treated with brivaracetam at 2 mg/kg/day (t(2)=10.000, p=0.010) but not vehicle (t(2)=1.964, p=0.188) showed a significant spatial bias for the target quadrant compared to the other control quadrants. Brivaracetam-treated rats (2 mg/kg/day) also spent significantly more time in the target quadrant than the vehicle-treated rats, t(4)=3.881, p=0.018. Brivaracetam-treated rats (2 mg/kg/day) spent significantly more time in the target annulus (area surrounding the location of the escape platform) than the vehicle-treated rats, t(4)=3.109, p=0.036.

FIG. 19A and FIG. 19B depict the effects of levetiracetam on fMRI activities in Dentate Gyrus/CA3 region of aMCI patients at a dose of 62.5 mg BID and 250 mg BID.

FIG. 20A and FIG. 20B show the difference between the aMCI (placebo) subjects and age-matched control (placebo) subjects in their performance of the explicit 3-alternative forced choice task described in Example 3 at a dose of 62.5 mg BID placebo and 250 mg BID placebo. Each bar represents the proportion of the subject responses (old, similar, or new) when presented with a lure image.

FIG. 21A and FIG. 21B show the difference between the same aMCI subjects with placebo treatment or with levetiracetam treatment (62.5 mg BID and 250 mg BID) in their performance of the explicit 3-alternative forced choice task described in Example 3. Each bar represents the proportion of the subjects' responses (old, similar, or new) when presented with a lure image.

FIG. 22 shows that administering levetiracetam at a dose of 10 mg/kg/day and vehicle in osmotic minipumps for four weeks in aged-impaired rats restores somatostatin in DG hilus.

FIG. 23 shows that administering levetiracetam at a dose of 10 mg/kg/day and vehicle in osmotic minipumps for four weeks in aged-impaired rats restores reelin in Entorhinal Cortex (EC2).

FIGS. 24A-24C depict the levetiracetam blood plasma levels for the aMCI patients at a dose of 62.5 mg BID, 125 mg BID, and 250 mg BID levetiracetam.

FIG. 25 is an XRPD pattern overlay of anhydrous polymorphic forms of Compound 1. The top diffractogram corresponds to anhydrous Form A, the second from the top corresponds to desolvated Form B, the third from the top corresponds to anhydrous Material D (as a mixture with Form A), and the bottom corresponds to anhydrous Form E.

FIG. 26 is an XRPD pattern overlay of the solvated polymorphic forms of Compound 1. The top diffractogram corresponds to methanolate Form C, and the bottom corresponds to monohydrate Form F.

FIGS. 27A and 27B depict the thermograms of anhydrous Form A. FIG. 27A (top) corresponds to the thermogravimetric analysis (TGA) curve and FIG. 27B (bottom) corresponds to the differential scanning calorimetry (DSC) curve.

FIG. 28 depicts the atomic displacement ellipsoid diagram of anhydrous Form A. Non-hydrogen atoms are represented by 50% probability anisotropic thermal ellipsoids.

FIG. 29 is an XRPD overlay of the experimental (top) and calculated (bottom) patterns for anhydrous Form A.

FIG. 30 depicts the dynamic vapor sorption isotherm of anhydrous Form A.

FIG. 31 depicts the indexed XRPD pattern of desolvated Form B.

FIG. 32 is an XRPD overlay of Material D taken initially after preparation (top) and after 7 weeks at ambient storage (middle). The XRPD pattern of Form A is provided as a reference (bottom).

FIGS. 33A and 33B depict the thermograms of Material D (as a mixture with Form A). FIG. 33A (top) corresponds to the TGA curve, and FIG. 33B (bottom) corresponds to the DSC curve.

FIG. 34 depicts the atomic displacement ellipsoid diagram of anhydrous Form E. Non-hydrogen atoms are represented by 50% probability anisotropic thermal ellipsoids.

FIG. 35 is an XRPD overlay of the experimental (top) and calculated (bottom) anhydrous Form E.

FIGS. 36A and 36B depict the thermograms of anhydrous Form E. FIG. 36A (top) corresponds to the TGA curve, and FIG. 36B (bottom) corresponds to the DSC curve.

FIG. 37 is an XRPD overlay of monohydrate Form F (top) and the HCl salt of Compound 1 (bottom) for reference.

FIG. 38 is the indexed XRPD pattern of monohydrate Form F.

FIGS. 39A and 39B depict the thermograms of monohydrate Form F. FIG. 39A (top) corresponds to the TGA curve, and FIG. 39B (bottom) corresponds to the DSC curve.

FIG. 40 depicts the dynamic vapor sorption (DVS) isotherm of monohydrate Form F.

FIG. 41 is the indexed XRPD pattern of methanolate Form C.

FIGS. 42A and 42B depict the thermograms of methanolate Form C. FIG. 42A (top) corresponds to the TGA curve, and FIG. 42B (bottom) corresponds to the DSC curve.

FIG. 43 is an XRPD overlay of crude Compound 1 (top), calculated Form A (middle), and experimental Form B (bottom). The * symbol denotes additional peaks not attributable to either Form A or Form B.

FIG. 44 is a graph showing the effect of Compound 1, as compared to vehicle control in aged-impaired rats using a Radial Arm Maze behavioral task. The graphs show the mean number of errors made by aged-impaired rats treated with varying doses of Compound 1 (2.5 mg/kg, 5 mg/kg, and 10 mg/kg).

FIGS. 45A and 45B are graphs showing the effect of Compound 1, as compared to vehicle control in aged-impaired rats using a Morris Water Maze behavioral task. FIG. 45A shows the amount of time spent in target quadrants after acute treatment with Compound 1 (10 mg/kg); FIG. 45B shows the amount of time spent in target quadrants after chronic treatment (12 weeks) with Compound 1 (10 mg/kg).

FIGS. 46A and 46B depict the effect of a combination of Compound 1 and levetiracetam, as compared to vehicle control, in aged-impaired rats using a Radial Arm Maze behavioral task. FIG. 46A shows the mean number of errors made by aged-impaired rats treated with a combination of Compound 1 at a dose of 2.5 mg/kg and levetiracetam at a dose of 2.5 mg/kg or a combination of Compound 1 at a dose of 5 mg/kg and levetiracetam at a dose of 2.5 mg/kg. FIG. 46B shows an isobolographic analysis of the combination of Compound 1 and levetiracetam to assess if synergy exists in the combination treatment versus treatment with Compound 1 or levetiracetam alone.

### DETAILED DESCRIPTION OF THE DISCLOSURE

GABA_{A} receptors (GABA_{A} R) are pentameric assemblies from a pool of different subunits (α1-6, β1-3, γ1-3, δ, ε, π, θ) that form a Cl⁻ permeable channel that is gated by the neurotransmitter γ-aminobutyric acid (GABA). Various pharmacological effects, including anxiety disorders, epilepsy, insomnia, pre-anesthetic sedation, and muscle relaxation, are mediated by different GABA_{A} subtypes.

Various studies have demonstrated that reduced GABA signaling is linked to various CNS disorders with cognitive impairment. For example, some previous studies have demonstrated a reduction of hippocampal expression of the α5 subunit of the GABA_{A} receptor in rats with age-related cognitive decline (See, International Patent Publication WO 2007/019312). And other studies have shown that positive allosteric modulators of α5-containing GABA_{A} R, GABA_{A} α5 receptor agonists are useful for the treatment of cognitive impairment associated with said CNS disorders, cognitive impairment associated with a brain cancer, a brain cancer, or Parkinson's disease psychosis. See, e.g., WO 2015/095783, WO 2018/130868, WO 2016/205739, WO 2018/130869, WO 2019/246300, and U.S. 62/950,886, all of which are specifically incorporated herein by reference.

Synaptic vesicle protein-2 (SV2) is a family of synaptic vesicle proteins, which consists of three members, designated SV2A, SV2B, and SV2C. SV2A is the most widely distributed family member, being expressed ubiquitously in the brain. The proteins are integral membrane proteins and have a low-level homology (20-30%) to the twelve transmembrane family of bacterial and fungal transporter proteins that transport sugar, citrate, and xenobiotics (Bajjalieh et al., Science, 257: 1271-1273 (1992)). SV2 family proteins are present in the brain and endocrine cells, and further are present in all synaptic and endocrine vesicles. SV2 proteins are reported to play a role in normal synaptic function and function in a maturation step of primed vesicles that converts the vesicles into a Ca²⁺— and synaptotagmin-responsive state (Sudhof et al., 2009). SV2 proteins are reported to enhance synaptic currents and increase the probability of transmitter release by maintaining the size of the readily releasable pool of vesicles (Custer et al., 2006). Various studies have shown that SV2A inhibitors, compounds that bind to SV2A and reduce synaptic function by reducing pre-synaptic vesicle release (See, e.g., Noyer et al. 1995; Fuks et al. 2003; Lynch et al. 2004; Gillard et al. 2006; Custer et al., 2006; Smedt et al., 2007; Yang et al., 2007; Meehan, “Levetiracetam has an activity-dependent effect on inhibitory transmission,” Epilepsia, 2012 Jan. 31; and Example 8 of WO 2001/62726, all of which are specifically incorporated herein by reference), may be effective in the treatment of cognitive impairment associated with CNS disorders. See, e.g., International Patent Application PCT/US2009/005647 (Pub. No. WO2010/044878), International Patent Application PCT/US2011/024256 (Pub. No. WO2011/100373), International Patent Application PCT/US2012/024556 (Pub. No. WO2012/109491), International Patent Application PCT/US2013/070144 (Pub. No. WO2014/078568), International Patent Application PCT/US2014/029170 (Pub. No. WO2014/144663), International Patent Application PCT/US2014/029362 (Pub. No. WO2014/144801), and International Patent Application PCT/US2016/033567 (Pub. No. WO2016/191288), all of which are specifically incorporated herein by reference.

Surprisingly, the combination of an SV2A inhibitor (e.g., levetiracetam, brivaracetam, or seletracetam), or a pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, was found to have a synergistic, super-additive effect on the treatment of cognitive impairment associated with CNS disorders such as age-related cognitive impairment, mild cognitive impairment (MCI), amnestic MCI (aMCI), age-associated memory impairment (AAMI), age related cognitive decline (ARCD), dementia, Alzheimer's disease (AD), prodromal AD, post-traumatic stress disorder (PTSD), schizophrenia, bipolar disorder, amyotrophic lateral sclerosis (ALS), cancer-therapy-related cognitive impairment, mental retardation, Parkinson's disease (PD), autism, compulsive behavior, and substance addiction. The effect of the combination on the treatment of said cognitive impairment was greater than what would have been anticipated had the effect been simply additive. Moreover, this effect should be observed in the treatment of cognitive impairment associated with a brain cancer, a brain cancer, and Parkinson's disease psychosis in a subject in need thereof. The synergistic effect is also surprising as the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, have different mechanisms of action.

This synergistic combination of the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, enables dosing at lower, subtherapeutic amounts, less than the amounts normally used when the SV2A inhibitor (e.g., levetiracetam, brivaracetam, or seletracetam), or the pharmaceutically acceptable salt, hydrate, solvate, polymorph, or isomer thereof, and GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are used alone to treat disorders involving cognitive dysfunction. Such lower dosing can alleviate or prevent possible negative side effects or toxicity associated with administration of the SV2A inhibitor (e.g., levetiracetam, brivaracetam, or seletracetam), or the pharmaceutically acceptable salt, hydrate, solvate, polymorph, or isomer thereof, and GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof. Additionally, despite the administration of lower doses, due to the synergy of the combination, efficacy is not lost but improves and a longer therapeutic effect may be observed. Enhancing therapeutic efficacy through use of the combination may result in improved treatment outcomes without the need to increase the doses administered or the frequency of administration. While increased dosing may be therapeutically advantageous in some circumstances, it can also sometimes exacerbate negative side effects or lead to toxicity, resulting in the need for new treatment regimes, particularly for therapeutics administered for chronic conditions or over a long period of time. Additionally, negative side effects and increased dosing frequency can lead to reduced patient compliance with treatment. As the combination of an SV2A inhibitor (e.g., levetiracetam, brivaracetam, or seletracetam), or a pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, is useful for the long term treatment of cognitive impairment associated with CNS disorders such as age-related cognitive impairment, mild cognitive impairment (MCI), amnestic MCI (aMCI), age-associated memory impairment (AAMI), age related cognitive decline (ARCD), dementia, Alzheimer's disease (AD), prodromal AD, post-traumatic stress disorder (PTSD), schizophrenia, bipolar disorder, amyotrophic lateral sclerosis (ALS), cancer-therapy-related cognitive impairment, mental retardation, Parkinson's disease (PD), autism, compulsive behavior, and substance addiction, the observed synergy is advantageous. Additionally, the combination is useful for treating cognitive impairment associated with a brain cancer, a brain cancer, and Parkinson's disease psychosis in a subject in need thereof.

### General Information

Unless otherwise defined herein, scientific and technical terms used in this application shall have the meanings that are commonly understood by those of ordinary skill in the art. Generally, nomenclature used in connection with, and techniques of, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art. See, e.g., “Principles of Neural Science,” McGraw-Hill Medical, New York, N.Y. (2000); Motulsky, “Intuitive Biostatistics,” Oxford University Press, Inc. (1995); Lodish et al., “Molecular Cell Biology, 4th ed.,” W.H. Freeman & Co., New York (2000); Griffiths et al., “Introduction to Genetic Analysis, 7th ed.,” W.H. Freeman & Co., N.Y. (1999); Gilbert et al., “Developmental Biology, 6th ed.,” Sinauer Associates, Inc., Sunderland, Mass. (2000).

Chemistry terms used herein are used according to conventional usage in the art, as exemplified by “The McGraw-Hill Dictionary of Chemical Terms,” Parker S., Ed., McGraw-Hill, San Francisco, Calif. (1985).

All publications, patents and published patent applications referred to in this application are specifically incorporated by reference herein. In case of conflict, the present specification, including its specific definitions, will control.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

The singular forms “a,” “an,” and “the” include the plurals unless the context clearly dictates otherwise.

“Including” is used to mean “including but not limited to.” “Including” and “including but not limited to” are used interchangeably.

“Patient,” “subject”, or “individual” are used interchangeably and may refer to either a human or a non-human animal. Patient, subject, or individual may include mammals, such as humans, primates, livestock animals (including bovines, porcines, etc.), companion animals (e.g., canines, felines, etc.) and rodents (e.g., mice and rats). In some embodiments, the patient, subject, or individual is a human.

“Cognitive function” or “cognitive status” may refer to any higher order intellectual brain process or brain state, respectively, involved in learning and/or memory including, but not limited to, attention, information acquisition, information processing, working memory, short-term memory, long-term memory, anterograde memory, retrograde memory, memory retrieval, discrimination learning, decision-making, inhibitory response control, attentional set-shifting, delayed reinforcement learning, reversal learning, the temporal integration of voluntary behavior, and expressing an interest in one's surroundings and self-care, speed of processing, reasoning and problem solving, and social cognition.

“Promoting” cognitive function may refer to affecting impaired cognitive function so that it more closely resembles the function of a normal subject. Cognitive function may be promoted to any detectable degree, but in humans may be promoted sufficiently to allow an impaired subject to carry out daily activities of normal life a level of proficiency as close as possible to a normal subject or an age-matched normal subject.

In some embodiments, “promoting” cognitive function in a subject affected by age-related cognitive may refer to affecting impaired cognitive function so that it more closely resembles the function of an aged-matched normal subject, or the function of a young adult subject. Cognitive function of that subject may be promoted to any detectable degree, but in humans may be promoted sufficiently to allow an impaired subject to carry out daily activities of normal life at a level of proficiency as close as possible to a normal subject or a young adult subject or an age-matched normal subject.

“Preserving” cognitive function may refer to affecting normal or impaired cognitive function such that it does not decline or does not fall below that observed in the subject upon first presentation or diagnosis or delays such decline.

“Improving” cognitive function may include promoting cognitive function and/or preserving cognitive function in a subject.

“Cognitive impairment” may refer to cognitive function in subjects that is not as robust as that expected in a normal subject. In some cases, cognitive function is reduced by about 5%, about 10%, about 30%, or more, compared to cognitive function expected in a normal subject. In some cases, “cognitive impairment” in subjects affected by aged-related cognitive impairment may refer to cognitive function in subjects that is not as robust as that expected in an aged-matched subject, or the function of a young adult subject (e.g., subjects with mean scores for a given age in a cognitive test).

“Treating cognitive impairment associated with a brain cancer” or “treating a brain cancer” in a patient in need thereof may refer to taking steps to obtain beneficial or desired results, including clinical results. Beneficial or desired clinical results may include, but are not limited to, improving cognitive function in a patient with a brain cancer; delaying or slowing the progression of a brain cancer or cognitive impairment in a patient with a brain cancer; reducing the rate of decline of cognitive function in a patient with a brain cancer; preventing or slowing the progression of a brain cancer or cognitive impairment associated with a brain cancer; or alleviation, amelioration, or slowing the progression of one or more symptoms associated with a brain cancer or cognitive impairment associated with a brain cancer.

“Treating Parkinson's disease psychosis” in a patient in need thereof may refer to taking steps to obtain beneficial or desired results, including clinical results. Beneficial or desired clinical results may include, but are not limited to, improving Parkinson's disease psychosis; delaying or slowing the progression of Parkinson's disease psychosis; preventing or slowing the progression of the Parkinson's disease psychosis; or alleviation, amelioration, or slowing the progression of one or more symptoms associated with Parkinson's disease psychosis.

“Treating cognitive impairment” may refer to taking steps to improve cognitive function in a subject with cognitive impairment so that the subject's performance in one or more cognitive tests is improved to any detectable degree or is prevented from further decline. That subject's cognitive function, after treatment of cognitive impairment, may more closely resemble the function of a normal subject. Treatment of cognitive impairment in humans may improve cognitive function to any detectable degree but may be improved sufficiently to allow the impaired subject to carry out daily activities of normal life at the same level of proficiency as a normal subject. In some cases, “treating cognitive impairment” may refer to taking steps to improve cognitive function in a subject with cognitive impairment so that the subject's performance in one or more cognitive tests is improved to any detectable degree or is prevented from further decline. That subject's cognitive function, after treatment of cognitive impairment, may more closely resemble the function of a normal subject. In some cases, “treating cognitive impairment” in a subject affected by age-related cognitive impairment may refer to taking steps to improve cognitive function in the subject so that the subject's cognitive function, after treatment of cognitive impairment, more closely resembles the function of an age-matched normal subject, or the function of a young adult subject. In some cases, “treating cognitive impairment” in a subject may refer to taking steps to delay or slow the progression of cognitive impairment in a subject with cognitive impairment. In some cases, “treating cognitive impairment” in a subject may refer to taking steps to reduce the rate of decline of cognitive function in a subject with cognitive impairment. Beneficial or desired clinical results include, but are not limited to, improving cognitive function; delaying or slowing the progression of cognitive impairment; reducing the rate of decline of cognitive function; preventing or slowing the progression of the disease or disorder; or alleviation, amelioration, or slowing the progression of one or more symptoms associated with cognitive impairment associated with CNS disorders, such as age-related cognitive impairment, mild cognitive impairment (MCI), amnestic MCI, age-associated memory impairment (AAMI), age related cognitive decline (ARCD), dementia, Alzheimer's disease (AD), prodromal AD, PTSD, schizophrenia or bipolar disorder (in particular, mania), amyotrophic lateral sclerosis (ALS), cancer therapy-related cognitive impairment, mental retardation, Parkinson's disease (PD), autism, compulsive behavior, or substance addiction. Treating age-related cognitive impairment further comprises slowing the conversion of age-related cognitive impairment into dementia (e.g., AD).

### Compounds Useful in the Methods, Uses, Pharmaceutical Compositions, and Combinations of the Disclosure

Compounds useful in the methods, uses, combinations, pharmaceutical compositions, combinations for use, or pharmaceutical compositions for use of the disclosure include synaptic vesicle glycoprotein 2A (SV2A) inhibitors, or pharmaceutically acceptable salts, hydrates, solvates, polymorphs, or isomers thereof, and GABA_{A} α5 receptor agonists, or pharmaceutically acceptable salts, hydrates, solvates, polymorphs, or isomers thereof.

### SV2A Inhibitors

“SV2A inhibitor” may refer to any compound that binds to SV2A and reduces synaptic function by reducing pre-synaptic vesicle release (See, e.g., Noyer et al. 1995; Fuks et al. 2003; Lynch et al. 2004; Gillard et al. 2006; Custer et al., 2006; Smedt et al., 2007; Yang et al., 2007; Meehan, “Levetiracetam has an activity-dependent effect on inhibitory transmission,” Epilepsia, 2012 Jan. 31; and Example 8 of WO 2001/62726, all of which are specifically incorporated herein by reference.) A compound may be an SV2A inhibitor even if it does not itself bind to SV2A, as long as it causes, or affects the ability of, another compound to bind SV2A or reduce synaptic function by reducing pre-synaptic vesicle release. SV2A inhibitors suitable for the methods, uses, pharmaceutical compositions, or combinations of the present disclosure include the specific SV2A inhibitors described herein, and pharmaceutically acceptable salts, hydrates, solvates, polymorphs, or isomers thereof.

In some embodiments of this disclosure, the SV2A inhibitor, or the pharmaceutically acceptable salt, hydrate, solvate, polymorph, or isomer thereof, is selected from the group consisting of levetiracetam, brivaracetam, and seletracetam, or pharmaceutically acceptable salts, solvates, hydrates, polymorphs, or isomers of any of the foregoing.

In some embodiments of this disclosure, the SV2A inhibitor, or the pharmaceutically acceptable salt, hydrate, solvate, polymorph, or isomer thereof, is levetiracetam, or a pharmaceutically acceptable salt, hydrate, solvate, polymorph, or isomer thereof. Levetiracetam refers to the compound (2S)-2-(2-oxopyrrolidin-1-yl)butanamide (International Union of Pure and Applied Chemistry (IUPAC) name). Levetiracetam is a widely used antiepileptic drug. Levetiracetam binds to a specific site in the CNS: the synaptic vesicle protein 2A (SV2A) (See, e.g., Noyer et al. 1995; Fuks et al. 2003; Lynch et al. 2004; Gillard et al. 2006) and has further been shown to directly inhibit synaptic activity and neurotransmission by inhibiting presynaptic neurotransmitter release (Yang et al., 2007). Levetiracetam is sold as the FDA approved antiepileptic drug Keppra. Typically, the therapeutically effective dose of levetiracetam (Keppra) is in a range of 1000-3000 mg/day.

In some embodiments of this disclosure, the SV2A inhibitor, or the pharmaceutically acceptable salt, hydrate, solvate, polymorph, or isomer thereof, is brivaracetam, or a pharmaceutically acceptable salt, hydrate, solvate, polymorph, or isomer thereof. Brivaracetam refers to the compound (2S)-2-[(4R)-2-oxo-4-propylpyrrolidin-1-yl]butanamide (IUPAC name). It has anticonvulsant activity and binds to SV2A in the brain.

In some embodiments of this disclosure, the SV2A inhibitor, or the pharmaceutically acceptable salt, hydrate, solvate, polymorph, or isomer thereof, is seletracetam, or a pharmaceutically acceptable salt, hydrate, solvate, polymorph, or isomer thereof. Seletracetam refers to the compound (2S)-2-[(4S)-4-(2,2-difluoroethenyl)-2-oxopyrrolidin-1-yl]butanamide (IUPAC name). It is an antiepileptic agent and binds to SV2A in the brain.

### GABA_{A} α5 Receptor Agonists

As used herein, a “α5-containing GABA_{A} receptor agonist,” “α5-containing GABA_{A} R agonist” or a “GABA_{A} α5 receptor agonist” and other variations as used herein refer to a compound that enhances the function of α5-containing GABA_{A} receptor (GABA_{A} R), i.e., a compound that increases GABA-gated Cl⁻ currents. In some embodiments, GABA_{A} α5 receptor agonist as used herein may refer to a positive allosteric modulator, which potentiates the activity of GABA. GABA_{A} α5 receptor agonists suitable for use in the present disclosure, include the α5-containing GABA_{A} receptor agonists of all formulas and specific GABA_{A} α5 receptor agonists described herein, or pharmaceutically acceptable salts, hydrates, solvates, polymorphs, or isomers thereof. Exemplary GABA_{A} α5 receptor agonists are disclosed in WO 2015/095783, WO 2016/205739, WO 2018/130868, WO 2018/130869, WO 2019/246300, and WO2021/127543

In some embodiments, the GABA_{A} α5 receptor agonist selected from the group consisting of:
i) a compound of formula I:

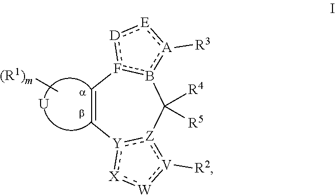

or a pharmaceutically acceptable salt, hydrate, solvate, polymorph, or isomer thereof, wherein:
U and the two carbon atoms designated by α and β together form a 5- or 6-membered aromatic ring having 0-2 nitrogen atoms;
A is C, CR⁶, or N;
B and F are each independently selected from the group consisting of C, CR⁶, and N, wherein B and F cannot both be N;
D is N, NR⁷, O, CR⁶ or C(R⁶)₂;
E is N, NR⁷, CR⁶ or C(R⁶)₂;
W is N, NR⁷, CR⁶ or C(R⁶)₂;
X is N, NR⁷, O, CR⁶ or C(R⁶)₂;
Y and Z are each independently selected from the group consisting of C, CR⁶, and N, wherein Y and Z cannot both be N;
V is C or CR⁶,
or when Z is C or CR⁶, V is C, CR⁶, or N;
wherein when the ring formed by X, Y, Z, V and W is

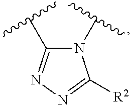

then R is —OR⁸, —SR⁸, —(CH₂)ₙOR⁸, —(CH₂)ₙO(CH₂)ₙR⁸, —(CH₂)_{P}R⁸ or —(CH₂)ₙN(R″)R¹⁰; and wherein R² is independently substituted with 0-5 R′;
m and n are independently integers selected from 0-4;
p is an integer selected from 2-4;
each occurrence of the bond “ [symbol] ” is independently either a single bond or a double bond;
each occurrence of R¹, R², R⁴, and R⁵ are each independently selected from the group consisting of:
   halogen, —R, —OR, —NO₂, —NCS, —CN, —CF₃, —OCF₂H —OCF₃, —SiR₃, —N(R)₂, —SR, —SOR, —SO₂R, —SO₂N(R)₂, —SO₃R, —(CR₂)₁₋₃R, —(CR₂)₁₋₃—OR, —(CR₂)₁₋₃—O(CR₂)₁₋₃—R, —(CR₂)₀₋₃—C(O)NR(CR₂)₀₋₃R, —(CR₂)₀₋₃—C(O)NR(CR₂)₀₋₃OR, —C(O)R, —C(O)C(O)R, —C(O)CH₂C(O)R, —C(S)R, —C(S)OR, —C(O)OR, —C(O)C(O)OR, —C(O)C(O)N(R)₂, —OC(O)R, —C(O)N(R)₂, —OC(O)N(R)₂, —C(S)N(R)₂, —(CR₂)₀₋₃NHC(O)R, —N(R)N(R)COR, —N(R)N(R)C(O)OR, —N(R)N(R)CON(R)₂, —N(R)SO₂R, —N(R)S O₂N(R)₂, —N(R)C(O)OR, —N(R)C(O)R, —N(R)C(S)R, —N(R)C(O)N(R)₂, —N(R)C(S)N(R)₂, —N(COR)COR, —N(OR)R, —C(═NH)N(R)₂, —C(O)N(OR)R, —C(═NOR)R, —OP(O)(OR)₂, —P(O)(R)₂, —P(O)(OR)₂, —P(O)(H)(OR), —C≡C—R⁸, —CH₂CF₃, and CHF₂;
each occurrence of R⁸ is independently —H, —(C1-C6) alkyl, —(C3-C6) cycloalkyl, —(C1-C6) alkyl-(C3-C6) cycloalkyl, —(C1-C6) alkyl-(C6-C10) aryl, —(C6-C10) aryl, -5-10 membered heteroaryl, or —(C1-C6) alkyl-5-10 membered heteroaryl;
   wherein each R⁸ excluding —H and —(C1-C6) alkyl is independently substituted by 0-5 of -halogen, —(C1-C6) alkyl, —CF₃, —OCF₃, or O—(C1-C6) alkyl;
R³ is absent or is selected from the group consisting of:
   halogen, —R, —OR, —NO₂, —NCS, —CN, —CF₃, —OCF₃, —SiR₃, —N(R)₂, —SR, —SOR, —SO₂R, —SO₂N(R)₂, —SO₃R, —(CR₂)₁₋₃R, —(CR₂)₁₋₃—OR, —(CR₂)₀₋₃—C(O)NR(CR₂)₀₋₃R, —(CR₂)₀₋₃—C(O)NR(CR₂)₀₋₃OR, —C(O)R, —C(O)C(O)R, —C(O)CH₂C(O)R, —C(S)R, —C(S)OR, —C(O)OR, —C(O)C(O)OR, —C(O)C(O)N(R)₂, —OC(O)R, —C(O)N(R)₂, —OC(O)N(R)₂, —C(S)N(R)₂, —(CR₂)₀₋₃NHC(O)R, —N(R)N(R)COR, —N(R)N(R)C(O)OR, —N(R)N(R)CON(R)₂, —N(R)SO₂R, —N(R)SO₂N(R)₂, —N(R)C(O)OR, —N(R)C(O)R, —N(R)C(S)R, —N(R)C(O)N(R)₂, —N(R)C(S)N(R)₂, —N(COR)COR, —N(OR)R, —C(═NH)N(R)₂, —C(O)N(OR)R, —C(═NOR)R, —OP(O)(OR)₂, —P(O)(R)₂, —P(O)(OR)₂, —P(O)(H)(OR), C≡C—R⁹, COOMe, COOEt, —(C1-C6)alkyl-C≡C—R¹⁰, CH₂—OR¹⁰, and CH₂—O—CH₂—R¹⁰;
wherein each of R⁹ is independently selected from the group consisting of —H, —(C1-C6) alkyl, —(C6-C10) aryl, -5-10 membered heteroaryl, —(C1-C6) alkyl-(C6-C10) aryl, —(C1-C6) alkyl-5-10 membered heteroaryl, —(C3-C6) cycloalkyl, —(C1-C6) alkyl-(C3-C6) cycloalkyl, —C(O)—(C6-C10) aryl, —(C3-C6)cycloalkyl-(C6-C10)aryl,

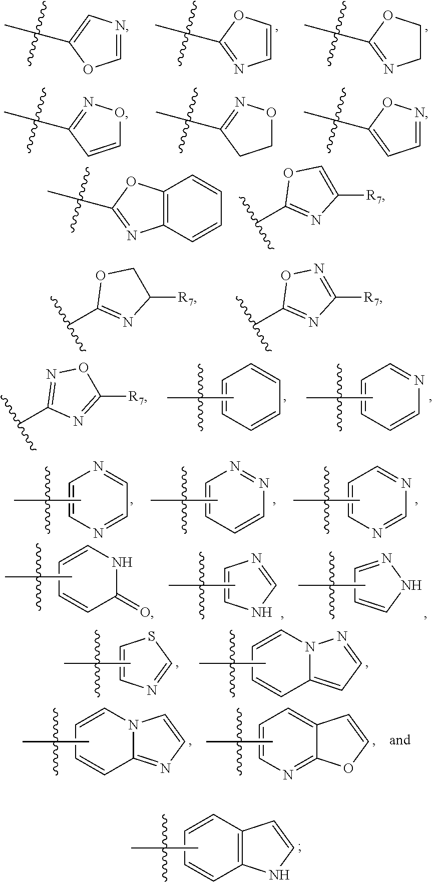

wherein each R⁹ is independently substituted with 0-5 R¹¹;
wherein each occurrence of R¹¹ is independently selected from the group consisting of -halogen, —CF₃, —OH, —OCF₃, OCHF₂, —O—(C1-C6)alkyl, —O—CH₂—(C3-C6)cycloalkyl, —CN, —SCH₃—(C6-C10) aryl, —(C1-C6)alkyl, and -5 to 10 membered heteroaryl,
wherein R¹⁰ is selected from the group consisting of —H, —(C1-C6) alkyl, —(C6-C10) aryl, -5-10 membered heteroaryl, —(C3-C6) cycloalkyl, —CH₂—(C3-C6) cycloalkyl, —CH₂—(C6-C10) aryl, and —CH₂-5-10-membered heteroaryl,
wherein each R¹⁰ is independently substituted with 0-5 R′;
wherein R₇ is selected from the group consisting of —(C1-C6)alkyl, —(C3-C6)cycloalkyl, -5 to 10 membered heteroaryl, —(C6-C10) aryl, —(C6-C10)aryl-(C1-C6)alkyl, and -5 to 10 membered heteroaryl-(C1-C6)alkyl, and -5-10 membered heteroaryl,
wherein each R₇ is independently substituted with 0-5 R′;
each R⁶ is independently —H or —(C1-C6)alkyl;
each R⁷ is independently —H or —(C1-C6)alkyl;
each R⁸ is independently —(C1-C6)alkyl, —(C3-C10)-cycloalkyl, (C6-C10)-aryl, or 5- to 10-membered heteroaryl, wherein each occurrence of R⁸ is independently substituted with 0-5 R′;
each R¹⁰ is independently —(C3-C10)-cycloalkyl, 3- to 10-membered heterocyclyl-, (C6-C10)-aryl, or 5- to 10-membered heteroaryl, wherein each occurrence of R¹⁰ is independently substituted with 0-5 R′;
each R is independently selected from the group consisting of:
   H—,
   (C1-C12)-aliphatic-,
   (C3-C10)-cycloalkyl-,
   (C3-C10)-cycloalkenyl-,
   [(C3-C10)-cycloalkyl]—(C1-C12)-aliphatic-,
   [(C3-C10)-cycloalkenyl]—(C1-C12)-aliphatic-,
   [(C3-C10)-cycloalkyl]-O—(C1-C12)-aliphatic-,
   [(C3-C10)-cycloalkenyl]-O—(C1-C12)-aliphatic-,
   (C6-C10)-aryl-,
   (C6-C10)-aryl-(C1-C12)aliphatic-,
   (C6-C10)-aryl-O—(C1-C12)aliphatic-,
   (C6-C10)-aryl-N(R″)—(C1-C12)aliphatic-,
   3- to 10-membered heterocyclyl-,
   (3- to 10-membered heterocyclyl)-(C1-C12)aliphatic-,
   (3- to 10-membered heterocyclyl)-O—(C1-C12)aliphatic-,
   (3- to 10-membered heterocyclyl)-N(R″)—(C1-C12)aliphatic-,
   5- to 10-membered heteroaryl-,
   (5- to 10-membered heteroaryl)-(C1-C12)-aliphatic-,
   (5- to 10-membered heteroaryl)-O—(C1-C12)-aliphatic-; and
   (5- to 10-membered heteroaryl)-N(R″)—(C1-C12)-aliphatic-;
wherein said heterocyclyl has 1-4 heteroatoms independently selected from the group consisting of N, NH, O, S, SO, and SO₂, and said heteroaryl has 1-4 heteroatoms independently selected from the group consisting of N, NH, O, and S;
wherein each occurrence of R is independently substituted with 0-5 R′;
or when two R groups are bound to the same atom, the two R groups may be taken together with the atom to which they are bound to form a 3- to 10-membered aromatic or non-aromatic ring having 0-4 heteroatoms independently selected from the group consisting of N, NH, O, S, SO, and SO₂, wherein said ring is optionally substituted with 0-5 R′, and wherein said ring is optionally fused to a (C6-C10)aryl, 5- to 10-membered heteroaryl, (C3-C10)cycloalkyl, or a 3- to 10-membered heterocyclyl;
wherein each occurrence of R′ is independently selected from the group consisting of halogen, —R″, —OR″, oxo, —CH₂OR″, —CH₂NR″₂, —C(O)N(R″)₂, —C(O)OR″, —NO₂, —NCS, —CN, —CF₃, —OCF₃ and —N(R″)₂;
wherein each occurrence of R″ is independently selected from the group consisting of H, —(C1-C6)-alkyl, —(C1-C6)-aliphatic, (C3-C6)-cycloalkyl, 3- to 6-membered heterocyclyl, 5- to 10-membered heteroaryl-, (C6-C10)-aryl-, (5- to 10-membered heteroaryl)-(C1-C6)-alkyl-, (C6-C10)-aryl-(C1-C6)-alkyl-, (5- to 10-membered heteroaryl)-O—(C1-C6)-alkyl-, and (C6-C10)-aryl-O—(C1-C6)-alkyl-, wherein each occurrence of R″ is independently substituted with 0-3 substituents selected from the group consisting of: halogen, —R^{o}, —OR^{o}, oxo, —CH₂OR^{o}, —CH₂N(R^{o})₂, —C(O)N(R^{o})₂, —C(O)OR^{o}, —NO₂, —NCS, —CN, —CF₃, —OCF₃ and —N(R^{o})₂, wherein each occurrence of R^{o} is independently selected from the group consisting of: —(C1-C6)-aliphatic, (C3-C6)-cycloalkyl, 3- to 6-membered heterocyclyl, 5- to 10-membered heteroaryl-, and (C6-C10)-aryl-;
ii) a compound of formula II:

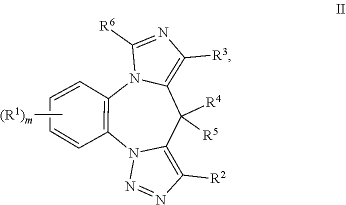

or a pharmaceutically acceptable salt, hydrate, solvate, polymorph, or isomer thereof, wherein:
m is 0-3;
each R¹ is independently selected from the group consisting of: halogen, —H, —(C1-C6)alkyl, —OH, -O((C1-C6)alkyl), —NO₂, —CN, —CF₃, —OCF₃, —OCHF₂, -OMe, —C≡C—R⁸, —CHF₂, —CH₂CF₃, —(C6-C10) aryl, —(C1-C6) alkyl-(C6-C10) aryl, -5-10 membered heteroaryl, —(C1-C6) alkyl-5-10 membered heteroaryl, and —(C3-C6) cycloalkyl; wherein R¹ is independently substituted with 0-5 R′;
R² is selected from the group consisting of:
   —H, halogen, —OH, —(C1-C6)aliphatic, —O((C1-C6)alkyl), —C(O)O((C1-C6)alkyl), —C(O)NR₂, —(CR₂)₁₋₃—OR, —(CR₂)₁₋₃-O(CR₂)₁₋₃—R, —OR⁹, —C(O)R⁸, —CH₂R⁸, —CH₃, —CH₂—OR⁸,
   (C6-C10)-aryl-,
   (C6-C10)-aryl-(C1-C12)aliphatic-,
      (C6-C10)-aryl-O—(C1-C12)aliphatic-,
   (C6-C10)-aryl-N(R″)—(C1-C12)aliphatic-,
   (5- to 10-membered heteroaryl)-(C1-C12)aliphatic-,
   (5- to 10-membered heteroaryl)-O—(C1-C12)aliphatic-,
   (5- to 10-membered heteroaryl)-N(R″)—(C1-C12)aliphatic-,
   (3- to 10-membered heterocyclyl)-(C1-C12)aliphatic-,
   (3- to 10-membered heterocyclyl)-O—(C1-C12)aliphatic-, and
   (3- to 10-membered heterocyclyl)-N(R″)—(C1-C12)aliphatic-,
   wherein R² is independently substituted with 0-5 R′;
R³ is selected from the group consisting of:
   (C1-C6)alkyl, —(C2-C6)alkenyl, —C≡CH, —C≡CR⁹, —CN, halogen, —SO₂((C6-C10)-aryl), —SO₂((C1-C6)alkyl), —C(O)N((C1-C6)alkyl)₂, —C(O)NH₂, —C(O)O((C1-C6)alkyl), —C(O)((C1-C6)alkyl), —(C6-C10)aryl, 5- to 10-membered heteroaryl, 5- to 10-membered heterocyclyl, —(C1-C6)alkyl-C≡C—R¹⁰, —CH₂—O—R¹⁰, —CH₂—O—CH₂—R¹⁰

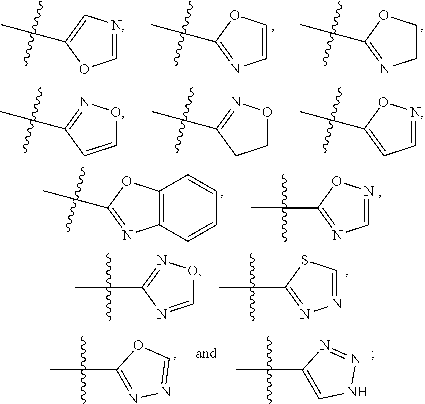

wherein each 5-member heterocycle or heteroaryl is substituted with 0-4 R₇;
wherein R³ is independently substituted with 0-5 R′;
R⁴ and R⁵ are each independently selected from the group consisting of —H, halogen, —(C1-C6)alkyl, or —(C1-C6) alkyl-(C6-C10) aryl; the (C6-C10)aryl being independently substituted with 0-5 halogen;
R⁶ is selected from the group consisting of —H and —(C1-C6)alkyl;
wherein R₇ is selected from the group consisting of —(C1-C6)alkyl, —(C3-C6)cycloalkyl, -5 to 10 membered heteroaryl, —(C6-C10) aryl, (C6-C10)aryl-(C1-C6)alkyl-, -5 to 10 membered heteroaryl-(C1-C6)alkyl, and -5-10 membered heteroaryl; wherein each R₇ is independently substituted with 0-5 R′;
wherein each R⁸ is independently selected from the group consisting of —H, —(C1-C6) alkyl, —(C3-C6) cycloalkyl, —(C1-C6)alkyl-(C3-C6)cycloalkyl, —(C1-C6)alkyl-(C6-C10)aryl, —(C6-C10) aryl, -5-10 membered heteroaryl, and —(C1-C6)alkyl-5-10 membered heteroaryl; wherein each R⁸ excluding —H and —(C1-C6) alkyl is independently substituted by 0-5 of -halogen, —(C1-C6) alkyl, —CF₃, —OCF₃, or O—(C1-C6) alkyl;
wherein R⁹ is selected from the group consisting of —H, —(C1-C6) alkyl, —(C6-C10)aryl, -5-10 membered heteroaryl, —(C1-C6)alkyl-(C6-C10) aryl, —(C1-C6) alkyl-5-10 membered heteroaryl, —(C3-C6) cycloalkyl, —(C1-C6) alkyl-(C3-C6) cycloalkyl, —C(O)—(C6-C10)aryl, 5-10 membered heterocycle,

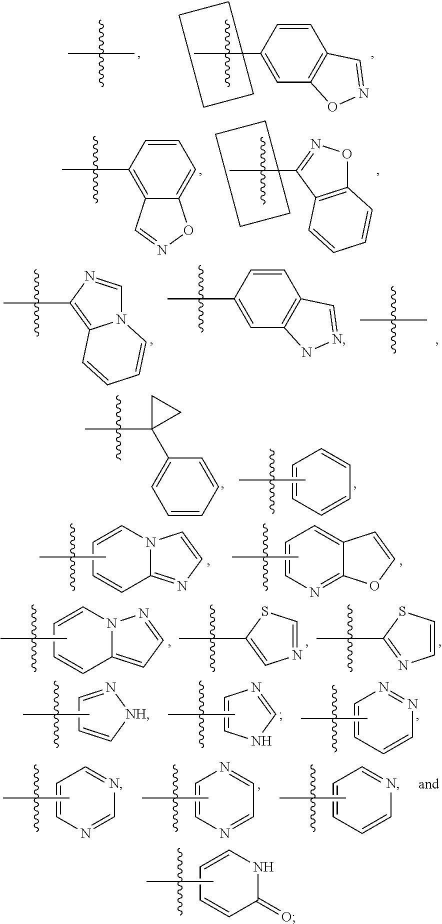

wherein each R⁹ is independently substituted with 0-5 R¹¹;
wherein R¹⁰ is selected from the group consisting of —H, halogen, —(C1-C6) alkyl, —(C6-C10) aryl, -5-10 membered heteroaryl, —(C3-C6) cycloalkyl, —CH₂—(C3-C6) cycloalkyl, —CH₂—(C6-C10) aryl, and —CH₂-5-10-membered heteroaryl,
wherein each R¹⁰ is substituted with 0-5 R′;
wherein each occurrence of R¹¹ is independently selected from the group consisting of -halogen, —CN, SCH₃, —CF₃, —OH, —OCF₃, OCHF₂, —O(C1-C6)alkyl, —(C6-C10) aryl, —(C1-C6)alkyl, and -5 to 10 membered heteroaryl;
each R is independently selected from the group consisting of:
   H—,
   (C1-C12)-aliphatic-,
   (C3-C10)-cycloalkyl-,
   (C3-C10)-cycloalkenyl-,
   [(C3-C10)-cycloalkyl]—(C1-C12)-aliphatic-,
   [(C3-C10)-cycloalkenyl]—(C1-C12)-aliphatic-,
   [(C3-C10)-cycloalkyl]-O—(C1-C12)-aliphatic-,
   [(C3-C10)-cycloalkenyl]-O—(C1-C12)-aliphatic-,
   (C6-C10)-aryl-,
   (C6-C10)-aryl-(C1-C12)aliphatic-,
   (C6-C10)-aryl-O—(C1-C12)aliphatic-,
   (C6-C10)-aryl-N(R″)—(C1-C12)aliphatic-,
   3- to 10-membered heterocyclyl-,
   (3- to 10-membered heterocyclyl)-(C1-C12)aliphatic-,
   (3- to 10-membered heterocyclyl)-O—(C1-C12)aliphatic-,
   (3- to 10-membered heterocyclyl)-N(R″)—(C1-C12)aliphatic-,
   5- to 10-membered heteroaryl-,
   (5- to 10-membered heteroaryl)-(C1-C12)-aliphatic-,
   (5- to 10-membered heteroaryl)-O—(C1-C12)-aliphatic-; and
   (5- to 10-membered heteroaryl)-N(R″)—(C1-C12)-aliphatic-;
wherein said heterocyclyl has 1-4 heteroatoms independently selected from the group consisting of N, NH, O, S, SO, and SO₂, and said heteroaryl has 1-4 heteroatoms independently selected from the group consisting of N, NH, O, and S;
wherein each occurrence of R is independently substituted with 0-5 R′;
or when two R groups bound to the same atom, the two R groups may be taken together with the atom to which they are bound to form a 3- to 10-membered aromatic or non-aromatic ring having 0-4 heteroatoms independently selected from the group consisting of N, NH, O, S, SO, and SO₂, wherein said ring is optionally substituted with 0-5 R′, and wherein said ring is optionally fused to a (C6-C10)aryl, 5- to 10-membered heteroaryl, (C3-C10)cycloalkyl, or a 3- to 10-membered heterocyclyl;
wherein each occurrence of R′ is independently selected from the group consisting of halogen, —R″, —OR″, oxo, —CH₂OR″, —CH₂NR″₂, —C(O)N(R″)₂, —C(O)OR″, —NO₂, —NCS, —CN, —CF₃, —OCF₃ and —N(R″)₂;
wherein each occurrence of R″ is independently selected from the group consisting of H, —(C1-C6)-alkyl, —(C1-C6)-aliphatic, (C3-C6)-cycloalkyl, 3- to 6-membered heterocyclyl, 5- to 10-membered heteroaryl-, (C6-C10)-aryl-, (5- to 10-membered heteroaryl)-(C1-C6)-alkyl-, (C6-C10)-aryl-(C1-C6)-alkyl-, (5- to 10-membered heteroaryl)-O—(C1-C6)-alkyl-, (C6-C10)-aryl-O—(C1-C6)-alkyl-, and (C6-C10)-aryl-O—(C1-C6)-alkyl-,
wherein each occurrence of R″ is independently substituted with 0-5 substituents selected from the group consisting of: halogen, —R^{o}, —OR^{o}, oxo, —CH₂OR^{o}, —CH₂N(R^{o})₂, —C(O)N(R^{o})₂, —C(O)OR^{o}, —NO₂, —NCS, —CN, —CF₃, —OCF₃ and —N(R^{o})₂, wherein each occurrence of R^{o} is independently selected from the group consisting of: —(C1-C6)-aliphatic, (C3-C6)-cycloalkyl, 3- to 6-membered heterocyclyl, 5- to 10-membered heteroaryl-, and (C6-C10)-aryl; and
iii) a compound of formula IV:

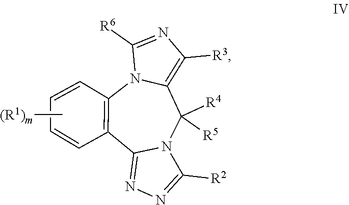

or a pharmaceutically acceptable salt, hydrate, solvate, polymorph, or isomer thereof, wherein:
m is 0-3;
each R¹ is independently selected from the group consisting of: halogen, —H, —(C1-C6)alkyl, —C≡C—R⁹, —OH, —O((C1-C6)alkyl), —NO₂, —CN, —CF₃, —OCF₃, —CHF₂, —CH₂CF₃, —(C6-C10) aryl, —(C1-C6) alkyl-(C6-C10) aryl, -5-10 membered heteroaryl, —(C1-C6) alkyl-5-10 membered heteroaryl, and —(C3-C6) cycloalkyl;
wherein R¹ is independently substituted with 0-5 R′;
R² is selected from the group consisting of —OR⁸, —SR⁸, —(CH₂)ₙOR⁸, —(CH₂)ₙO(CH₂)ₙR⁸, —(CH₂)_{P}R⁸ and —(CH₂)ₙN(R″)R¹⁰, wherein n is an integer selected from 0-4; p is an integer selected from 2-4;
wherein R² is independently substituted with 0-5 R′;
each R³ is independently selected from the group consisting of:
   —H, —CN, halogen, —(C1-C6)aliphatic, —CH═CR⁹, —C≡CR⁹, —SO₂((C1-C6)alkyl), —C(O)N((C1-C6)alkyl)₂), —C(O)NH((C1-C6)aliphatic), (C6-C10)-aryl-(C1-C12)aliphatic-, —C(O)((C1-C6)alkyl), —C(O)O((C1-C6)alkyl), 5- or 6-membered heterocyclyl, 5- or 6-membered heteroaryl, —CH₂—O—R¹⁰, —CH₂—O—CH₂—R¹⁰

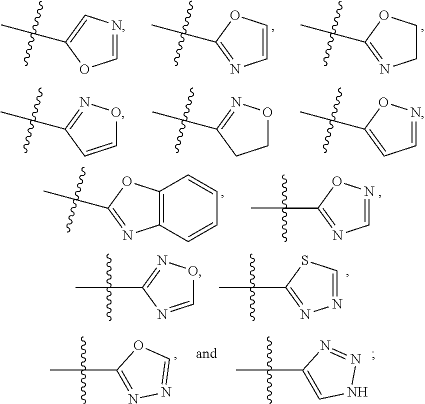

wherein each 5-10-membered heterocycle or heteroaryl are substituted with 0-3 R₇;
   wherein R³ is independently substituted with 0-5 R′;
R⁴ and R⁵ are each independently selected from the group consisting of —H, halogen and —(C1-C6)alkyl;
R⁶ is selected from the group consisting of —H and —(C1-C6)alkyl;
R₇ is selected from the group consisting of —(C1-C6)alkyl, —(C3-C6)cycloalkyl, -5 to 10 membered heteroaryl, —(C6-C10) aryl, —(C6-C10)aryl-(C1-C6)alkyl, and -5 to 10 membered heteroaryl-(C1-C6)alkyl, and -5-10 membered heteroaryl;
wherein each R₇ is independently substituted with 0-5 R′;
R⁸ is independently selected from the group consisting of —H, —(C1-C6)alkyl, —(C3-C10)-cycloalkyl, (C6-C10)-aryl, or 5- to 10-membered heteroaryl, 5-10 membered heteroaryl-(C1-C6) alkyl-, —(C1-C6) alkyl-(C6-C10) aryl, and —(C1-C6) alkyl-(C3-C6) cycloalkyl;
wherein each occurrence of R⁸ is independently substituted with 0-5 R′;
wherein R⁹ is selected from the group consisting of —H, —(C1-C6) alkyl, —(C3-C6) cycloalkyl, —(C1-C6) alkyl-(C3-C6) cycloalkyl, —(C1-C6)alkyl-(C6-C10) aryl, —(C6-C10)aryl, -5-10 membered heteroaryl, —(C1-C6)alkyl-5-10 membered heteroaryl, 5-10 membered heterocycle, —C(O)—(C6-C10) aryl,

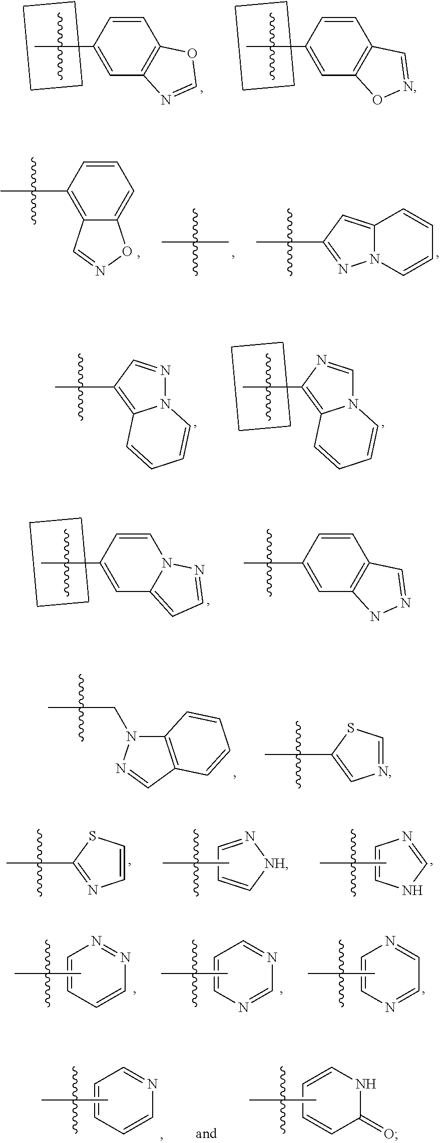

wherein each wherein each R⁹ is independently substituted with 0-5 R¹¹;
R¹⁰ is selected from the group consisting of —H, —(C1-C6) alkyl, —(C3-C10)-cycloalkyl, 3- to 10-membered heterocyclyl-, (C6-C10)-aryl, 5- to 10-membered heteroaryl, —CH₂—(C3-C6) cycloalkyl, —CH₂—(C6-C10) aryl, and —CH₂-5-10-membered heteroaryl,
   wherein each occurrence of R¹⁰ is independently substituted with 0-5 R′;
wherein each occurrence of R¹¹ is independently selected from the group consisting of -halogen, —CF₃, —OCF₃, OCF₂H, —O—(C1-C6)alkyl, —(C6-C10) aryl, —(C1-C6)alkyl, —O—CH₂—(C3-C6)cycloalkyl, and -5 to 10 membered heteroaryl;
wherein each occurrence of R′ is independently selected from the group consisting of halogen, —R″, —OR″, oxo, —CH₂OR″, —CH₂NR″₂, —C(O)N(R″)₂, —C(O)OR″, —NO₂, —NCS, —CN, —CF₃, —OCF₃ and —N(R″)₂;
wherein each occurrence of R″ is independently selected from the group consisting of H, —(C1-C6)-aliphatic, —(C1-C6)-alkyl, (C3-C6)-cycloalkyl, 3- to 6-membered heterocyclyl, 5- to 10-membered heteroaryl-, (C6-C10)-aryl-, (5- to 10-membered heteroaryl)-(C1-C6)-alkyl-, (C6-C10)-aryl-(C1-C6)-alkyl-, (5- to 10-membered heteroaryl)-O—(C1-C6)-alkyl-, and (C6-C10)-aryl-O—(C1-C6)-alkyl-;
wherein each occurrence of R″ is independently substituted with 0-5 R¹¹ independently selected from the group consisting of: halogen, —R^{o}, —OR^{o}, oxo, —CH₂OR^{o}, —CH₂N(R^{o})₂, —C(O)N(R^{o})₂, —C(O)OR^{o}, —NO₂, —NCS, —CN, —CF₃, —OCF₃ and —N(R^{o})₂, wherein each occurrence of R^{o} is independently selected from: —(C1-C6)-aliphatic, (C3-C6)-cycloalkyl, 3- to 6-membered heterocyclyl, 5- to 10-membered heteroaryl-, and (C6-C10)-aryl-.

In some embodiments, the GABA_{A} α5 receptor agonist, or the pharmaceutically acceptable salt, hydrate, solvate, polymorph, or isomer thereof, is a compound of Formula I, or a pharmaceutically acceptable salt, hydrate, solvate, polymorph, or isomer thereof. In some embodiments, the GABA_{A} α5 receptor agonist, or the pharmaceutically acceptable salt, hydrate, solvate, polymorph, or isomer thereof, is a compound of Formula II, or a pharmaceutically acceptable salt, hydrate, solvate, polymorph, or isomer thereof. In some embodiments, the GABA_{A} α5 receptor agonist, or the pharmaceutically acceptable salt, hydrate, solvate, polymorph, or isomer thereof, is a compound of Formula IV, or a pharmaceutically acceptable salt, hydrate, solvate, polymorph, or isomer thereof.

In some embodiments, the GABA_{A} α5 receptor agonist, or the pharmaceutically acceptable salt, hydrate, solvate, polymorph, or isomer thereof, is a compound of Formula I, or a pharmaceutically acceptable salt, hydrate, solvate, polymorph, or isomer thereof, selected from the group consisting of:

| Compound | |
|---|---|
| No. | Structure |
| 1 | 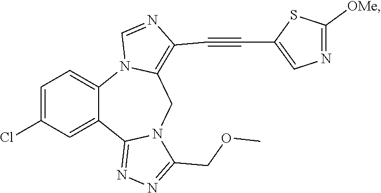 [SMILES: COCc1nnc2n1Cc1c(C#Cc3cnc(OC)s3)ncn1-c1ccc(Cl)cc1-2] |
| 2 | 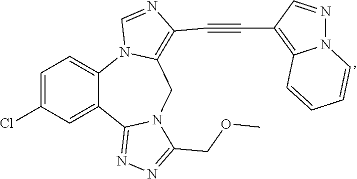 [SMILES: COCc1nnc2n1Cc1c(C#Cc3cnn4ccccc34)ncn1-c1ccc(Cl)cc1-2] |
| 3 | 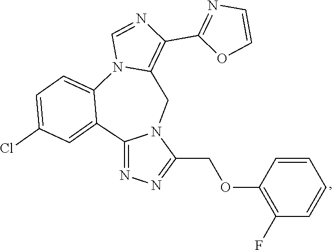 [SMILES: Fc1ccccc1OCc1nnc2n1Cc1c(-c3ncco3)ncn1-c1ccc(Cl)cc1-2] |
| 4 | 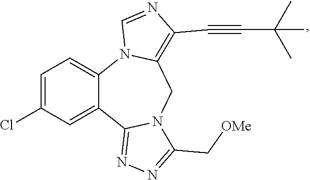 |
| 5 | 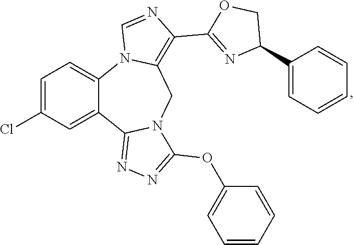 [SMILES: Clc1ccc2c(c1)-c1nnc(Oc3ccccc3)n1Cc1c(C3=N[C@H](c4ccccc4)CO3)ncn1-2] |
| 6 | 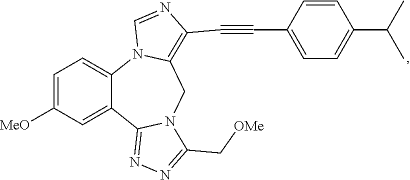 [SMILES: COCc1nnc2n1Cc1c(C#Cc3ccc(C(C)C)cc3)ncn1-c1ccc(OC)cc1-2] |
| 7 | 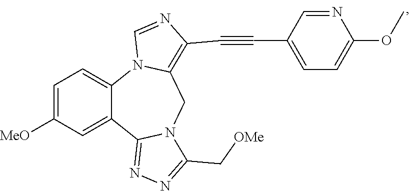 [SMILES: COCc1nnc2n1Cc1c(C#Cc3ccc(OC)nc3)ncn1-c1ccc(OC)cc1-2] |
| 8 | 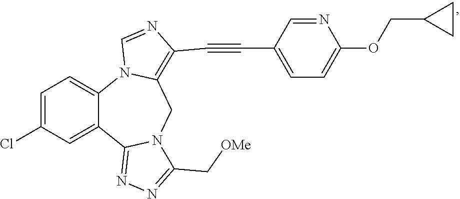 [SMILES: COCc1nnc2n1Cc1c(C#Cc3ccc(OCC4CC4)nc3)ncn1-c1ccc(Cl)cc1-2] |
| 9 | 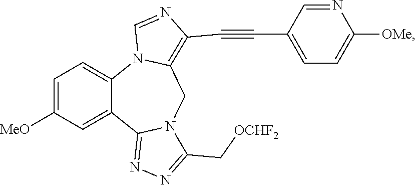 [SMILES: COc1ccc2c(c1)-c1nnc(COC(F)F)n1Cc1c(C#Cc3ccc(OC)nc3)ncn1-2] |
| 10 | 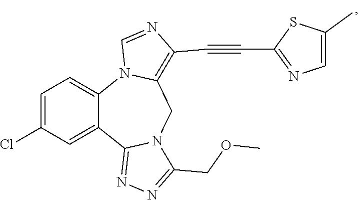 [SMILES: COCc1nnc2n1Cc1c(C#Cc3ncc(C)s3)ncn1-c1ccc(Cl)cc1-2] |
| 11 | 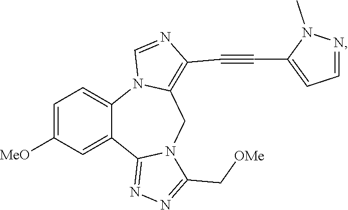 [SMILES: COCc1nnc2n1Cc1c(C#Cc3ccnn3C)ncn1-c1ccc(OC)cc1-2] |
| 12 | 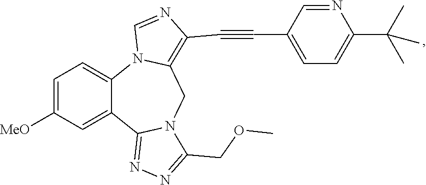 [SMILES: COCc1nnc2n1Cc1c(C#Cc3ccc(C(C)(C)C)nc3)ncn1-c1ccc(OC)cc1-2] |
| 13 | 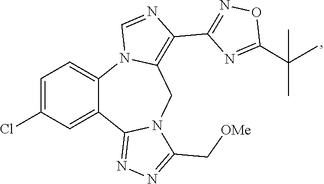 [SMILES: COCc1nnc2n1Cc1c(-c3noc(C(C)(C)C)n3)ncn1-c1ccc(Cl)cc1-2] |
| 14 | 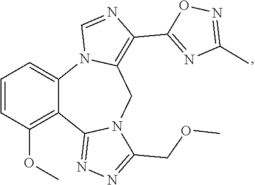 [SMILES: COCc1nnc2n1Cc1c(-c3nc(C)no3)ncn1-c1cccc(OC)c1-2] |
| 15 | 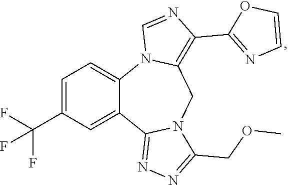 [SMILES: COCc1nnc2n1Cc1c(-c3ncco3)ncn1-c1ccc(C(F)(F)F)cc1-2] |
| 16 | 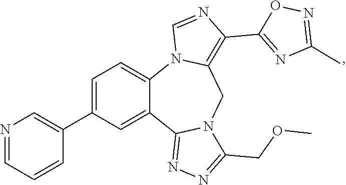 [SMILES: COCc1nnc2n1Cc1c(-c3nc(C)no3)ncn1-c1ccc(-c3cccnc3)cc1-2] |
| 17 | 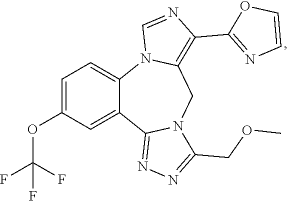 [SMILES: COCc1nnc2n1Cc1c(-c3ncco3)ncn1-c1ccc(OC(F)(F)F)cc1-2] |
| 18 | 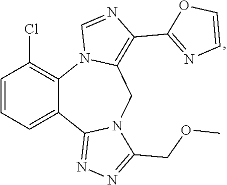 [SMILES: COCc1nnc2n1Cc1c(-c3ncco3)ncn1-c1c(Cl)cccc1-2] |
| 19 | 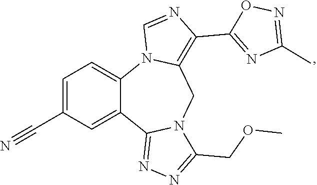 [SMILES: COCc1nnc2n1Cc1c(-c3nc(C)no3)ncn1-c1ccc(C#N)cc1-2] |
| 20 | 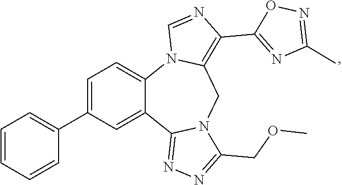 [SMILES: COCc1nnc2n1Cc1c(-c3nc(C)no3)ncn1-c1ccc(-c3ccccc3)cc1-2] |
| 21 | 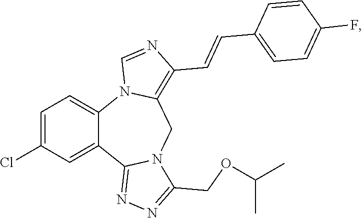 [SMILES: CC(C)OCc1nnc2n1Cc1c(/C=C/c3ccc(F)cc3)ncn1-c1ccc(Cl)cc1-2] |
| 22 | 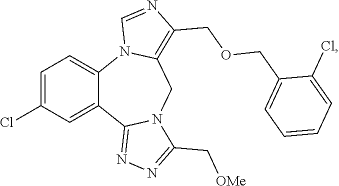 [SMILES: COCc1nnc2n1Cc1c(COCc3ccccc3Cl)ncn1-c1ccc(Cl)cc1-2] |
| 23 | 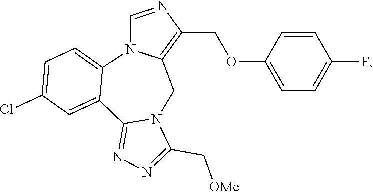 [SMILES: COCc1nnc2n1Cc1c(COc3ccc(F)cc3)ncn1-c1ccc(Cl)cc1-2] |
| 24 | 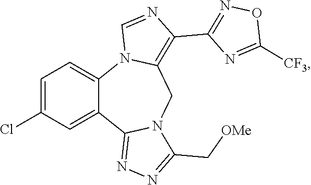 [SMILES: COCc1nnc2n1Cc1c(-c3noc(C(F)(F)F)n3)ncn1-c1ccc(Cl)cc1-2] |
| 25 | 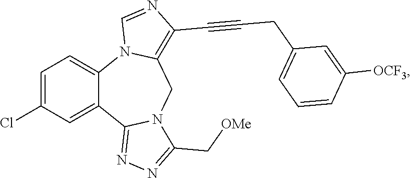 [SMILES: COCc1nnc2n1Cc1c(C#CCc3cccc(OC(F)(F)F)c3)ncn1-c1ccc(Cl)cc1-2] |
| 26 | 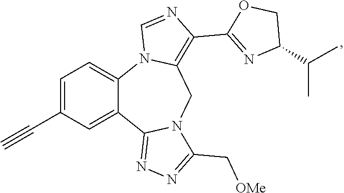 [SMILES: C#Cc1ccc2c(c1)-c1nnc(COC)n1Cc1c(C3=N[C@@H](C(C)C)CO3)ncn1-2] |
| 27 | 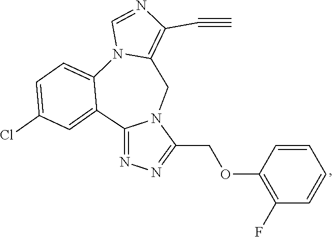 [SMILES: C#Cc1ncn2c1Cn1c(COc3ccccc3F)nnc1-c1cc(Cl)ccc1-2] |
| 28 | 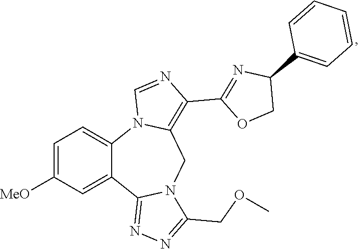 [SMILES: COCc1nnc2n1Cc1c(C3=N[C@@H](c4ccccc4)CO3)ncn1-c1ccc(OC)cc1-2] |
| 29 | 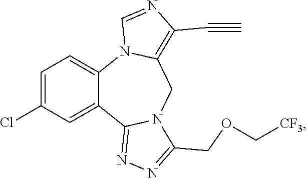 [SMILES: C#Cc1ncn2c1Cn1c(COCC(F)(F)F)nnc1-c1cc(Cl)ccc1-2] |
| 30 | 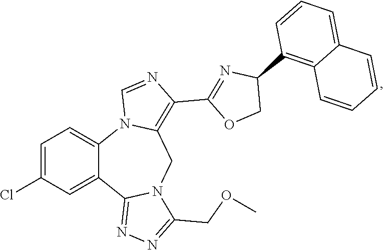 [SMILES: COCc1nnc2n1Cc1c(C3=N[C@@H](c4cccc5ccccc45)CO3)ncn1-c1ccc(Cl)cc1-2] |
| 31 | 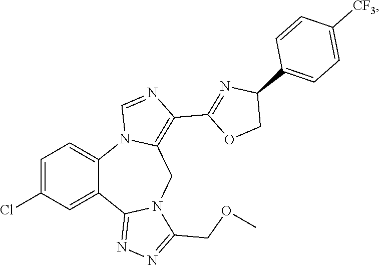 [SMILES: COCc1nnc2n1Cc1c(C3=N[C@@H](c4ccc(C(F)(F)F)cc4)CO3)ncn1-c1ccc(Cl)cc1-2] |
| 32 | 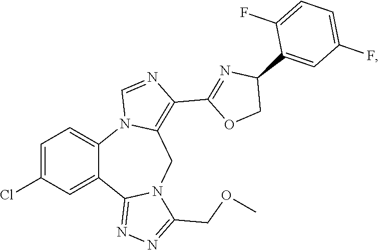 [SMILES: COCc1nnc2n1Cc1c(C3=N[C@@H](c4cc(F)ccc4F)CO3)ncn1-c1ccc(Cl)cc1-2] |
| 33 | 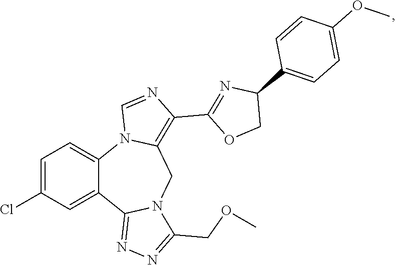 [SMILES: COCc1nnc2n1Cc1c(C3=N[C@@H](c4ccc(OC)cc4)CO3)ncn1-c1ccc(Cl)cc1-2] |
| 34 | 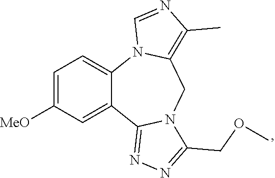 [SMILES: COCc1nnc2n1Cc1c(C)ncn1-c1ccc(OC)cc1-2] |
| 35 | 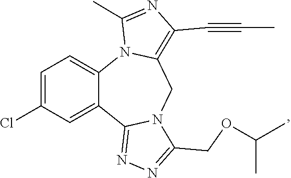 [SMILES: CC#Cc1nc(C)n2c1Cn1c(COC(C)C)nnc1-c1cc(Cl)ccc1-2] |
| 36 | 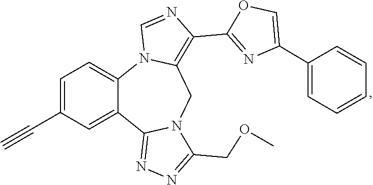 [SMILES: C#Cc1ccc2c(c1)-c1nnc(COC)n1Cc1c(-c3nc(-c4ccccc4)co3)ncn1-2] |
| 37 | 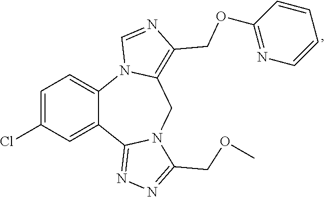 [SMILES: COCc1nnc2n1Cc1c(COc3ccccn3)ncn1-c1ccc(Cl)cc1-2] |
| 38 | 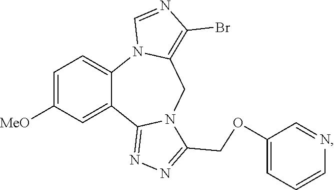 [SMILES: COc1ccc2c(c1)-c1nnc(COc3cccnc3)n1Cc1c(Br)ncn1-2] |
| 39 | 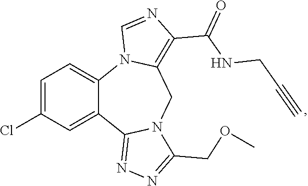 [SMILES: C#CCNC(=O)c1ncn2c1Cn1c(COC)nnc1-c1cc(Cl)ccc1-2] |
| 40 | 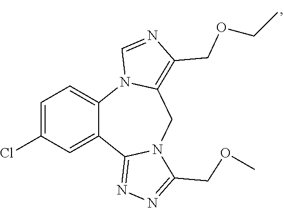 [SMILES: CCOCc1ncn2c1Cn1c(COC)nnc1-c1cc(Cl)ccc1-2] |
| 41 | 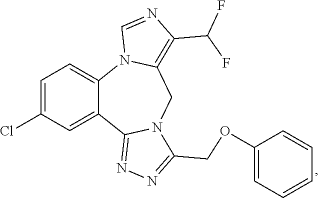 [SMILES: FC(F)c1ncn2c1Cn1c(COc3ccccc3)nnc1-c1cc(Cl)ccc1-2] |
| 42 | 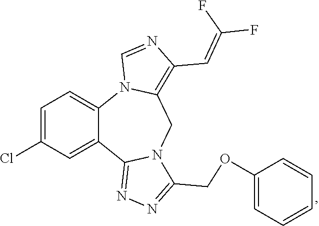 [SMILES: FC(F)=Cc1ncn2c1Cn1c(COc3ccccc3)nnc1-c1cc(Cl)ccc1-2] |
| 43 | 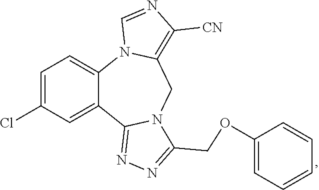 [SMILES: N#Cc1ncn2c1Cn1c(COc3ccccc3)nnc1-c1cc(Cl)ccc1-2] |
| 44 | 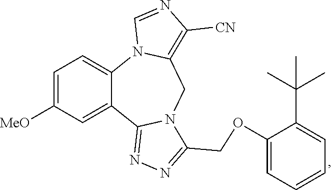 [SMILES: COc1ccc2c(c1)-c1nnc(COc3ccccc3C(C)(C)C)n1Cc1c(C#N)ncn1-2] |
| 45 | 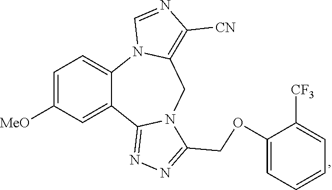 [SMILES: COc1ccc2c(c1)-c1nnc(COc3ccccc3C(F)(F)F)n1Cc1c(C#N)ncn1-2] |
| 46 | 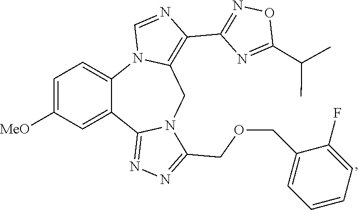 [SMILES: COc1ccc2c(c1)-c1nnc(COCc3ccccc3F)n1Cc1c(-c3noc(C(C)C)n3)ncn1-2] |
| 47 | 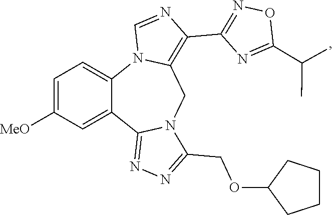 |
| 48 | 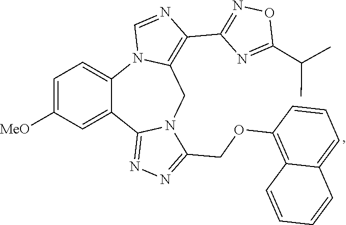 [SMILES: COc1ccc2c(c1)-c1nnc(COc3cccc4ccccc34)n1Cc1c(-c3noc(C(C)C)n3)ncn1-2] |
| 49 | 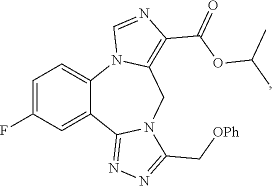 |
| 50 | 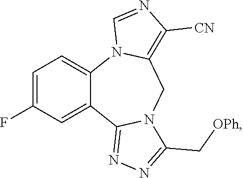 [SMILES: N#Cc1ncn2c1Cn1c(COc3ccccc3)nnc1-c1cc(F)ccc1-2] |
| 51 | 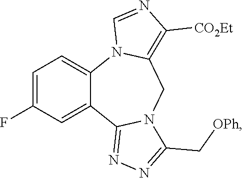 [SMILES: CCOC(=O)c1ncn2c1Cn1c(COc3ccccc3)nnc1-c1cc(F)ccc1-2] |
| 52 | 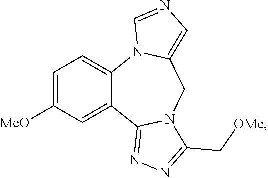 [SMILES: COCc1nnc2n1Cc1cncn1-c1ccc(OC)cc1-2] |
| 53 | 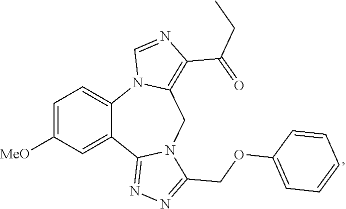 [SMILES: CCC(=O)c1ncn2c1Cn1c(COc3ccccc3)nnc1-c1cc(OC)ccc1-2] |
| 54 | 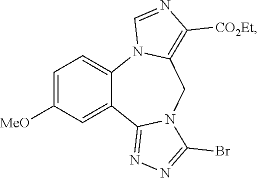 [SMILES: CCOC(=O)c1ncn2c1Cn1c(Br)nnc1-c1cc(OC)ccc1-2] |
| 55 | 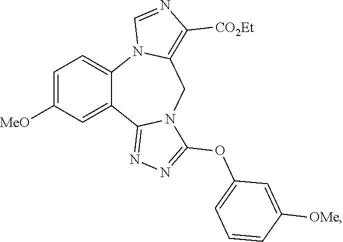 [SMILES: CCOC(=O)c1ncn2c1Cn1c(Oc3cccc(OC)c3)nnc1-c1cc(OC)ccc1-2] |
| 56 | 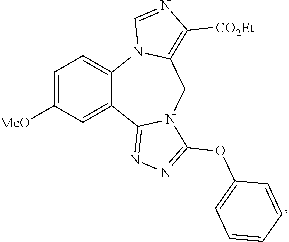 [SMILES: CCOC(=O)c1ncn2c1Cn1c(Oc3ccccc3)nnc1-c1cc(OC)ccc1-2] |
| 57 | 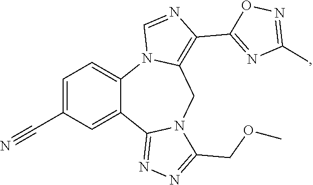 |
| 58 | 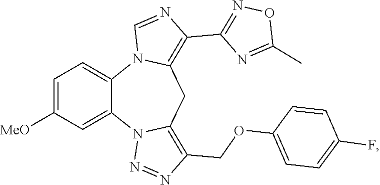 [SMILES: COc1ccc2c(c1)-n1nnc(COc3ccc(F)cc3)c1Cc1c(-c3noc(C)n3)ncn1-2] |
| 59 | 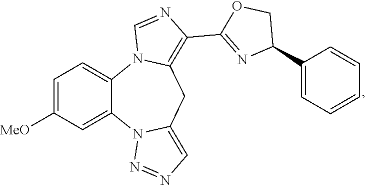 [SMILES: COc1ccc2c(c1)-n1nncc1Cc1c(C3=N[C@H](c4ccccc4)CO3)ncn1-2] |
| 60 | 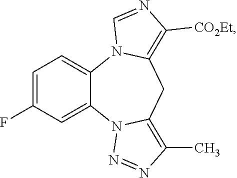 [SMILES: CCOC(=O)c1ncn2c1Cc1c(C)nnn1-c1cc(F)ccc1-2] |
| 61 | 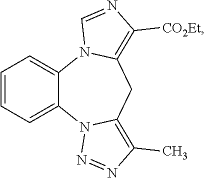 [SMILES: CCOC(=O)c1ncn2c1Cc1c(C)nnn1-c1ccccc1-2] |
| 62 | 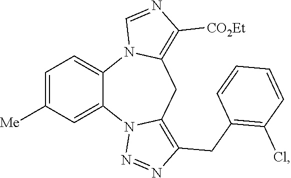 [SMILES: CCOC(=O)c1ncn2c1Cc1c(Cc3ccccc3Cl)nnn1-c1cc(C)ccc1-2] |
| 63 | 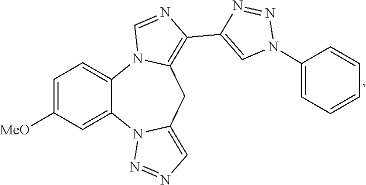 [SMILES: COc1ccc2c(c1)-n1nncc1Cc1c(-c3cn(-c4ccccc4)nn3)ncn1-2] |
| 64 | 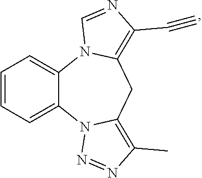 |
| 65 | 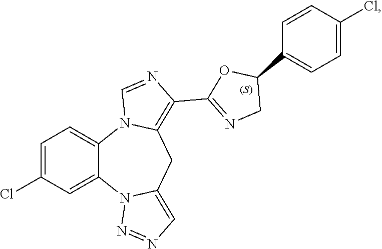 |
| 66 | 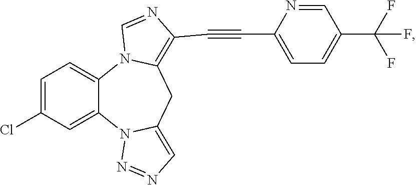 [SMILES: FC(F)(F)c1ccc(C#Cc2ncn3c2Cc2cnnn2-c2cc(Cl)ccc2-3)nc1] |
| 67 | 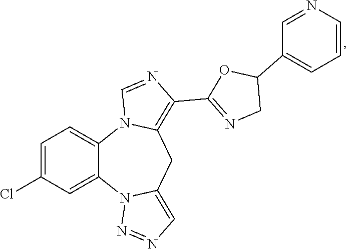 [SMILES: Clc1ccc2c(c1)-n1nncc1Cc1c(C3=NCC(c4cccnc4)O3)ncn1-2] |
| 68 | 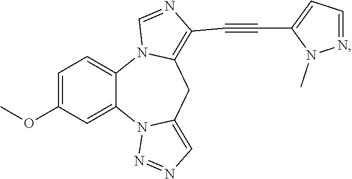 [SMILES: COc1ccc2c(c1)-n1nncc1Cc1c(C#Cc3ccnn3C)ncn1-2] |
| 69 | 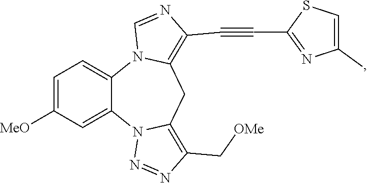 |
| 70 | 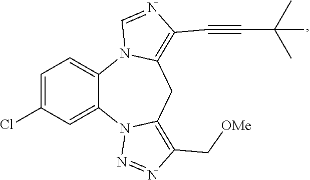 |
| 71 | 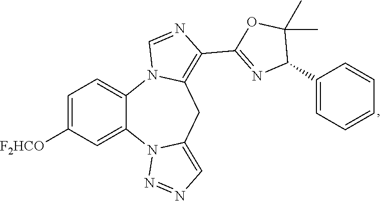 [SMILES: CC1(C)OC(c2ncn3c2Cc2cnnn2-c2cc(OC(F)F)ccc2-3)=N[C@H]1c1ccccc1] |
| 72 | 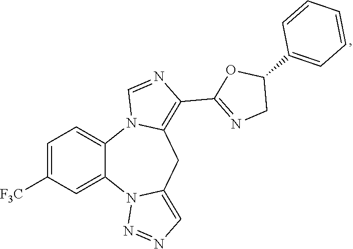 [SMILES: FC(F)(F)c1ccc2c(c1)-n1nncc1Cc1c(C3=NC[C@@H](c4ccccc4)O3)ncn1-2] |
| 73 | 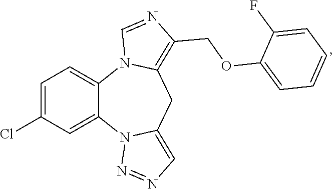 [SMILES: Fc1ccccc1OCc1ncn2c1Cc1cnnn1-c1cc(Cl)ccc1-2] |
| 74 | 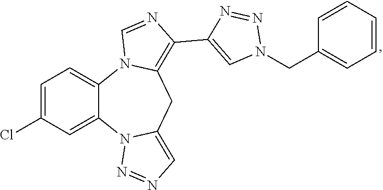 [SMILES: Clc1ccc2c(c1)-n1nncc1Cc1c(-c3cn(Cc4ccccc4)nn3)ncn1-2] |
| 75 | 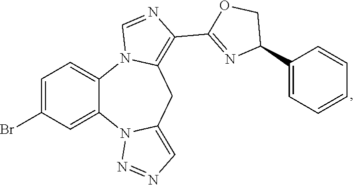 [SMILES: Brc1ccc2c(c1)-n1nncc1Cc1c(C3=N[C@H](c4ccccc4)CO3)ncn1-2] |
| 76 | 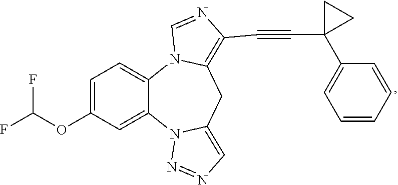 [SMILES: FC(F)Oc1ccc2c(c1)-n1nncc1Cc1c(C#CC3(c4ccccc4)CC3)ncn1-2] |
| 77 | 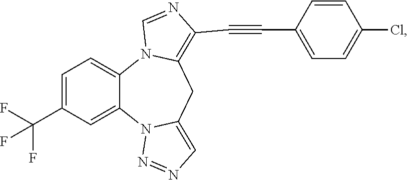 [SMILES: FC(F)(F)c1ccc2c(c1)-n1nncc1Cc1c(C#Cc3ccc(Cl)cc3)ncn1-2] |
| 78 | 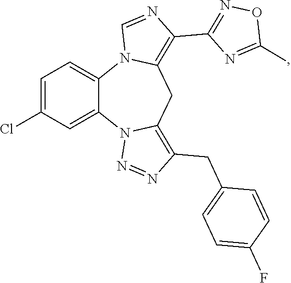 |
| 79 | 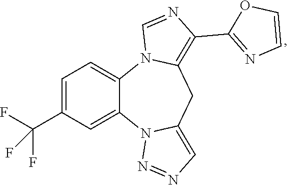 [SMILES: FC(F)(F)c1ccc2c(c1)-n1nncc1Cc1c(-c3ncco3)ncn1-2] |
| 80 | 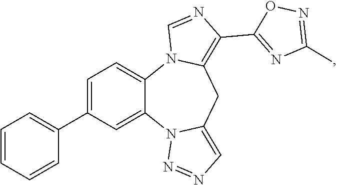 |
| 81 | 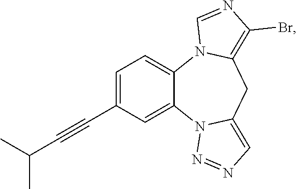 [SMILES: CC(C)C#Cc1ccc2c(c1)-n1nncc1Cc1c(Br)ncn1-2] |
| 82 | 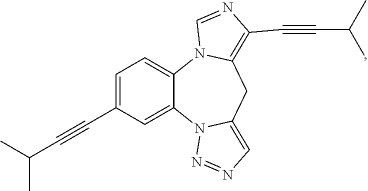 |
| 83 | 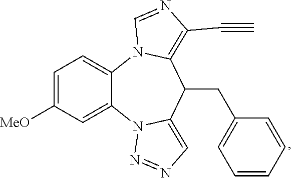 [SMILES: C#Cc1ncn2c1C(Cc1ccccc1)c1cnnn1-c1cc(OC)ccc1-2] |
| 84 | 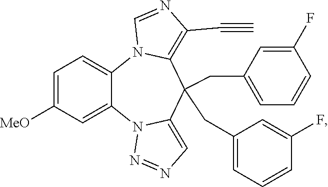 [SMILES: C#Cc1ncn2c1C(Cc1cccc(F)c1)(Cc1cccc(F)c1)c1cnnn1-c1cc(OC)ccc1-2] |
| 85 | 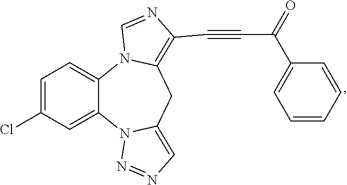 [SMILES: O=C(C#Cc1ncn2c1Cc1cnnn1-c1cc(Cl)ccc1-2)c1ccccc1] |
| 86 | 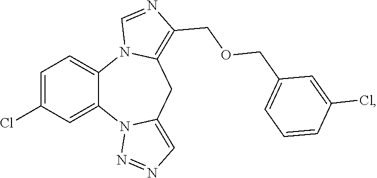 [SMILES: Clc1cccc(COCc2ncn3c2Cc2cnnn2-c2cc(Cl)ccc2-3)c1] |
| 87 | 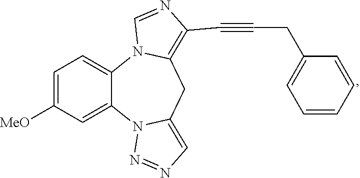 [SMILES: COc1ccc2c(c1)-n1nncc1Cc1c(C#CCc3ccccc3)ncn1-2] |
| 88 | 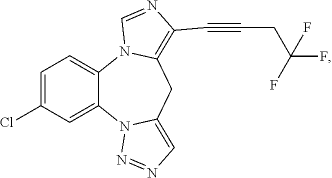 [SMILES: FC(F)(F)CC#Cc1ncn2c1Cc1cnnn1-c1cc(Cl)ccc1-2] |
| 89 | 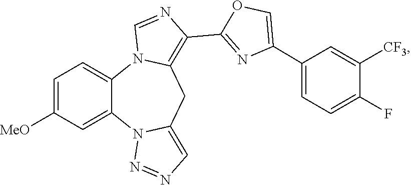 [SMILES: COc1ccc2c(c1)-n1nncc1Cc1c(-c3nc(-c4ccc(F)c(C(F)(F)F)c4)co3)ncn1-2] |
| 90 | 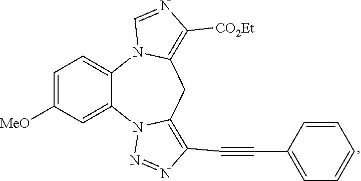 [SMILES: CCOC(=O)c1ncn2c1Cc1c(C#Cc3ccccc3)nnn1-c1cc(OC)ccc1-2] |
| 91 | 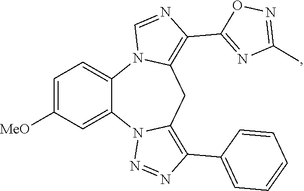 |
| 92 | 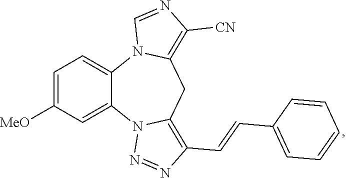 [SMILES: COc1ccc2c(c1)-n1nnc(/C=C/c3ccccc3)c1Cc1c(C#N)ncn1-2] |
| 93 | 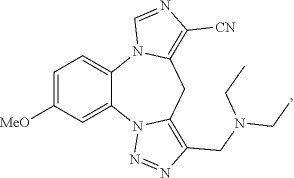 |
| 94 | 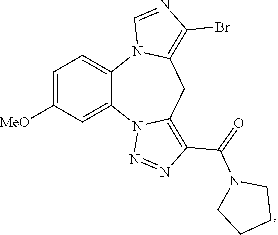 [SMILES: COc1ccc2c(c1)-n1nnc(C(=O)N3CCCC3)c1Cc1c(Br)ncn1-2] |
| 95 | 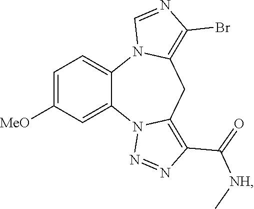 [SMILES: CNC(=O)c1nnn2c1Cc1c(Br)ncn1-c1ccc(OC)cc1-2] |
| 96 | 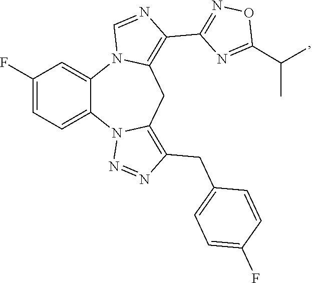 |
| 97 | 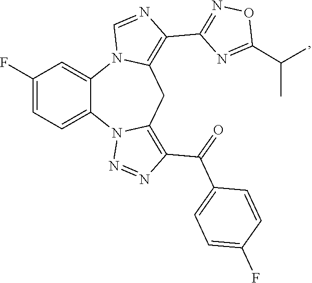 |
| 98 | 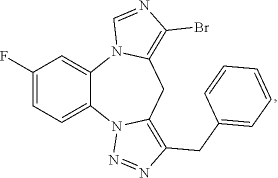 [SMILES: Fc1ccc2c(c1)-n1cnc(Br)c1Cc1c(Cc3ccccc3)nnn1-2] |
| 99 | 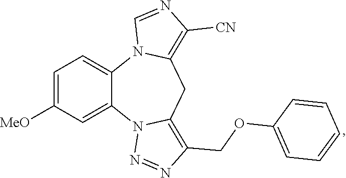 [SMILES: COc1ccc2c(c1)-n1nnc(COc3ccccc3)c1Cc1c(C#N)ncn1-2] |
| 100 | 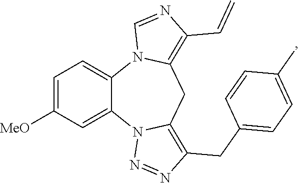 |
| 101 | 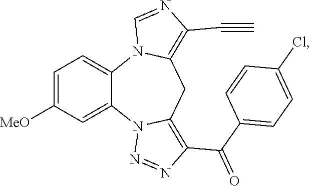 [SMILES: C#Cc1ncn2c1Cc1c(C(=O)c3ccc(Cl)cc3)nnn1-c1cc(OC)ccc1-2] |
| 102 | 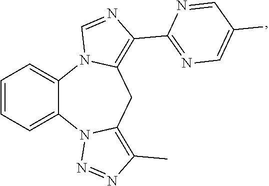 |
| 103 | 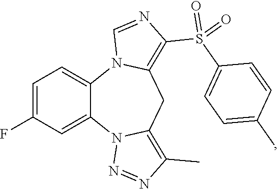 |
| 104 | 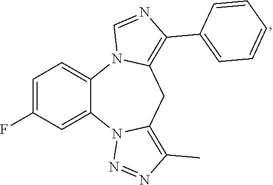 [SMILES: Cc1nnn2c1Cc1c(-c3ccccc3)ncn1-c1ccc(F)cc1-2] |
| 105 | 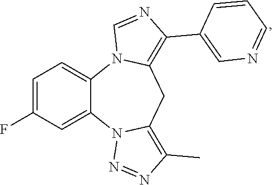 [SMILES: Cc1nnn2c1Cc1c(-c3cccnc3)ncn1-c1ccc(F)cc1-2] |
| 106 | 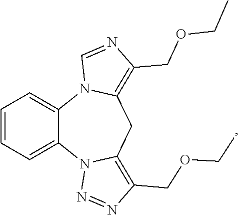 |
| 107 | 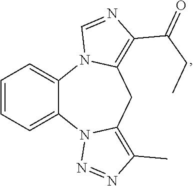 [SMILES: CCC(=O)c1ncn2c1Cc1c(C)nnn1-c1ccccc1-2] |
| 108 | 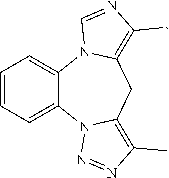 |
| 109 | 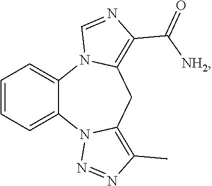 [SMILES: Cc1nnn2c1Cc1c(C(N)=O)ncn1-c1ccccc1-2] |
| 110 | 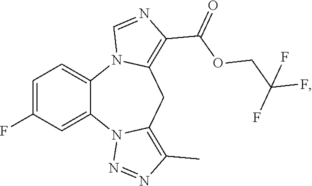 [SMILES: Cc1nnn2c1Cc1c(C(=O)OCC(F)(F)F)ncn1-c1ccc(F)cc1-2] |
| 111 | 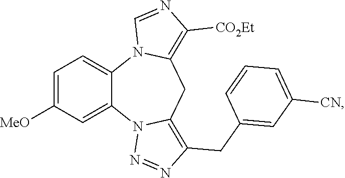 [SMILES: CCOC(=O)c1ncn2c1Cc1c(Cc3cccc(C#N)c3)nnn1-c1cc(OC)ccc1-2] |
| 112 | 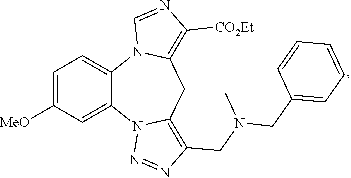 [SMILES: CCOC(=O)c1ncn2c1Cc1c(CN(C)Cc3ccccc3)nnn1-c1cc(OC)ccc1-2] |
| 113 | 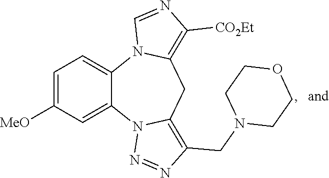 [SMILES: CCOC(=O)c1ncn2c1Cc1c(CN3CCOCC3)nnn1-c1cc(OC)ccc1-2] |
| 114 | 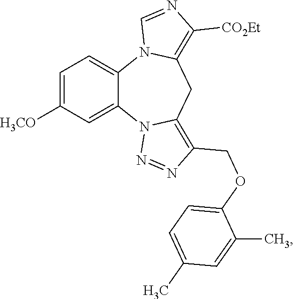 [SMILES: CCOC(=O)c1ncn2c1Cc1c(COc3ccc(C)cc3C)nnn1-c1cc(OC)ccc1-2] |

or a pharmaceutically acceptable salt, hydrate, solvate, polymorph, or isomer of any of the foregoing. In some embodiments, the compound of Formula I, or the pharmaceutically acceptable salt, hydrate, solvate, polymorph, or isomer thereof, is Compound 1, or a pharmaceutically acceptable salt, hydrate, solvate, polymorph, or isomer thereof.

The compounds of the disclosure also include crystalline forms of the GABA_{A} α5 receptor agonists, or pharmaceutically acceptable salts, hydrates, solvates, or isomers thereof, that may be useful in the methods, uses, combinations, pharmaceutical compositions, combinations for use, or pharmaceutical compositions for use of the disclosure. Such crystalline forms include Compound 1, Form A (polymorph crystalline form); Compound 1, Form B (polymorph crystalline form); Compound 1, Form C (solvate crystalline form); Compound 1, Form E (polymorph crystalline form); and Compound 1, Form F (hydrate crystalline form). In some embodiments, the methods, uses, combinations, pharmaceutical compositions, combinations for use, or pharmaceutical compositions for use of the disclosure may comprise one or more crystalline forms selected from the group consisting of Compound 1, Form A; Compound 1, Form B; Compound 1, Form C; Compound 1, Form E; and Compound 1, Form F. In some embodiments, the crystalline form is Compound 1, Form A. In some embodiments, the crystalline form is Compound 1, Form B. In some embodiments, the crystalline form is Compound 1, Form C. In some embodiments, the crystalline form is Compound 1, Form E. In some embodiments, the crystalline form is Compound 1, Form F.

The compounds of the disclosure also include crystalline forms of the GABA_{A} α5 receptor agonists, or pharmaceutically acceptable salts, hydrates, solvates, or isomers thereof, that may be useful in the methods, uses, combinations, pharmaceutical compositions, combinations for use, or pharmaceutical compositions for use of the disclosure. Such crystalline forms include Compound 1, Form A (polymorph crystalline form); Compound 1, Form B (polymorph crystalline form); Compound 1, Form C (solvate crystalline form); Compound 1, Form E (polymorph crystalline form); and Compound 1, Form F (hydrate crystalline form). In some embodiments, the methods, uses, combinations, pharmaceutical compositions, combinations for use, or pharmaceutical compositions for use of the disclosure may comprise one or more crystalline forms selected from the group consisting of Compound 1, Form A; Compound 1, Form B; Compound 1, Form C; Compound 1, Form E; and Compound 1, Form F. In some embodiments, the crystalline form is Compound 1, Form A. In some embodiments, the crystalline form is Compound 1, Form B. In some embodiments, the crystalline form is Compound 1, Form C. In some embodiments, the crystalline form is Compound 1, Form E. In some embodiments, the crystalline form is Compound 1, Form F. Such crystalline forms include Compound 1 which has been found to exist in at least 5 crystalline polymorphic forms (i.e., Form A, Form B, Form C, Material D, Form E and Form F). In some embodiments, the methods, uses, combinations, pharmaceutical compositions, combinations for use, or pharmaceutical compositions for use may comprise crystalline form of Compound 1, wherein the crystalline form corresponds to Form A, Form B, Form C, Material D, Form E or Form F, or any mixtures thereof. In some embodiments, the crystalline form is an anhydrous crystalline form of Compound 1, wherein the crystalline form corresponds to Form A, Form B, Material D or Form E. In some embodiments, the crystalline form is a solvated crystalline form of Compound 1, wherein the crystalline form corresponds to Form C or Form F. In certain such embodiments, the solvated crystalline form of Compound 1 is a methanolate or a hydrate.

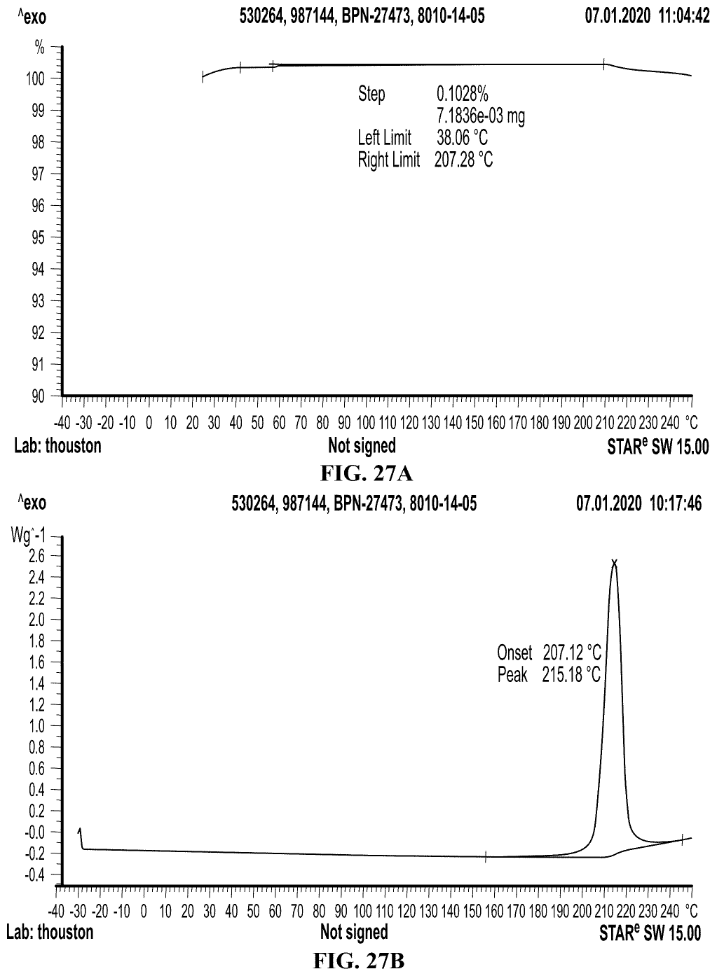

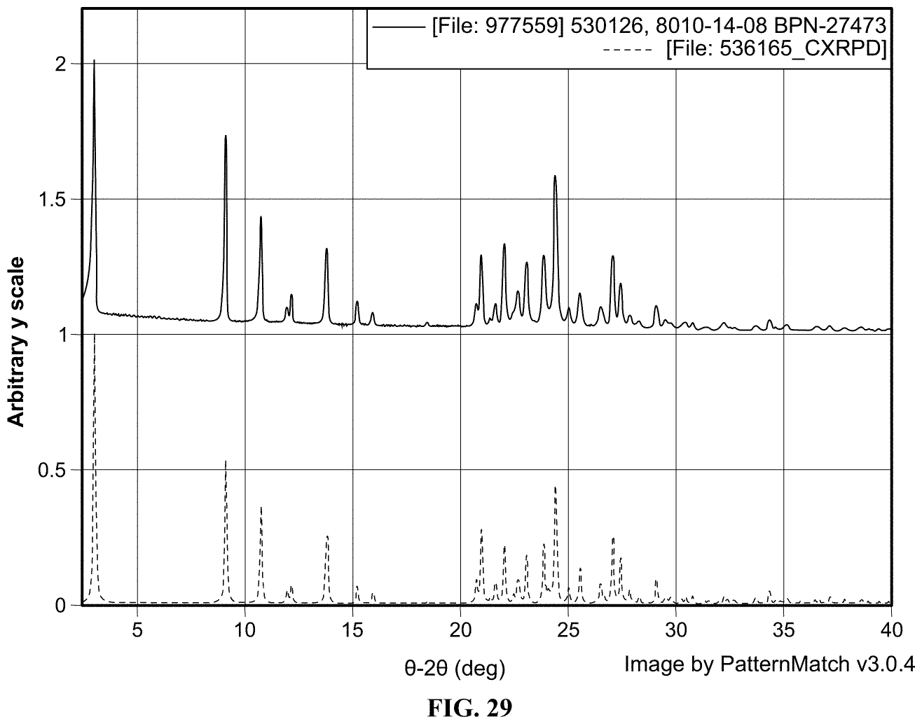

In some embodiments, the crystalline form is Compound 1, Form A characterized by an x-ray powder diffraction (XRPD) pattern substantially as set forth in FIG. 29 having at least one of 3.0 and/or 21.0 degrees 2θ±0.2 degrees 2θ and further comprising one or more of additional peaks selected from 9.1, 10.7, 13.8, 22.0, 23.1, 23.9, 24.4, and 27.1 degrees 2θ±0.2 degrees 2θ. In some embodiments, the crystalline form is Compound 1, Form A characterized by a C2/c single crystal x-ray diffraction space group. In some embodiments, the crystalline form is Compound 1, Form A characterized by a single crystal x-ray diffraction unit cell having the parameters: a=58.1415(14) Å, b=4.03974(8) Å, c=17.1204(3) Å, α=90°, β=90.261(2°), γ=90°, V=4021.15(14) Å³. In some embodiments, the crystalline form is Compound 1, Form A characterized by a differential scanning calorimetry (DSC) curve substantially as set forth in FIG. 27B. In some embodiments, the crystalline form is Compound 1, Form A characterized by a differential scanning calorimetry (DSC) curve having an exotherm with an onset at about 207° C. In some embodiments, the crystalline form is Compound 1, Form A characterized by two or more of: (a) an x-ray powder diffraction (XRPD) pattern substantially as set forth in FIG. 29; (b) a C2/c single crystal x-ray diffraction space group; (c) a single crystal x-ray diffraction unit cell having the parameters: a=58.1415(14) Å, b=4.03974(8) Å, c=17.1204(3) Å, α=90°, β=90.261(2°), γ=90°, V=4021.15(14) Å³; (d) a differential scanning calorimetry (DSC) curve substantially as set forth in FIG. 27B; and (e) a differential scanning calorimetry (DSC) curve having an exotherm with an onset at about 207° C.

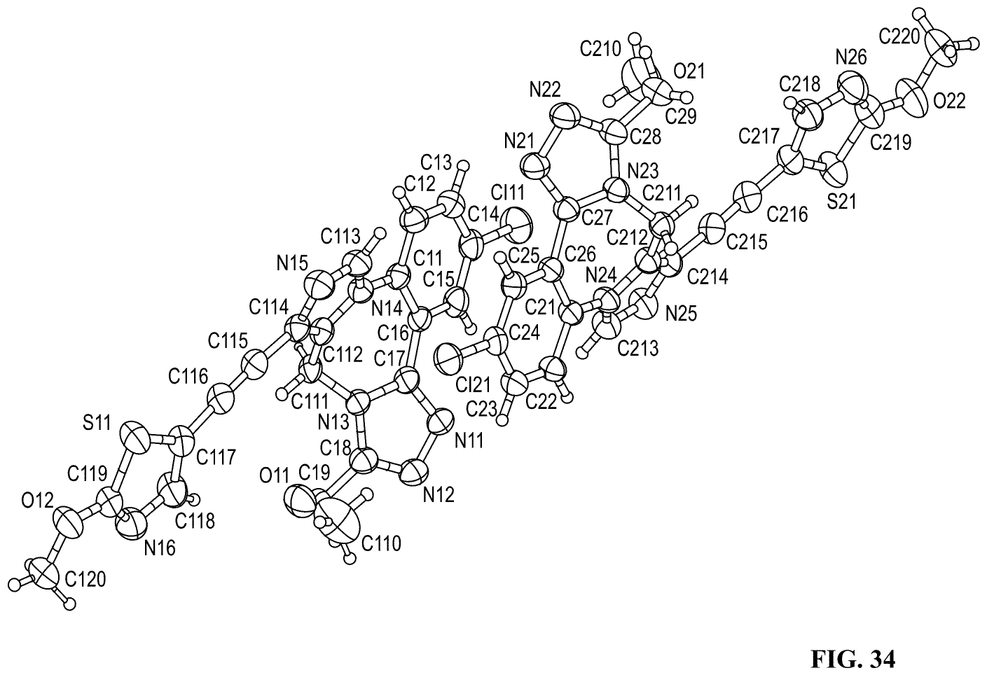

In some embodiments, the crystalline form is Compound 1, Form B characterized by an x-ray powder diffraction (XRPD) pattern substantially as set forth in FIG. 34, having at least one peak selected from 13.0 and/or 15.3 degrees 2θ±0.2 degrees 2θ and further comprising one or more of additional peaks selected from 7.0, 9.3, 10.2, 10.4, 12.5, 13.6, 14.0, 22.0, 23.0, 23.6, and 27.3 degrees 2θ±0.2 degrees 2θ. In some embodiments, the crystalline form is Compound 1, Form B characterized by a monoclinic single crystal x-ray diffraction unit cell. In some embodiments, the crystalline form is Compound 1, Form B characterized by a single crystal x-ray diffraction formula unit volume of about 497 Å³. In some embodiments, the crystalline form is Compound 1, Form B characterized by a differential scanning calorimetry (DSC) curve having an exotherm with an onset at about 190° C. In some embodiments, the crystalline form is Compound 1, Form B characterized by two or more of: (a) an x-ray powder diffraction (XRPD) pattern substantially as set forth in FIG. 34; (b) a single crystal x-ray diffraction formula unit volume of about 497 Å³; and (c) a differential scanning calorimetry (DSC) curve having an exotherm with an onset at about 190° C.

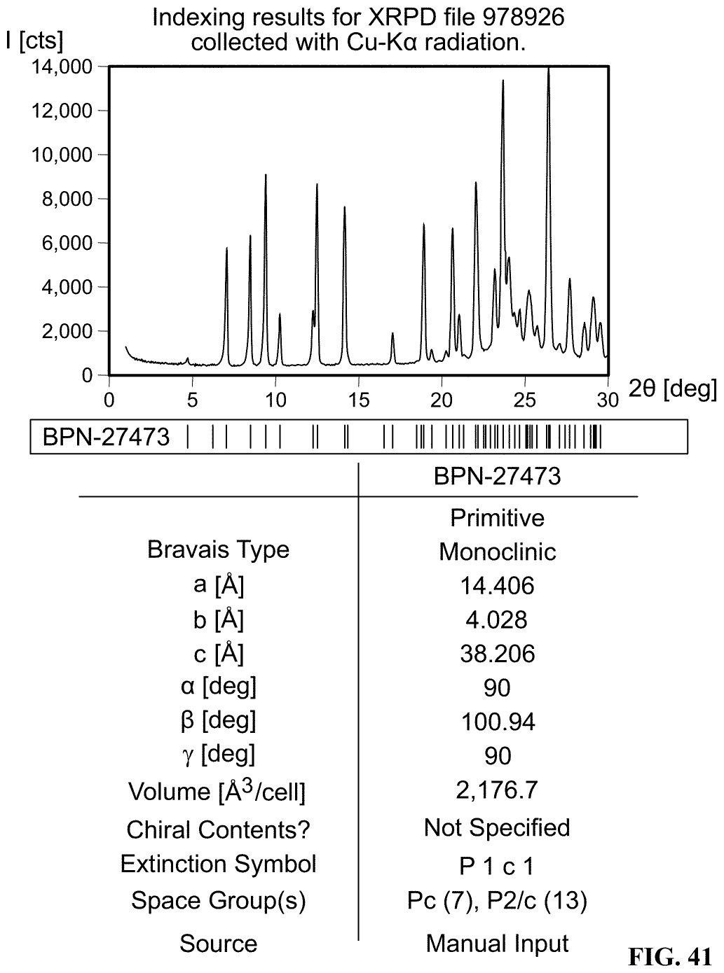

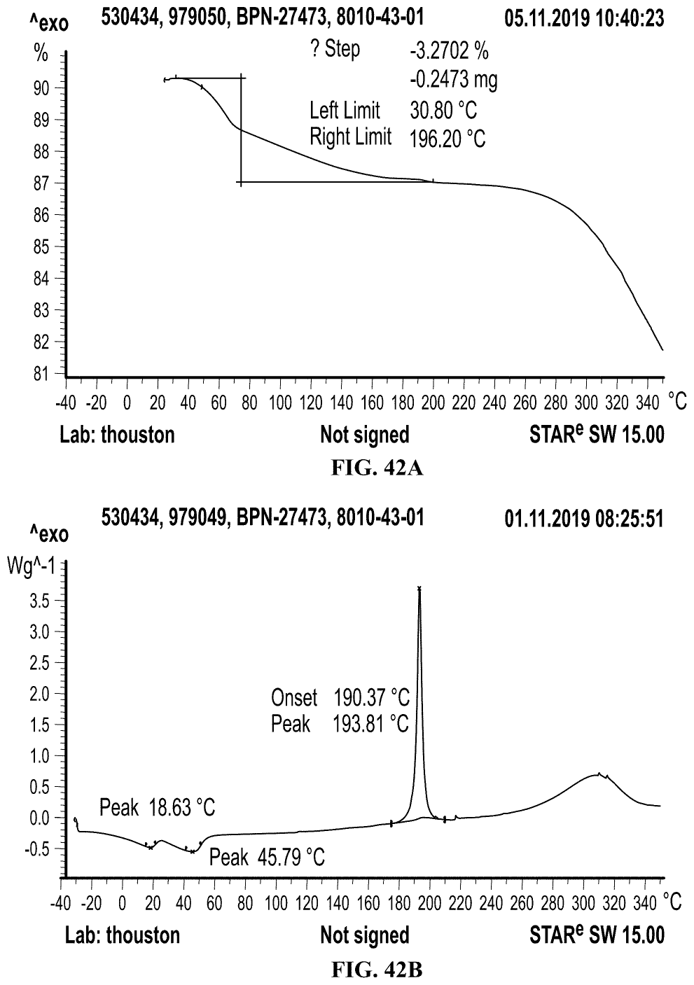

In some embodiments, the crystalline form is Compound 1, Form C characterized by an x-ray powder diffraction (XRPD) pattern substantially as set forth in FIG. 41, having at least one peak selected from 8.5, and/or 18.9 degrees 2θ±0.2 degrees 2θ and further comprising one or more of additional peaks selected from 7.1, 9.4, 10.3, 12.3, 12.5, 14.2, 20.7, 22.1, 23.2, 23.7, 24.0, and 26.4 degrees 2θ±0.2 degrees 2θ. In some embodiments, the crystalline form is Compound 1, Form C characterized by a monoclinic single crystal x-ray diffraction unit cell. In some embodiments, the crystalline form is Compound 1, Form C characterized by a single crystal x-ray diffraction formula unit volume of about 544 Å³. In some embodiments, the crystalline form is Compound 1, Form C characterized by a differential scanning calorimetry (DSC) curve substantially as set forth in FIG. 42B. In some embodiments, the crystalline form is Compound 1, Form C characterized by a differential scanning calorimetry (DSC) curve having an exotherm with an onset at about 190° C. In some embodiments, the crystalline form is Compound 1, Form C characterized by two or more of: (a) an x-ray powder diffraction (XRPD) pattern substantially as set forth in FIG. 41; (b) a monoclinic single crystal x-ray diffraction unit cell; (c) a single crystal x-ray diffraction formula unit volume of about 544 Å³; (d) a differential scanning calorimetry (DSC) curve substantially as set forth in FIG. 42B; and (e) a differential scanning calorimetry (DSC) curve having an exotherm with an onset at about 190° C.

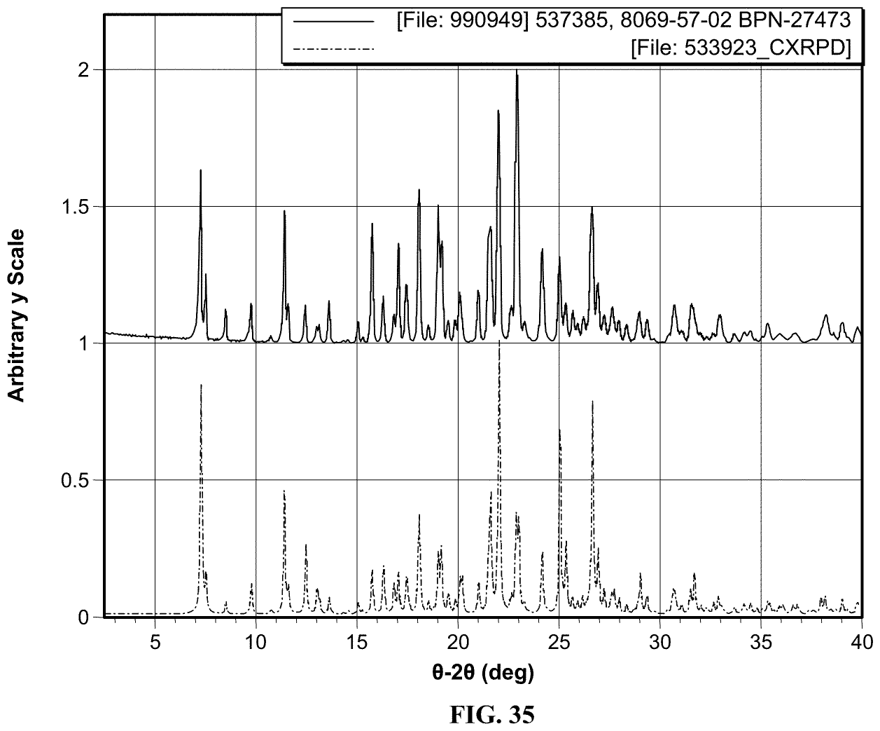

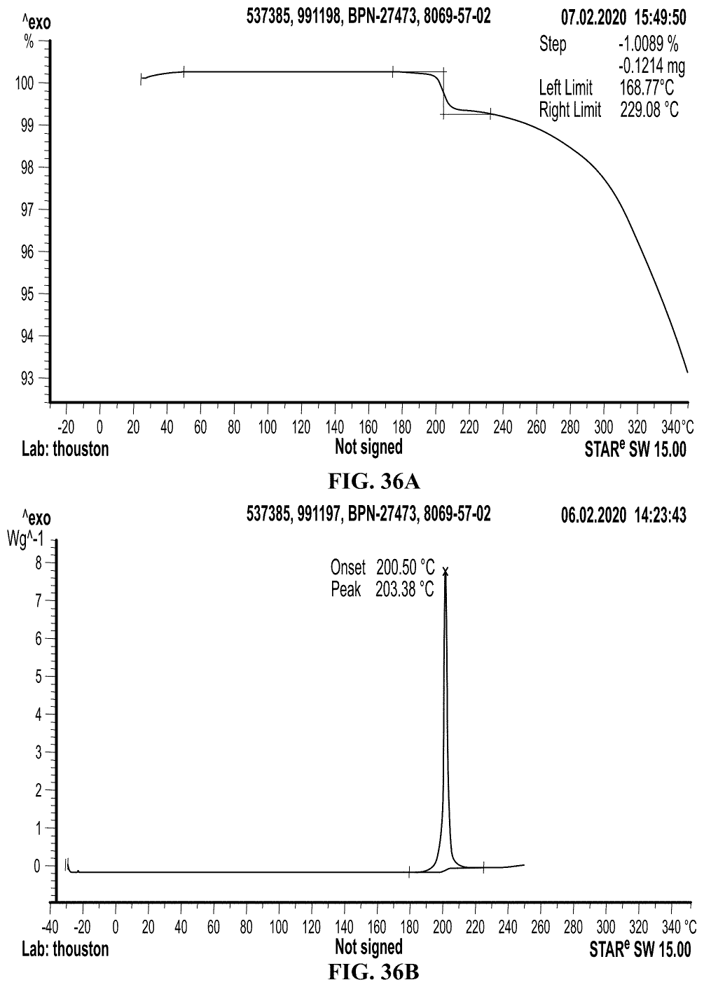

In some embodiments, the crystalline form is Compound 1, Form E characterized by an x-ray powder diffraction (XRPD) pattern substantially as set forth in FIG. 35, having at least one peak selected from 11.4, 18.1, and/or 21.6 degrees 2θ±0.2 degrees 2θ and further comprising one or more of additional peaks selected from 7.2, 22.0, 23.0, 24.2, 25.0, and 26.6 degrees 2θ±0.2 degrees 2θ. In some embodiments, the crystalline form is Compound 1, Form E characterized by a P2₁/n single crystal x-ray diffraction space group. In some embodiments, the crystalline form is Compound 1, Form E characterized by a single crystal x-ray diffraction unit cell having the parameters: a=11.83974(13) Å, b=23.5195(2) Å, c=14.48807(17) Å, α=90°, β=101.5333(11°), γ=90°, V=3952.96(7) Å³. In some embodiments, the crystalline form is Compound 1, Form E characterized by a differential scanning calorimetry (DSC) curve substantially as set forth in FIG. 36B. In some embodiments, the crystalline form is Compound 1, Form E characterized by a differential scanning calorimetry (DSC) curve having an exotherm with an onset at about 201° C. In some embodiments, the crystalline form is Compound 1, Form E characterized by two or more of (a) an x-ray powder diffraction (XRPD) pattern substantially as set forth in FIG. 35; (b) a P2₁/n single crystal x-ray diffraction space group; (c) a single crystal x-ray diffraction unit cell having the parameters: a=11.83974(13) Å, b=23.5195(2) Å, c=14.48807(17) Å, α=90°, β=101.5333(11°), γ=90°, V=3952.96(7) Å³; (d) a differential scanning calorimetry (DSC) curve substantially as set forth in FIG. 36B; and (e) a differential scanning calorimetry (DSC) curve having an exotherm with an onset at about 201° C.

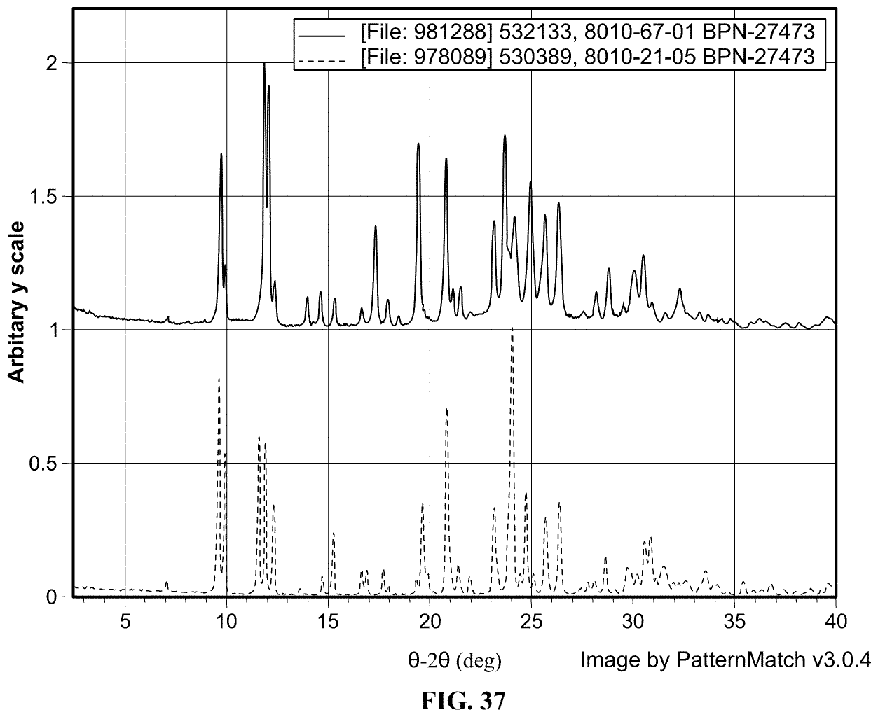

In some embodiments, the crystalline form is Compound 1, Form F characterized by an x-ray powder diffraction (XRPD) pattern substantially as set forth in FIG. 37, having at least one peak selected from 9.9, 11.9, 17.3, 19.4, and/or 25.7 degrees 2θ±0.2 degrees 2θ and further comprising one or more of additional peaks selected from 9.7, 12.1, 20.8, 23.2, 23.7, 24.2, 25.0, and 26.4 degrees 2θ±0.2 degrees 2θ. In some embodiments, the crystalline form is Compound 1, Form F characterized by a triclinic single crystal x-ray diffraction unit cell. In some embodiments, the crystalline form is Compound 1, Form F characterized by a single crystal x-ray diffraction formula unit volume of about 511 Å³. In some embodiments, the crystalline form is Compound 1, Form F characterized by a differential scanning calorimetry (DSC) curve having an exotherm at above about 120° C. In some embodiments, the crystalline form is Compound 1, Form F characterized by two or more of: (a) an x-ray powder diffraction (XRPD) pattern substantially as set forth in FIG. 37; (b) a triclinic single crystal x-ray diffraction unit cell; (c) a single crystal x-ray diffraction formula unit volume of about 511 Å³; and (d) a differential scanning calorimetry (DSC) curve having an exotherm at above about 120° C.

The disclosure includes pharmaceutically acceptable salts of the compounds described herein. Representative “pharmaceutically acceptable salts” include, but are not limited to, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, sethionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

“Pharmaceutically acceptable salt” also includes both acid and base addition salts. “Pharmaceutically acceptable acid addition salt” may refer to those salts which retain the biological effectiveness and properties of the free bases, which are not biologically or otherwise undesirable, and which are formed with inorganic acids such as, but are not limited to, hydrohalic acids, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, hydroxyacetic acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, l-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” may refer to those salts which retain the biological effectiveness and properties of the free acids, which are not biologically or otherwise undesirable. These salts may be prepared from addition of an inorganic base or an organic base to the free acid. Salts derived from inorganic bases include, but are not limited to, alkali and earth alkaline metal salts, sodium, potassium, lithium, ammonium, calcium, magnesium, iron, zinc, copper, manganese, aluminum salts, and the like. Salts derived from organic bases include, but are not limited to, salts of N-methyl-D-glucamine; primary, secondary, and tertiary amines; substituted amines including naturally occurring substituted amines, cyclic amines; basic ion exchange resins; isopropylamine; trimethylamine; diethylamine; triethylamine; tripropylamine; diethanolamine; ethanolamine; deanol; 2-dimethylaminoethanol; 2-diethylaminoethanol; dicyclohexylamine; amino acids; lysine; arginine; histidine; caffeine; procaine; hydrabamine; choline; betaine; benethamine; benzathine; ethylenediamine; glucosamine; methylglucamine; theobromine; triethanolamine; tromethamine; purines; piperazine; piperidine; N-ethylpiperidine; polyamine resins; and the like.

Conversely, said salt forms can be converted into the free forms by treatment with an appropriate base or acid.

SV2A inhibitors, or the pharmaceutically acceptable salts, polymorphs, or isomers thereof, described herein or GABA_{A} α5 receptor agonists, or pharmaceutically acceptable salts, polymorphs, or isomers thereof, described herein can be in the form of a solvate, which is included within the scope of the present disclosure. Such solvates include, for example, hydrates, alcoholates and the like. See, e.g., WO 01/062726.

As used herein, “hydrate” may refer to a combination of water with a compound wherein the water retains its molecular state as water and is either absorbed, adsorbed or contained within a crystal lattice of the compound.

As used herein, “polymorph” may refer to different crystalline forms of the same compound and other solid state molecular forms including pseudo-polymorphs, such as hydrates (e.g., bound water present in the crystalline structure) and solvates (e.g., bound solvents other than water) of the same compound. Different crystalline polymorphs have different crystal structures due to a different packing of the molecules in the lattice. This results in a different crystal symmetry and/or unit cell parameters, which directly influences its physical properties such the X-ray diffraction characteristics of crystals or powders. A different polymorph, for example, will in general diffract at a different set of angles and will give different values for the intensities. Therefore, X-ray powder diffraction can be used to identify different polymorphs, or a solid form that comprises more than one polymorph, in a reproducible and reliable way. Crystalline polymorphic forms are of interest to the pharmaceutical industry and especially to those involved in the development of suitable dosage forms. If the polymorphic form is not held constant during clinical or stability studies, the exact dosage form used or studied may not be comparable from one lot to another. It is also desirable to have processes for producing a compound with the selected polymorphic form in high purity when the compound is used in clinical studies or commercial products since Impurities present may produce undesired toxicological effects. Certain polymorphic forms may exhibit enhanced thermodynamic stability or may be more readily manufactured in high purity in large quantities, and thus are more suitable for inclusion in pharmaceutical formulations. Certain polymorphs may display other advantageous physical properties such as lack of hygroscopic tendencies, improved solubility, and enhanced rates of dissolution due to different lattice energies.

This application contemplates all the isomers of the compounds of the disclosure. “Isomer,” as used herein, includes optical isomers (such as stereoisomers, e.g., enantiomers and diastereoisomers), Z (zusammen) or E (entgegen) isomers, and tautomers. Many of the compounds useful in the methods, uses, combinations, pharmaceutical compositions, combinations for use, or pharmaceutical compositions for use of the disclosure have at least one stereogenic center in their structure. This stereogenic center may be present in a R or a S configuration, said R and S notation is used in correspondence with the rules described in Pure Appl. Chem. (1976), 45, 11-30. The disclosure also relates to all stereoisomeric forms such as enantiomeric and diastereoisomeric forms of the compounds or mixtures thereof (including all possible mixtures of stereoisomers). See, e.g., WO 01/062726. Furthermore, certain compounds which contain alkenyl groups may exist as Z (zusammen) or E (entgegen) isomers. In each instance, the disclosure includes both mixture and separate individual isomers. Multiple substituents on a piperidinyl or the azepanyl ring can also stand in either cis or trans relationship to each other with respect to the plane of the piperidinyl or the azepanyl ring. Some of the compounds may also exist in tautomeric forms. Such forms, although not explicitly indicated, are intended to be included within the scope of the present disclosure. With respect to the methods, uses, combinations, pharmaceutical compositions, combinations for use, or pharmaceutical compositions for use of the present disclosure, reference to a compound or compounds is intended to encompass that compound in each of its possible isomeric forms and mixtures thereof unless the particular isomeric form is referred to specifically. See, e.g., WO 01/062726.

“Aliphatic” as used herein may refer to a straight chained or branched alkyl, alkenyl or alkynyl. It is understood that alkenyl or alkynyl embodiments need at least two carbon atoms in the aliphatic chain. Aliphatic groups typically contain from 1 (or 2) to 12 carbons, such as from 1 (or 2) to 4 carbons.

“Aryl” as used herein may refer to a monocyclic or bicyclic carbocyclic aromatic ring system. Aryl as used herein includes a (C6-C12)-aryl-. For example, aryl as used herein can be a C6-C10 monocyclic or C8-C12 bicyclic carbocyclic aromatic ring system. In some embodiments, aryl as used herein can be a (C6-C10)-aryl-. Phenyl (or Ph) is an example of a monocyclic aromatic ring system. Bicyclic aromatic ring systems include systems wherein both rings are aromatic, e.g., naphthyl, and systems wherein only one of the two rings is aromatic, e.g., tetralin.

“Heterocyclic” as used herein may refer to a monocyclic or bicyclic non-aromatic ring system having 1 to 4 heteroatom or heteroatom groups selected from O, N, NH, S, SO, or SO₂ in a chemically stable arrangement. Heterocyclic as used herein includes a 3- to 12-membered heterocyclyl- having 1-4 heteroatoms independently selected from O, N, NH, S, SO, or SO₂. For example, heterocyclic as used herein can be a 3- to 10-membered monocyclic or 8- to 12-membered bicyclic non-aromatic ring system having 1 to 4 heteroatom or heteroatom groups selected from O, N, NH, S, SO, or SO₂ in a chemically stable arrangement. In some embodiments, heterocyclic as used herein can be a 3- to 10-membered heterocyclyl- having 1-4 heteroatoms independently selected from O, N, NH, S, SO, or SO₂. In a bicyclic non-aromatic ring system embodiment of “heterocyclyl,” one or both rings may contain said heteroatom or heteroatom groups. In another bicyclic “heterocyclyl” embodiment, one of the two rings may be aromatic. In yet another heterocyclic ring system embodiment, a non-aromatic heterocyclic ring may optionally be fused to an aromatic carbocycle.

Examples of heterocyclic rings include 3-1H-benzimidazol-2-one, 3-(l-alkyl)-benzimidazol-2-one, 2-tetrahydrofuranyl, 3-tetrahydrofuranyl, 2-tetrahydrothiophenyl, 3-tetrahydrothiophenyl, 2-morpholino, 3-morpholino, 4-morpholino, 2-thiomorpholino, 3-thiomorpholino, 4-thiomorpholino, 1-pyrrolidinyl, 2-pyrrolidinyl, 3-pyrrolidinyl, 1-tetrahydropiperazinyl, 2-tetrahydropiperazinyl, 3-tetrahydropiperazinyl, 1-piperidinyl, 2-piperidinyl, 3-piperidinyl, 1-pyrazolinyl, 3-pyrazolinyl, 4-pyrazolinyl, 5-pyrazolinyl, 1-piperidinyl, 2-piperidinyl, 3-piperidinyl, 4-piperidinyl, 2-thiazolidinyl, 3-thiazolidinyl, 4-thiazolidinyl, 1-imidazolidinyl, 2-imidazolidinyl, 4-imidazolidinyl, 5-imidazolidinyl, indolinyl, tetrahydroquinolinyl, tetrahydroisoquinolinyl, benzothiolane, benzodithiane, and 1,3-dihydro-imidazol-2-one.

“Heteroaryl” as used herein may refer to a monocyclic or bicyclic aromatic ring system having 1 to 4 heteroatom or heteroatom groups selected from O, N, NH or S in a chemically stable arrangement. Heteroaryl as used herein includes a 5- to 12-membered heteroaryl having 1-4 heteroatoms independently selected from O, N, NH or S. In some embodiments, heteroaryl as used herein can be a 5- to 10-membered heteroaryl having 1-4 heteroatoms independently selected from O, N, NH or S. For example, heteroaryl as used herein can be a 5- to 10-membered monocyclic or 8- to 12-membered bicyclic aromatic ring system having 1 to 4 heteroatom or heteroatom groups selected from O, N, NH or S in one or both rings in a chemically stable arrangement. In such a bicyclic aromatic ring system embodiment of “heteroaryl”:
both rings are aromatic; and
one or both rings may contain said heteroatom or heteroatom groups.

Examples of heteroaryl rings include 2-furanyl, 3-furanyl, N-imidazolyl, 2-imidazolyl, 4-imidazolyl, 5-imidazolyl, benzimidazolyl, 3-isoxazolyl, 4-isoxazolyl, 5-isoxazolyl, 2-oxazolyl, 4-oxazolyl, 5-oxazolyl, N-pyrrolyl, 2-pyrrolyl, 3-pyrrolyl, 2-pyridyl, 3-pyridyl, 4-pyridyl, 2-pyrimidinyl, 4-pyrimidinyl, 5-pyrimidinyl, pyridazinyl (e.g., 3-pyridazinyl), 2-thiazolyl, 4-thiazolyl, 5-thiazolyl, tetrazolyl (e.g., 5-tetrazolyl), triazolyl (e.g., 2-triazolyl and 5-triazolyl), 2-thienyl, 3-thienyl, benzofuryl, benzothiophenyl, indolyl (e.g., 2-indolyl), pyrazolyl (e.g., 2-pyrazolyl), isothiazolyl, 1,2,3-oxadiazolyl, 1,2,5-oxadiazolyl, 1,2,4-oxadiazolyl, 1,2,3-triazolyl, 1,2,3-thiadiazolyl, 1,3,4-thiadiazolyl, 1,2,5-thiadiazolyl, purinyl, pyrazinyl, 1,3,5-triazinyl, quinolinyl (e.g., 2-quinolinyl, 3-quinolinyl, 4-quinolinyl), and isoquinolinyl (e.g., 1-isoquinolinyl, 3-isoquinolinyl, or 4-isoquinolinyl).

“Cycloalkyl or cycloalkenyl” may refer to a monocyclic or fused or bridged bicyclic carbocyclic ring system that is not aromatic. For example, cycloalkyl or cycloalkenyl as used herein can be a C3-C10 monocyclic or fused or bridged C8-C12 bicyclic carbocyclic ring system that is not aromatic. Cycloalkenyl rings have one or more units of unsaturation. Preferred cycloalkyl or cycloalkenyl groups include cyclopropyl, cyclobutyl, cyclopentyl, cyclohexyl, cyclohexenyl, cycloheptyl, cycloheptenyl, norbornyl, adamantyl and decalinyl.

“Heteroaralkyl” may refer to an alkyl in which a heteroaryl group is substituted for an alkyl H atom. For example, the alkyl group may be any straight chain hydrocarbon, and can include from 1 to 12 carbon atoms (e.g., methyl, ethyl, propyl, butyl, pentyl, hexyl, heptyl, octyl, nonyl, decyl, undecyl and dodecyl), wherein said alkyl group can be substituted with any heteroaryl group, including but not limited to, 2-furanyl, 3-furanyl, N-imidazolyl, 2-imidazolyl, 4-imidazolyl, 5-imidazolyl, benzimidazolyl, 3-isoxazolyl, 4-isoxazolyl, 5-isoxazolyl, 2-oxazolyl, 4-oxazolyl, 5-oxazolyl, N-pyrrolyl, 2-pyrrolyl, 3-pyrrolyl, 2-pyridyl, 3-pyridyl, 4-pyridyl, 2-pyrimidinyl, 4-pyrimidinyl, 5-pyrimidinyl, pyridazinyl (e.g., 3-pyridazinyl), 2-thiazolyl, 4-thiazolyl, 5-thiazolyl, tetrazolyl (e.g., 5-tetrazolyl), triazolyl (e.g., 2-triazolyl and 5-triazolyl), 2-thienyl, 3-thienyl, benzofuryl, benzothiophenyl, indolyl (e.g., 2-indolyl), pyrazolyl (e.g., 2-pyrazolyl), isothiazolyl, 1,2,3-oxadiazolyl, 1,2,5-oxadiazolyl, 1,2,4-oxadiazolyl, 1,2,3-triazolyl, 1,2,3-thiadiazolyl, 1,3,4-thiadiazolyl, 1,2,5-thiadiazolyl, purinyl, pyrazinyl, 1,3,5-triazinyl, quinolinyl (e.g., 2-quinolinyl, 3-quinolinyl, 4-quinolinyl), and isoquinolinyl (e.g., 1-isoquinolinyl, 3-isoquinolinyl, or 4-isoquinolinyl.

When a substituted moiety is described without indicating the atom via which such moiety is bonded to a substituent, then the substituent may be bonded via any appropriate atom in such moiety. For example, for a substituted 5- to 10-membered heteroaryl, a substituent on the heteroaryl can be bonded to any of the ring-forming atoms of the heteroaryl ring that are substitutable (i.e., atoms bound to one or more hydrogen atoms).

When a bond to a substituent is shown to cross a bond connecting two atoms in a ring, then such substituent may be bonded to any of the ring-forming atoms in that ring that are substitutable (i.e., atoms bound to one or more hydrogen atoms), unless otherwise specified or otherwise implicit from the context. For example, when a R group is defined as a pyridine, and said pyridine is depicted as follows:

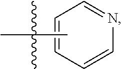

the pyridine ring may be bound to the benzodiazepine derivative through any one of the ring carbon atoms in the pyridine ring. As another example, when a R group is defined as a pyrazole, and said pyrazole is depicted as follows:

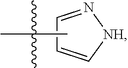

the pyrazole ring may be bound to the benzodiazepine derivative through any one of the ring carbon atoms of the pyrazole ring, or to the sp³ N-atom.

As used herein, the carbon atom designations may have the indicated integer and any intervening integer. For example, the number of carbon atoms in a (C1-C4)-alkyl group is 1, 2, 3, or 4. It should be understood that these designations refer to the total number of atoms in the appropriate group. For example, in a (C3-C10)-heterocyclyl the total number of carbon atoms and heteroatoms is 3 (as in aziridine), 4, 5, 6 (as in morpholine), 7, 8, 9, or 10.

### Pharmaceutical Compositions and Combinations of the Disclosure

In one aspect, the disclosure provides pharmaceutical compositions and combinations comprising an SV2A inhibitor (as used throughout any one or more of the SV2A inhibitors of this disclosure for example, insome embodiments the SV2A inhibitor is levetiracetam, in some embodiments the SV2A inhibitor is brivaracetam, and in some embodiments the SV2A inhibitor is seletracetam), or the pharmaceutically acceptable salt, hydrate, solvate, isomer, or polymorph in combination with the GABA_{A} α5 receptor agonist (as used throughout any one or more of the GABA_{A} α5 receptor agonists of the disclosure, for example, in some embodiments one of a compound of Formula I, a compound of Formula II, or a compound of Formula IV; one of the specified compounds 1-740, one of Compounds 1-114; or one of Compound 1, Form A; Compound 1, Form B; Compound 1, Form C; Compound 1, Form E; or Compound 1, Form F), or the pharmaceutically acceptable salt, hydrate, solvate, polymorph, or isomer thereof. In particular, in some embodiments, the pharmaceutical compositions and combinations of this disclosure comprise levetiracetam and one of the specific GABA_{A} α5 receptor agonists referred to above, brivaracetam and one of the specific GABA_{A} α5 receptor agonists referred to above, and seletracetam and one of the specific GABA_{A} α5 receptor agonists referred to above. For example, in some embodiments of this disclosure the pharmaceutical composition or combination thereof may comprise, levetiracetam and Compound 1, brivaracetam and Compound 1, brivaracetam and compound 17, and so on.

The disclosure provides for pharmaceutical compositions comprising the compounds disclosed herein. In some embodiments, the pharmaceutical composition of the disclosure comprises an SV2A inhibitor (e.g., levetiracetam, brivaracetam, or seletracetam), or a pharmaceutically acceptable salt, hydrate, solvate, polymorph, or isomer thereof. In some embodiments, the pharmaceutical composition of the disclosure comprises a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof. In some embodiments, the pharmaceutical composition of the disclosure comprises both an SV2A inhibitor (e.g., levetiracetam, brivaracetam, or seletracetam), or a pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof. In some embodiments, the pharmaceutical composition of the disclosure comprises levetiracetam, or a pharmaceutically acceptable salt, hydrate, solvate, polymorph, or isomer thereof. In some embodiments, the pharmaceutical composition of the disclosure comprises levetiracetam, or a pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof. In some embodiments, the pharmaceutical composition of the disclosure comprises brivaracetam, or a pharmaceutically acceptable salt, hydrate, solvate, polymorph, or isomer thereof. In some embodiments, the pharmaceutical composition of the disclosure comprises brivaracetam, or a pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof. In some embodiments, the pharmaceutical composition of the disclosure comprises seletracetam, or a pharmaceutically acceptable salt, hydrate, solvate, polymorph, or isomer thereof. In some embodiments, the pharmaceutical composition of the disclosure comprises seletracetam, or a pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof. In some embodiments, the GABA_{A} α5 receptor agonist, or the pharmaceutically acceptable salt, hydrate, solvate, polymorph, or isomer thereof, is selected from the group consisting of: a compound of Formula I, or a pharmaceutically acceptable salt, hydrate, solvate, polymorph, or isomer thereof; a compound of Formula II, or a pharmaceutically acceptable salt, hydrate, solvate, polymorph, or isomer thereof; and a compound of Formula IV, or a pharmaceutically acceptable salt, hydrate, solvate, polymorph, or isomer thereof. In some embodiments, the GABA_{A} α5 receptor agonist, or the pharmaceutically acceptable salt, hydrate, solvate, polymorph, or isomer thereof, is a compound of Formula I, or a pharmaceutically acceptable salt, hydrate, solvate, polymorph, or isomer thereof. In some embodiments, the GABA_{A} α5 receptor agonist, or the pharmaceutically acceptable salt, hydrate, solvate, polymorph, or isomer thereof, is a compound of Formula II, or a pharmaceutically acceptable salt, hydrate, solvate, polymorph, or isomer thereof. In some embodiments, the GABA_{A} α5 receptor agonist, or the pharmaceutically acceptable salt, hydrate, solvate, polymorph, or isomer thereof, is a compound of Formula IV, or a pharmaceutically acceptable salt, hydrate, solvate, polymorph, or isomer thereof. In some embodiments, the compound of Formula I, or the pharmaceutically acceptable salt, hydrate, solvate, polymorph, or isomer thereof, is selected from the group consisting of one or more of Compounds 1-114, or a pharmaceutically acceptable salt, hydrate, solvate, polymorph, or isomer of any of the foregoing. In some embodiments, the compound of Formula I, or the pharmaceutically acceptable salt, hydrate, solvate, polymorph, or isomer thereof, is Compound 1, or a pharmaceutically acceptable salt, hydrate, solvate, polymorph, or isomer thereof. In some embodiments, the pharmaceutical composition of the disclosure may comprise one or more crystalline forms selected from the group consisting of one or more of Compound 1, Form A; Compound 1, Form B; Compound 1, Form C; Compound 1, Form E; and Compound 1, Form F. In some embodiments, the crystalline form is Compound 1, Form A.

The disclosure also provides for combinations comprising the compounds and pharmaceutical compositions disclosed herein. In some embodiments, the combination of the disclosure comprises an SV2A inhibitor (e.g., levetiracetam, brivaracetam, or seletracetam), or a pharmaceutically acceptable salt, hydrate, solvate, polymorph, or isomer thereof. In some embodiments, the combination of the disclosure comprises a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof. In some embodiments, the combination of the disclosure comprises both an SV2A inhibitor (e.g., levetiracetam, brivaracetam, or seletracetam), or a pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof. In certain such embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are packaged together. In some embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are packaged separately. Combinations of the disclosure also encompass formulations of the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, together in one formulation or in separate formulations.

In some embodiments, the combination of the disclosure comprises levetiracetam, or a pharmaceutically acceptable salt, hydrate, solvate, polymorph, or isomer thereof. In some embodiments, the combination of the disclosure comprises levetiracetam, or a pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof. In some embodiments, the combination of the disclosure comprises brivaracetam, or a pharmaceutically acceptable salt, hydrate, solvate, polymorph, or isomer thereof. In some embodiments, the combination of the disclosure comprises brivaracetam, or a pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof. In some embodiments, the combination of the disclosure comprises seletracetam, or a pharmaceutically acceptable salt, hydrate, solvate, polymorph, or isomer thereof. In some embodiments, the combination of the disclosure comprises seletracetam, or a pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof. In some embodiments, the GABA_{A} α5 receptor agonist, or the pharmaceutically acceptable salt, hydrate, solvate, polymorph, or isomer thereof, is selected from the group consisting of: a compound of Formula I, or a pharmaceutically acceptable salt, hydrate, solvate, polymorph, or isomer thereof; a compound of Formula II, or a pharmaceutically acceptable salt, hydrate, solvate, polymorph, or isomer thereof; and a compound of Formula IV, or a pharmaceutically acceptable salt, hydrate, solvate, polymorph, or isomer thereof. In some embodiments, the GABA_{A} α5 receptor agonist, or the pharmaceutically acceptable salt, hydrate, solvate, polymorph, or isomer thereof, is a compound of Formula I, or a pharmaceutically acceptable salt, hydrate, solvate, polymorph, or isomer thereof. In some embodiments, the GABA_{A} α5 receptor agonist, or the pharmaceutically acceptable salt, hydrate, solvate, polymorph, or isomer thereof, is a compound of Formula II, or a pharmaceutically acceptable salt, hydrate, solvate, polymorph, or isomer thereof. In some embodiments, the GABA_{A} α5 receptor agonist, or the pharmaceutically acceptable salt, hydrate, solvate, polymorph, or isomer thereof, is a compound of Formula IV, or a pharmaceutically acceptable salt, hydrate, solvate, polymorph, or isomer thereof. In some embodiments, the compound of Formula I, or the pharmaceutically acceptable salt, hydrate, solvate, polymorph, or isomer thereof, is selected from the group consisting of one of Compounds 1-114, or a pharmaceutically acceptable salt, hydrate, solvate, polymorph, or isomer of any of the foregoing. In some embodiments, the compound of Formula I, or the pharmaceutically acceptable salt, hydrate, solvate, polymorph, or isomer thereof, is Compound 1, or a pharmaceutically acceptable salt, hydrate, solvate, polymorph, or isomer thereof. In some embodiments, the combination of the disclosure may comprise one or more crystalline forms selected from the group consisting of one of Compound 1, Form A; Compound 1, Form B; Compound 1, Form C; Compound 1, Form E; and Compound 1, Form F. In some embodiments, the crystalline form is Compound 1, Form A.

In some embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are together in one formulation. In some embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are separate. In some embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are packaged together. In some embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are packaged separately.

In some embodiments, the combination of the disclosure comprises a pharmaceutical composition comprising an SV2A inhibitor (e.g., levetiracetam, brivaracetam, or seletracetam), or a pharmaceutically acceptable salt, hydrate, solvate, polymorph, or isomer thereof. In some embodiments, the combination of the disclosure comprises a pharmaceutical composition comprising a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof. In some embodiments, the combination of the disclosure comprises a pharmaceutical composition comprising both an SV2A inhibitor (e.g., levetiracetam, brivaracetam, or seletracetam), or a pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof. In some embodiments, the combination of the disclosure comprises a pharmaceutical composition comprising an SV2A inhibitor (e.g., levetiracetam, brivaracetam, or seletracetam), or a pharmaceutically acceptable salt, hydrate, solvate, polymorph, or isomer thereof (i.e., a first pharmaceutical composition), and a pharmaceutical composition comprising a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof (i.e., a second pharmaceutical composition). In certain such embodiments, the first and second pharmaceutical compositions are formulated separately. In certain such embodiments, the first and second pharmaceutical compositions are packaged together. In some embodiments, the first and second pharmaceutical compositions are packaged separately. In some embodiments, the first and second pharmaceutical compositions are formulated together.

In some embodiments, the combination of the disclosure comprises a pharmaceutical composition comprising levetiracetam, or a pharmaceutically acceptable salt, hydrate, solvate, polymorph, or isomer thereof. In some embodiments, the combination of the disclosure comprises a pharmaceutical composition comprising both levetiracetam, or a pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof. In some embodiments, the combination of the disclosure comprises a pharmaceutical composition comprising levetiracetam, or a pharmaceutically acceptable salt, hydrate, solvate, polymorph, or isomer thereof (i.e., a first pharmaceutical composition), and a pharmaceutical composition comprising a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof (i.e., a second pharmaceutical composition). In some embodiments, the combination of the disclosure comprises a pharmaceutical composition comprising brivaracetam, or a pharmaceutically acceptable salt, hydrate, solvate, polymorph, or isomer thereof. In some embodiments, the combination of the disclosure comprises a pharmaceutical composition comprising both brivaracetam, or a pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof. In some embodiments, the combination of the disclosure comprises a pharmaceutical composition comprising brivaracetam, or a pharmaceutically acceptable salt, hydrate, solvate, polymorph, or isomer thereof (i.e., a first pharmaceutical composition), and a pharmaceutical composition comprising a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof (i.e., a second pharmaceutical composition). In some embodiments, the combination of the disclosure comprises a pharmaceutical composition comprising seletracetam, or a pharmaceutically acceptable salt, hydrate, solvate, polymorph, or isomer thereof. In some embodiments, the combination of the disclosure comprises a pharmaceutical composition comprising both seletracetam, or a pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof. In some embodiments, the combination of the disclosure comprises a pharmaceutical composition comprising seletracetam, or a pharmaceutically acceptable salt, hydrate, solvate, polymorph, or isomer thereof (i.e., a first pharmaceutical composition), and a pharmaceutical composition comprising a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof (i.e., a second pharmaceutical composition). In some embodiments, the first and second pharmaceutical compositions are formulated separately. In certain such embodiments, the first and second pharmaceutical compositions are packaged together. In some embodiments, the first and second pharmaceutical compositions are packaged separately. In some embodiments, the first and second pharmaceutical compositions are formulated together.

In some embodiments, the pharmaceutical composition or combination (or a component thereof, such as a SV2A inhibitor (e.g., levetiracetam, brivaracetam, or seletracetam), or a pharmaceutically acceptable salt, hydrate, solvate, polymorph, or isomer thereof, and/or a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof) is in a solid form. In some embodiments, the pharmaceutical composition or combination (or a component thereof) is in a liquid form. In some embodiments, the pharmaceutical composition or combination (or a component thereof) is in an aqueous solution. In some embodiments, the pharmaceutical composition or combination (or a component thereof) is in a suspension form. In some embodiments, the pharmaceutical composition or combination (or a component thereof) is in a unit dosage form. In some embodiments, the pharmaceutical composition or combination (or a component thereof) is in a capsule or tablet form. In some embodiments, the pharmaceutical composition or combination (or a component thereof) is for oral administration.

A “component” of a combination of the disclosure may include a SV2A inhibitor (e.g., levetiracetam, brivaracetam, or seletracetam), or a pharmaceutically acceptable salt, hydrate, solvate, polymorph, or isomer thereof, and/or a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof. A “component” of a combination of the disclosure may also include a pharmaceutical composition comprising a SV2A inhibitor (e.g., levetiracetam, brivaracetam, or seletracetam), or a pharmaceutically acceptable salt, hydrate, solvate, polymorph, or isomer thereof, and/or a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof. A “component” of a combination of the disclosure may further include other agents, such as agents that serve to enhance and/or complement the effectiveness of the SV2A inhibitor (e.g., levetiracetam, brivaracetam, or seletracetam), or the pharmaceutically acceptable salt, hydrate, solvate, polymorph, or isomer thereof, and/or the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof.

In some embodiments, the SV2A inhibitor (e.g., levetiracetam, brivaracetam, or seletracetam), or the pharmaceutically acceptable salt, hydrate, solvate, polymorph, or isomer thereof, is formulated together with the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof. In some embodiments, the SV2A inhibitor (e.g., levetiracetam, brivaracetam, or seletracetam), or the pharmaceutically acceptable salt, hydrate, solvate, polymorph, or isomer thereof, is formulated together with the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, in a single pharmaceutical composition. In some embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, may be present in a single dosage unit form (e.g., combined together in one capsule, tablet, powder, or liquid, etc.).

The pharmaceutical composition or combination described herein can comprise more than one SV2A inhibitor (e.g., levetiracetam, brivaracetam, or seletracetam), or pharmaceutically acceptable salts, hydrates, solvates, polymorphs, or isomers thereof (e.g., 2, 3, 4, or 5 SV2A inhibitors, or pharmaceutically acceptable salts, hydrates, solvates, polymorphs, or isomers thereof), and/or more than one GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or pharmaceutically acceptable salts, hydrates, solvates, polymorphs, or isomers thereof (e.g., 2, 3, 4, or 5 GABA_{A} α5 receptor agonists, or pharmaceutically acceptable salts, hydrates, solvates, polymorphs, or isomers thereof). In some embodiments, the pharmaceutical composition or combination described herein comprises more than one SV2A inhibitor (e.g., levetiracetam, brivaracetam, or seletracetam), or pharmaceutically acceptable salts, hydrates, solvates, polymorphs, or isomers thereof (e.g., 2, 3, 4, or 5 SV2A inhibitors, or pharmaceutically acceptable salts, hydrates, solvates, polymorphs, or isomers thereof). In some embodiments, the pharmaceutical composition or combination described herein comprises more than one GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or pharmaceutically acceptable salts, hydrates, solvates, polymorphs, or isomers thereof (e.g., 2, 3, 4, or 5 GABA_{A} α5 receptor agonists, or pharmaceutically acceptable salts, hydrates, solvates, polymorphs, or isomers thereof). In some embodiments, the pharmaceutical composition or combination described herein comprises more than one SV2A inhibitor (e.g., levetiracetam, brivaracetam, or seletracetam), or pharmaceutically acceptable salts, hydrates, solvates, polymorphs, or isomers thereof (e.g., 2, 3, 4, or 5 SV2A inhibitors, or pharmaceutically acceptable salts, hydrates, solvates, polymorphs, or isomers thereof), and more than one GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or pharmaceutically acceptable salts, hydrates, solvates, polymorphs, or isomers thereof (e.g., 2, 3, 4, or 5 GABA_{A} α5 receptor agonists, or pharmaceutically acceptable salts, hydrates, solvates, polymorphs, or isomers thereof). In some embodiments, the pharmaceutical composition or combination described herein comprises more than one SV2A inhibitor (e.g., levetiracetam, brivaracetam, or seletracetam), or pharmaceutically acceptable salts, hydrates, solvates, polymorphs, or isomers thereof (e.g., 2, 3, 4, or 5 SV2A inhibitors, or pharmaceutically acceptable salts, hydrates, solvates, polymorphs, or isomers thereof), and only one GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof. In some embodiments, the pharmaceutical composition or combination described herein comprises only one SV2A inhibitor (e.g., levetiracetam, brivaracetam, or seletracetam), or a pharmaceutically acceptable salt, hydrate, solvate, polymorph, or isomer thereof, and more than one GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or pharmaceutically acceptable salts, hydrates, solvates, polymorphs, or isomers thereof (e.g., 2, 3, 4, or 5 GABA_{A} α5 receptor agonists, or pharmaceutically acceptable salts, hydrates, solvates, polymorphs, or isomers thereof). In some embodiments, the pharmaceutical composition or combination described herein comprises only one SV2A inhibitor (e.g., levetiracetam, brivaracetam, or seletracetam), or a pharmaceutically acceptable salt, hydrate, solvate, polymorph, or isomer thereof, and only one GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof.

In some embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are formulated separately. In certain such embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are packaged together. In some embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are packaged separately. In some embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are formulated in separate pharmaceutical compositions. In certain such embodiments, the separate pharmaceutical compositions are packaged together. In some embodiments, the separate pharmaceutical compositions are packaged separately. In some embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, may be present in discrete dosage unit forms.

The pharmaceutical compositions and combinations described herein can further comprise other agents that serve to enhance and/or complement the effectiveness of the SV2A inhibitor (e.g., levetiracetam, brivaracetam, or seletracetam), or the pharmaceutically acceptable salt, hydrate, solvate, polymorph, or isomer thereof, and/or the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof. The pharmaceutical compositions and combinations may also comprise additional agents known to be useful for treating cognitive impairment. Examples of such agents include antipsychotics, memantine, and acetylcholine esterase inhibitors. These additional agents may be in a single pharmaceutical composition with the SV2A inhibitor (e.g., levetiracetam, brivaracetam, or seletracetam), or the pharmaceutically acceptable salt, hydrate, solvate, polymorph, or isomer thereof, and/or the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof. In some embodiments, these additional agents may be formulated in separate pharmaceutical compositions than the SV2A inhibitor (e.g., levetiracetam, brivaracetam, or seletracetam), or the pharmaceutically acceptable salt, hydrate, solvate, polymorph, or isomer thereof, and/or the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof. In certain such embodiments, the separate pharmaceutical compositions are packaged together. In some embodiments, the separate pharmaceutical compositions are packaged separately.

In some embodiments, the pharmaceutical compositions and combinations of the disclosure comprise more than one additional agent (e.g., antipsychotics, memantine, and acetylcholine esterase inhibitors). In some embodiments, the pharmaceutical compositions and combinations of the disclosure comprise more than two additional agents (e.g., antipsychotics, memantine, and acetylcholine esterase inhibitors). In some embodiments, the pharmaceutical compositions and combinations of the disclosure comprise more than three additional agents (e.g., antipsychotics, memantine, and acetylcholine esterase inhibitors). In some embodiments, the pharmaceutical compositions and combinations of the disclosure comprise more than four additional agents (e.g., antipsychotics, memantine, and acetylcholine esterase inhibitors). In some embodiments, the pharmaceutical compositions and combinations of the disclosure comprise more than five additional agents (e.g., antipsychotics, memantine, and acetylcholine esterase inhibitors). In some embodiments, the pharmaceutical compositions and combinations of the disclosure comprise one additional agent (e.g., antipsychotics, memantine, and acetylcholine esterase inhibitors). In some embodiments, the pharmaceutical compositions and combinations of the disclosure comprise two additional agents (e.g., antipsychotics, memantine, and acetylcholine esterase inhibitors). In some embodiments, the pharmaceutical compositions and combinations of the disclosure comprise three additional agents (e.g., antipsychotics, memantine, and acetylcholine esterase inhibitors). In some embodiments, the pharmaceutical compositions and combinations of the disclosure comprise four additional agents (e.g., antipsychotics, memantine, and acetylcholine esterase inhibitors). In some embodiments, the pharmaceutical compositions and combinations of the disclosure comprise five additional agents (e.g., antipsychotics, memantine, and acetylcholine esterase inhibitors).

“Antipsychotic”, “antipsychotic agent”, “antipsychotic drug”, or “antipsychotic compound” may refer to (1) a typical or an atypical antipsychotic; (2) an agent that is selected from dopaminergic agents, glutamatergic agents, NMDA receptor positive allosteric modulators, glycine reuptake inhibitors, glutamate reuptake inhibitor, metabotropic glutamate receptors (mGluRs) agonists or positive allosteric modulators (PAMs) (e.g., mGluR2/3 agonists or PAMs), glutamate receptor glur5 positive allosteric modulators (PAMs), Ml muscarinic acetylcholine receptor (mAChR) positive allosteric modulators (PAMs), histamine H3 receptor antagonists, AMPA/kainate receptor antagonists, ampakines (CX-516), glutathione prodrugs, noradrenergic agents, serotonin receptor modulators, cholinergic agents, cannabinoid CB1 antagonists, neurokinin 3 antagonists, neurotensin agonists, MAO B inhibitors, PDE10 inhibitors, nNOS inhibits, neurosteroids, and neurotrophic factors, alpha-7 agonists or positive allosteric modulators (PAMs)PAMs, serotonin 2C agonists; and/or (3) an agent that is useful in treating one or more signs or symptoms of schizophrenia or bipolar disorder (in particular, mania).

“Typical antipsychotics”, as used herein, may refer to conventional antipsychotics, which produce antipsychotic effects as well as movement related adverse effects related to disturbances in the nigrostriatal dopamine system. These extrapyramidal side effects (EPS) include Parkinsonism, akathisia, tardive dyskinesia and dystonia. See Baldessarini and Tarazi in Goodman & Gilman's The Pharmacological Basis of Therapeutics 10 Edition, 2001, pp. 485-520.

“Atypical antipsychotics”, as used herein, may refer to antipsychotic drugs that produce antipsychotic effects with little or no EPS and include, but are not limited to, aripiprazole, asenapine, clozapine, iloperidone, olanzapine, lurasidone, paliperidone, quetiapine, risperidone and ziprasidone. “Atypical” antipsychotics differ from conventional antipsychotics in their pharmacological profiles. While conventional antipsychotics are characterized principally by D₂ dopamine receptor blockade, atypical antipsychotics show antagonist effects on multiple receptors including the 5HTₐ and 5HT_{c} serotonin receptors and varying degrees of receptor affinities. Atypical antipsychotic drugs are commonly referred to as serotonin/dopamine antagonists, reflecting the influential hypothesis that greater affinity for the 5HT₂ receptor than for the D₂ receptor underlies “atypical” antipsychotic drug action or “second generation” antipsychotic drugs. However, the atypical antipsychotics often display side effects, including, but not limited to, weight gain, diabetes (e.g., type II diabetes mellitus), hyperlipidemia, QTc interval prolongation, myocarditis, sexual side effects, extrapyramidal side effects and cataract. Thus, atypical antipsychotics do not represent a homogeneous class, given their differences in the context of both alleviation of clinical symptoms and their potential for inducing side effects such as the ones listed above. Further, the common side effects of the atypical antipsychotics as described above often limit the antipsychotic doses that can be used for these agents.

Memantine is chemically known as 3,5-dimethyladamantan-1-amine or 3,5-dimethyltricyclo[3.3.1.1^{3,7}]decan-1-amine, which is an uncompetitive N-methyl-D-aspartate (NMDA) receptor antagonist with moderate affinity. The proprietary names for memantine include: Axura® and Akatinol® (Merz), Namenda® (Forest Laboratories), Ebixa® and Abixa® (Lundbeck), and Memox® (Unipharm). Memantine is approved for the treatment of moderate to severe Alzheimer's disease (AD) in the United States at a dose of up to 28 mg/day. Derivatives or analogs of memantine, which include compounds that structurally or chemically resemble memantine, are also useful in the present disclosure. Such derivatives or analogs of memantine include, but are not limited to those compounds disclosed in U.S. Pat. Nos. 3,391,142; 4,122,193; 4,273,774; and 5,061,703; U.S. Patent Application Publication US20040087658, US20050113458, US20060205822, US20090081259, US20090124659, and US20100227852; EP Patent Application Publication EP2260839A2; EP Patent EP1682109B1; and PCT Application Publication WO2005079779, all of which are incorporated herein by reference. Memantine, as used in the present disclosure, includes memantine and its derivatives and analogs, as well as hydrates, polymorphs, prodrugs, salts, and solvates thereof. Memantine, as used herein, also includes a composition comprising memantine or a derivative or an analog or a pharmaceutically acceptable salt, hydrate, solvate, polymorph, or prodrug thereof, wherein the composition optionally further comprises at least one additional therapeutic agent (such as a therapeutic agent useful for treating a CNS disorder or cognitive impairments associated thereof). In some embodiments, the memantine composition suitable for use in the present disclosure comprises memantine and a second therapeutic agent that is donepezil (under the trade name Aricept).

“Acetylcholinesterase inhibitor” or “AChE-I” as used herein may refer to an agent that inhibits the ability of the cholinesterase enzyme to break down the neurotransmitter acetylcholine, thereby increasing the concentration and duration of acetylcholine, mainly in brain synapses or neuromuscular junctions. AChE-Is suitable for use in this application may include, for example, the subcategories of (i) reversible non-competitive inhibitors or reversible competitive inhibitors, (ii) irreversible, and (iii) quasi-irreversible inhibitors. Donepezil is an example of an AChE-I.

In some embodiments of disclosure, the SV2A inhibitor (e.g., levetiracetam, brivaracetam, or seletracetam), or pharmaceutically acceptable salts, hydrates, solvates, polymorphs, or isomers thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or pharmaceutically acceptable salts, hydrates, solvates, polymorphs, or isomers thereof, are formulated with a pharmaceutically acceptable carrier. Pharmaceutically acceptable carriers that may be used in these pharmaceutical compositions or combinations (or a component thereof, such as a SV2A inhibitor (e.g., levetiracetam, brivaracetam, or seletracetam), or a pharmaceutically acceptable salt, hydrate, solvate, polymorph, or isomer thereof, and/or a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof) include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Examples of suitable aqueous and non-aqueous carriers which may be employed in the pharmaceutical compositions or combinations (or components thereof) of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

In some embodiments, no carrier is used. For example, the SV2A inhibitor (e.g., levetiracetam, brivaracetam, or seletracetam), or a pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, can be administered alone or as a component of a pharmaceutical composition or combination. The SV2A inhibitor (e.g., levetiracetam, brivaracetam, or seletracetam), or a pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, may be formulated for administration in any convenient way for use in medicine.

The pharmaceutical compositions or combinations (or components thereof) of the disclosure (e.g., pharmaceutical compositions or combinations (or components thereof) comprising a SV2A inhibitor (e.g., levetiracetam, brivaracetam, or seletracetam), or a pharmaceutically acceptable salt, hydrate, solvate, polymorph, or isomer thereof, and/or a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof) may be specifically formulated for administration by any suitable route as described herein and known in the art. The pharmaceutical compositions or combinations (or components thereof) of the disclosure may be specifically formulated for topical, systemic, and local administration. Pharmaceutical compositions or combinations (or components thereof) of the disclosure for parental administration (e.g., subcutaneously, intravenously, arterially, intradermally, intramuscularly, intraperitoneally) or intraspinal or intracerebral administration include sterile aqueous and nonaqueous injectable solutions, dispersions, suspensions or emulsions as well as sterile powders to be reconstituted in sterile injectable solutions or dispersions prior to use, which may contain antioxidants, buffers, bacteriostats, solutes which render the pharmaceutical composition or combination (or a component thereof) isotonic with the blood of the intended recipient, or suspending or thickening agents. When administered parenterally, the compound of the disclosure (e.g., a SV2A inhibitor (e.g., levetiracetam, brivaracetam, or seletracetam), or a pharmaceutically acceptable salt, hydrate, solvate, polymorph, or isomer thereof, and/or a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof) may be in a pyrogen-free, physiologically acceptable form. Techniques and formulations generally may be found in Remington's Pharmaceutical Sciences, Meade Publishing Co., Easton, Pa.

Pharmaceutical compositions or combinations (or components thereof) of the disclosure for intraoral and oral delivery (including sublingual and buccal administration, e.g. Danckwerts et al, and oral) include but are not limited to bioadhesive polymers, tablets, patches, thin films, liquids and semisolids (see e.g., Smart et al).

The pharmaceutical composition or combination (or a component thereof) of the present disclosure (e.g., a pharmaceutical composition or combination (or a component thereof) comprising a SV2A inhibitor (e.g., levetiracetam, brivaracetam, or seletracetam), or a pharmaceutically acceptable salt, hydrate, solvate, polymorph, or isomer thereof, and/or a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof) may be in a solid dosage form such as a capsule, tablet, medicinal dragée, pill, lozenge, cachet, powder, troche, wafer, or granule. In solid dosage forms for oral administration (capsules, tablets, pills, medicinal dragées, powders, granules, and the like), one or more compounds of the disclosure (e.g., a SV2A inhibitor (e.g., levetiracetam, brivaracetam, or seletracetam), or a pharmaceutically acceptable salt, hydrate, solvate, polymorph, or isomer thereof, and/or a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof) may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions or combinations (or components thereof) of the disclosure may also comprise buffering agents. Solid pharmaceutical compositions or combinations (or components thereof) of a similar type may also be employed as fillers in soft and hard-filled gelatin capsules using such excipients as lactose or milk sugars, as well as high molecular weight polyethylene glycols and the like.

The pharmaceutical composition or combination (a or component thereof) of the disclosure (e.g., a pharmaceutical composition or combination (or a component thereof) comprising a SV2A inhibitor (e.g., levetiracetam, brivaracetam, or seletracetam), or a pharmaceutically acceptable salt, hydrate, solvate, polymorph, or isomer thereof, and/or a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof) may also be in an aqueous or non-aqueous liquid dosage form including solution, emulsion, microemulsion, suspension, syrup, pastille, or elixir. In some embodiments, the pharmaceutical composition or combination (or a component thereof) of the disclosure is in an aqueous solution. In some embodiments, the pharmaceutical composition or combination (or a component thereof) of the disclosure is in a suspension form. Where appropriate, the pharmaceutical composition or combination (or a component thereof) of the disclosure may be prepared with coatings such as enteric coatings or they may be formulated so as to provide extended release (e.g., a controlled release, a prolonged release, a sustained release, a delayed release, or a slow release) of one or more compound of the disclosure (e.g., a SV2A inhibitor (e.g., levetiracetam, brivaracetam, or seletracetam), or a pharmaceutically acceptable salt, hydrate, solvate, polymorph, or isomer thereof, and/or a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof) according to methods well known in the art. Liquid dosage forms may also comprise inert diluents commonly used in the art, such as water or other solvents, solubilizing agents and emulsifiers, such as ethyl alcohol (ethanol), isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, oral pharmaceutical compositions or combinations (or components thereof) can also include adjuvants such as wetting agents, emulsifying and suspending agents, sweetening, flavoring, coloring, perfuming, and preservative agents. Suspensions, in addition to the compounds of the disclosure (e.g., a SV2A inhibitor (e.g., levetiracetam, brivaracetam, or seletracetam), or a pharmaceutically acceptable salt, hydrate, solvate, polymorph, or isomer thereof, and/or a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof), may comprise suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

Pharmaceutical compositions or combinations (or components thereof) of the disclosure for respiratory delivery (pulmonary and nasal delivery) include but are not limited to a variety of pressurized metered dose inhalers, dry powder inhalers, nebulizers, aqueous mist inhalers, drops, solutions, suspensions, sprays, powders, gels, ointments, and specialized systems such as liposomes and microspheres (see e.g. Owens et al, “Alternative Routes of Insulin Delivery” and Martini et al). Pharmaceutical compositions or combinations (or components thereof) of the disclosure for transdermal delivery include but are not limited to colloids, patches, and microemulsions.

Other suitable administration forms for the pharmaceutical compositions or combinations (or components thereof) of the disclosure include depot injectable formulations, suppositories, sprays, ointments, cremes, gels, inhalants, dermal patches, implants, devices, formulations for ocular administration, etc.

The pharmaceutical compositions or combinations (or components thereof) of the disclosure may also comprise adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the pharmaceutical compositions or combinations (or components thereof). In addition, prolonged absorption of the injectable pharmaceutical form may be brought about by the inclusion of agents which delay absorption, such as aluminum monostearate and gelatin.

Pharmaceutical compositions or combinations (or components thereof) of the disclosure can be prepared by methods well known in the art of pharmacy, see, e.g., Goodman et al., 2001; Ansel, et al., 2004; Stoklosa et al., 2001; and Bustamante, et al., 1993.

In certain embodiments of the disclosure, a pharmaceutical composition comprising an SV2A inhibitor (e.g., levetiracetam, brivaracetam, or seletracetam), or the pharmaceutically acceptable salt, hydrate, solvate, polymorph, or isomer thereof, comprises the SV2A inhibitor (e.g., levetiracetam, brivaracetam, or seletracetam), or the pharmaceutically acceptable salt, hydrate, solvate, polymorph, or isomer thereof, in an amount of 0.07 mg-60 mg, 0.07 mg-350 mg, 25 mg-60 mg, 25 mg-125 mg, 50 mg-250 mg, 5 mg-140 mg, 0.7 mg-180 mg, 125 mg-240 mg, 3 mg-50 mg, or 3 mg-60 mg. In some embodiments, a pharmaceutical composition comprising an SV2A inhibitor (e.g., levetiracetam, brivaracetam, or seletracetam), or the pharmaceutically acceptable salt, hydrate, solvate, polymorph, or isomer thereof, comprises the SV2A inhibitor (e.g., levetiracetam, brivaracetam, or seletracetam), or the pharmaceutically acceptable salt, hydrate, solvate, polymorph, or isomer thereof, in an amount of 0.05 mg-35 mg. In some embodiments, a pharmaceutical composition comprising an SV2A inhibitor (e.g., levetiracetam, brivaracetam, or seletracetam), or the pharmaceutically acceptable salt, hydrate, solvate, polymorph, or isomer thereof, comprises the SV2A inhibitor (e.g., levetiracetam, brivaracetam, or seletracetam), or the pharmaceutically acceptable salt, hydrate, solvate, polymorph, or isomer thereof, in an amount of 7 mg-350 mg. In some embodiments, a pharmaceutical composition comprising an SV2A inhibitor (e.g., levetiracetam, brivaracetam, or seletracetam), or the pharmaceutically acceptable salt, hydrate, solvate, polymorph, or isomer thereof, comprises the SV2A inhibitor (e.g., levetiracetam, brivaracetam, or seletracetam), or the pharmaceutically acceptable salt, hydrate, solvate, polymorph, or isomer thereof, in an amount of 0.07 mg-35 mg. In some embodiments, a pharmaceutical composition comprising an SV2A inhibitor (e.g., levetiracetam, brivaracetam, or seletracetam), or the pharmaceutically acceptable salt, hydrate, solvate, polymorph, or isomer thereof, comprises the SV2A inhibitor (e.g., levetiracetam, brivaracetam, or seletracetam), or the pharmaceutically acceptable salt, hydrate, solvate, polymorph, or isomer thereof, in an amount of 7 mg-35 mg. In some embodiments, a pharmaceutical composition comprising an SV2A inhibitor (e.g., levetiracetam, brivaracetam, or seletracetam), or the pharmaceutically acceptable salt, hydrate, solvate, polymorph, or isomer thereof, comprises the SV2A inhibitor (e.g., levetiracetam, brivaracetam, or seletracetam), or the pharmaceutically acceptable salt, hydrate, solvate, polymorph, or isomer thereof, in an amount of 50 mg-350 mg. In some embodiments, a pharmaceutical composition comprising an SV2A inhibitor (e.g., levetiracetam, brivaracetam, or seletracetam), or the pharmaceutically acceptable salt, hydrate, solvate, polymorph, or isomer thereof, comprises the SV2A inhibitor (e.g., levetiracetam, brivaracetam, or seletracetam), or the pharmaceutically acceptable salt, hydrate, solvate, polymorph, or isomer thereof, in an amount of 190 mg-220 mg. In some embodiments, a pharmaceutical composition comprising an SV2A inhibitor (e.g., levetiracetam, brivaracetam, or seletracetam), or the pharmaceutically acceptable salt, hydrate, solvate, polymorph, or isomer thereof, comprises the SV2A inhibitor (e.g., levetiracetam, brivaracetam, or seletracetam), or the pharmaceutically acceptable salt, hydrate, solvate, polymorph, or isomer thereof, in an amount of 190 mg-240 mg. In some embodiments, a pharmaceutical composition comprising an SV2A inhibitor (e.g., levetiracetam, brivaracetam, or seletracetam), or the pharmaceutically acceptable salt, hydrate, solvate, polymorph, or isomer thereof, comprises the SV2A inhibitor (e.g., levetiracetam, brivaracetam, or seletracetam), or the pharmaceutically acceptable salt, hydrate, solvate, polymorph, or isomer thereof, in an amount of about 220 mg. In some embodiments, the pharmaceutical composition further comprises a GABA_{A} α5 receptor agonist (e.g., a compound of Formula I, Formula II, or Formula IV; compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof.

In certain embodiments of the disclosure, a pharmaceutical composition comprising an SV2A inhibitor (e.g., levetiracetam, brivaracetam, or seletracetam), or the pharmaceutically acceptable salt, hydrate, solvate, polymorph, or isomer thereof, comprises an amount of the SV2A inhibitor (e.g., levetiracetam, brivaracetam, or seletracetam), or the pharmaceutically acceptable salt, hydrate, solvate, polymorph, or isomer thereof, of 0.05 mg-35 mg, 0.07 mg-60 mg, 0.07 mg-350 mg, 25 mg-60 mg, 25 mg-125 mg, 50 mg-250 mg, 5 mg-15 mg, 5 mg-30 mg, 5 mg-140 mg, 0.7 mg-180 mg, 125 mg-240 mg, 3 mg-50 mg, or 0.07 mg-50 mg, 3 mg-60 mg, or about 0.1 mg-500 mg, 0.1 mg-350 mg, 0.7 mg-350 mg, 3 mg-300 mg, 3 mg-150 mg, 3 mg-110 mg, 7 mg-70 mg, 70 mg-350 mg, 100 mg-300 mg, or 125 mg-250 mg. In other embodiments, the amount of the SV2A inhibitor (e.g., levetiracetam, brivaracetam, or seletracetam), or the pharmaceutically acceptable salt, hydrate, solvate, polymorph, or isomer thereof, is less than 500 mg, less than 350 mg, less than 300 mg, less than 250 mg, less than 200 mg, less than 150 mg, less than 110 mg, less than 100 mg, less than 70 mg, less than 50 mg, less than 35 mg, less than 10 mg, less than 7 mg, less than 5 mg, less than 3 mg, less than 1 mg, less than 0.7 mg, less than 0.5 mg, less than 0.1 mg, less than 0.07 mg, or less than 0.05 mg. In some embodiments, the pharmaceutical composition further comprises a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof.

In certain embodiments of the disclosure, a pharmaceutical composition comprising an SV2A inhibitor (e.g., levetiracetam, brivaracetam, or seletracetam), or the pharmaceutically acceptable salt, hydrate, solvate, polymorph, or isomer thereof, comprises an amount of the SV2A inhibitor (e.g., levetiracetam, brivaracetam, or seletracetam), or the pharmaceutically acceptable salt, hydrate, solvate, polymorph, or isomer thereof, of 0.1 mg-500 mg, 0.1 mg-300 mg, 0.7 mg-300 mg, 3 mg-300 mg, 3 mg-150 mg, 3 mg-110 mg, 7 mg-70 mg, 7 mg-300 mg, 70 mg-300 mg, 100 mg-300 mg, 125 mg-250 mg, 0.5 mg-50 mg, 0.5 mg-75 mg, 0.5 mg-100 mg, 0.5 mg-150 mg, 0.5 mg-200 mg, 0.5 mg-225 mg, 0.5 mg-250 mg, 0.5 mg-300 mg, 1.5 mg-50 mg, 1.5 mg-75 mg, 1.5 mg-100 mg, 1.5 mg-150 mg, 1.5 mg-200 mg, 1.5 mg-225 mg, 1.5 mg-250 mg, 1.5 mg-300 mg, 3 mg-50 mg, 3 mg-75 mg, 3 mg-100 mg, 3 mg-150 mg, 3 mg-200 mg, 3 mg-225 mg, 3 mg-250 mg, 3 mg-300 mg, 5 mg-50 mg, 5 mg-75 mg, 5 mg-100 mg, 5 mg-150 mg, 5 mg-200 mg, 5 mg-225 mg, 5 mg-250 mg, 5 mg-300 mg, 7 mg-50 mg, 7 mg-75 mg, 7 mg-100 mg, 7 mg-150 mg, 7 mg-200 mg, 7 mg-225 mg, 7 mg-250 mg, 7 mg-300 mg, 15 mg-50 mg, 15 mg-75 mg, 15 mg-100 mg, 15 mg-150 mg, 15 mg-200 mg, 15 mg-225 mg, 15 mg-250 mg, 15 mg-300 mg, 30 mg-50 mg, 30 mg-75 mg, 30 mg-100 mg, 30 mg-150 mg, 30 mg-200 mg, 30 mg-225 mg, 30 mg-250 mg, or 30 mg-300 mg. In some embodiments, the pharmaceutical composition further comprises a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof.

In certain embodiments of the disclosure, a pharmaceutical composition comprising a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, comprises an amount of the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, of 0.05 mg to 5000 mg or 5 mg to 1000 mg. In some embodiments, the pharmaceutical composition may comprise about 0.5 mg, about 5 mg, about 20 mg, about 50 mg, about 75 mg, about 100 mg, about 150 mg, about 250 mg, about 500 mg, about 750 mg, about 1000 mg, about 1250 mg, about 2500 mg, about 3500 mg, or 5000 mg of the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof. In some embodiments, the pharmaceutical composition further comprises an SV2A inhibitor (e.g., levetiracetam, brivaracetam, or seletracetam), or a pharmaceutically acceptable salt, hydrate, solvate, polymorph, or isomer thereof.

In certain embodiments of the disclosure, a combination comprising an SV2A inhibitor (e.g., levetiracetam, brivaracetam, or seletracetam), or the pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, comprises the SV2A inhibitor (e.g., levetiracetam, brivaracetam, or seletracetam), or the pharmaceutically acceptable salt, hydrate, solvate, polymorph, or isomer thereof, in an amount of 0.07 mg-60 mg, 0.07 mg-350 mg, 25 mg-60 mg, 25 mg-125 mg, 50 mg-250 mg, 5 mg-140 mg, 0.7 mg-180 mg, 125 mg-240 mg, 3 mg-50 mg, or 3 mg-60 mg. In some embodiments, a combination comprising an SV2A inhibitor (e.g., levetiracetam, brivaracetam, or seletracetam), or the pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, comprises the SV2A inhibitor (e.g., levetiracetam, brivaracetam, or seletracetam), or the pharmaceutically acceptable salt, hydrate, solvate, polymorph, or isomer thereof, in an amount of 0.05 mg-35 mg. In some embodiments, a combination comprising an SV2A inhibitor (e.g., levetiracetam, brivaracetam, or seletracetam), or the pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, comprises the SV2A inhibitor (e.g., levetiracetam, brivaracetam, or seletracetam), or the pharmaceutically acceptable salt, hydrate, solvate, polymorph, or isomer thereof, in an amount of 7 mg-350 mg. In some embodiments, a combination comprising an SV2A inhibitor (e.g., levetiracetam, brivaracetam, or seletracetam), or the pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, comprises the SV2A inhibitor (e.g., levetiracetam, brivaracetam, or seletracetam), or the pharmaceutically acceptable salt, hydrate, solvate, polymorph, or isomer thereof, in an amount of 0.07 mg-35 mg. In some embodiments, a combination comprising an SV2A inhibitor (e.g., levetiracetam, brivaracetam, or seletracetam), or the pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, comprises the SV2A inhibitor (e.g., levetiracetam, brivaracetam, or seletracetam), or the pharmaceutically acceptable salt, hydrate, solvate, polymorph, or isomer thereof, in an amount of 7 mg-35 mg. In some embodiments, a combination comprising an SV2A inhibitor (e.g., levetiracetam, brivaracetam, or seletracetam), or the pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, comprises the SV2A inhibitor (e.g., levetiracetam, brivaracetam, or seletracetam), or the pharmaceutically acceptable salt, hydrate, solvate, polymorph, or isomer thereof, in an amount of 50 mg-350 mg. In some embodiments, a combination comprising an SV2A inhibitor (e.g., levetiracetam, brivaracetam, or seletracetam), or the pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, comprises the SV2A inhibitor (e.g., levetiracetam, brivaracetam, or seletracetam), or the pharmaceutically acceptable salt, hydrate, solvate, polymorph, or isomer thereof, in an amount of 190 mg-220 mg. In some embodiments, a combination comprising an SV2A inhibitor (e.g., levetiracetam, brivaracetam, or seletracetam), or the pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, comprises the SV2A inhibitor (e.g., levetiracetam, brivaracetam, or seletracetam), or the pharmaceutically acceptable salt, hydrate, solvate, polymorph, or isomer thereof, in an amount of 190 mg-240 mg. In some embodiments, a combination comprising an SV2A inhibitor (e.g., levetiracetam, brivaracetam, or seletracetam), or the pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, comprises the SV2A inhibitor (e.g., levetiracetam, brivaracetam, or seletracetam), or the pharmaceutically acceptable salt, hydrate, solvate, polymorph, or isomer thereof, in an amount of about 220 mg.

In certain embodiments of the disclosure, a combination comprising an SV2A inhibitor (e.g., levetiracetam, brivaracetam, or seletracetam), or the pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, comprises an amount of the SV2A inhibitor (e.g., levetiracetam, brivaracetam, or seletracetam), or the pharmaceutically acceptable salt, hydrate, solvate, polymorph, or isomer thereof, of 0.05 mg-35 mg, 0.07 mg-60 mg, 0.07 mg-350 mg, 25 mg-60 mg, 25 mg-125 mg, 50 mg-250 mg, 5 mg-15 mg, 5 mg-30 mg, 5 mg-140 mg, 0.7 mg-180 mg, 125 mg-240 mg, 3 mg-50 mg, or 0.07 mg-50 mg, 3 mg-60 mg, or about 0.1 mg-500 mg, 0.1 mg-350 mg, 0.7 mg-350 mg, 3 mg-300 mg, 3 mg-150 mg, 3 mg-110 mg, 7 mg-70 mg, 70 mg-350 mg, 100 mg-300 mg, or 125 mg-250 mg. In other embodiments, the amount of the SV2A inhibitor (e.g., levetiracetam, brivaracetam, or seletracetam), or the pharmaceutically acceptable salt, hydrate, solvate, polymorph, or isomer thereof, is less than 500 mg, less than 350 mg, less than 300 mg, less than 250 mg, less than 200 mg, less than 150 mg, less than 110 mg, less than 100 mg, less than 70 mg, less than 50 mg, less than 35 mg, less than 10 mg, less than 7 mg, less than 5 mg, less than 3 mg, less than 1 mg, less than 0.7 mg, less than 0.5 mg, less than 0.1 mg, less than 0.07 mg, or less than 0.05 mg.

In certain embodiments of the disclosure, a combination comprising an SV2A inhibitor (e.g., levetiracetam, brivaracetam, or seletracetam), or the pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, comprises an amount of the SV2A inhibitor (e.g., levetiracetam, brivaracetam, or seletracetam), or the pharmaceutically acceptable salt, hydrate, solvate, polymorph, or isomer thereof, of 0.1 mg-500 mg, 0.1 mg-300 mg, 0.7 mg-300 mg, 3 mg-300 mg, 3 mg-150 mg, 3 mg-110 mg, 7 mg-70 mg, 7 mg-300 mg, 70 mg-300 mg, 100 mg-300 mg, 125 mg-250 mg, 0.5 mg-50 mg, 0.5 mg-75 mg, 0.5 mg-100 mg, 0.5 mg-150 mg, 0.5 mg-200 mg, 0.5 mg-225 mg, 0.5 mg-250 mg, 0.5 mg-300 mg, 1.5 mg-50 mg, 1.5 mg-75 mg, 1.5 mg-100 mg, 1.5 mg-150 mg, 1.5 mg-200 mg, 1.5 mg-225 mg, 1.5 mg-250 mg, 1.5 mg-300 mg, 3 mg-50 mg, 3 mg-75 mg, 3 mg-100 mg, 3 mg-150 mg, 3 mg-200 mg, 3 mg-225 mg, 3 mg-250 mg, 3 mg-300 mg, 5 mg-50 mg, 5 mg-75 mg, 5 mg-100 mg, 5 mg-150 mg, 5 mg-200 mg, 5 mg-225 mg, 5 mg-250 mg, 5 mg-300 mg, 7 mg-50 mg, 7 mg-75 mg, 7 mg-100 mg, 7 mg-150 mg, 7 mg-200 mg, 7 mg-225 mg, 7 mg-250 mg, 7 mg-300 mg, 15 mg-50 mg, 15 mg-75 mg, 15 mg-100 mg, 15 mg-150 mg, 15 mg-200 mg, 15 mg-225 mg, 15 mg-250 mg, 15 mg-300 mg, 30 mg-50 mg, 30 mg-75 mg, 30 mg-100 mg, 30 mg-150 mg, 30 mg-200 mg, 30 mg-225 mg, 30 mg-250 mg, or 30 mg-300 mg.

In certain embodiments of the disclosure, a combination comprising a pharmaceutical composition comprising an SV2A inhibitor (e.g., levetiracetam, brivaracetam, or seletracetam), or the pharmaceutically acceptable salt, hydrate, solvate, polymorph, or isomer thereof, comprises the SV2A inhibitor (e.g., levetiracetam, brivaracetam, or seletracetam), or the pharmaceutically acceptable salt, hydrate, solvate, polymorph, or isomer thereof, in an amount of 0.07 mg-60 mg, 0.07 mg-350 mg, 25 mg-60 mg, 25 mg-125 mg, 50 mg-250 mg, 5 mg-140 mg, 0.7 mg-180 mg, 125 mg-240 mg, 3 mg-50 mg, or 3 mg-60 mg. In some embodiments, a combination comprising a pharmaceutical composition comprising an SV2A inhibitor (e.g., levetiracetam, brivaracetam, or seletracetam), or the pharmaceutically acceptable salt, hydrate, solvate, polymorph, or isomer thereof, comprises the SV2A inhibitor (e.g., levetiracetam, brivaracetam, or seletracetam), or the pharmaceutically acceptable salt, hydrate, solvate, polymorph, or isomer thereof, in an amount of 0.05 mg-35 mg. In some embodiments, a combination comprising a pharmaceutical composition comprising an SV2A inhibitor (e.g., levetiracetam, brivaracetam, or seletracetam), or the pharmaceutically acceptable salt, hydrate, solvate, polymorph, or isomer thereof, comprises the SV2A inhibitor (e.g., levetiracetam, brivaracetam, or seletracetam), or the pharmaceutically acceptable salt, hydrate, solvate, polymorph, or isomer thereof, in an amount of 7 mg-350 mg. In some embodiments, a combination comprising a pharmaceutical composition comprising an SV2A inhibitor (e.g., levetiracetam, brivaracetam, or seletracetam), or the pharmaceutically acceptable salt, hydrate, solvate, polymorph, or isomer thereof, comprises the SV2A inhibitor (e.g., levetiracetam, brivaracetam, or seletracetam), or the pharmaceutically acceptable salt, hydrate, solvate, polymorph, or isomer thereof, in an amount of 0.07 mg-35 mg. In some embodiments, a combination comprising a pharmaceutical composition comprising an SV2A inhibitor (e.g., levetiracetam, brivaracetam, or seletracetam), or the pharmaceutically acceptable salt, hydrate, solvate, polymorph, or isomer thereof, comprises the SV2A inhibitor (e.g., levetiracetam, brivaracetam, or seletracetam), or the pharmaceutically acceptable salt, hydrate, solvate, polymorph, or isomer thereof, in an amount of 7 mg-35 mg. In some embodiments, a combination comprising a pharmaceutical composition comprising an SV2A inhibitor (e.g., levetiracetam, brivaracetam, or seletracetam), or the pharmaceutically acceptable salt, hydrate, solvate, polymorph, or isomer thereof, comprises the SV2A inhibitor (e.g., levetiracetam, brivaracetam, or seletracetam), or the pharmaceutically acceptable salt, hydrate, solvate, polymorph, or isomer thereof, in an amount of 50 mg-350 mg. In some embodiments, a combination comprising a pharmaceutical composition comprising an SV2A inhibitor (e.g., levetiracetam, brivaracetam, or seletracetam), or the pharmaceutically acceptable salt, hydrate, solvate, polymorph, or isomer thereof, comprises the SV2A inhibitor (e.g., levetiracetam, brivaracetam, or seletracetam), or the pharmaceutically acceptable salt, hydrate, solvate, polymorph, or isomer thereof, in an amount of 190 mg-220 mg. In some embodiments, a combination comprising a pharmaceutical composition comprising an SV2A inhibitor (e.g., levetiracetam, brivaracetam, or seletracetam), or the pharmaceutically acceptable salt, hydrate, solvate, polymorph, or isomer thereof, comprises the SV2A inhibitor (e.g., levetiracetam, brivaracetam, or seletracetam), or the pharmaceutically acceptable salt, hydrate, solvate, polymorph, or isomer thereof, in an amount of 190 mg-240 mg. In some embodiments, a combination comprising a pharmaceutical composition comprising an SV2A inhibitor (e.g., levetiracetam, brivaracetam, or seletracetam), or the pharmaceutically acceptable salt, hydrate, solvate, polymorph, or isomer thereof, comprises the SV2A inhibitor (e.g., levetiracetam, brivaracetam, or seletracetam), or the pharmaceutically acceptable salt, hydrate, solvate, polymorph, or isomer thereof, in an amount of about 220 mg. In some embodiments, the combination further comprises a pharmaceutical composition comprising a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof.

In certain embodiments of the disclosure, a combination comprising a pharmaceutical composition comprising an SV2A inhibitor (e.g., levetiracetam, brivaracetam, or seletracetam), or the pharmaceutically acceptable salt, hydrate, solvate, polymorph, or isomer thereof, comprises an amount of the SV2A inhibitor (e.g., levetiracetam, brivaracetam, or seletracetam), or the pharmaceutically acceptable salt, hydrate, solvate, polymorph, or isomer thereof, of 0.05 mg-35 mg, 0.07 mg-60 mg, 0.07 mg-350 mg, 25 mg-60 mg, 25 mg-125 mg, 50 mg-250 mg, 5 mg-15 mg, 5 mg-30 mg, 5 mg-140 mg, 0.7 mg-180 mg, 125 mg-240 mg, 3 mg-50 mg, or 0.07 mg-50 mg, 3 mg-60 mg, or about 0.1 mg-500 mg, 0.1 mg-350 mg, 0.7 mg-350 mg, 3 mg-300 mg, 3 mg-150 mg, 3 mg-110 mg, 7 mg-70 mg, 70 mg-350 mg, 100 mg-300 mg, or 125 mg-250 mg. In other embodiments, the amount of the SV2A inhibitor (e.g., levetiracetam, brivaracetam, or seletracetam), or the pharmaceutically acceptable salt, hydrate, solvate, polymorph, or isomer thereof, is less than 500 mg, less than 350 mg, less than 300 mg, less than 250 mg, less than 200 mg, less than 150 mg, less than 110 mg, less than 100 mg, less than 70 mg, less than 50 mg, less than 35 mg, less than 10 mg, less than 7 mg, less than 5 mg, less than 3 mg, less than 1 mg, less than 0.7 mg, less than 0.5 mg, less than 0.1 mg, less than 0.07 mg, or less than 0.05 mg. In some embodiments, the combination further comprises a pharmaceutical composition comprising a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof.

In certain embodiments of the disclosure, a combination comprising a pharmaceutical composition comprising an SV2A inhibitor (e.g., levetiracetam, brivaracetam, or seletracetam), or the pharmaceutically acceptable salt, hydrate, solvate, polymorph, or isomer thereof, comprises an amount of the SV2A inhibitor (e.g., levetiracetam, brivaracetam, or seletracetam), or the pharmaceutically acceptable salt, hydrate, solvate, polymorph, or isomer thereof, of 0.1 mg-500 mg, 0.1 mg-300 mg, 0.7 mg-300 mg, 3 mg-300 mg, 3 mg-150 mg, 3 mg-110 mg, 7 mg-70 mg, 7 mg-300 mg, 70 mg-300 mg, 100 mg-300 mg, 125 mg-250 mg, 0.5 mg-50 mg, 0.5 mg-75 mg, 0.5 mg-100 mg, 0.5 mg-150 mg, 0.5 mg-200 mg, 0.5 mg-225 mg, 0.5 mg-250 mg, 0.5 mg-300 mg, 1.5 mg-50 mg, 1.5 mg-75 mg, 1.5 mg-100 mg, 1.5 mg-150 mg, 1.5 mg-200 mg, 1.5 mg-225 mg, 1.5 mg-250 mg, 1.5 mg-300 mg, 3 mg-50 mg, 3 mg-75 mg, 3 mg-100 mg, 3 mg-150 mg, 3 mg-200 mg, 3 mg-225 mg, 3 mg-250 mg, 3 mg-300 mg, 5 mg-50 mg, 5 mg-75 mg, 5 mg-100 mg, 5 mg-150 mg, 5 mg-200 mg, 5 mg-225 mg, 5 mg-250 mg, 5 mg-300 mg, 7 mg-50 mg, 7 mg-75 mg, 7 mg-100 mg, 7 mg-150 mg, 7 mg-200 mg, 7 mg-225 mg, 7 mg-250 mg, 7 mg-300 mg, 15 mg-50 mg, 15 mg-75 mg, 15 mg-100 mg, 15 mg-150 mg, 15 mg-200 mg, 15 mg-225 mg, 15 mg-250 mg, 15 mg-300 mg, 30 mg-50 mg, 30 mg-75 mg, 30 mg-100 mg, 30 mg-150 mg, 30 mg-200 mg, 30 mg-225 mg, 30 mg-250 mg, or 30 mg-300 mg. In some embodiments, the combination further comprises a pharmaceutical composition comprising a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof.

In certain embodiments of the disclosure, a combination comprising a pharmaceutical composition comprising a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, and an SV2A inhibitor (e.g., levetiracetam, brivaracetam, or seletracetam), or the pharmaceutically acceptable salt, hydrate, solvate, polymorph, or isomer thereof, comprises an amount of the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, of 0.05 mg to 5000 mg or 5 mg to 1000 mg. In some embodiments, the combination may comprise a pharmaceutical composition comprising about 0.5 mg, about 5 mg, about 20 mg, about 50 mg, about 75 mg, about 100 mg, about 150 mg, about 250 mg, about 500 mg, about 750 mg, about 1000 mg, about 1250 mg, about 2500 mg, about 3500 mg, or 5000 mg of the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof. In some embodiments, the combination further comprises a pharmaceutical composition comprising an SV2A inhibitor (e.g., levetiracetam, brivaracetam, or seletracetam), or a pharmaceutically acceptable salt, hydrate, solvate, polymorph, or isomer thereof.

In certain embodiments of the disclosure, a combination comprising a pharmaceutical composition comprising an SV2A inhibitor (e.g., levetiracetam, brivaracetam, or seletracetam), or the pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, comprises the SV2A inhibitor (e.g., levetiracetam, brivaracetam, or seletracetam), or the pharmaceutically acceptable salt, hydrate, solvate, polymorph, or isomer thereof, in an amount of 0.07 mg-60 mg, 0.07 mg-350 mg, 25 mg-60 mg, 25 mg-125 mg, 50 mg-250 mg, 5 mg-140 mg, 0.7 mg-180 mg, 125 mg-240 mg, 3 mg-50 mg, or 3 mg-60 mg. In some embodiments, a combination comprising a pharmaceutical composition comprising an SV2A inhibitor (e.g., levetiracetam, brivaracetam, or seletracetam), or the pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, comprises the SV2A inhibitor (e.g., levetiracetam, brivaracetam, or seletracetam), or the pharmaceutically acceptable salt, hydrate, solvate, polymorph, or isomer thereof, in an amount of 0.05 mg-35 mg. In some embodiments, a combination comprising a pharmaceutical composition comprising an SV2A inhibitor (e.g., levetiracetam, brivaracetam, or seletracetam), or the pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, comprises the SV2A inhibitor (e.g., levetiracetam, brivaracetam, or seletracetam), or the pharmaceutically acceptable salt, hydrate, solvate, polymorph, or isomer thereof, in an amount of 7 mg-350 mg. In some embodiments, a combination comprising a pharmaceutical composition comprising an SV2A inhibitor (e.g., levetiracetam, brivaracetam, or seletracetam), or the pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, comprises the SV2A inhibitor (e.g., levetiracetam, brivaracetam, or seletracetam), or the pharmaceutically acceptable salt, hydrate, solvate, polymorph, or isomer thereof, in an amount of 0.07 mg-35 mg. In some embodiments, a combination comprising a pharmaceutical composition comprising an SV2A inhibitor (e.g., levetiracetam, brivaracetam, or seletracetam), or the pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, comprises the SV2A inhibitor (e.g., levetiracetam, brivaracetam, or seletracetam), or the pharmaceutically acceptable salt, hydrate, solvate, polymorph, or isomer thereof, in an amount of 7 mg-35 mg. In some embodiments, a combination comprising a pharmaceutical composition comprising an SV2A inhibitor (e.g., levetiracetam, brivaracetam, or seletracetam), or the pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, comprises the SV2A inhibitor (e.g., levetiracetam, brivaracetam, or seletracetam), or the pharmaceutically acceptable salt, hydrate, solvate, polymorph, or isomer thereof, in an amount of 50 mg-350 mg. In some embodiments, a combination comprising a pharmaceutical composition comprising an SV2A inhibitor (e.g., levetiracetam, brivaracetam, or seletracetam), or the pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, comprises the SV2A inhibitor (e.g., levetiracetam, brivaracetam, or seletracetam), or the pharmaceutically acceptable salt, hydrate, solvate, polymorph, or isomer thereof, in an amount of 190 mg-220 mg. In some embodiments, a combination comprising a pharmaceutical composition comprising an SV2A inhibitor (e.g., levetiracetam, brivaracetam, or seletracetam), or the pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, comprises the SV2A inhibitor (e.g., levetiracetam, brivaracetam, or seletracetam), or the pharmaceutically acceptable salt, hydrate, solvate, polymorph, or isomer thereof, in an amount of 190 mg-240 mg. In some embodiments, a combination comprising a pharmaceutical composition comprising an SV2A inhibitor (e.g., levetiracetam, brivaracetam, or seletracetam), or the pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, comprises the SV2A inhibitor (e.g., levetiracetam, brivaracetam, or seletracetam), or the pharmaceutically acceptable salt, hydrate, solvate, polymorph, or isomer thereof, in an amount of about 220 mg.

In certain embodiments of the disclosure, a combination comprising a pharmaceutical composition comprising an SV2A inhibitor (e.g., levetiracetam, brivaracetam, or seletracetam), or the pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, comprises an amount of the SV2A inhibitor (e.g., levetiracetam, brivaracetam, or seletracetam), or the pharmaceutically acceptable salt, hydrate, solvate, polymorph, or isomer thereof, of 0.05 mg-35 mg, 0.07 mg-60 mg, 0.07 mg-350 mg, 25 mg-60 mg, 25 mg-125 mg, 50 mg-250 mg, 5 mg-15 mg, 5 mg-30 mg, 5 mg-140 mg, 0.7 mg-180 mg, 125 mg-240 mg, 3 mg-50 mg, or 0.07 mg-50 mg, 3 mg-60 mg, or about 0.1 mg-500 mg, 0.1 mg-350 mg, 0.7 mg-350 mg, 3 mg-300 mg, 3 mg-150 mg, 3 mg-110 mg, 7 mg-70 mg, 70 mg-350 mg, 100 mg-300 mg, or 125 mg-250 mg. In other embodiments, the amount of the SV2A inhibitor (e.g., levetiracetam, brivaracetam, or seletracetam), or the pharmaceutically acceptable salt, hydrate, solvate, polymorph, or isomer thereof, is less than 500 mg, less than 350 mg, less than 300 mg, less than 250 mg, less than 200 mg, less than 150 mg, less than 110 mg, less than 100 mg, less than 70 mg, less than 50 mg, less than 35 mg, less than 10 mg, less than 7 mg, less than 5 mg, less than 3 mg, less than 1 mg, less than 0.7 mg, less than 0.5 mg, less than 0.1 mg, less than 0.07 mg, or less than 0.05 mg.

In certain embodiments of the disclosure, a combination comprising a pharmaceutical composition comprising an SV2A inhibitor (e.g., levetiracetam, brivaracetam, or seletracetam), or the pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, comprises an amount of the SV2A inhibitor (e.g., levetiracetam, brivaracetam, or seletracetam), or the pharmaceutically acceptable salt, hydrate, solvate, polymorph, or isomer thereof, of 0.1 mg-500 mg, 0.1 mg-300 mg, 0.7 mg-300 mg, 3 mg-300 mg, 3 mg-150 mg, 3 mg-110 mg, 7 mg-70 mg, 7 mg-300 mg, 70 mg-300 mg, 100 mg-300 mg, 125 mg-250 mg, 0.5 mg-50 mg, 0.5 mg-75 mg, 0.5 mg-100 mg, 0.5 mg-150 mg, 0.5 mg-200 mg, 0.5 mg-225 mg, 0.5 mg-250 mg, 0.5 mg-300 mg, 1.5 mg-50 mg, 1.5 mg-75 mg, 1.5 mg-100 mg, 1.5 mg-150 mg, 1.5 mg-200 mg, 1.5 mg-225 mg, 1.5 mg-250 mg, 1.5 mg-300 mg, 3 mg-50 mg, 3 mg-75 mg, 3 mg-100 mg, 3 mg-150 mg, 3 mg-200 mg, 3 mg-225 mg, 3 mg-250 mg, 3 mg-300 mg, 5 mg-50 mg, 5 mg-75 mg, 5 mg-100 mg, 5 mg-150 mg, 5 mg-200 mg, 5 mg-225 mg, 5 mg-250 mg, 5 mg-300 mg, 7 mg-50 mg, 7 mg-75 mg, 7 mg-100 mg, 7 mg-150 mg, 7 mg-200 mg, 7 mg-225 mg, 7 mg-250 mg, 7 mg-300 mg, 15 mg-50 mg, 15 mg-75 mg, 15 mg-100 mg, 15 mg-150 mg, 15 mg-200 mg, 15 mg-225 mg, 15 mg-250 mg, 15 mg-300 mg, 30 mg-50 mg, 30 mg-75 mg, 30 mg-100 mg, 30 mg-150 mg, 30 mg-200 mg, 30 mg-225 mg, 30 mg-250 mg, or 30 mg-300 mg.

In certain embodiments of the disclosure, a combination comprising a pharmaceutical composition comprising a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, and an SV2A inhibitor (e.g., levetiracetam, brivaracetam, or seletracetam), or the pharmaceutically acceptable salt, hydrate, solvate, polymorph, or isomer thereof, comprises an amount of the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, of 0.05 mg to 5000 mg or 5 mg to 1000 mg. In some embodiments, the combination may comprise a pharmaceutical composition comprising about 0.5 mg, about 5 mg, about 20 mg, about 50 mg, about 75 mg, about 100 mg, about 150 mg, about 250 mg, about 500 mg, about 750 mg, about 1000 mg, about 1250 mg, about 2500 mg, about 3500 mg, or 5000 mg of the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof.

In certain embodiments of the disclosure, a combination comprising a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, and an SV2A inhibitor (e.g., levetiracetam, brivaracetam, or seletracetam), or the pharmaceutically acceptable salt, hydrate, solvate, polymorph, or isomer thereof, comprises an amount of the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, of 0.05 mg to 5000 mg or 5 mg to 1000 mg. In some embodiments, the combination may comprise about 0.5 mg, about 5 mg, about 20 mg, about 50 mg, about 75 mg, about 100 mg, about 150 mg, about 250 mg, about 500 mg, about 750 mg, about 1000 mg, about 1250 mg, about 2500 mg, about 3500 mg, or 5000 mg of the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof.

In some embodiments, the compound or pharmaceutical composition of the disclosure is in an extended release form (e.g., a controlled release form, a prolonged release form, a sustained release form, a delayed release form, or a slow release form). “Extended release” is widely recognized in the art of pharmaceutical sciences and may refer to a controlled release of an active compound from a dosage form to an environment over (throughout or during) an extended period of time, e.g., greater than or equal to one hour. An extended release dosage form will release a compound of the disclosure at substantially constant rate over an extended period of time or a substantially constant amount of a compound of the disclosure will be released incrementally over an extended period of time. “Extended release” as used herein may include “controlled release,” “prolonged release,” “sustained release,” “delayed release,” or “slow release” as these terms are used in the pharmaceutical sciences. In some embodiments, the extended release dosage may be administered in the form of a patch or a pump. “Extended release dosage form” or “extended release form”, as used herein, may refer to a dosage form that comprises one or more active ingredients, where the release of at least one of the active ingredient, when placed in water or other biological fluids or solvents, may occur over an extended period, such as a period of at least about 1 day, at least about 2 days, at least about 3 days, at least about 4 days, at least about 5 days, at least about 10 days, at least about 20 days, at least about 30 days, at least about 60 days, at least about 90 days, or at least about 150 days. Alternatively, the compound of the disclosure or pharmaceutical composition may be in an “immediate release” form or in a “non-extended release” form.

In some embodiments of the present disclosure, only one of the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, is in an extended release dosage form (e.g., a controlled release form, a prolonged release form, a sustained release form, a delayed release form, or a slow release form). In some embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are each in an extended release dosage form (e.g., a controlled release form, a prolonged release form, a sustained release form, a delayed release form, or a slow release form). In some embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, may be formulated together or in separate formulations. In certain such embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, may be together in one pharmaceutical composition or separate in two pharmaceutical compositions.

In some embodiments, the pharmaceutical composition of the disclosure (e.g., a pharmaceutical composition comprising an SV2A inhibitor (e.g., levetiracetam, brivaracetam, or seletracetam), or a pharmaceutically acceptable salt, hydrate, solvate, polymorph, or isomer thereof; a pharmaceutical composition comprising a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof; or a pharmaceutical composition comprising both an SV2A inhibitor (e.g., levetiracetam, brivaracetam, or seletracetam), or a pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof) is an extended release form (e.g., a controlled release form, a prolonged release form, a sustained release form, a delayed release form, or a slow release form). In some embodiments, the pharmaceutical composition of the disclosure (e.g., a pharmaceutical composition comprising an SV2A inhibitor (e.g., levetiracetam, brivaracetam, or seletracetam), or a pharmaceutically acceptable salt, hydrate, solvate, polymorph, or isomer thereof; a pharmaceutical composition comprising a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof; or a pharmaceutical composition comprising both an SV2A inhibitor (e.g., levetiracetam, brivaracetam, or seletracetam), or a pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof) is an immediate release form. In some embodiments, the pharmaceutical composition of the disclosure (e.g., a pharmaceutical composition comprising an SV2A inhibitor (e.g., levetiracetam, brivaracetam, or seletracetam), or a pharmaceutically acceptable salt, hydrate, solvate, polymorph, or isomer thereof; a pharmaceutical composition comprising a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof; or a pharmaceutical composition comprising both an SV2A inhibitor (e.g., levetiracetam, brivaracetam, or seletracetam), or a pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof) is in a non-extended release form. In some embodiments wherein the pharmaceutical composition comprises an SV2A inhibitor (e.g., levetiracetam, brivaracetam, or seletracetam), or a pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, the compounds are in the same release form (e.g., both are in an extended release form (e.g., a controlled release form, a prolonged release form, a sustained release form, a delayed release form, or a slow release form)). In some embodiments wherein the pharmaceutical composition comprises an SV2A inhibitor (e.g., levetiracetam, brivaracetam, or seletracetam), or a pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, the compounds are in different release forms (e.g., one is in an extended release form (e.g., a controlled release form, a prolonged release form, a sustained release form, a delayed release form, or a slow release form) and the other is in an immediate release form).

In some embodiments of the combinations of the disclosure, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are in extended release forms (e.g., a controlled release form, a prolonged release form, a sustained release form, a delayed release form, or a slow release form). In some embodiments of the combinations of the disclosure, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are in immediate release forms. In some embodiments of the combinations of the disclosure, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are in non-extended release forms. In some embodiments of the combinations of the disclosure, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are in the same release form (e.g., both are in an extended release form (e.g., a controlled release form, a prolonged release form, a sustained release form, a delayed release form, or a slow release form)). In some embodiments of the combinations of the disclosure, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are in different release forms (e.g., one is in an extended release form (e.g., a controlled release form, a prolonged release form, a sustained release form, a delayed release form, or a slow release form) and the other is in an immediate release form). In some embodiments of the combinations of the disclosure, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are formulated together. In some embodiments of the combinations of the disclosure, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are formulated separately, and may be packaged together or separately. In some embodiments of the combinations of the disclosure, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are in a single pharmaceutical composition. In some embodiments of the combinations of the disclosure, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are formulated in separate pharmaceutical compositions. In certain such embodiments, the separate pharmaceutical compositions are packaged together. In some embodiments, the separate pharmaceutical compositions are packaged separately.

In some embodiments, the SV2A inhibitor (e.g., levetiracetam, brivaracetam, or seletracetam), or the pharmaceutically acceptable salt, hydrate, solvate, polymorph, or isomer thereof, is in an extended release form (e.g., a controlled release form, a prolonged release form, a sustained release form, a delayed release form, or a slow release form). In some embodiments, the SV2A inhibitor (e.g., levetiracetam, brivaracetam, or seletracetam), or the pharmaceutically acceptable salt, hydrate, solvate, polymorph, or isomer thereof, is an immediate release form. In some embodiments, the SV2A inhibitor (e.g., levetiracetam, brivaracetam, or seletracetam), or the pharmaceutically acceptable salt, hydrate, solvate, polymorph, or isomer thereof, is in a non-extended release form.

In some embodiments, the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, is in an extended release form (e.g., a controlled release form, a prolonged release form, a sustained release form, a delayed release form, or a slow release form). In some embodiments, the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, is an immediate release form. In some embodiments, the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, is in a non-extended release form.

In some embodiments, the SV2A inhibitor (e.g., levetiracetam, brivaracetam, or seletracetam), or the pharmaceutically acceptable salt, hydrate, solvate, polymorph, or isomer thereof, in an extended release form (e.g., a controlled release form, a prolonged release form, a sustained release form, a delayed release form, or a slow release form) is formulated together with the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof. In some embodiments, the SV2A inhibitor (e.g., levetiracetam, brivaracetam, or seletracetam), or the pharmaceutically acceptable salt, hydrate, solvate, polymorph, or isomer thereof, in an extended release form (e.g., a controlled release form, a prolonged release form, a sustained release form, a delayed release form, or a slow release form) is formulated together with the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, in a single pharmaceutical composition.

In some embodiments, the SV2A inhibitor (e.g., levetiracetam, brivaracetam, or seletracetam), or the pharmaceutically acceptable salt, hydrate, solvate, polymorph, or isomer thereof, in an extended release form (e.g., a controlled release form, a prolonged release form, a sustained release form, a delayed release form, or a slow release form) and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are formulated separately and may be packaged together or separately. In some embodiments, the SV2A inhibitor (e.g., levetiracetam, brivaracetam, or seletracetam), or the pharmaceutically acceptable salt, hydrate, solvate, polymorph, or isomer thereof, in an extended release form (e.g., a controlled release form, a prolonged release form, a sustained release form, a delayed release form, or a slow release form) and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are formulated in separate pharmaceutical compositions. In certain such embodiments, the separate pharmaceutical compositions are packaged together. In some embodiments, the separate pharmaceutical compositions are packaged separately.

In some embodiments, the SV2A inhibitor (e.g., levetiracetam, brivaracetam, or seletracetam), or the pharmaceutically acceptable salt, hydrate, solvate, polymorph, or isomer thereof, in an immediate release form is formulated together with the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof. In some embodiments, the SV2A inhibitor (e.g., levetiracetam, brivaracetam, or seletracetam), or the pharmaceutically acceptable salt, hydrate, solvate, polymorph, or isomer thereof, in an immediate release form is formulated together with the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, in a single pharmaceutical composition.

In some embodiments, the SV2A inhibitor (e.g., levetiracetam, brivaracetam, or seletracetam), or the pharmaceutically acceptable salt, hydrate, solvate, polymorph, or isomer thereof, in an immediate release and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are formulated separately and may be packaged together or separately. In some embodiments, the SV2A inhibitor (e.g., levetiracetam, brivaracetam, or seletracetam), or the pharmaceutically acceptable salt, hydrate, solvate, polymorph, or isomer thereof, in an immediate release and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are formulated in separate pharmaceutical compositions. In certain such embodiments, the separate pharmaceutical compositions are packaged together. In some embodiments, the separate pharmaceutical compositions are packaged separately.

In some embodiments, the SV2A inhibitor (e.g., levetiracetam, brivaracetam, or seletracetam), or the pharmaceutically acceptable salt, hydrate, solvate, polymorph, or isomer thereof, in a non-extended release form is formulated together with the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof. In some embodiments, the SV2A inhibitor (e.g., levetiracetam, brivaracetam, or seletracetam), or the pharmaceutically acceptable salt, hydrate, solvate, polymorph, or isomer thereof, in a non-extended release form is formulated together with the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, in a single pharmaceutical composition.

In some embodiments, the SV2A inhibitor (e.g., levetiracetam, brivaracetam, or seletracetam), or the pharmaceutically acceptable salt, hydrate, solvate, polymorph, or isomer thereof, in a non-extended release form and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are formulated separately and may be packaged together or separately. In some embodiments, the SV2A inhibitor (e.g., levetiracetam, brivaracetam, or seletracetam), or the pharmaceutically acceptable salt, hydrate, solvate, polymorph, or isomer thereof, in a non-extended release form and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are formulated in separate pharmaceutical compositions. In certain such embodiments, the separate pharmaceutical compositions are packaged together. In some embodiments, the separate pharmaceutical compositions are packaged separately.

In some embodiments, the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, in an extended release form (e.g., a controlled release form, a prolonged release form, a sustained release form, a delayed release form, or a slow release form) is formulated together with the SV2A inhibitor (e.g., levetiracetam, brivaracetam, or seletracetam), or the pharmaceutically acceptable salt, hydrate, solvate, polymorph, or isomer thereof. In some embodiments, the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, in an extended release form (e.g., a controlled release form, a prolonged release form, a sustained release form, a delayed release form, or a slow release form) is formulated together with the SV2A inhibitor (e.g., levetiracetam, brivaracetam, or seletracetam), or the pharmaceutically acceptable salt, hydrate, solvate, polymorph, or isomer thereof, in a single pharmaceutical composition.

In some embodiments, the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, in an extended release form (e.g., a controlled release form, a prolonged release form, a sustained release form, a delayed release form, or a slow release form) and the SV2A inhibitor (e.g., levetiracetam, brivaracetam, or seletracetam), or the pharmaceutically acceptable salt, hydrate, solvate, polymorph, or isomer thereof, are formulated separately and may be packaged together or separately. In some embodiments, the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, in an extended release form (e.g., a controlled release form, a prolonged release form, a sustained release form, a delayed release form, or a slow release form) and the SV2A inhibitor (e.g., levetiracetam, brivaracetam, or seletracetam), or the pharmaceutically acceptable salt, hydrate, solvate, polymorph, or isomer thereof, are formulated in separate pharmaceutical compositions. In certain such embodiments, the separate pharmaceutical compositions are packaged together. In some embodiments, the separate pharmaceutical compositions are packaged separately.

In some embodiments, the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, in an immediate release form is formulated together with the SV2A inhibitor (e.g., levetiracetam, brivaracetam, or seletracetam), or the pharmaceutically acceptable salt, hydrate, solvate, polymorph, or isomer thereof. In some embodiments, the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, in an immediate release form is formulated together with the SV2A inhibitor (e.g., levetiracetam, brivaracetam, or seletracetam), or the pharmaceutically acceptable salt, hydrate, solvate, polymorph, or isomer thereof, in a single pharmaceutical composition.

In some embodiments, the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, in an immediate release and the SV2A inhibitor (e.g., levetiracetam, brivaracetam, or seletracetam), or the pharmaceutically acceptable salt, hydrate, solvate, polymorph, or isomer thereof, are formulated separately. In some embodiments, the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, in an immediate release and the SV2A inhibitor (e.g., levetiracetam, brivaracetam, or seletracetam), or the pharmaceutically acceptable salt, hydrate, solvate, polymorph, or isomer thereof, are formulated in separate pharmaceutical compositions. In certain such embodiments, the separate pharmaceutical compositions are packaged together. In some embodiments, the separate pharmaceutical compositions are packaged separately.

In some embodiments, the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, in a non-extended release form is formulated together with the SV2A inhibitor (e.g., levetiracetam, brivaracetam, or seletracetam), or the pharmaceutically acceptable salt, hydrate, solvate, polymorph, or isomer thereof. In some embodiments, the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, in a non-extended release form is formulated together with the SV2A inhibitor (e.g., levetiracetam, brivaracetam, or seletracetam), or the pharmaceutically acceptable salt, hydrate, solvate, polymorph, or isomer thereof, in a single pharmaceutical composition.

In some embodiments, the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, in a non-extended release form and the SV2A inhibitor (e.g., levetiracetam, brivaracetam, or seletracetam), or the pharmaceutically acceptable salt, hydrate, solvate, polymorph, or isomer thereof, are formulated separately and may be packaged together or separately. In some embodiments, the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, in a non-extended release form and the SV2A inhibitor (e.g., levetiracetam, brivaracetam, or seletracetam), or the pharmaceutically acceptable salt, hydrate, solvate, polymorph, or isomer thereof, are formulated in separate pharmaceutical compositions. In certain such embodiments, the separate pharmaceutical compositions are packaged together. In some embodiments, the separate pharmaceutical compositions are packaged separately.

In some embodiments wherein the SV2A inhibitor (e.g., levetiracetam, brivaracetam, or seletracetam), or the pharmaceutically acceptable salt, hydrate, solvate, polymorph, or isomer thereof, is in an extended release form (e.g., a controlled release form, a prolonged release form, a sustained release form, a delayed release form, or a slow release form), the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, is also in an extended release form (e.g., a controlled release form, a prolonged release form, a sustained release form, a delayed release form, or a slow release form). In some embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are formulated together. In some embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are in a single pharmaceutical composition. In some embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are formulated separately and may be packaged together or separately. In some embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are formulated in separate pharmaceutical compositions. In certain such embodiments, the separate pharmaceutical compositions are packaged together. In some embodiments, the separate pharmaceutical compositions are packaged separately.

In some embodiments wherein the SV2A inhibitor (e.g., levetiracetam, brivaracetam, or seletracetam), or the pharmaceutically acceptable salt, hydrate, solvate, polymorph, or isomer thereof, is in an extended release form (e.g., a controlled release form, a prolonged release form, a sustained release form, a delayed release form, or a slow release form), the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, is in a non-extended release form. In certain such embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are formulated together. In some embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are in a single pharmaceutical composition. In some embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are formulated separately and may be packaged together or separately. In some embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are formulated in separate pharmaceutical compositions. In certain such embodiments, the separate pharmaceutical compositions are packaged together. In some embodiments, the separate pharmaceutical compositions are packaged separately.

In some embodiments wherein the SV2A inhibitor (e.g., levetiracetam, brivaracetam, or seletracetam), or the pharmaceutically acceptable salt, hydrate, solvate, polymorph, or isomer thereof, is in an extended release form (e.g., a controlled release form, a prolonged release form, a sustained release form, a delayed release form, or a slow release form), the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, is in an immediate release form. In certain such embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are formulated together. In some embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are in a single pharmaceutical composition. In some embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are formulated separately and may be packaged together or separately. In some embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are formulated in separate pharmaceutical compositions. In certain such embodiments, the separate pharmaceutical compositions are packaged together. In some embodiments, the separate pharmaceutical compositions are packaged separately.

In some embodiments wherein the SV2A inhibitor (e.g., levetiracetam, brivaracetam, or seletracetam), or the pharmaceutically acceptable salt, hydrate, solvate, polymorph, or isomer thereof, is in an immediate form, the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, is also in an extended release form (e.g., a controlled release form, a prolonged release form, a sustained release form, a delayed release form, or a slow release form). In certain such embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are formulated together. In some embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are in a single pharmaceutical composition. In some embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are formulated separately and may be packaged together or separately. In some embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are formulated in separate pharmaceutical compositions. In certain such embodiments, the separate pharmaceutical compositions are packaged together. In some embodiments, the separate pharmaceutical compositions are packaged separately.

In some embodiments wherein the SV2A inhibitor (e.g., levetiracetam, brivaracetam, or seletracetam), or the pharmaceutically acceptable salt, hydrate, solvate, polymorph, or isomer thereof, is in an immediate release form, the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, is in a non-extended release form. In some embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are formulated together. In some embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are in a single pharmaceutical composition. In some embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are formulated separately and may be packaged together or separately. In some embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are formulated in separate pharmaceutical compositions. In certain such embodiments, the separate pharmaceutical compositions are packaged together. In some embodiments, the separate pharmaceutical compositions are packaged separately.

In some embodiments wherein the SV2A inhibitor (e.g., levetiracetam, brivaracetam, or seletracetam), or the pharmaceutically acceptable salt, hydrate, solvate, polymorph, or isomer thereof, is in an immediate release form, the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, is in an immediate release form. In some embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are formulated together. In some embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are in a single pharmaceutical composition. In some embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are formulated separately and may be packaged together or separately. In some embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are formulated in separate pharmaceutical compositions. In certain such embodiments, the separate pharmaceutical compositions are packaged together. In some embodiments, the separate pharmaceutical compositions are packaged separately.

In some embodiments wherein the SV2A inhibitor (e.g., levetiracetam, brivaracetam, or seletracetam), or the pharmaceutically acceptable salt, hydrate, solvate, polymorph, or isomer thereof, is in a non-extended release form, the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, is also in an extended release form (e.g., a controlled release form, a prolonged release form, a sustained release form, a delayed release form, or a slow release form). In some embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are formulated together. In some embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are in a single pharmaceutical composition. In some embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are formulated separately and may be packaged together or separately. In some embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are formulated in separate pharmaceutical compositions. In certain such embodiments, the separate pharmaceutical compositions are packaged together. In some embodiments, the separate pharmaceutical compositions are packaged separately.

In some embodiments wherein the SV2A inhibitor (e.g., levetiracetam, brivaracetam, or seletracetam), or the pharmaceutically acceptable salt, hydrate, solvate, polymorph, or isomer thereof, is in a non-extended release form, the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, is in a non-extended release form. In some embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are formulated together. In some embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are in a single pharmaceutical composition. In some embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are formulated separately and may be packaged together or separately. In some embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are formulated in separate pharmaceutical compositions. In certain such embodiments, the separate pharmaceutical compositions are packaged together. In some embodiments, the separate pharmaceutical compositions are packaged separately.

In some embodiments wherein the SV2A inhibitor (e.g., levetiracetam, brivaracetam, or seletracetam), or the pharmaceutically acceptable salt, hydrate, solvate, polymorph, or isomer thereof, is in a non-extended release form, the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, is in an immediate release form. In some embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are formulated together. In some embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are in a single pharmaceutical composition. In some embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are formulated separately and may be packaged together or separately. In some embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are formulated in separate pharmaceutical compositions. In certain such embodiments, the separate pharmaceutical compositions are packaged together. In some embodiments, the separate pharmaceutical compositions are packaged separately.

### Methods and Uses of the Disclosure

The methods, uses, pharmaceutical compositions for use, or combinations for use of this disclosure may comprise administration or use of an SV2A inhibitor (e.g., levetiracetam, brivaracetam, or seletracetam), or a pharmaceutically acceptable salt, hydrate, solvate, polymorph, or isomer thereof, in combination with a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, as well as administration or use of one or more pharmaceutical compositions of the disclosure.

The methods, uses, pharmaceutical compositions, combinations, pharmaceutical compositions for use, or combinations for use of the disclosure are useful for treating cognitive impairment, delaying or slowing the progression of cognitive impairment, or reducing the rate of decline of cognitive function, in a subject suffering from cognitive impairment or decline of cognitive function associated with a central nervous system (CNS) disorder, or at risk thereof, by administering to said subject a therapeutically effective amount of an SV2A inhibitor (e.g., levetiracetam, brivaracetam, or seletracetam), or a pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof. In some embodiments, the methods, uses, pharmaceutical compositions, combinations, pharmaceutical compositions for use, or combinations for use of the disclosure are useful for treating cognitive impairment in said subject. In some embodiments, the methods, uses, pharmaceutical compositions, combinations, pharmaceutical compositions for use, or combinations for use of the disclosure are useful for improving cognitive function in said subject. In some embodiments, the methods, uses, pharmaceutical compositions, combinations, pharmaceutical compositions for use, or combinations for use of the disclosure are useful for delaying or slowing the progression of cognitive impairment in said subject. In some embodiments, the methods, uses, pharmaceutical compositions, combinations, pharmaceutical compositions for use, or combinations for use of the disclosure are useful for reducing the rate of decline of cognitive function in said subject. In some embodiments, the methods, uses, pharmaceutical compositions, combinations, pharmaceutical compositions for use, or combinations for use of the disclosure are useful for preventing or slowing the progression of the cognitive impairment in said subject. In other embodiments, the methods, uses, pharmaceutical compositions, combinations, pharmaceutical compositions for use, or combinations for use of the disclosure are useful for alleviating, ameliorating, or slowing the progression of one or more symptoms associated with the cognitive impairment in said subject.

Additionally, the methods, uses, combinations, pharmaceutical compositions, combinations for use, or pharmaceutical compositions for use are useful for treating cognitive impairment associated with a brain cancer or for treating a brain cancer itself in a subject in need thereof. Additionally, the methods, uses, combinations, pharmaceutical compositions, combinations for use, or pharmaceutical compositions for use are useful for treating Parkinson's disease psychosis in a subject in need thereof.

In some embodiments of the methods, uses, combinations, pharmaceutical compositions, combinations for use, or pharmaceutical compositions for use of the disclosure, the SV2A inhibitor, or the pharmaceutically acceptable salt, hydrate, solvate, polymorph, or isomer thereof, is levetiracetam, or a pharmaceutically acceptable salt, hydrate, solvate, polymorph, or isomer thereof. In some embodiments, the SV2A inhibitor, or the pharmaceutically acceptable salt, hydrate, solvate, polymorph, or isomer thereof, is brivaracetam, or a pharmaceutically acceptable salt, hydrate, solvate, polymorph, or isomer thereof. In some embodiments, the SV2A inhibitor, or the pharmaceutically acceptable salt, hydrate, solvate, polymorph, or isomer thereof, is seletracetam, or a pharmaceutically acceptable salt, hydrate, solvate, polymorph, or isomer thereof. In some embodiments, the GABA_{A} α5 receptor agonist, or the pharmaceutically acceptable salt, hydrate, solvate, polymorph, or isomer thereof, is selected from the group consisting of: a compound of Formula I, or a pharmaceutically acceptable salt, hydrate, solvate, polymorph, or isomer thereof; a compound of Formula II, or a pharmaceutically acceptable salt, hydrate, solvate, polymorph, or isomer thereof; and a compound of Formula IV, or a pharmaceutically acceptable salt, hydrate, solvate, polymorph, or isomer thereof. In some embodiments, the GABA_{A} α5 receptor agonist, or the pharmaceutically acceptable salt, hydrate, solvate, polymorph, or isomer thereof, is a compound of Formula I, or a pharmaceutically acceptable salt, hydrate, solvate, polymorph, or isomer thereof. In some embodiments, the GABA_{A} α5 receptor agonist, or the pharmaceutically acceptable salt, hydrate, solvate, polymorph, or isomer thereof, is a compound of Formula II, or a pharmaceutically acceptable salt, hydrate, solvate, polymorph, or isomer thereof. In some embodiments, the GABA_{A} α5 receptor agonist, or the pharmaceutically acceptable salt, hydrate, solvate, polymorph, or isomer thereof, is a compound of Formula IV, or a pharmaceutically acceptable salt, hydrate, solvate, polymorph, or isomer thereof. In some embodiments, the compound of Formula I, or the pharmaceutically acceptable salt, hydrate, solvate, polymorph, or isomer thereof, is selected from the group consisting of Compounds 1-114, or a pharmaceutically acceptable salt, hydrate, solvate, polymorph, or isomer of any of the foregoing. In some embodiments, the compound of Formula I, or the pharmaceutically acceptable salt, hydrate, solvate, polymorph, or isomer thereof, is Compound 1, or a pharmaceutically acceptable salt, hydrate, solvate, polymorph, or isomer thereof. In some embodiments, the methods, uses, combinations, pharmaceutical compositions, combinations for use, or pharmaceutical compositions for use of the disclosure may comprise one or more crystalline forms selected from the group consisting of Compound 1, Form A; Compound 1, Form B; Compound 1, Form C; Compound 1, Form E; and Compound 1, Form F. In some embodiments, the crystalline form is Compound 1, Form A.

In some embodiments, the cognitive impairment is associated with a CNS disorder, such as age-related cognitive impairment, MCI, AAMI, ARCD. In certain such embodiments, the MCI is amnestic MCI. In some embodiments, the cognitive impairment is associated with a CNS disorder, such as age-related cognitive impairment. In some embodiments, the cognitive impairment is associated with a CNS disorder, such as MCI. In some embodiments, the cognitive impairment is associated with dementia, Alzheimer's disease (AD), prodromal AD, post-traumatic stress disorder (PTSD), schizophrenia, bipolar disorder, amyotrophic lateral sclerosis, cancer-therapy-related cognitive impairment, mental retardation, Parkinson's disease, autism, compulsive behavior, or substance addiction. In some embodiments, the cognitive impairment is associated with a brain cancer. In some embodiments, the cognitive impairment is associated with dementia. In some embodiments, the dementia is Alzheimer's disease. In some embodiments, the CNS disorder is schizophrenia, amyotrophic lateral sclerosis (ALS), post-traumatic stress disorder (PTSD), mental retardation, Parkinson's disease (PD), autism, compulsive behavior, substance addiction, bipolar disorder, or cancer-therapy-related cognitive impairment. In some embodiments, the subject that suffers from cognitive impairment or decline of cognitive function is a human.

In some embodiments, methods, uses, combinations, pharmaceutical compositions, combinations for use, or pharmaceutical compositions for use are useful for treating cognitive impairment associated with a brain cancer or for treating a brain cancer itself in a subject in need thereof. In some embodiments, the methods, uses, combinations, pharmaceutical compositions, combinations for use, or pharmaceutical compositions for use are useful for treating Parkinson's disease psychosis in a subject in need thereof. In some embodiments, the subject in need thereof is a human.

The disclosure provides for a method of treating cognitive impairment associated with a central nervous system (CNS) disorder in a subject in need thereof or at risk thereof, the method comprising administering to the subject an SV2A inhibitor (e.g., levetiracetam, brivaracetam, or seletracetam), or a pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof. In certain such embodiments, a therapeutically effective amount of the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, is administered. The disclosure provides for a method of treating cognitive impairment associated with a central nervous system (CNS) disorder in a subject in need thereof or at risk thereof, the method comprising administering to the subject a combination or pharmaceutical composition of the disclosure. In certain such embodiments, a therapeutically effective amount of the combination or pharmaceutical composition of the disclosure is administered.

The disclosure provides for a method of treating cognitive impairment associated with a brain cancer in a subject in need thereof or at risk thereof, the method comprising administering to the subject an SV2A inhibitor (e.g., levetiracetam, brivaracetam, or seletracetam), or a pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof. In certain such embodiments, a therapeutically effective amount of the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, is administered. The disclosure provides for a method of treating cognitive impairment associated with a brain cancer in a subject in need thereof or at risk thereof, the method comprising administering to the subject a combination or pharmaceutical composition of the disclosure. In certain such embodiments, a therapeutically effective amount of the combination or pharmaceutical composition of the disclosure is administered.

The disclosure provides for a method of treating a brain cancer in a subject in need thereof or at risk thereof, the method comprising administering to the subject an SV2A inhibitor (e.g., levetiracetam, brivaracetam, or seletracetam), or a pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof. In certain such embodiments, a therapeutically effective amount of the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, is administered. The disclosure provides for a method of treating a brain cancer in a subject in need thereof or at risk thereof, the method comprising administering to the subject a combination or pharmaceutical composition of the disclosure. In certain such embodiments, a therapeutically effective amount of the combination or pharmaceutical composition of the disclosure is administered.

The disclosure provides for a method of treating Parkinson's disease psychosis in a subject in need thereof or at risk thereof, the method comprising administering to the subject an SV2A inhibitor (e.g., levetiracetam, brivaracetam, or seletracetam), or a pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof. In certain such embodiments, a therapeutically effective amount of the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, is administered. The disclosure provides for a method of treating a Parkinson's disease psychosis in a subject in need thereof or at risk thereof, the method comprising administering to the subject a combination or pharmaceutical composition of the disclosure. In certain such embodiments, a therapeutically effective amount of the combination or pharmaceutical composition of the disclosure is administered.

The disclosure provides for use of an SV2A inhibitor (e.g., levetiracetam, brivaracetam, or seletracetam), or a pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, for treating cognitive impairment associated with a central nervous system (CNS) disorder in a subject in need thereof or at risk thereof. In certain such embodiments, a therapeutically effective amount of the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, is used. The disclosure provides for use of a combination or pharmaceutical composition of the disclosure for treating cognitive impairment associated with a central nervous system (CNS) disorder in a subject in need thereof or at risk thereof. In certain such embodiments, a therapeutically effective amount of the combination or pharmaceutical composition of the disclosure is used.

The disclosure provides for use of an SV2A inhibitor (e.g., levetiracetam, brivaracetam, or seletracetam), or a pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, for treating cognitive impairment associated with a brain cancer in a subject in need thereof or at risk thereof. In certain such embodiments, a therapeutically effective amount of the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, is used. The disclosure provides for use of a combination or pharmaceutical composition of the disclosure for treating cognitive impairment associated with a brain cancer in a subject in need thereof or at risk thereof. In certain such embodiments, a therapeutically effective amount of the combination or pharmaceutical composition of the disclosure is used.

The disclosure provides for use of an SV2A inhibitor (e.g., levetiracetam, brivaracetam, or seletracetam), or a pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, for treating a brain cancer in a subject in need thereof or at risk thereof. In certain such embodiments, a therapeutically effective amount of the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, is used. The disclosure provides for use of a combination or pharmaceutical composition of the disclosure for treating a brain cancer in a subject in need thereof or at risk thereof. In certain such embodiments, a therapeutically effective amount of the combination or pharmaceutical composition of the disclosure is used.

The disclosure provides for use of an SV2A inhibitor (e.g., levetiracetam, brivaracetam, or seletracetam), or a pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, for treating Parkinson's disease psychosis in a subject in need thereof or at risk thereof. In certain such embodiments, a therapeutically effective amount of the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, is used. The disclosure provides for use of a combination or pharmaceutical composition of the disclosure for treating Parkinson's disease psychosis in a subject in need thereof or at risk thereof. In certain such embodiments, a therapeutically effective amount of the combination or pharmaceutical composition of the disclosure is used.

The disclosure provides for use of an SV2A inhibitor (e.g., levetiracetam, brivaracetam, or seletracetam), or a pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, in the manufacture of a medicament. In certain such embodiments, a therapeutically effective amount of the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, is used. The disclosure provides for use of a combination or pharmaceutical composition of the disclosure in the manufacture of a medicament. In certain such embodiments, a therapeutically effective amount of the combination or pharmaceutical composition of the disclosure is used.

The disclosure provides for use of an SV2A inhibitor (e.g., levetiracetam, brivaracetam, or seletracetam), or a pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, in the manufacture of a medicament for treating cognitive impairment associated with a central nervous system (CNS) disorder in a subject in need thereof or at risk thereof. In certain such embodiments, a therapeutically effective amount of the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, is used. The disclosure provides for use of a combination or pharmaceutical composition of the disclosure in the manufacture of a medicament for treating cognitive impairment associated with a central nervous system (CNS) disorder in a subject in need thereof or at risk thereof. In certain such embodiments, a therapeutically effective amount of the combination or pharmaceutical composition of the disclosure is used.

The disclosure provides for use of an SV2A inhibitor (e.g., levetiracetam, brivaracetam, or seletracetam), or a pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, in the manufacture of a medicament for treating cognitive impairment associated with a brain cancer in a subject in need thereof or at risk thereof. In certain such embodiments, a therapeutically effective amount of the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, is used. The disclosure provides for use of a combination or pharmaceutical composition of the disclosure in the manufacture of a medicament for treating cognitive impairment associated with a brain cancer in a subject in need thereof or at risk thereof. In certain such embodiments, a therapeutically effective amount of the combination or pharmaceutical composition of the disclosure is used.

The disclosure provides for use of an SV2A inhibitor (e.g., levetiracetam, brivaracetam, or seletracetam), or a pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, in the manufacture of a medicament for treating a brain cancer in a subject in need thereof or at risk thereof. In certain such embodiments, a therapeutically effective amount of the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, is used. The disclosure provides for use of a combination or pharmaceutical composition of the disclosure in the manufacture of a medicament for treating a brain cancer in a subject in need thereof or at risk thereof. In certain such embodiments, a therapeutically effective amount of the combination or pharmaceutical composition of the disclosure is used.

The disclosure provides for use of an SV2A inhibitor (e.g., levetiracetam, brivaracetam, or seletracetam), or a pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, in the manufacture of a medicament for treating Parkinson's disease psychosis in a subject in need thereof or at risk thereof. In certain such embodiments, a therapeutically effective amount of the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, is used. The disclosure provides for use of a combination or pharmaceutical composition of the disclosure in the manufacture of a medicament for treating Parkinson's disease psychosis in a subject in need thereof or at risk thereof. In certain such embodiments, a therapeutically effective amount of the combination or pharmaceutical composition of the disclosure is used.

The disclosure provides an SV2A inhibitor (e.g., levetiracetam, brivaracetam, or seletracetam), or a pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, for use in treating cognitive impairment associated with a central nervous system (CNS) disorder in a subject in need thereof or at risk thereof. In certain such embodiments, a therapeutically effective amount of the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, is used. The disclosure provides a combination or pharmaceutical composition of the disclosure for use in treating cognitive impairment associated with a central nervous system (CNS) disorder in a subject in need thereof or at risk thereof. In certain such embodiments, a therapeutically effective amount of the combination or pharmaceutical composition of the disclosure is used.

The disclosure provides an SV2A inhibitor (e.g., levetiracetam, brivaracetam, or seletracetam), or a pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, for use in treating cognitive impairment associated with a brain cancer in a subject in need thereof or at risk thereof. In certain such embodiments, a therapeutically effective amount of the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, is used. The disclosure provides a combination or pharmaceutical composition of the disclosure for use in treating cognitive impairment associated with a brain cancer in a subject in need thereof or at risk thereof. In certain such embodiments, a therapeutically effective amount of the combination or pharmaceutical composition of the disclosure is used.

The disclosure provides an SV2A inhibitor (e.g., levetiracetam, brivaracetam, or seletracetam), or a pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, for use in treating a brain cancer in a subject in need thereof or at risk thereof. In certain such embodiments, a therapeutically effective amount of the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, is used. The disclosure provides a combination or pharmaceutical composition of the disclosure for use in treating a brain cancer in a subject in need thereof or at risk thereof. In certain such embodiments, a therapeutically effective amount of the combination or pharmaceutical composition of the disclosure is used.

The disclosure provides an SV2A inhibitor (e.g., levetiracetam, brivaracetam, or seletracetam), or a pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, for use in treating Parkinson's disease psychosis in a subject in need thereof or at risk thereof. In certain such embodiments, a therapeutically effective amount of the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, is used. The disclosure provides a combination or pharmaceutical composition of the disclosure for use in treating Parkinson's disease psychosis in a subject in need thereof or at risk thereof. In certain such embodiments, a therapeutically effective amount of the combination or pharmaceutical composition of the disclosure is used.

In some embodiments of the methods, uses, pharmaceutical compositions for use, or combinations for use of the disclosure, the combined treatment has a longer or improved therapeutic effect in the subject than is attained by administering the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, in the absence of the SV2A inhibitor (e.g., levetiracetam, brivaracetam, or seletracetam), or the pharmaceutically acceptable salt, hydrate, solvate, polymorph, or isomer thereof, by at least about 1.5×, or 2.0×, or 2.5×, or 3.0×, or 3.5×, or 4.0×, or 4.5×, or 5.0×, or 5.5×, or 6.0×, or 6.5×, or 7.0×, or 7.5×, or 8.0×, or 8.5×, or 9.0×, or 9.5×, or 10×, or greater than about 10×.

In some embodiments of the methods, uses, pharmaceutical compositions for use, or combinations for use of the disclosure, the combined treatment has a longer or improved therapeutic effect in the subject than is attained by administering the SV2A inhibitor (e.g., levetiracetam, brivaracetam, or seletracetam), or the pharmaceutically acceptable salt, hydrate, solvate, polymorph, or isomer thereof, in the absence of the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, by at least about 1.5×, or 2.0×, or 2.5×, or 3.0×, or 3.5×, or 4.0×, or 4.5×, or 5.0×, or 5.5×, or 6.0×, or 6.5×, or 7.0×, or 7.5×, or 8.0×, or 8.5×, or 9.0×, or 9.5×, or 10×, or greater than about 10×.

In accordance with another aspect of the present disclosure, there are provided methods, uses, pharmaceutical compositions, combinations, pharmaceutical compositions for use, or combinations for use for increasing the therapeutic index of a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, in a method of treating or for use in treating cognitive impairment associated with a central nervous system (CNS) disorder in a subject in need or at risk thereof, comprising administering an SV2A inhibitor (e.g., levetiracetam, brivaracetam, or seletracetam), or a pharmaceutically acceptable salt, hydrate, solvate, polymorph, or isomer thereof, in combination with a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, to said subject.

In accordance with another aspect of the present disclosure, there are provided methods, uses, pharmaceutical compositions, combinations, pharmaceutical compositions for use, or combinations for use for increasing the therapeutic index of a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, in a method of treating or for use in treating cognitive impairment associated with a brain cancer or for treating a brain cancer itself in a subject in need or at risk thereof, comprising administering an SV2A inhibitor (e.g., levetiracetam, brivaracetam, or seletracetam), or a pharmaceutically acceptable salt, hydrate, solvate, polymorph, or isomer thereof, in combination with a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, to said subject.

In accordance with another aspect of the present disclosure, there are provided methods, uses, pharmaceutical compositions, combinations, pharmaceutical compositions for use, or combinations for use for increasing the therapeutic index of a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, in a method of treating or for use in treating Parkinson's disease psychosis in a subject in need or at risk thereof, comprising administering an SV2A inhibitor (e.g., levetiracetam, brivaracetam, or seletracetam), or a pharmaceutically acceptable salt, hydrate, solvate, polymorph, or isomer thereof, in combination with a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, to said subject.

In some embodiments, the therapeutic index of the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, is greater than the therapeutic index of the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, when administered in the absence of the SV2A inhibitor (e.g., levetiracetam, brivaracetam, or seletracetam), or the pharmaceutically acceptable salt, hydrate, solvate, polymorph, or isomer thereof, by at least about 1.5×, or 2.0×, or 2.5×, or 3.0×, or 3.5×, or 4.0×, or 4.5×, or 5.0×, or 5.5×, or 6.0×, or 6.5×, or 7.0×, or 7.5×, or 8.0×, or 8.5×, or 9.0×, or 9.5×, or 10×, or greater than about 10×.

In accordance with another aspect of the present disclosure, there are provided methods, uses, pharmaceutical compositions, combinations, pharmaceutical compositions for use, or combinations for use for increasing the therapeutic index of an SV2A inhibitor (e.g., levetiracetam, brivaracetam, or seletracetam), or a pharmaceutically acceptable salt, hydrate, solvate, polymorph, or isomer thereof, in a method of treating or for use in treating cognitive impairment associated with central nervous system (CNS) disorder in a subject in need or at risk thereof, comprising administering an SV2A inhibitor (e.g., levetiracetam, brivaracetam, or seletracetam), or a pharmaceutically acceptable salt, hydrate, solvate, polymorph, or isomer thereof, in combination with a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, to said subject.

In accordance with another aspect of the present disclosure, there are provided methods, uses, pharmaceutical compositions, combinations, pharmaceutical compositions for use, or combinations for use for increasing the therapeutic index of an SV2A inhibitor (e.g., levetiracetam, brivaracetam, or seletracetam), or a pharmaceutically acceptable salt, hydrate, solvate, polymorph, or isomer thereof, in a method of treating or for use in treating cognitive impairment associated with a brain cancer or for treating a brain cancer itself in a subject in need or at risk thereof, comprising administering an SV2A inhibitor (e.g., levetiracetam, brivaracetam, or seletracetam), or a pharmaceutically acceptable salt, hydrate, solvate, polymorph, or isomer thereof, in combination with a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, to said subject.

In accordance with another aspect of the present disclosure, there are provided methods, uses, pharmaceutical compositions, combinations, pharmaceutical compositions for use, or combinations for use for increasing the therapeutic index of an SV2A inhibitor (e.g., levetiracetam, brivaracetam, or seletracetam), or a pharmaceutically acceptable salt, hydrate, solvate, polymorph, or isomer thereof, in a method of treating or for use in treating Parkinson's disease psychosis in a subject in need or at risk thereof, comprising administering an SV2A inhibitor (e.g., levetiracetam, brivaracetam, or seletracetam), or a pharmaceutically acceptable salt, hydrate, solvate, polymorph, or isomer thereof, in combination with a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, to said subject.

In some embodiments, the therapeutic index of the SV2A inhibitor (e.g., levetiracetam, brivaracetam, or seletracetam), or the pharmaceutically acceptable salt, hydrate, solvate, polymorph, or isomer thereof, is greater than the therapeutic index of the SV2A inhibitor (e.g., levetiracetam, brivaracetam, or seletracetam), or the pharmaceutically acceptable salt, hydrate, solvate, polymorph, or isomer thereof, when administered in the absence of a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, by at least about 1.5×, or 2.0×, or 2.5×, or 3.0×, or 3.5×, or 4.0×, or 4.5×, or 5.0×, or 5.5×, or 6.0×, or 6.5×, or 7.0×, or 7.5×, or 8.0×, or 8.5×, or 9.0×, or 9.5×, or 10×, or greater than about 10×.

The disclosure provides a method of increasing the therapeutic index of an SV2A inhibitor (e.g., levetiracetam, brivaracetam, or seletracetam), or a pharmaceutically acceptable salt, hydrate, solvate, isomer, or polymorph thereof, in a method of treating cognitive impairment associated with a central nervous system (CNS) disorder in a subject in need thereof or at risk thereof, the method comprising administering to the subject a pharmaceutical composition or combination of the disclosure. In certain such embodiments, the therapeutic index of the SV2A inhibitor (e.g., levetiracetam, brivaracetam, or seletracetam), or the pharmaceutically acceptable salt, hydrate, solvate, polymorph, or isomer thereof, is greater than the therapeutic index of the SV2A inhibitor (e.g., levetiracetam, brivaracetam, or seletracetam), or the pharmaceutically acceptable salt, hydrate, solvate, polymorph, or isomer thereof, when administered in the absence of the GABA_{A} α5 agonist, or the pharmaceutically acceptable salt, hydrate, solvate, polymorph, or prodrug thereof, by at least about 1.5×, or about 2.0×, or about 2.5×, or about 3.0×, or about 3.5×, or about 4.0×, or about 4.5×, or about 5.0×, or about 5.5×, or about 6.0×, or about 6.5×, or about 7.0×, or about 7.5×, or about 8.0×, or about 8.5×, or about 9.0×, or about 9.5×, or about 10×, or greater than about 10×.

The disclosure provides a method of increasing the therapeutic index of a GABA_{A} α5 receptor agonist, or a pharmaceutically acceptable salt, hydrate, solvate, isomer, or polymorph thereof, in a method of treating cognitive impairment associated with a central nervous system (CNS) disorder in a subject in need thereof or at risk thereof, the method comprising administering to the subject a pharmaceutical composition or combination of the disclosure. In certain such embodiments, the therapeutic index of the GABA_{A} α5 receptor agonist, or the pharmaceutically acceptable salt, hydrate, solvate, polymorph, or isomer thereof, is greater than the therapeutic index of the GABA_{A} α5 receptor agonist, or the pharmaceutically acceptable salt, hydrate, solvate, polymorph, or isomer thereof, when administered in the absence of the SV2A inhibitor (e.g., levetiracetam, brivaracetam, or seletracetam), or the pharmaceutically acceptable salt, hydrate, solvate, polymorph, or isomer thereof, by at least about 1.5×, or about 2.0×, or about 2.5×, or about 3.0×, or about 3.5×, or about 4.0×, or about 4.5×, or about 5.0×, or about 5.5×, or about 6.0×, or about 6.5×, or about 7.0×, or about 7.5×, or about 8.0×, or about 8.5×, or about 9.0×, or about 9.5×, or about 10×, or greater than about 10×.

The various CNS disorders with cognitive impairment (e.g., age-related cognitive impairment, MCI, AAMI, ARCD, amnestic MCI, dementia, Alzheimer's disease (AD), prodromal AD, post traumatic stress disorder (PTSD), schizophrenia, bipolar disorder, amyotrophic lateral sclerosis, cancer-therapy-related cognitive impairment, mental retardation, Parkinson's disease, autism, compulsive behavior, or substance addiction) may have a variety of etiologies. However, the symptom of cognitive impairment in each of the disorders may have overlapping causes. Thus, a method, use, pharmaceutical composition, combination, pharmaceutical composition for use, or combination for use that treats cognitive impairment in one CNS disorder may also treat cognitive impairment in another.

In some of the embodiments of the methods, uses, pharmaceutical compositions for use, or combinations for use of the disclosure, the effect of the treatment, the progression of cognitive impairment, or the rate of decline of cognitive function is measured by detecting the difference between the levels of reelin in the subject prior to and after the administration or use step.

In some of the embodiments of the methods, uses, pharmaceutical compositions for use, or combinations for use of the disclosure, the effect of the treatment, the progression of cognitive impairment, or the rate of decline of cognitive function is measured by detecting the difference between the levels of somatostatin in the subject prior to and after the administration or use step.

### Methods of Assessing Cognitive Impairment

Animal models serve as an important resource for developing and evaluating treatments for cognitive impairment associated with CNS disorders or brain cancers. Features that characterize cognitive impairment in animal models typically extend to cognitive impairment in humans. Efficacy in such animal models is, thus, expected to be predictive of efficacy in humans. The extent of cognitive impairment in an animal model for a CNS disorder or a brain cancer, and the efficacy of a method of treatment for said CNS disorder or a brain cancer may be tested and confirmed with the use of a variety of cognitive tests.

In animal model systems, cognitive function may be measured in various conventional ways known in the art, including using a Morris Water Maze (MWM), Barnes circular maze, elevated radial arm maze, T maze, or any other mazes in which the animals use spatial information. Cognitive function can be assessed by reversal learning, extradimensional set shifting, conditional discrimination learning, and assessments of reward expectancy. Other tests known in the art may also be used to assess cognitive function, such as novel object recognition and odor recognition tasks. In animals, cognitive function may also be measured with electrophysiological techniques.

A Radial Arm Maze (RAM) behavioral task is one example of a cognitive test, specifically testing spatial memory (Chappell et al. Neuropharmacology 37: 481-487, 1998). The RAM apparatus consists of, e.g., eight equidistantly spaced arms. A maze arm projects from each facet of a center platform. A food well is located at the distal end of each arm. Food is used as a reward. Blocks can be positioned to prevent entry to any arm. Numerous extra maze cues surrounding the apparatus may also be provided. After habituation and training phases, spatial memory of the subjects may be tested in the RAM under control or test compound-treated conditions. As a part of the test, subjects are pretreated before trials with a vehicle control or one of a range of dosages of the test compound. At the beginning of each trial, a subset of the arms of the eight-arm maze is blocked. Subjects are allowed to obtain food on the unblocked arms to which access is permitted during this initial “information phase” of the trial. Subjects are then removed from the maze for a delay period, e.g., a 60 second delay, a 15 minute delay, a one-hour delay, a two-hour delay, a six hour delay, a 24 hour delay, or longer) between the information phase and the subsequent “retention test,” during which the barriers on the maze are removed, thus allowing access to all eight arms. After the delay period, subjects are placed back onto the center platform (with the barriers to the previously blocked arms removed) and allowed to obtain the remaining food rewards during this retention test phase of the trial. The identity and configuration of the blocked arms vary across trials. The number of “errors” the subjects make during the retention test phase is tracked. An error occurs in the trial if the subjects entered an arm from which food had already been retrieved in the pre-delay component of the trial, or if it re-visits an arm in the post-delay session that had already been visited. A fewer number of errors would indicate better spatial memory. The number of errors made by the test subject, under various test compound treatment regimes, can then be compared for efficacy of the test compound in treating cognitive impairment associated with CNS disorders or brain cancers.

Another cognitive test that may be used to assess the effects of a test compound on the cognitive impairment of a CNS disorder model or a brain cancer animal is the Morris water maze (MWM). A water maze is a pool surrounded with a novel set of patterns relative to the maze. The training protocol for the water maze may be based on a modified water maze task that has been shown to be hippocampal-dependent (de Hoz et al., Eur. J. Neurosci., 22:745-54, 2005; Steele and Morris, Hippocampus 9:118-36, 1999). The subject is trained to locate a submerged escape platform hidden underneath the surface of the pool. During the training trial, a subject is released in the maze (pool) from random starting positions around the perimeter of the pool. The starting position varies from trial to trial. If the subject does not locate the escape platform within a set time, the experimenter guides and places the subject on the platform to “teach” the location of the platform. After a delay period following the last training trial, a retention test in the absence of the escape platform is given to assess spatial memory. The subject's level of preference for the location of the (now absent) escape platform, as measured by, e.g., the time spent in that location or the number of crossings of that location made by the mouse, indicates better spatial memory, i.e., treatment of cognitive impairment. The preference for the location of the escape platform under different treatment conditions, can then be compared for efficacy of the test compound in treating cognitive impairment associated with CNS disorders or brain cancers.

There are various tests known in the art for assessing cognitive function in humans, for example and without limitation, the clinical global impression of change scale (CIBIC-plus scale); the Mini Mental State Exam (MMSE); the Neuropsychiatric Inventory (NPI); the Clinical Dementia Rating Scale (CDR); the Cambridge Neuropsychological Test Automated Battery (CANTAB); the Sandoz Clinical Assessment-Geriatric (SCAG), the Buschke Selective Reminding Test (Buschke and Fuld, 1974); the Verbal Paired Associates subtest; the Logical Memory subtest; the Visual Reproduction sub test of the Wechsler Memory Scale-Revised (WMS-R) (Wechsler, 1997); the Benton Visual Retention Test, or MATRICS consensus neuropsychological test battery which includes tests of working memory, speed of processing, attention, verbal learning, visual learning, reasoning and problem solving and social cognition. See Folstein et al., J Psychiatric Res 12: 189-98, (1975); Robbins et al., Dementia 5: 266-81, (1994); Rey, L'examen clinique en psychologie, (1964); Kluger et al., J Geriatr Psychiatry Neurol 12:168-79, (1999); Marquis et al., 2002 and Masur et al., 1994. Also see Buchanan, R. W., Keefe, R. S. E., Umbricht, D., Green, M. F., Laughren, T., and Marder, S. R. (2011), The FDA-NIMH-MATRICS guidelines for clinical trial design of cognitive-enhancing drugs: what do we know 5 years later? Schizophr. Bull. 37, 1209-1217. Another example of a cognitive test in humans is the explicit 3-alternative forced choice task. In this test, subjects are presented with color photographs of common objects consisting of a mix of three types of image pairs: similar pairs, identical pairs and unrelated foils. The second of the pair of similar objects is referred to as the “lure.” These image pairs are fully randomized and presented individually as a series of images. Subjects are instructed to make a judgment as to whether the objects seen are new, old or similar. A “similar” response to the presentation of a lure stimulus indicates successful memory retrieval by the subject. By contrast, calling the lure stimulus “old” or “new” indicates that correct memory retrieval did not occur.

In addition to assessing cognitive performance, the progression of age-related cognitive impairment and dementia, as well as the conversion of age-related cognitive impairment into dementia, may be monitored by assessing surrogate changes in the brain of the subject. Surrogate changes include, without limitation, changes in regional brain volumes, perforant path degradation, and changes seen in brain function through resting state fMRI (R-fMRI), positron emission tomography (PET), single photon emission computed Tomography (SPECT), fluorodeoxyglucose positron emission tomography (FDG-PET), or any other imaging technique that allows one to measure brain function. Examples of regional brain volumes useful in monitoring the progression of age-related cognitive impairment and dementia include reduction of hippocampal volume and reduction in volume or thickness of entorhinal cortex. These volumes may be measured in a subject by, for example, MRI. Aisen et al., Alzheimer's & Dementia 6:239-246 (2010). Perforant path degradation has been shown to be linked to age, as well as reduced cognitive function. For example, older adults with more perforant path degradation tend to perform worse in hippocampus-dependent memory tests. Perforant path degradation may be monitored in subjects through ultrahigh-resolution diffusion tensor imaging (DTI). Yassa et al., PNAS 107:12687-12691 (2010). Resting-state fMRI (R-fMRI) involves imaging the brain during rest, and recording large-amplitude spontaneous low-frequency (<0.1 Hz) fluctuations in the fMRI signal that are temporally correlated across functionally related areas. Seed-based functional connectivity, independent component analyses, and/or frequency-domain analyses of the signals are used to reveal functional connectivity between brain areas, particularly those areas whose connectivity increase or decrease with age, as well as the extent of cognitive impairment and/or dementia. FDG-PET uses the uptake of FDG as a measure of regional metabolic activity in the brain. Decline of FDG uptake in regions such as the posterior cingulated cortex, temporoparietal cortex, and prefrontal association cortex has been shown to relate to the extent of cognitive decline and dementia. Aisen et al., Alzheimer's & Dementia 6:239-246 (2010), Herholz et al., Neuroimage 17:302-316 (2002).

### Age-Related Cognitive Impairment, Age-Associated Memory Impairment, and Age-Related Cognitive Decline

This disclosure provides methods, uses, combinations, pharmaceutical compositions, combinations for use, or pharmaceutical compositions for use useful for treating age-related cognitive impairment or the risk thereof using an SV2A inhibitor (e.g., levetiracetam, brivaracetam, or seletracetam), or a pharmaceutically acceptable salt, hydrate, solvate, polymorph, or isomer thereof, in combination with a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof. In certain embodiments, treatment comprises improving cognitive function in patients with age-related cognitive impairment. In certain embodiments, treatment comprises slowing or delaying the progression of age-related cognitive impairment. In certain embodiments, treatment comprises reducing the rate of decline of cognitive function associated with age-related cognitive impairment. In certain embodiments, treatment comprises preventing or slowing the progression, of age-related cognitive impairment. In certain embodiments, treatment comprises alleviation, amelioration or slowing the progression, of one or more symptoms associated with age-related cognitive impairment. In certain embodiments, treatment of age-related cognitive impairment comprises slowing the conversion of age-related cognitive impairment into dementia (e.g., AD).

The methods, uses, combinations, pharmaceutical compositions, combinations for use, or pharmaceutical compositions for use may be useful in human patients in clinical applications useful for treating age-related cognitive impairment (ARCD) and age-associated memory impairment (AAMI) or for the risk thereof. The dose of the pharmaceutical composition or combination and dosage interval for the method or use is, as described herein, one that is safe and efficacious in those applications.

“Age-related cognitive impairment” may refer to cognitive impairment in aged subjects, wherein their cognitive function is not as robust as that expected in an age-matched normal subject or as that expected in young adult subjects. In some cases, cognitive function is reduced by about 5%, about 10%, about 30%, or more, compared to cognitive function expected in an age-matched normal subject. In some cases, cognitive function is as expected in an age-matched normal subject, but reduced by about 5%, about 10%, about 30%, about 50%, or more, compared to cognitive function expected in a young adult subject. Age-related impaired cognitive function may be associated with age-related mild cognitive impairment (MCI) (including amnestic MCI and non-amnestic MCI), age-associated memory impairment (AAMI), and age-related cognitive decline (ARCD).

In some embodiments, a subject to be treated by the methods, uses, combinations, pharmaceutical compositions, combinations for use, or pharmaceutical compositions for use of this disclosure exhibits age-related cognitive impairment or is at risk of such impairment. In some embodiments, the age-related cognitive impairment may include, without limitation, age-associated memory impairment (AAMI) and age-related cognitive decline (ARCD).

“Age-associated memory impairment (AAMI)” may refer to a decline in memory due to aging. A patient may be considered to have AAMI if he or she is at least 50 years old and meets all of the following criteria: (1) the patient has noticed a decline in memory performance; (2) the patient performs worse on a standard test of memory compared to young adults; (3) all other obvious causes of memory decline, except normal aging, have been ruled out (in other words, the memory decline cannot be attributed to other causes such as a recent heart attack or head injury, depression, adverse reactions to medication, Alzheimer's disease, etc.).

“Age-related cognitive decline (ARCD)” may refer to declines in memory and cognitive abilities that are a normal consequence of aging in humans (e.g., Craik & Salthouse, 1992). This is also true in virtually all mammalian species. Age-associated memory impairment may refer to older persons with objective memory declines relative to their younger years, but cognitive functioning that is normal relative to their age peers (Crook et al., 1986). Age-consistent memory decline is a less pejorative label which emphasizes that these are normal developmental changes (Crook, 1993; Larrabee, 1996), are not pathophysiological (Smith et al., 1991), and rarely progress to overt dementia (Youngjohn & Crook, 1993). The DSM-IV (1994) has codified the diagnostic classification of ARCD.

Animal models serve as an important resource for developing and evaluating treatments for such age-related cognitive impairments. Features that characterize age-related cognitive impairment in animal models typically extend to age-related cognitive impairment in humans. Efficacy in such animal models is, thus, expected to be predictive of efficacy in humans.

Various animal models of age-related cognitive impairment are known in the art. For example, extensive behavioral characterization has identified a naturally occurring form of cognitive impairment in an outbred strain of aged Long-Evans rats (Charles River Laboratories; Gallagher et al., Behav. Neurosci. 107:618-626, (1993)). In a behavioral assessment with the Morris Water Maze (MWM), rats learn and remember the location of an escape platform guided by a configuration of spatial cues surrounding the maze. The cognitive basis of performance is tested in probe trials using measures of the animal's spatial bias in searching for the location of the escape platform. Aged rats in the study population have no difficulty swimming to a visible platform, but an age-dependent impairment is detected when the platform is camouflaged, requiring the use of spatial information. Performance for individual aged rats in the outbred Long-Evans strain varies greatly. For example, a proportion of those rats perform on a par with young adults. However, approximately 40-50% fall outside the range of young performance. This variability among aged rats reflects reliable individual differences. Thus, within the aged population some animals are cognitively impaired and designated aged-impaired (AI) and other animals are not impaired and are designated aged-unimpaired (AU). See, e.g., Colombo et al., Proc. Natl. Acad. Sci. 94: 14195-14199, (1997); Gallagher and Burwell, Neurobiol. Aging 10: 691-708, (1989); Gallagher et al. Behav. Neurosci. 107:618-626, (1993); Rapp and Gallagher, Proc. Natl. Acad. Sci. 93: 9926-9930, (1996); Nicolle et al., Neuroscience 74: 741-756, (1996); Nicolle et al., J. Neurosci. 19: 9604-9610, (1999); International Patent Publication WO2007/019312 and International Patent Publication WO 2004/048551. Such an animal model of age-related cognitive impairment may be used to assay the effectiveness of the methods, uses, combinations, pharmaceutical compositions, combinations for use, or pharmaceutical compositions for use this disclosure in treating age-related cognitive impairment.

The efficacy of the methods, uses, combinations, pharmaceutical compositions, combinations for use, or pharmaceutical compositions for use of this disclosure in treating age-related cognitive impairment may be assessed using a variety of cognitive tests, including the Morris water maze and the radial arm maze, as discussed herein.

### Mild Cognitive Impairment

In some embodiments, a subject to be treated by the methods, uses, combinations, pharmaceutical compositions, combinations for use, or pharmaceutical compositions for use of this disclosure exhibits MCI or is at risk of such impairment. The methods, uses, combinations, pharmaceutical compositions, combinations for use, or pharmaceutical compositions for use may be useful in human patients in clinical applications useful for treating MCI (including amnestic MCI and non-amnestic MCI).

This disclosure provides methods, uses, combinations, pharmaceutical compositions, combinations for use, or pharmaceutical compositions for use useful for treating mild cognitive impairment or the risk thereof using an SV2A inhibitor (e.g., levetiracetam, brivaracetam, or seletracetam), or a pharmaceutically acceptable salt, hydrate, solvate, polymorph, or isomer thereof, in combination with a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof. In certain embodiments, treatment comprises improving cognitive function in patients with mild cognitive impairment. In certain embodiments, treatment comprises slowing or delaying the progression of mild cognitive impairment. In certain embodiments, treatment comprises reducing the rate of decline of cognitive function associated with mild cognitive impairment. In certain embodiments, treatment comprises preventing or slowing the progression, of mild cognitive impairment. In certain embodiments, treatment comprises alleviation, amelioration or slowing the progression, of one or more symptoms associated with mild cognitive impairment.

“Mild cognitive impairment” or “MCI” may refer to a condition characterized by isolated memory impairment and relatively normal functional abilities unaccompanied by other cognitive abnormalities. One set of criteria for a clinical characterization of MCI specifies the following characteristics: (1) memory complaint (as reported by patient, informant, or physician), (2) normal activities of daily living (ADLs), (3) normal global cognitive function, (4) abnormal memory for age (defined as scoring more than 1.5 standard deviations below the mean for a given age), and (5) absence of indicators of dementia (as defined by DSM-IV guidelines). Petersen et al., Srch. Neurol. 56: 303-308 (1999); Petersen, “Mild cognitive impairment: Aging to Alzheimer's Disease,” Oxford University Press, N.Y. (2003). MCI may be amnestic MCI or non-amnestic MCI.

Diagnosis of MCI may entail an objective assessment of cognitive impairment, which can be garnered through the use of well-established neuropsychological tests, including the Mini Mental State Examination (MMSE), the Cambridge Neuropsychological Test Automated Battery (CANTAB) and individual tests such as Rey Auditory Verbal Learning Test (AVLT), Logical Memory Subtest of the revised Wechsler Memory Scale (WMS-R) and the New York University (NYU) Paragraph Recall Test. See Folstein et al., J Psychiatric Res 12: 189-98 (1975); Robbins et al., Dementia 5: 266-81 (1994); Kluger et al., J Geriatric Psychiatry Neurol 12:168-79 (1999). The models described herein for assessing the efficacy of a treatment for age-related cognitive impairment may also be used for assessing the efficacy of a treatment for mild cognitive impairment.

### Dementia

This disclosure also provides methods, uses, combinations, pharmaceutical compositions, combinations for use, or pharmaceutical compositions for use useful for treating cognitive impairment associated with dementia using an SV2A inhibitor (e.g., levetiracetam, brivaracetam, or seletracetam), or a pharmaceutically acceptable salt, hydrate, solvate, polymorph, or isomer thereof, in combination with a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof. In certain embodiments, treatment comprises improving cognitive function in patients with dementia. In certain embodiments, treatment comprises slowing or delaying the progression of cognitive impairment associated with dementia. In certain embodiments, treatment comprises reducing the rate of decline of cognitive function associated with dementia. In certain embodiments, treatment comprises preventing or slowing the progression of cognitive impairment associated with dementia. In certain embodiments, the dementia is Alzheimer's disease (AD), vascular dementia, dementia with Lewy bodies, or frontotemporal dementia. The methods, uses, combinations, pharmaceutical compositions, combinations for use, or pharmaceutical compositions for use may be useful for human patients in clinical applications in treating cognitive impairment associated with dementia. The dose of the pharmaceutical composition or combination and dosage interval for the method or use is, as described herein, one that is safe and efficacious in those applications.

“Cognitive impairment” associated with AD or related to AD or in AD may refer to cognitive function in subjects that is not as robust as that expected in subjects who have not been diagnosed AD using conventional methodologies and standards.

Alzheimer's disease (AD) may be characterized by memory deficits in its early phase. Later symptoms include impaired judgment, disorientation, confusion, behavior changes, trouble speaking, and motor deficits. Histologically, AD may be characterized by beta-amyloid plaques and tangles of protein tau.

Vascular dementia may be caused by strokes. Symptoms overlap with those of AD, but without the focus on memory impairment.

Dementia with Lewy bodies may be characterized by abnormal deposits of alpha-synuclein that form inside neurons in the brain. Cognitive impairment may be similar to AD, including impairments in memory and judgment and behavior changes.

Frontotemporal dementia may be characterized by gliosis, neuronal loss, superficial spongiform degeneration in the frontal cortex and/or anterior temporal lobes, and Picks' bodies. Symptoms may include changes in personality and behavior, including a decline in social skills and language expression/comprehension.

Animal models serve as an important resource for developing and evaluating treatments for dementia. Features that characterize dementia in animal models typically extend to dementia in humans. Thus, efficacy in such animal models is expected to be predictive of efficacy in humans. Various animal models of dementia are known in the art, such as the PDAPP, Tg2576, APP23, TgCRND8, J20, hPS2 Tg, and APP+PS1 transgenic mice. Sankaranarayanan, Curr. Top. Medicinal Chem. 6: 609-627, 2006; Kobayashi et al. Genes Brain Behav. 4:173-196. 2005; Ashe and Zahns, Neuron. 66:631-45,2010. Such animal models of dementia may be used to assay the effectiveness of the methods, uses, combinations, pharmaceutical compositions, combinations for use, or pharmaceutical compositions for use of this disclosure in treating dementia.

The efficacy of the methods, uses, combinations, pharmaceutical compositions, combinations for use, or pharmaceutical compositions for use of this disclosure in treating cognitive impairment associated with dementia may be assessed in animals models of dementia, as well as human subjects with dementia, using a variety of cognitive tests known in the art, as discussed herein.

### Post-Traumatic Stress Disorder

This disclosure also provides methods, uses, combinations, pharmaceutical compositions, combinations for use, or pharmaceutical compositions for use useful for treating cognitive impairment associated with post traumatic stress disorder (PTSD) using an SV2A inhibitor (e.g., levetiracetam, brivaracetam, or seletracetam), or a pharmaceutically acceptable salt, hydrate, solvate, polymorph, or isomer thereof, in combination with a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof. In certain embodiments, treatment comprises improving cognitive function in patients with PTSD. In certain embodiments, treatment comprises slowing or delaying the progression of cognitive impairment associated with PTSD. In certain embodiments, treatment comprises reducing the rate of decline of cognitive function associated with PTSD. In certain embodiments, treatment comprises preventing or slowing the progression of cognitive impairment associated with PTSD. In certain embodiments, treatment comprises alleviation, amelioration, or slowing the progression of cognitive impairment associated with PTSD. The methods, uses, combinations, pharmaceutical compositions, combinations for use, or pharmaceutical compositions for use may be useful for human patients in clinical applications in treating cognitive impairment associated with PTSD. The dose of the pharmaceutical composition or combination and dosage interval for the method or use is, as described herein, one that is safe and efficacious in those applications.

“Post-traumatic stress disorder (PTSD)” may refer to an anxiety disorder characterized by an immediate or delayed response to a catastrophic event, characterized by re-experiencing the trauma, psychic numbing or avoidance of stimuli associated with the trauma, and increased arousal. Re-experiencing phenomena may include intrusive memories, flashbacks, nightmares, and psychological or physiological distress in response to trauma reminders. Such responses may produce anxiety and can have significant impact, both chronic and acute, on a patient's quality of life and physical and emotional health. PTSD may also be associated with impaired cognitive performance, and older individuals with PTSD have greater decline in cognitive performance relative to control patients.

Patients with PTSD (and, to a lesser degree, trauma-exposed patients without PTSD) have smaller hippocampal volumes (Woon el al., Prog. Neuro-Psychopharm. & Biological Psych. 34, 1181-1188; Wang et al., Arch. Gen. Psychiatry 67:296-303,2010). PTSD is also associated with impaired cognitive performance. Older individuals with PTSD have greater declines in cognitive performance relative to control patients (Yehuda et al, Bio. Psych. 60: 714-721, 2006) and have a greater likelihood of developing dementia (Yaffe et al., Arch. Gen. Psych. 678:608-613,2010).

Animal models serve as an important resource for developing and evaluating treatments for cognitive impairment associated with PTSD. Features that characterize PTSD in animal models typically extend to PTSD in humans. Thus, efficacy in such animal models is expected to be predictive of efficacy in humans. Various animal models of PTSD are known in the art.

One rat model of PTSD is Time-dependent sensitization (TDS). TDS involves exposure of the animal to a severely stressful event followed by a situational reminder of the prior stress. The following is an example of TDS. Rats are placed in a restrainer, then placed in a swim tank and made to swim for a period of time, e.g., 20 min. Following this, each rat is then immediately exposed to a gaseous anesthetic until loss of consciousness, and finally dried. The animals are left undisturbed for a number of days, e.g., one week. The rats are then exposed to a “restress” session consisting of an initial stressor, e.g., a swimming session in the swim tank (Liberzon et al., Psychoneuroendocrinology 22: 443-453, 1997; Harvery et al., Psychopharmacology 175:494-502, 2004). TDS results in an enhancement of the acoustic startle response (ASR) in the rat, which is comparable to the exaggerated acoustic startle that is a prominent symptom of PTSD (Khan and Liberzon, Psychopharmacology 172: 225-229, 2004). Such animal models of PTSD may be used to assay the effectiveness of the methods, uses, combinations, pharmaceutical compositions, combinations for use, or pharmaceutical compositions for use of this disclosure in treating PTSD.

The efficacy of the methods, uses, combinations, pharmaceutical compositions, combinations for use, or pharmaceutical compositions for use of this disclosure in treating PTSD, or cognitive impairment associated with PTSD, may also be assessed in animals models of PTSD, as well as human subjects with PTSD, using a variety of cognitive tests known in the art, as discussed herein.

### Schizophrenia and Bipolar Disorder

This disclosure provides methods, uses, combinations, pharmaceutical compositions, combinations for use, or pharmaceutical compositions for use useful for treating cognitive impairment associated with schizophrenia or bipolar disorder (in particular, mania) using an SV2A inhibitor (e.g., levetiracetam, brivaracetam, or seletracetam), or a pharmaceutically acceptable salt, hydrate, solvate, polymorph, or isomer thereof, in combination with a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof. In certain embodiments, treatment comprises improving cognitive function in patients with schizophrenia. In certain embodiments, treatment comprises slowing or delaying the progression of cognitive impairment associated with schizophrenia. In certain embodiments, treatment comprises reducing the rate of decline of cognitive function associated with schizophrenia. In certain embodiments, treatment comprises preventing or slowing the progression of cognitive impairment associated with schizophrenia or bipolar disorder (in particular, mania).

“Schizophrenia” may refer to a chronic debilitating disorder, characterized by a spectrum of psychopathology, including positive symptoms such as aberrant or distorted mental representations (e.g., hallucinations, delusions), negative symptoms characterized by diminution of motivation and adaptive goal-directed action (e.g., anhedonia, affective flattening, avolition), and cognitive impairment. While abnormalities in the brain are proposed to underlie the full spectrum of psychopathology in schizophrenia, currently available antipsychotics are largely ineffective in treating cognitive impairments in patients.

“Bipolar disorder,” “BP,” “manic depressive disorder,” or “manic depressive illness” may refer to a chronic psychological/mood disorder which can be characterized by significant mood changes including periods of depression and euphoric manic periods. BP may be diagnosed by a skilled physician based on personal and medical history, interview consultation and physical examinations. “Mania” or “manic periods” or other variants may refer to periods where an individual exhibits some or all of the following characteristics: racing thoughts, rapid speech, elevated levels of activity and agitation as well as an inflated sense of self-esteem, euphoria, poor judgment, insomnia, impaired concentration and aggression.

Schizophrenia is characterized by a wide spectrum of psychopathology, including positive symptoms such as aberrant or distorted mental representations (e.g., hallucinations, delusions), negative symptoms characterized by diminution of motivation and adaptive goal-directed action (e.g., anhedonia, affective flattening, avolition), and cognitive impairment. In certain embodiments, treatment comprises alleviation, amelioration or slowing the progression of cognitive impairment associated with schizophrenia. Further, there are a number of other psychiatric diseases such as schizotypal and schizoaffective disorder, other acute- and chronic psychoses and bipolar disorder (in particular, mania), which have an overlapping symptomatology with schizophrenia. In some embodiments, treatment comprises alleviation, amelioration or slowing the progression of cognitive impairment associated with bipolar disorder (in particular, mania). The methods, uses, combinations, pharmaceutical compositions, combinations for use, or pharmaceutical compositions for use may be useful for human patients in clinical applications in treating cognitive impairment associated with schizophrenia or bipolar disorder (in particular, mania). The dose of the pharmaceutical composition or combination and dosage interval for the method or use is, as described herein, one that is safe and efficacious in those applications.

Cognitive impairments are associated with schizophrenia. They precede the onset of psychosis and are present in non-affected relatives. The cognitive impairments associated with schizophrenia constitute a good predictor for functional outcome and are a core feature of the disorder. Cognitive features in schizophrenia reflect dysfunction in frontal cortical and hippocampal circuits. Patients with schizophrenia also present hippocampal pathologies such as reductions in hippocampal volume, reductions in neuronal size and dysfunctional hyperactivity. An imbalance in excitation and inhibition in these brain regions has also been documented in schizophrenic patients suggesting that drugs targeting inhibitory mechanisms could be therapeutic. See, e.g., Guidotti et al., Psychopharmacology 180: 191-205, 2005; Zierhut, Psych. Res. Neuroimag. 183:187-194, 2010; Wood et al., NeuroImage 52:62-63, 2010; Vinkers et al., Expert Opin. Investig. Drugs 19:1217-1233, 2009; Young et al., Pharmacol. Ther. 122:150-202, 2009.

Animal models serve as an important resource for developing and evaluating treatments for cognitive impairment associated with schizophrenia. Features that characterize schizophrenia in animal models typically extend to schizophrenia in humans. Thus, efficacy in such animal models is expected to be predictive of efficacy in humans. Various animal models of schizophrenia are known in the art.

One animal model of schizophrenia is protracted treatment with methionine. Methionine-treated mice exhibit deficient expression of GAD67 in frontal cortex and hippocampus, similar to those reported in the brain of postmortem schizophrenia patients. They also exhibit prepulse inhibition of startle and social interaction deficits (Tremonlizzo et al., PNAS, 99: 17095-17100,2002). Another animal model of schizophrenia is methylaoxymethanol acetate (MAM)-treatment in rats. Pregnant female rats are administered MAM (20 mg/kg, intraperitoneal) on gestational day 17. MAM-treatment recapitulate a pathodevelopmental process to schizophrenia-like phenotypes in the offspring, including anatomical changes, behavioral deficits and altered neuronal information processing. More specifically, MAM-treated rats display a decreased density of parvalbumin-positive GABAergic interneurons in portions of the prefrontal cortex and hippocampus. In behavioral tests, MAM-treated rats display reduced latent inhibition. Latent inhibition is a behavioral phenomenon where there is reduced learning about a stimulus to which there has been prior exposure with any consequence. This tendency to disregard previously benign stimuli and reduce the formation of association with such stimuli is believed to prevent sensory overload. Low latent inhibition is indicative of psychosis. Latent inhibition may be tested in rats in the following manner. Rats are divided into two groups. One group is pre-exposed to a tone over multiple trials. The other group has no tone presentation. Both groups are then exposed to an auditory fear conditioning procedure, in which the same tone is presented concurrently with a noxious stimulus, e.g. an electric shock to the foot. Subsequently, both groups are presented with the tone, and the rats' change in locomotor activity during tone presentation is monitored. After the fear conditioning the rats respond to the tone presentation by strongly reducing locomotor activity. However, the group that has been exposed to the tone before the conditioning period displays robust latent inhibition: the suppression of locomotor activity in response to tone presentation is reduced. MAM-treated rats, by contrast show impaired latent inhibition. That is, exposure to the tone previous to the fear conditioning procedure has no significant effect in suppressing the fear conditioning. (see Lodge et al, J. Neurosci., 29:2344-2354, 2009). Such animal models of schizophrenia may be used to assay the effectiveness of the methods, uses, combinations, pharmaceutical compositions, combinations for use, or pharmaceutical compositions for use of the disclosure in treating cognitive impairment associated with schizophrenia or bipolar disorder (in particular, mania).

MAM-treated rats display a significantly enhanced locomotor response (or aberrant locomotor activity) to low dose D-amphetamine administration. The MAM-treated rats also display a significantly greater number of spontaneously firing ventral tegmental dopamine (DA) neurons. These results are believed to be a consequence of excessive hippocampal activity because in MAM-treated rats, the ventral hippocampus (vHipp) inactivation (e.g., by intra-vHipp administration of a sodium channel blocker, tetrodotoxin (TTX), to MAM rats) completely reversed the elevated DA neuron population activity and also normalized the augmented amphetamine-induced locomotor behavior. The correlation of hippocampal dysfunction and the hyper-responsivity of the DA system is believed to underlie the augmented response to amphetamine in MAM-treated animals and psychosis in schizophrenia patients. See Lodge D. J. et al. Neurobiology of Disease (2007), 27(42), 11424-11430. The use of MAM-treated rats in the above study may be suitable for use to assay the effectiveness of the methods, uses, combinations, pharmaceutical compositions, combinations for use, or pharmaceutical compositions for use of the present disclosure in treating cognitive impairment associated with schizophrenia or bipolar disorder (in particular, mania). For example, the methods, uses, combinations, pharmaceutical compositions, combinations for use, or pharmaceutical compositions for use of this disclosure may be evaluated, using MAM-treated animals, for their effects on the central hippocampus (vHipp) regulation, on the elevated DA neuron population activity and on the hyperactive locomotor response to amphetamine in the MAM-treated animals.

In MAM-treated rats, hippocampal (HPC) dysfunction leads to dopamine system hyperactivity. A benzodiazepine-positive allosteric modulator (PAM), selective for the α5 subunit of the GABA_{A} receptor, SH-053-2′F—R—CH₃, is tested for its effects on the output of the hippocampal (HPC). The effect of SH-053-2′F—R—CH₃ on the hyperactive locomotor response to amphetamine in MAM-treated animals is also examined. The α5 GABA_{A} receptor PAM reduces the number of spontaneously active DA neurons in the ventral tegmental area (VTA) of MAM rats to levels observed in saline-treated rats (control group), both when administered systemically and when directly infused into the ventral HPC. Moreover, HPC neurons in both saline-treated and MAM-treated animals show diminished cortical-evoked responses following the α5 GABA_{A} receptor PAM treatment. In addition, the increased locomotor response to amphetamine observed in MAM-treated rats is reduced following the α5 GABA_{A} receptor PAM treatment. See Gill K. M et al. Neuropsychopharmacology (2011), 1-9. The use of MAM-treated rats in the above study may be suitable for use in the present disclosure to assay the effectiveness of the methods, uses, combinations, pharmaceutical compositions, combinations for use, or pharmaceutical compositions for use of the disclosure in treating cognitive impairment associated with schizophrenia or bipolar disorder (in particular, mania). For example, the methods, uses, combinations, pharmaceutical compositions, combinations for use, or pharmaceutical compositions for use of this disclosure may be evaluated, using MAM-treated animals, for their effects on the output of the hippocampal (HPC) and on the hyperactive locomotor response to amphetamine in the MAM-treated animals.

Administration of MAM to pregnant rats on embryonic day 15 (E15) severely impairs spatial memory or the ability to learn the spatial location of four items on an eight-arm radial maze in the offspring. In addition, embryonic day 17 (E17) MAM-treated rats are able to reach the level of performance of control rats at the initial stages of training but are unable to process and retrieve spatial information when a 30-min delay is interposed, indicating a significant impairment in working memory. See Gourevitch R. et al. (2004). Behav. Pharmacol, 15, 287-292. Such animal models of schizophrenia may be used to assay the effectiveness of the methods, uses, combinations, pharmaceutical compositions, combinations for use, or pharmaceutical compositions for use of the disclosure in treating cognitive impairment associated with schizophrenia or bipolar disorder (in particular, mania).

Apomorphine-induced climbing (AIC) and stereotype (AIS) in mice is another animal model useful in this disclosure. Compounds, pharmaceutical compositions, and combinations are administered to mice at a desired dose level (e.g., via intraperitoneal administration). Subsequently, e.g., thirty minutes later, experimental mice are challenges with apomorphine (e.g., with 1 mg/kg sc). Five minutes after the apomorphine injection, the sniffing-licking-gnawing syndrome (stereotyped behavior) and climbing behavior induced by apomorphine are scored and recorded for each animal. Readings can be repeated every 5 min during a 30-min test session. Scores for each animal are totaled over the 30-min test session for each syndrome (stereotyped behavior and climbing). If an effect reached at least of 50% inhibition, and ID₅₀ value (95% confidence interval) is calculated using a nonlinear least squares calculation with inverse prediction. Mean climbing and stereotype scores can be expressed as a percent of control values observed in vehicle treated (e.g., saline-treated) mice that receive apomorphine. See Grauer S. M. et al. Psychopharmacology (2009) 204, 37-48. This mice model may be used to assay the effectiveness of the methods, uses, combinations, pharmaceutical compositions, combinations for use, or pharmaceutical compositions for use of the disclosure in treating cognitive impairment associated with schizophrenia or bipolar disorder (in particular, mania).

The efficacy of the methods, uses, combinations, pharmaceutical compositions, combinations for use, or pharmaceutical compositions for use of this disclosure in treating cognitive impairment associated with schizophrenia may also be assessed in animal models of schizophrenia or bipolar disorder (in particular, mania), as well as human subjects with schizophrenia, using a variety of cognitive tests known in the art, as discussed herein.

### Amyotrophic Lateral Sclerosis (ALS)

This disclosure additionally provides methods, uses, combinations, pharmaceutical compositions, combinations for use, or pharmaceutical compositions for use useful for treating amyotrophic lateral sclerosis, ALS, using an SV2A inhibitor (e.g., levetiracetam, brivaracetam, or seletracetam), or a pharmaceutically acceptable salt, hydrate, solvate, polymorph, or isomer thereof, in combination with a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof. In certain embodiments, treatment comprises improving cognitive function in patients with ALS. In certain embodiments, treatment comprises slowing or delaying the progression of cognitive impairment associated with ALS. In certain embodiments, treatment comprises reducing the rate of decline of cognitive function associated with ALS. In certain embodiments, treatment comprises preventing or slowing the progression of cognitive impairment associated with ALS. In certain embodiments, treatment comprises alleviation, amelioration or slowing the progression of cognitive impairment associated with ALS. The methods, uses, combinations, pharmaceutical compositions, combinations for use, or pharmaceutical compositions for use may be useful for human patients in clinical applications in treating cognitive impairment associated with ALS. The dose of the pharmaceutical composition or combination and dosage interval for the method or use is, as described herein, one that is safe and efficacious in those applications.

“Amyotrophic lateral sclerosis,” also known as ALS, may refer to a progressive, fatal, neurodegenerative disease characterized by a degeneration of motor neurons, the nerve cells in the central nervous system that control voluntary muscle movement. ALS may also be characterized by neuronal degeneration in the entorhinal cortex and hippocampus, memory deficits, and neuronal hyperexcitability in different brain areas such as the cortex.

In addition to the degeneration of motor neurons, ALS is characterized by neuronal degeneration in the entorhinal cortex and hippocampus, memory deficits, and neuronal hyperexcitability in different brain areas such as the cortex.

The efficacy of the methods, uses, combinations, pharmaceutical compositions, combinations for use, or pharmaceutical compositions for use of this disclosure in treating cognitive impairment associated with ALS, may also be assessed in animal models of ALS, as well as human subjects with ALS, using a variety of cognitive tests known in the art, as discussed herein.

### Cancer Therapy-Related Cognitive Impairment

This disclosure additionally provides methods, uses, combinations, pharmaceutical compositions, combinations for use, or pharmaceutical compositions for use useful for treating cancer therapy-related cognitive impairment using an SV2A inhibitor (e.g., levetiracetam, brivaracetam, or seletracetam), or a pharmaceutically acceptable salt, hydrate, solvate, polymorph, or isomer thereof, in combination with a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof. In certain embodiments, treatment comprises improving cognitive function in patients with cancer therapy-related cognitive impairment. In certain embodiments, treatment comprises slowing or delaying the progression of cancer therapy-related cognitive impairment. In certain embodiments, treatment comprises reducing the rate of decline of cognitive function associated with cancer therapy-related cognitive impairment. In certain embodiments, treatment comprises preventing or slowing the progression, of cancer therapy-related cognitive impairment. In certain embodiments, treatment comprises alleviation, amelioration or slowing the progression, of one or more symptoms associated with cancer therapy-related cognitive impairment. The methods, uses, combinations, pharmaceutical compositions, combinations for use, or pharmaceutical compositions for use may be useful for human patients in clinical applications in treating cancer therapy-related cognitive impairment. The dose of the pharmaceutical composition or combination and dosage interval for the method or use is, as described herein, one that is safe and efficacious in those applications.

“Cancer therapy-related cognitive impairment” may refer to cognitive impairment that develops in subjects that are treated with cancer therapies such as chemotherapy and radiation. Cytotoxicity and other adverse side-effects on the brain of cancer therapies may result in cognitive impairment in such functions as memory, learning and attention.

Therapies that are used in cancer treatment, including chemotherapy, radiation, or combinations thereof, can cause cognitive impairment in patients, in such functions as memory, learning and attention. Cytotoxicity and other adverse side-effects on the brain of cancer therapies are the basis for this form of cognitive impairment, which can persist for decades. (Dietrich et al., Oncologist 13:1285-95,2008; Soussain et al., Lancet 374:1639-51, 2009).

Cognitive impairment following cancer therapies reflects dysfunction in frontal cortical and hippocampal circuits that are essential for normal cognition. In animal models, exposure to either chemotherapy or radiation adversely affects performance on tests of cognition specifically dependent on these brain systems, especially the hippocampus (Kim et al., J. Radiat. Res. 49:517-526, 2008; Yang et al., Neurobiol. Learning and Mem. 93:487-494, 2010). Thus, drugs targeting these cortical and hippocampal systems could be neuroprotective in patients receiving cancer therapies and efficacious in treating symptoms of cognitive impairment that may last beyond the interventions used as cancer therapies.

Animal models serve as an important resource for developing and evaluating treatments for cancer therapy-related cognitive impairment. Features that characterize cancer therapy-related cognitive impairment in animal models typically extend to cancer therapy-related cognitive impairment in humans. Thus, efficacy in such animal models is expected to be predictive of efficacy in humans. Various animal models of cancer therapy-related cognitive impairment are known in the art.

Examples of animal models of cancer therapy-related cognitive impairment include treating animals with anti-neoplastic agents such as cyclophosphamide (CYP) or with radiation, e.g., ⁶⁰Co gamma-rays. (Kim et al., J. Radiat. Res. 49:517-526, 2008; Yang et al, Neurobiol. Learning and Mem. 93:487-494,2010). The cognitive function of animal models of cancer therapy-related cognitive impairment may then be tested with cognitive tests to assay the effectiveness of the methods, uses, combinations, pharmaceutical compositions, combinations for use, or pharmaceutical compositions for use of the disclosure in treating cancer therapy-related cognitive impairment. The efficacy of the methods, uses, combinations, pharmaceutical compositions, combinations for use, or pharmaceutical compositions for use of this disclosure in treating cancer therapy-related cognitive impairment, as well as human subjects with cancer therapy-related cognitive impairment, using a variety of cognitive tests known in the art, as discussed herein.

### Parkinson's Disease (PD)

This disclosure provides methods, uses, combinations, pharmaceutical compositions, combinations for use, or pharmaceutical compositions for use useful for treating cognitive impairment associated with PD or PD psychosis using an SV2A inhibitor (e.g., levetiracetam, brivaracetam, or seletracetam), or a pharmaceutically acceptable salt, hydrate, solvate, polymorph, or isomer thereof, in combination with a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable, salt, hydrate, solvate, polymorph, or isomer thereof. In certain embodiments, treatment comprises preventing or slowing the progression of cognitive impairment associated with PD or PD psychosis. In certain embodiments, treatment comprises alleviation, amelioration, or slowing the progression of cognitive impairment associated with PD or PD psychosis. In certain embodiments, the symptom to be treated is cognitive impairment. In certain embodiments, the symptom to be treated is Parkinson's disease psychosis. For example, methods, uses, combinations, pharmaceutical compositions, combinations for use, or pharmaceutical compositions for use of the disclosure can be useful to improve the motor/cognitive impairments symptomatic of Parkinson's disease. Moreover, methods, uses, combinations, pharmaceutical compositions, combinations for use, or pharmaceutical compositions for use of the disclosure may be useful for treating the memory impairment symptomatic of Parkinson's disease. Additionally, methods, uses, combinations, pharmaceutical compositions, combinations for use, or pharmaceutical compositions for use of the disclosure may be useful for treating Parkinson's disease psychosis.

Parkinson's disease (PD) is a neurological disorder that may be characterized by a decrease of voluntary movements. The afflicted patient has reduction of motor activity and slower voluntary movements compared to the normal individual. The patient may have characteristic “mask” face, a tendency to hurry while walking, bent over posture and generalized weakness of the muscles. There is a typical “lead-pipe” rigidity of passive movements. Another important feature of the disease is the tremor of the extremities occurring at rest and decreasing during movements.

Parkinson's disease psychosis is experienced by about one third of PD patients and significantly affects the patient's quality of life. Psychosis is characterized by hallucinations, delusions, and other sensory disturbances including illusions and “sense of presence” hallucinations. The underlying cause of psychosis in PD patients is not well understood. However, the occurrence of cognitive impairment in PD patients has been identified as a risk factor associated with the development of psychosis (Laura B. Zahodne and Hubert H. Fernandez, Drugs Aging. 2008, 25(8), 665-682).

Parkinson's disease, the etiology of which is unknown, belongs to a group of the most common movement disorders named parkinsonism, which affects approximately one person per one thousand. These other disorders grouped under the name of parkinsonism may result from viral infection, syphilis, arteriosclerosis and trauma and exposure to toxic chemicals and narcotics. Nonetheless, it is believed that the inappropriate loss of synaptic stability may lead to the disruption of neuronal circuits and to brain diseases. Whether as the result of genetics, drug use, the aging process, viral infections, or other various causes, dysfunction in neuronal communication is considered the underlying cause for many neurologic diseases, such as PD (Myrrhe van Spronsen and Casper C. Hoogenraad, Curr. Neurol. Neurosci Rep. 2010, 10, 207-214).

Regardless of the cause of the disease, the main pathologic feature is degeneration of dopaminergic cells in basal ganglia, especially in substantia nigra. Due to premature death of the dopamine containing neurons in substantia nigra, the largest structure of the basal ganglia, the striatum, wall have reduced input from substantia nigra resulting in decreased dopamine release. The understanding of the underlying pathology led to the introduction of the first successful treatment which can alleviate Parkinson's disease. Virtually all approaches to the therapy of the disease are based on dopamine replacement. Drugs currently used in the treatment can be converted into dopamine after crossing the blood brain barrier, or they can boost the synthesis of dopamine and reduce its breakdown. Unfortunately, the main pathologic event, degeneration of the ceils in substantia nigra, is not helped. The disease continues to progress and frequently after a certain length of time, dopamine replacement treatment will lose its effectiveness.

There are a number of animal models for PD. Exemplary animal models for PD include the reserpine model, the methamphetamine model, the 6-hydroxydopamine (6-OHDA) model, the 1-methyl-4-phenyl-1,2,3,6-tetrahydropyridine (MPTP) model, the paraquat (PQ)-Maneb model, the rotenone model, the 3-nitrotyrosine model and genetic models using transgenic mice. Transgenic models include mice that over express α-synuclein, express human mutant forms of α-synuclein, or mice that express LRKK2 mutations. See review of these models by Ranjita B. et al. (Ranjita B. et al. BioEssays 2002, 24, 308-318). Additional information regarding these animal models is readily available from Jackson Laboratories (see also http://research.jax.org/grs/parkinsons.html), as well as in numerous publications disclosing the use of these validated models.

The efficacy of the methods, uses, combinations, pharmaceutical compositions, combinations for use, or pharmaceutical compositions for use of this disclosure in treating PD psychosis, or cognitive impairment associated with PD, may be assessed in any of the animal models of PD disclosed herein, as well as human subjects with PD, using a variety of cognitive tests known in the art, as discussed herein.

### Autism

This disclosure provides methods, uses, combinations, pharmaceutical compositions, combinations for use, or pharmaceutical compositions for use useful for treating autism using an SV2A inhibitor (e.g., levetiracetam, brivaracetam, or seletracetam), or a pharmaceutically acceptable salt, hydrate, solvate, polymorph, or isomer thereof, in combination with a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof. In certain embodiments, treatment comprises preventing or slowing the progression of cognitive impairment associated with autism. In certain embodiments, treatment comprises alleviation, amelioration, or slowing the progression of cognitive impairment associated with autism. In certain embodiments, the cognitive impairment associated with autism is cognitive deficit. For example, methods, uses, combinations, pharmaceutical compositions, combinations for use, or pharmaceutical compositions for use of the disclosure can be useful to improve the motor/cognitive deficits symptomatic of autism.

“Autism”, as used herein, may refer to an autism spectrum disorder characterized by a neural development disorder leading to impaired social interaction and communication by restricted and repetitive behavior. “Autism Spectrum Disorder” may refer to a group of developmental disabilities that includes autism; Asperger syndrome; pervasive developmental disorder not otherwise specified (PDD-NOS or atypical autism); Rett syndrome; and childhood disintegrative disorder.

Autism is a neurodevelopmental disorder characterized by dysfunction in three core behavioral dimensions: repetitive behaviors, social deficits, and cognitive deficits. The repetitive behavior domain involves compulsive behaviors, unusual attachments to objects, rigid adherence to routines or rituals, and repetitive motor mannerisms such as stereotypies and self-stimulatory behaviors. The social deficit dimension involves deficits in reciprocal social interactions, lack of eye contact, diminished ability to carry on conversation, and impaired daily interaction skills. The cognitive deficits can include language abnormalities. Autism is a disabling neurological disorder that affects thousands of Americans and encompasses a number of subtypes, with various putative causes and few documented ameliorative treatments. The disorders of the autistic spectrum may be present at birth, or may have later onset, for example, at ages two or three. There are no clear-cut biological markers for autism. Diagnosis of the disorder is made by considering the degree to which the child matches the behavioral syndrome, which is characterized by poor communicative abilities, peculiarities in social and cognitive capacities, and maladaptive behavioral patterns. The dysfunction in neuronal communication is considered one of the underlying causes for autism (Myrrhe van Spronsen and Casper C. Hoogenraad, Curr. Neurol. Neurosci. Rep. 2010, 10, 207-214).

### Mental Retardation

The present disclosure contemplates the treatment of cognitive impairment associated with mild mental retardation, moderate mental retardation, severe mental retardation, profound mental retardation, and mental retardation severity unspecified. Such mental retardation may be, but is not required to be, associated with chromosomal changes, (for example Down Syndrome due to trisomy 21), heredity, pregnancy and perinatal problems, and other severe mental disorders. This disclosure provides methods, uses, combinations, pharmaceutical compositions, combinations for use, or pharmaceutical compositions for use useful for treating cognitive impairment associated with mental retardation an SV2A inhibitor (e.g., levetiracetam, brivaracetam, or seletracetam), or a pharmaceutically acceptable salt, hydrate, solvate, polymorph, or isomer thereof, in combination with a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof. In certain embodiments, treatment comprises preventing or slowing the progression of cognitive impairment associated with mental retardation. In certain embodiments, treatment comprises alleviation, amelioration, or slowing the progression of cognitive impairment/cognitive deficit associated with mental retardation. For example, methods, uses, combinations, pharmaceutical compositions, combinations for use, or pharmaceutical compositions for use of the disclosure can be useful to improve the motor/cognitive impairments symptomatic of mental retardation.

Mental retardation is a generalized disorder characterized by significantly impaired cognitive function and deficits in adaptive behaviors. Mental retardation is often defined as an Intelligence Quotient (IQ) score of less than 70. Inborn causes are among many underlying causes for mental retardation. The dysfunction in neuronal communication is also considered one of the underlying causes for mental retardation (Myrrhe van Spronsen and Casper C. Hoogenraad, Curr. Neurol. Neurosci. Rep. 2010, 10, 207-214).

In some instances, mental retardation includes, but are not limited to, Down syndrome, velocardiofacial syndrome, fetal alcohol syndrome, Fragile X syndrome, Klinefelter's syndrome, neurofibromatosis, congenital hypothyroidism, Williams syndrome, phenylketonuria (PKU), Smith-Lemli-Opitz syndrome, Prader-Willi syndrome, Phelan-MeDermid syndrome, Mowat-Wilson syndrome, ciliopathy, Lowe syndrome and siderium type X-linked mental retardation. Down syndrome is a disorder that includes a combination of birth defects, including some degree of mental retardation, characteristic facial features and, often, heart defects, increased infections, problems with vision and hearing, and other health problems. Fragile X syndrome is a prevalent form of inherited mental retardation, occurring with a frequency of 1 in 4,000 males and 1 in 8,000 females. The syndrome is also characterized by developmental delay, hyperactivity, attention deficit disorder, and, autistic-like behavior. There is no effective treatment for fragile X syndrome.

Several animal models have been developed for mental retardation. For example, a knockout mouse model has been developed for Fragile X syndrome. Fragile X syndrome is a common form of mental retardation caused by the absence of the FMR1 protein, FMRP. Two homologs of FMRP have been identified, FXR1P and FXR2P. FXR2P shows high expression in brain and testis, like FMRP. Both Fxr2 and Fmr1 knockout mice, and Fmr1/Fxr2 double knockout mice are believed to be useful models for mental retardation such as Fragile X syndrome. See, Bontekoe C. J. M. et al. Hum. Mol. Genet. 2002, 11 (5): 487-498. The efficacy of the methods, uses, combinations, pharmaceutical compositions, combinations for use, or pharmaceutical compositions for use of this disclosure in treating cognitive deficit/impairment associated with mental retardation may be assessed in the these mouse models and other animal models developed for mental retardation, as well as human subjects with mental retardation, using a variety of cognitive tests known in the art, as discussed herein.

### Compulsive Behavior (Obsessive Compulsive Disorder)

This disclosure provides methods, uses, combinations, pharmaceutical compositions, combinations for use, or pharmaceutical compositions for use useful for treating cognitive impairment associated with OCD using an SV2A inhibitor (e.g., levetiracetam, brivaracetam, or seletracetam), or a pharmaceutically acceptable salt, hydrate, solvate, polymorph, or isomer thereof, in combination with a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof. In certain embodiments, treatment comprises preventing or slowing the progression of cognitive impairment associated with OCD. In certain embodiments, treatment comprises alleviation, amelioration, or slowing the progression of cognitive impairment/cognitive deficit associated with OCD. For example, methods, uses, combinations, pharmaceutical compositions, combinations for use, or pharmaceutical compositions for use of the disclosure can be useful to treat the cognitive deficits in OCD, and/or to improve cognitive function in patients with OCD. A quinpirole-sensitized rat model has been developed for OCD. The compulsive checking behavior of the quinpirole-sensitized rats is subject to interruption, which is an attribute characteristic of OCD compulsions. The efficacy of the methods, uses, combinations, pharmaceutical compositions, combinations for use, or pharmaceutical compositions for use of this disclosure in treating cognitive deficits associated with OCD, may be assessed in this rat model and other animal models developed for OCD, as well as human subjects with OCD, using a variety of cognitive tests known in the art, as discussed herein.

Obsessive compulsive disorder (“OCD”) is a mental condition that is most commonly characterized by intrusive, repetitive unwanted thoughts (obsessions) resulting in compulsive behaviors and mental acts that an individual feels driven to perform (compulsion). Current epidemiological data indicates that OCD is the fourth most common mental disorder in the United States. Some studies suggest the prevalence of OCD is between one and three percent, although the prevalence of clinically recognized OCD is much lower, suggesting that many individuals with the disorder may not be diagnosed. Patients with OCD are often diagnosed by a psychologist, psychiatrist, or psychoanalyst according to the Diagnostic and Statistical Manual of Mental Disorders, 4^{th} edition text revision (DSM-IV-TR) (2000) diagnostic criteria that include characteristics of obsessions and compulsions. Characteristics of obsession include: (1) recurrent and persistent thoughts, impulses, or images that are experienced as intrusive and that cause marked anxiety or distress; (2) the thoughts, impulses, or images are not simply excessive worries about real-life problems; and (3) the person attempts to ignore or suppress such thoughts, impulses, or images, or to neutralize them with some other thought or action. The person recognizes that the obsessional thoughts, impulses, or images are a product of his or her own mind and are not based in reality. Characteristics of compulsion include: (1) repetitive behaviors or mental acts that the person feels driven to perform in response to an obsession, or according to rules that must be applied rigidly; (2) the behaviors or mental acts are aimed, at preventing or reducing distress or preventing some dreaded event or situation; however, these behaviors or mental acts are not actually connected to the issue, or they are excessive.

Individuals with OCD typically perform tasks (or compulsion) to seek relief from obsession-related anxiety. Repetitive behaviors such as handwashing, counting, checking, or cleaning are often performed with the hope of preventing obsessive thoughts or making them go away. Performing these “rituals,” however, only provides temporary relief. People with OCD may also be diagnosed with a spectrum of other mental disorders, such as generalized anxiety disorder, anorexia nervosa, panic attack, or schizophrenia.

The dysfunction in neuronal communication is considered one of the underlying causes for obsession disorder (Myrrhe van Spronsen and Casper C. Hoogenraad, Curr. Neurol. Neurosci. Rep. 2010, 10, 207-214). Studies suggest that OCD may be related to abnormal levels of a neurotransmitter called serotonin. The first-line treatment of OCD consists of behavioral therapy, cognitive therapy, and medications. Medications for treatment include serotonin reuptake inhibitors (SRIs) such as paroxetine (Seroxat™, Paxil®, Xetanor™, ParoMerek™, Rexetin™), sertraline (Zoloft®, Stimuloton™), fluoxetine (Prozac®), Rioxetin™), escitalopram (Lexapro®), and fluvoxamine (Luvox®)) as well as the tricyclic antidepressants, in particular clomipramine (Anafranil®). Benzodiazepines are also useful in treatment. As much as 40% to 60% of the patients, however, fail to adequately respond to the SRI therapy and an even greater proportion of patients fail to experience complete remission of their symptoms.

### Substance Addiction

This disclosure provides methods, uses, combinations, pharmaceutical compositions, combinations for use, or pharmaceutical compositions for use useful for treating cognitive impairment associated with substance addiction an SV2A inhibitor (e.g., levetiracetam, brivaracetam, or seletracetam), or a pharmaceutically acceptable salt, hydrate, solvate, polymorph, or isomer thereof, in combination with a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof. In certain embodiments, treatment comprises preventing or slowing the progression of cognitive impairment associated with substance addiction. In certain embodiments, treatment comprises alleviation, amelioration, or slowing the progression cognitive impairment associated with substance addiction. For example, methods, uses, combinations, pharmaceutical compositions, combinations for use, or pharmaceutical compositions for use of the disclosure can be useful to treat the cognitive impairment and/or to improve cognitive function in patients with substance addiction.

Substance addiction (e.g., drug substance addiction, alcohol substance addiction) is a mental disorder. The substance addiction is not triggered, instantaneously upon exposure to substance of abuse. Rather, it involves multiple, complex neural adaptations that develop with different time courses ranging from hours to days to months (Kauer J. A. Nat. Rev. Neurosci. 2007, 8, 844-858), The path to substance addiction generally begins with the voluntary use of one or more controlled substances, such as narcotics, barbiturates, methamphetamines, alcohol, nicotine, and any of a variety of other such controlled substances. Over time, with extended use of the controlled substance(s), the voluntary ability to abstain from the controlled substance(s) is compromised due to the effects of prolonged use on brain function, and thus on behavior. As such, substance addiction generally is characterized by compulsive substance craving, seeking and use that persist even in the face of negative consequences. The cravings may represent changes in the underlying neurobiology of the patient which likely must be addressed in a meaningful way if recovery is to be obtained. Substance addiction is also characterized in many cases by withdrawal symptoms, which for some substances are life threatening (e.g., alcohol, barbiturates) and in others can result in substantial morbidity (which may include nausea, vomiting, fever, dizziness, and profuse sweating), distress, and decreased ability to obtain recovery. For example, alcoholism, also known as alcohol dependence, is one such substance addiction. Alcoholism is primarily characterized by four symptoms, which include cravings, loss of control, physical dependence and tolerance. These symptoms also may characterize substance addictions to other controlled substances. The craving for alcohol, as well as other controlled substances, often is as strong as the need for food or water. Thus, an alcoholic may continue to drink despite serious family, health and/or legal ramifications.

Recent work exploring the effects of abusing alcohol, central stimulants, and opiates on the central nervous system (CNS) have demonstrated a variety of adverse effects related to mental health, including substance-induced impairments in cognition. See, Nyberg F. Cognitive Impairments in Drug Addicts, Chapter 9. In several laboratories and clinics substantial damages of brain function are seen to result from these drugs. Among the harmful effects of the abusing drugs on brain are those contributing to accelerated obsolescence. An observation that has received special attention during recent years is that chronic drug users display pronounced impairment in brain areas associated with executive and memory function. A remarked neuroadaptation caused by addictive drugs, such as alcohol, central stimulants and opiates involves diminished neurogenesis in the subgranular zone (SGZ) of the hippocampus. Indeed, it has been proposed that decreased adult neurogenesis in the SGZ could modify the hippocampal function in such a way that it contributes to relapse and a maintained addictive behavior. It also raises the possibility that decreased neurogenesis may contribute to cognitive deficits elicited by these abusing drugs.

Several animal models have been developed to study substance addiction. For example, a genetically selected Marchigian Sardinian alcohol-preferring (msP) rat models was developed to study the neurobiology of alcoholism. See, Ciccocioppo R. et al. Substance addiction Biology 2006, 11, 339-355. The efficacy of the methods, uses, combinations, pharmaceutical compositions, combinations for use, or pharmaceutical compositions for use of this disclosure in treating cognitive impairment associated with substance addiction, may also be assessed in animal models of substance addiction, as well as human subjects with substance addiction, using a variety of cognitive tests known in the art, as discussed herein.

### Brain Cancer

The disclosure provides methods, uses, combinations, pharmaceutical compositions, combinations for use, or pharmaceutical compositions for use useful for treating a brain cancer (for example, brain tumors as described herein) using a α5-containing GABA_{A} receptor positive allosteric modulator, such as one selected from the compounds or pharmaceutically acceptable salts, hydrates, solvates, polymorphs, isomers, or combinations thereof as described herein. In certain embodiments, treatment comprises preventing or slowing the progression of a brain cancer. In certain embodiments, treatment comprises alleviation, amelioration, or slowing the progression of one or more symptoms associated with a brain cancer. In certain embodiments, the symptom to be treated is cognitive impairment. For example, methods, uses, combinations, pharmaceutical compositions, combinations for use, or pharmaceutical compositions for use of the disclosure can be useful to treat the cognitive impairment and/or to improve cognitive function in patients with a brain cancer. In some embodiments of the disclosure, there is provided a method of, use for, pharmaceutical composition for use for, or a combination for use for preserving or improving cognitive function in a subject with a brain cancer, the method or use comprising the step of administering to said subject a therapeutically effective amount of a compound of the disclosure or a pharmaceutically acceptable salt, hydrate, solvate, polymorph, isomer, or combination thereof. In some embodiments, the brain tumor is medulloblastoma.

Brain cancer is the growth of abnormal cells in the tissues of the brain usually related to the growth of malignant brain tumors. Brain tumors grow and press on the nearby areas of the brain which can stop that part of the brain from working the way it should. Brain cancer rarely spreads into other tissues outside of the brain. The grade of tumor, based on how abnormal the cancer cells look under a microscope, may be used to tell the difference between slow- and fast-growing tumors. Brain tumors are classified according to the kind of cell from which the tumor seems to originate. Diffuse, fibrillary astrocytomas are the most common type of primary brain tumor in adults. These tumors are divided histopathologically into three grades of malignancy: World Health Organization (WHO) grade II astrocytoma, WHO grade III anaplastic astrocytoma and WHO grade IV glioblastoma multiforme (GBM). WHO grade II astocytomas are the most indolent of the diffuse astrocytoma spectrum. Astrocytomas display a tendency to infiltrate the surrounding brain, confounding therapeutic attempts at local control. These invasive abilities are often apparent in low-grade as well as high-grade tumors.

Glioblastoma multiforme is the most malignant stage of astrocytoma, with survival times of less than 2 years for most patients. Histologically, these tumors are characterized by dense cellularity, high proliferation indices, endothelial proliferation and focal necrosis. The highly proliferative nature of these lesions likely results from multiple mitogenic effects. One of the hallmarks of GBM is endothelial proliferation. A host of angiogenic growth factors and their receptors are found in GBMs.

There are biologic subsets of astrocytomas, which may reflect the clinical heterogeneity observed in these tumors. These subsets include brain stem gliomas, which are a form of pediatric diffuse, fibrillary astrocytoma that often follow a malignant course. Brain stem GBMs share genetic features with those adult GBMs that affect younger patients. Pleomorphic xanthoastrocytoma (PXA) is a superficial, low-grade astrocytic tumor that predominantly affects young adults. While these tumors have a bizarre histological appearance, they are typically slow-growing tumors that may be amenable to surgical cure. Some PXAs, however, may recur as GBM. Pilocytic astrocytoma is the most common astrocytic tumor of childhood and differs clinically and histopathologically from the diffuse, fibrillary astrocytoma that affects adults. Pilocytic astrocytomas do not have the same genomic alterations as diffuse, fibrillary astrocytomas. Subependymal giant cell astrocytomas (SEGA) are periventricular, low-grade astrocytic tumors that are usually associated with tuberous sclerosis (TS) and are histologically identical to the so-called “candle-gutterings” that line the ventricles of TS patients. Similar to the other tumorous lesions in TS, these are slowly growing and may be more akin to hamartomas than true neoplasms. Desmoplastic cerebral astrocytoma of infancy (DCAI) and desmoplastic infantile ganglioglioma (DIGG) are large, superficial, usually cystic, benign astrocytomas that affect children in the first year or two of life.

Oligodendrogliomas and oligoastrocytomas (mixed gliomas) are diffuse, usually cerebral tumors that are clinically and biologically most closely related to the diffuse, fibrillary astrocytomas. The tumors, however, are far less common than astrocytomas and have generally better prognoses than the diffuse astrocytomas. Oligodendrogliomas and oligoastrocytomas may progress, either to WHO grade III anaplastic oligodendroglioma or anaplastic oligoastrocytoma, or to WHO grade IV GBM. Thus, the genetic changes that lead to oligodendroglial tumors constitute yet another pathway to GBM.

Ependymomas are a clinically diverse group of gliomas that vary from aggressive intraventricular tumors of children to benign spinal cord tumors in adults. Transitions of ependymoma to GBM are rare. Choroid plexus tumors are also a varied group of tumors that preferentially occur in the ventricular system, ranging from aggressive supratentorial intraventricular tumors of children to benign cerebellopontine angle tumors of adults. Choroid plexus tumors have been reported occasionally in patients with Li-Fraumeni syndrome and von Hippel-Lindau (VHL) disease.

Medulloblastomas are highly malignant, primitive tumors that arise in the posterior fossa, primarily in children. Medulloblastoma is the most common childhood malignant brain tumor. The most lethal medulloblastoma subtype exhibits a high expression of the GABA_{A} receptor α5 subunit gene and MYC amplification. See, e.g., J Biomed Nanotechnol. 2016 June; 12(6): 1297-302.

Meningiomas are common intracranial tumors that arise in the meninges and compress the underlying brain. Meningiomas are usually benign, but some “atypical” meningiomas may recur locally, and some meningiomas are frankly malignant and may invade the brain or metastasize. Atypical and malignant meningiomas are not as common as benign meningiomas. Schwannomas are benign tumors that arise on peripheral nerves. Schwannomas may arise on cranial nerves, particularly the vestibular portion of the eighth cranial nerve (vestibular schwannomas, acoustic neuromas) where they present as cerebellopontine angle masses. Hemangioblastomas are tumors of uncertain origin that are composed of endothelial cells, pericytes and so-called stromal cells. These benign tumors most frequently occur in the cerebellum and spinal cord of young adults. Multiple hemangioblastomas are characteristic of von Hippel-Lindau disease (VHL). Hemangiopericytomas (HPCs) are dural tumors which may display locally aggressive behavior and may metastasize. The histogenesis of dural-based hemangiopericytoma (HPC) has long been debated, with some authors classifying it as a distinct entity and others classifying it as a subtype of meningioma.

### Modes of Administration of the Compounds, Combinations, and Pharmaceutical Compositions of the Disclosure

The methods, uses, pharmaceutical compositions, combinations, pharmaceutical compositions for use, or combinations for use of the disclosure are useful for treating cognitive impairment, delaying or slowing the progression of cognitive impairment, or reducing the rate of decline of cognitive function, in a subject suffering from cognitive impairment or decline of cognitive function associated with a central nervous system (CNS) disorder, or at risk thereof, by administering to said subject a therapeutically effective amount of an SV2A inhibitor (e.g., levetiracetam, brivaracetam, or seletracetam), or a pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof. In some embodiments, the SV2A inhibitor, or a pharmaceutically acceptable salt, hydrate, solvate, polymorph, or isomer thereof, is selected from the group consisting of levetiracetam, seletracetam, and brivaracetam, or pharmaceutically acceptable salts, solvates, hydrates, polymorphs, or isomers thereof. In other embodiments, the SV2A inhibitor, or the pharmaceutically acceptable salt, hydrate, solvate, polymorph, or isomer thereof, is levetiracetam, or a pharmaceutically acceptable salt, hydrate, solvate, polymorph, or isomer thereof. In other embodiments, the SV2A inhibitor, or the pharmaceutically acceptable salt, hydrate, solvate, polymorph, or isomer thereof, is brivaracetam, or a pharmaceutically acceptable salt, hydrate, solvate, polymorph, or isomer thereof. In other embodiments, the SV2A inhibitor, or the pharmaceutically acceptable salt, hydrate, solvate, polymorph, or isomer thereof, is seletracetam, or a pharmaceutically acceptable salt, hydrate, solvate, polymorph, or isomer thereof. In some embodiments, the GABA_{A} α5 receptor agonist, or the pharmaceutically acceptable salt, hydrate, solvate, polymorph, or isomer thereof, is selected from the group consisting of: a compound of Formula I, or a pharmaceutically acceptable salt, hydrate, solvate, polymorph, or isomer thereof; a compound of Formula II, or a pharmaceutically acceptable salt, hydrate, solvate, polymorph, or isomer thereof; and a compound of Formula IV, or a pharmaceutically acceptable salt, hydrate, solvate, polymorph, or isomer thereof. In some embodiments, the GABA_{A} α5 receptor agonist, or the pharmaceutically acceptable salt, hydrate, solvate, polymorph, or isomer thereof, is selected from the group consisting of: a compound of Formula I, or a pharmaceutically acceptable salt, hydrate, solvate, polymorph, or isomer thereof; a compound of Formula II, or a pharmaceutically acceptable salt, hydrate, solvate, polymorph, or isomer thereof; and a compound of Formula IV, or a pharmaceutically acceptable salt, hydrate, solvate, polymorph, or isomer thereof. In some embodiments, the GABA_{A} α5 receptor agonist, or the pharmaceutically acceptable salt, hydrate, solvate, polymorph, or isomer thereof, is a compound of Formula I, or a pharmaceutically acceptable salt, hydrate, solvate, polymorph, or isomer thereof. In some embodiments, the GABA_{A} α5 receptor agonist, or the pharmaceutically acceptable salt, hydrate, solvate, polymorph, or isomer thereof, is a compound of Formula II, or a pharmaceutically acceptable salt, hydrate, solvate, polymorph, or isomer thereof. In some embodiments, the GABA_{A} α5 receptor agonist, or the pharmaceutically acceptable salt, hydrate, solvate, polymorph, or isomer thereof, is a compound of Formula IV, or a pharmaceutically acceptable salt, hydrate, solvate, polymorph, or isomer thereof. In some embodiments, the compound of Formula I, or the pharmaceutically acceptable salt, hydrate, solvate, polymorph, or isomer thereof, is selected from the group consisting of Compounds 1-114, or a pharmaceutically acceptable salt, hydrate, solvate, polymorph, or isomer of any of the foregoing. In some embodiments, the compound of Formula I, or the pharmaceutically acceptable salt, hydrate, solvate, polymorph, or isomer thereof, is Compound 1, or a pharmaceutically acceptable salt, hydrate, solvate, polymorph, or isomer thereof. In some embodiments, the methods, uses, combinations, pharmaceutical compositions, combinations for use, or pharmaceutical compositions for use of the disclosure may comprise one or more crystalline forms selected from the group consisting of Compound 1, Form A; Compound 1, Form B; Compound 1, Form C; Compound 1, Form E; and Compound 1, Form F. In some embodiments, the crystalline form is Compound 1, Form A. Further, the present disclosure provides methods, uses, combinations, pharmaceutical compositions, combinations for use, or pharmaceutical compositions for use useful for treating cognitive impairment associated with a brain cancer or for treating a brain cancer itself in a subject in need thereof. Additionally, the present disclosure provides methods, uses, combinations, pharmaceutical compositions, combinations for use, or pharmaceutical compositions for use useful for treating Parkinson's disease psychosis in a subject in need thereof. In some embodiments, the subject that suffers from cognitive impairment or decline of cognitive function is a human.

In some embodiments of the combinations of the disclosure, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are in a single pharmaceutical composition. In some embodiments of the combinations of the disclosure, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are formulated in separate pharmaceutical compositions. In certain such embodiments, the separate pharmaceutical compositions are packaged together. In some embodiments, the separate pharmaceutical compositions are packaged separately. Combinations of the disclosure also encompass formulation of the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, together in one formulation or in separate formulations.

In some embodiments of the disclosure, the SV2A inhibitor (e.g., levetiracetam, brivaracetam, or seletracetam), or the pharmaceutically acceptable salt, hydrate, solvate, polymorph, or isomer thereof, and/or GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are administered at doses that are subtherapeutic as compared to the doses at which they are therapeutically effective when administered in the absence of the other. The use of the SV2A inhibitors (e.g., levetiracetam, brivaracetam, or seletracetam), or pharmaceutically acceptable salts, hydrates, solvates, polymorphs, or isomers thereof, in combination with the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, reduces the amount of the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, and the SV2A inhibitor (e.g., levetiracetam, brivaracetam, or seletracetam), or the pharmaceutically acceptable salt, hydrate, solvate, polymorph, or isomer thereof, necessary for the treatment of CNS disorders involving cognitive dysfunction and other affective disorders, including MCI, amnestic MCI, AAMI, ARCE, dementia, AD, PTSD, schizophrenia, bipolar disorder, amyotrophic lateral sclerosis, cancer-therapy-related cognitive impairment, mental retardation, Parkinson's disease, autism, compulsive behavior, and substance addiction; treatment of cognitive impairment associated with a brain cancer or treatment of a brain cancer itself; or Parkinson's disease psychosis. In some embodiments, the subject that suffers such CNS disorders involving cognitive dysfunction and other affective disorders is a human patient, and thus the side effects caused by the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are reduced without diminishing efficacy. Further, the efficacy of a combination of the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, exceeds the efficacy of either compound administered alone at its optimal dose and thus is an improved treatment for CNS disorders associated with cognitive impairment.

“Administering” or “administration of” a compound, pharmaceutical composition, or combination of the disclosure to a subject can be carried out using one of a variety of methods known to those skilled in the art. For example, a compound, pharmaceutical composition, or combination of the disclosure can be administered, intravenously, arterially, intradermally, intramuscularly, intraperitoneally, intravenously, subcutaneously, ocularly, sublingually, orally (by ingestion), buccally, intranasally (by inhalation), intraspinally, intracerebrally, and transdermally (by absorption, e.g., through a skin duct). In some embodiments, the administration is extended release (e.g., a controlled release, a prolonged release, a sustained release, a delayed release, or a slow release). Alternatively, the administration may be immediate release or non-extended release. A compound, pharmaceutical composition, or combination of the disclosure can also appropriately be introduced by rechargeable or biodegradable polymeric devices or other devices, e.g., patches and pumps, or pharmaceutical compositions, which provide for the extended release (e.g., a controlled release, a prolonged release, a sustained release, a delayed release, or a slow release) of the compound. Administering can also be performed, for example, once, a plurality of times, and/or over one or more extended periods. In some aspects, the administration includes both direct administration, including self-administration, and indirect administration, including the act of prescribing a compound, combination, or composition of the disclosure. For example, as used herein, a physician who instructs a patient to self-administer a compound, combination, or composition of the disclosure, or to have the compound, combination, or composition of the disclosure administered by another and/or who provides a patient with a prescription for a compound, combination, or composition of the disclosure is administering the compound, combination, or composition of the disclosure to the patient.

Appropriate methods of administering a compound, pharmaceutical composition, or combination of the disclosure to a subject will also depend, for example, on the age of the subject, whether the subject is active or inactive at the time of administering, whether the subject is cognitively impaired at the time of administering, the extent of the impairment, and the chemical and biological properties of the compound, pharmaceutical composition, or combination of the disclosure (e.g., solubility, digestibility, bioavailability, stability and toxicity). In some embodiments, a compound, pharmaceutical composition, or combination of the disclosure is administered orally, e.g., to a subject by ingestion, or intravenously, e.g., to a subject by injection. In some embodiments, the orally administered compound or combination is in an extended release pharmaceutical composition (e.g., a controlled release, a prolonged release, a sustained release, a delayed release, or a slow release) or administered using a device for such extended release (e.g., a controlled release, a prolonged release, a sustained release, a delayed release, or a slow release).

A “therapeutically effective amount” of a compound, pharmaceutical composition, or combination of the disclosure is an amount of the compound, pharmaceutical composition, or combination of the disclosure that, when administered to a subject may have the intended therapeutic effect, e.g., improving cognitive function, or delaying or slowing the progression of cognitive impairment, or reducing the rate of decline of cognitive function in a subject (e.g., a patient having cognitive impairment or decline of cognitive function associated with a CNS disorder or a brain cancer); improving cognitive function in a patient with a brain cancer, delaying or slowing the progression of a brain cancer or cognitive impairment in a patient with a brain cancer, reducing the rate of decline of cognitive function in a patient with a brain cancer, preventing or slowing the progression of the disease or disorder, or alleviation, amelioration, or slowing the progression of one or more symptoms associated with cognitive impairment associated with a brain cancer; and improving Parkinson's disease psychosis, delaying or slowing the progression of Parkinson's disease psychosis; preventing or slowing the progression of the disease or disorder, or alleviation, amelioration, or slowing the progression of one or more symptoms associated with Parkinson's disease psychosis. The full therapeutic effect does not necessarily occur by administration of one dose and may occur only after administration of a series of doses. Thus, a therapeutically effective amount may be administered in one or more administrations. The precise therapeutically effective amount needed for a subject will depend upon, for example, the subject's size, health and age, the nature and extent of the cognitive impairment or decline of cognitive function, and the therapeutics or combination of therapeutics selected for administration, and the mode of administration. The skilled worker can readily determine the effective amount for a given situation by routine experimentation.

“Subtherapeutic amount” may refer to an amount administered of an compound of the disclosure that is less than the therapeutic amount, that is, less than the amount normally used when said compound is administered alone (e.g., individually and in the absence of other therapeutic compounds) to treat disorders involving cognitive dysfunction.

It will be appreciated that compounds used in the pharmaceutical compositions, uses, combinations, pharmaceutical compositions for use, combinations for use, or methods of this disclosure may readily penetrate the blood-brain barrier when peripherally administered. Compounds which cannot penetrate the blood-brain barrier, however, can still be effectively administered directly into the central nervous system, e.g., by an intraventricular or other neuro-compatible route.

In some embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, or pharmaceutical compositions comprising any of the foregoing, are administered simultaneously or sequentially. In some embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, or pharmaceutical compositions comprising any of the foregoing, are administered simultaneously. In some embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, or pharmaceutical compositions comprising any of the foregoing, are administered sequentially.

As used herein, administration of an SV2A inhibitor (e.g., levetiracetam, brivaracetam, or seletracetam), or a pharmaceutically acceptable salt, hydrate, solvate, polymorph, or isomer thereof, and a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, “in combination,” “combination,” or “together” includes simultaneous administration and/or administration at different times, such as sequential administration. It also includes administration in a single pharmaceutical composition or in separate pharmaceutical compositions packaged together or separately. In certain such embodiments, the separate pharmaceutical compositions are packaged together. In some embodiments, the separate pharmaceutical compositions are packaged separately. Combination encompasses administration of the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, as a co-pharmaceutical composition (single pharmaceutical composition) or, alternatively, as separate pharmaceutical compositions used/administered together. Combination also encompasses administration of the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, together in one formulation or in separate formulations.

As used herein, combination may include administration of any of the disclosed pharmaceutical compositions or disclosed compounds by any route of administration. For example, the SV2A inhibitor (e.g., levetiracetam, brivaracetam, or seletracetam), or the pharmaceutically acceptable salt, hydrate, solvate, polymorph, or isomer thereof, may be formulated/administered in an extended release form (e.g., a controlled release form, a prolonged release form, a sustained release form, a delayed release form, or a slow release form), which may be administered together or separately with the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, wherein the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, may or may not be in an extended release form (e.g., a controlled release form, a prolonged release form, a sustained release form, a delayed release form, or a slow release form) itself. In some embodiments, the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, may be formulated/administered in an extended release form (e.g., a controlled release form, a prolonged release form, a sustained release form, a delayed release form, or a slow release form), which may be administered together or separately with an SV2A inhibitor (e.g., levetiracetam, brivaracetam, or seletracetam), or a pharmaceutically acceptable salt, hydrate, solvate, polymorph, or isomer thereof, wherein the SV2A inhibitor (e.g., levetiracetam, brivaracetam, or seletracetam), or the pharmaceutically acceptable salt, hydrate, solvate, polymorph, or isomer thereof, may or may not be in an extended release form (e.g., a controlled release form, a prolonged release form, a sustained release form, a delayed release form, or a slow release form) itself. In some embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are administered via different routes. In some embodiments, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are administered via the same route.

“Simultaneous administration,” as used herein, may mean that the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are administered with a time separation of no more than about 15 minutes, and in some embodiments no more than about 10 minutes. When the compounds of the disclosure, or the pharmaceutical compositions comprising said compounds, are administered simultaneously, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, may be in the same dosage unit (e.g., a single dosage unit form comprising both the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof) or in discrete dosage units (e.g., the SV2A inhibitor (e.g., levetiracetam, brivaracetam, or seletracetam), or the pharmaceutically acceptable salt, hydrate, solvate, polymorph, or isomer thereof, is in one dosage unit form and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, is in another dosage unit form), optionally in the same container or package or in separate containers or packages. Simultaneous administration of the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, or pharmaceutical compositions comprising any of the foregoing, can optionally be combined with supplemental doses of the SV2A inhibitor (e.g., levetiracetam, brivaracetam, or seletracetam), or the pharmaceutically acceptable salt, hydrate, solvate, polymorph, or isomer thereof, and/or the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, or pharmaceutical compositions comprising any of the foregoing. Simultaneous administration may also include administration of additional agents known to be useful for treating cognitive impairment in a manner similar to that detailed above. Examples of such agents include antipsychotics, memantine, and acetylcholine esterase inhibitors.

“Sequential administration” as used herein may mean that the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, or pharmaceutical compositions comprising any of the foregoing, are administered with a time separation of more than about 15 minutes, and in some embodiments more than about one hour, or up to 12 hours. Either the SV2A inhibitor (e.g., levetiracetam, brivaracetam, or seletracetam), or the pharmaceutically acceptable salt, hydrate, solvate, polymorph, or isomer thereof, or the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, or pharmaceutical compositions comprising any of the foregoing, may be administered first. For sequential administration, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, or pharmaceutical compositions comprising any of the foregoing, may be in discrete dosage unit forms, optionally in the same container or package or in separate containers or packages. Sequential administration may also include administration of additional agents known to be useful for treating cognitive impairment in a manner similar to that detailed above. Examples of such agents include antipsychotics, memantine, and acetylcholine esterase inhibitors.

In accordance with this disclosure, the SV2A inhibitor (e.g., levetiracetam, brivaracetam, or seletracetam), or the pharmaceutically acceptable salt, hydrate, solvate, polymorph, or isomer thereof, or a pharmaceutical composition comprising any of the foregoing, in combination with the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, or a pharmaceutical composition comprising any of the foregoing, can be administered in an extended release form (e.g., a controlled release form, a prolonged release form, a sustained release form, a delayed release form, or a slow release form).

In accordance with this disclosure, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, or pharmaceutical compositions comprising any of the foregoing, can be administered to a subject via any suitable route or routes. In some embodiments, the compounds, combinations, or pharmaceutical compositions of the disclosure are administered orally; however, administration intravenously, subcutaneously, arterially, intradermally, intramuscularly, intraspinally, intracerebrally, rectally, intrathoracically, intraperitoneally, intraventricularly, sublingually, buccally, transdermally, topically, ocularly, intranasally, or by inhalation is also contemplated. The compounds, pharmaceutical compositions, and combinations can be administered orally, for example, in the form of tablets, troches, capsules, elixirs, suspensions, syrups, wafers, or the like, prepared by art recognized procedures. In certain embodiments, the SV2A inhibitor (e.g., levetiracetam, brivaracetam, or seletracetam), or the pharmaceutically acceptable salt, hydrate, solvate, polymorph, or isomer thereof, in combination with the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, or pharmaceutical compositions comprising any of the foregoing, can be administered to a subject via the same route. For example, the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, or pharmaceutical compositions comprising any of the foregoing, are both administered orally. In certain embodiments, the SV2A inhibitor (e.g., levetiracetam, brivaracetam, or seletracetam), or the pharmaceutically acceptable salt, hydrate, solvate, polymorph, or isomer thereof, in combination with the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, or pharmaceutical compositions comprising any of the foregoing, can be administered to a subject via different routes. For example, the SV2A inhibitor (e.g., levetiracetam, brivaracetam, or seletracetam), or the pharmaceutically acceptable salt, hydrate, solvate, polymorph, or isomer thereof, or pharmaceutical compositions comprising any of the foregoing, is administered intravenously and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, is administered orally.

Dosage schedules of the compounds, combinations, and pharmaceutical compositions of the disclosure according to the methods and uses of the disclosure will vary according to the particular compound, combinations, or pharmaceutical compositions of the disclosure selected, the route of administration, the nature of the condition being treated, the age, and condition of the patient, the course, or stage of treatment, and will ultimately be at the discretion of the attending physician. It will be understood that the amount of the SV2A inhibitor (e.g., levetiracetam, brivaracetam, or seletracetam), or the pharmaceutically acceptable salt, hydrate, solvate, polymorph, or isomer thereof, in combination with the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, or pharmaceutical compositions comprising any of the foregoing, that is administered will be amounts effective to produce a desired biological effect, such as beneficial results, including clinical results, e.g., an amount that normalizes neural activity in areas of the brain that exhibit aberrant activity (including, but not limited to DG, CA3 and/or entorhinal cortex) and/or results in an improvement in cognitive function). It will be understood that an effective amount can be administered in more than one dose and over a course of treatment.

If administered by an implant, a device, or an extended release formulation, the SV2A inhibitor (e.g., levetiracetam, brivaracetam, or seletracetam), or the pharmaceutically acceptable salt, hydrate, solvate, polymorph, or isomer thereof, in combination with the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, or pharmaceutical compositions comprising any of the foregoing, can be administered one time, or one or more times periodically throughout the lifetime of the patient as necessary. Other administration intervals intermediate to or shorter than these dosage intervals for clinical applications may also be used and may be determined by one skilled in the art following the methods of this disclosure.

Desired duration of administration of the SV2A inhibitor (e.g., levetiracetam, brivaracetam, or seletracetam), or the pharmaceutically acceptable salt, hydrate, solvate, polymorph, or isomer thereof, in combination with the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, or pharmaceutical compositions comprising any of the foregoing, can be determined by routine experimentation by one skilled in the art. For example, the SV2A inhibitor (e.g., levetiracetam, brivaracetam, or seletracetam), or the pharmaceutically acceptable salt, hydrate, solvate, polymorph, or isomer thereof, in combination with the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, or pharmaceutical compositions comprising any of the foregoing, may be administered for a period of 1-4 weeks, 1-3 months, 3-6 months, 6-12 months, 1-2 years, or more, up to the lifetime of the patient.

In certain embodiments of the disclosure, the interval of administration of the compound, combination, or pharmaceutical composition of the disclosure is 12 hours (twice daily). In certain embodiments of the disclosure, the interval of administration of the compound, combination, or pharmaceutical composition of the disclosure is 24 hours (once daily). Administration at less frequent intervals, such as once every 6 hours, may also be used. Other doses higher than, intermediate to, or less than these doses may also be used and may be determined by one skilled in the art following the methods of this disclosure. For repeated administrations over several days or weeks or longer, depending on the condition, the treatment is sustained until a sufficient level of cognitive function is achieved.

In certain embodiments of the disclosure, the dose of the SV2A inhibitor (e.g., levetiracetam, brivaracetam, or seletracetam), or the pharmaceutically acceptable salt, hydrate, solvate, polymorph, or isomer thereof, is 0.1 to 5 mg/kg/day. In certain embodiments of the disclosure, the dose of the SV2A inhibitor (e.g., levetiracetam, brivaracetam, or seletracetam), or the pharmaceutically acceptable salt, hydrate, solvate, polymorph, or isomer thereof, is 7 mg/day to 350 mg/day.

In certain embodiments of the disclosure, the dose of the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, is between 0.0001 mg/kg/day and 100 mg/kg/day. In certain embodiments of the disclosure, the dose of the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, is between 0.007 mg/day and 7000 mg/day. In some embodiments, the interval of administration of the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, or a pharmaceutical composition comprising any of the foregoing, is once every 12 hours (twice daily) or 24 hours (once daily). Administration at less frequent intervals, such as once every 6 hours, may also be used. Other doses higher than, intermediate to, or less than these doses may also be used and may be determined by one skilled in the art following the methods of this disclosure. For repeated administrations over several days or weeks or longer, depending on the condition, the treatment is sustained until a sufficient level of cognitive function is achieved. In some embodiments, the GABA_{A} α5 receptor agonist, or the pharmaceutically acceptable salt, hydrate, solvate, polymorph, or isomer thereof, is selected from the group consisting of: a compound of Formula I, or a pharmaceutically acceptable salt, hydrate, solvate, polymorph, or isomer thereof; a compound of Formula II, or a pharmaceutically acceptable salt, hydrate, solvate, polymorph, or isomer thereof; and a compound of Formula IV, or a pharmaceutically acceptable salt, hydrate, solvate, polymorph, or isomer thereof. In some embodiments, the GABA_{A} α5 receptor agonist, or the pharmaceutically acceptable salt, hydrate, solvate, polymorph, or isomer thereof, is a compound of Formula I, or a pharmaceutically acceptable salt, hydrate, solvate, polymorph, or isomer thereof. In some embodiments, the GABA_{A} α5 receptor agonist, or the pharmaceutically acceptable salt, hydrate, solvate, polymorph, or isomer thereof, is a compound of Formula II, or a pharmaceutically acceptable salt, hydrate, solvate, polymorph, or isomer thereof. In some embodiments, the GABA_{A} α5 receptor agonist, or the pharmaceutically acceptable salt, hydrate, solvate, polymorph, or isomer thereof, is a compound of Formula IV, or a pharmaceutically acceptable salt, hydrate, solvate, polymorph, or isomer thereof. In some embodiments, the compound of Formula I, or the pharmaceutically acceptable salt, hydrate, solvate, polymorph, or isomer thereof, is selected from the group consisting of Compounds 1-114, or a pharmaceutically acceptable salt, hydrate, solvate, polymorph, or isomer of any of the foregoing. In some embodiments, the compound of Formula I, or the pharmaceutically acceptable salt, hydrate, solvate, polymorph, or isomer thereof, is Compound 1, or a pharmaceutically acceptable salt, hydrate, solvate, polymorph, or isomer thereof. In some embodiments, one or more crystalline forms selected from the group consisting of Compound 1, Form A; Compound 1, Form B; Compound 1, Form C; Compound 1, Form E; and Compound 1, Form F may be administered. In some embodiments, the crystalline form is Compound 1, Form A.

In some embodiments, the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, or a pharmaceutical composition comprising any of the foregoing, administered is in an extended release form (e.g., a controlled release form, a prolonged release form, a sustained release form, a delayed release form, or a slow release form). In some embodiments, the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, or a pharmaceutical composition comprising any of the foregoing, is administered once or twice daily.

In the methods, uses, pharmaceutical compositions, combinations, pharmaceutical compositions for use, or combinations for use of the disclosure, the SV2A inhibitor (e.g., levetiracetam, brivaracetam, or seletracetam), or the pharmaceutically acceptable salt, hydrate, solvate, polymorph, or isomer thereof, may be administered at doses as disclosed, for example, in U.S. patent application Ser. No. 12/580,464 (Pub. No. US-2010-0099735), U.S. patent application Ser. No. 13/287,531 (Pub. No. US-2012-0046336), U.S. patent application Ser. No. 13/370,253 (Pub. No. US-2012-0214859), International Patent Application PCT/US2009/005647 (Pub. No. WO2010/044878), International Patent Application PCT/US2012/024556 (Pub. No. WO2012/109491), International Patent Application PCT/US2014/029170 (Pub. No. WO2014/144663), U.S. Patent Application 61/105,847, U.S. Patent Application 61/152,631, U.S. Patent Application 61/175,536, U.S. Patent Application 61/441,251, and U.S. Patent Application 61/794,909.

In some embodiments, the interval of administration of the SV2A inhibitor (e.g., levetiracetam, brivaracetam, or seletracetam), or the pharmaceutically acceptable salt, hydrate, solvate, polymorph, or isomer thereof, or a pharmaceutical composition comprising any of the foregoing, is once every 12 hours (twice daily) or 24 hours (once daily). Administration at less frequent intervals, such as once every 6 hours, may also be used. Other doses higher than, intermediate to, or less than these doses may also be used and may be determined by one skilled in the art following the methods of this disclosure. For repeated administrations over several days or weeks or longer, depending on the condition, the treatment is sustained until a sufficient level of cognitive function is achieved.

In certain embodiments of the disclosure, the SV2A inhibitor (e.g., levetiracetam, brivaracetam, or seletracetam), or the pharmaceutically acceptable salt, hydrate, solvate, polymorph, or isomer thereof, is administered at a daily dose of 0.001 to 5 mg/kg, about 0.001 to 0.5 mg/kg, about 0.01 to 0.5 mg/kg, about 0.1 to 5 mg/kg, or 1 to 2 mg/kg, or 2 to 4 mg/kg, or 2 to 3 mg/kg, or 3 to 4 mg/kg, or 0.2 to 0.4 mg/kg, or 0.2 to 0.3 mg/kg, or 0.3 to 0.4 mg/kg, or 0.1 to 0.2 mg/kg, or 0.01 to 2.5 mg/kg, or 0.1 to 2.5 mg/kg, or 0.4 to 2.5 mg/kg, or 0.6 to 1.8 mg/kg, or 0.5 to 2 mg/kg, or 0.8 to 1.6, or 0.8 to 3.6, or 0.5 to 4 mg/kg, or 0.04 to 2.5 mg/kg, or 0.06 to 1.8 mg/kg, or 0.05 to 3 mg/kg or 0.08 to 1.6 mg/kg, or 0.08 to 3.6 or 0.05 to 2 mg/kg, or 0.01 to 1 mg/kg, or 0.001 to 1 mg/kg, or 0.5 to 5 mg/kg, or 0.05 to 0.5 mg/kg, or 0.8 mg/kg, or 1.6 mg/kg, or 3.6 mg/kg, or 0.08 mg/kg, or 0.16 mg/kg, or 0.36 mg/kg. In some embodiments, the SV2A inhibitor (e.g., levetiracetam, brivaracetam, or seletracetam), or the pharmaceutically acceptable salt, hydrate, solvate, polymorph, or isomer thereof, is administered at a daily dose of 0.1 mg/kg to 0.2 mg/kg, or 0.01 mg/kg to 2.5 mg/kg, or 0.1 mg/kg to 2.5 mg/kg, or 0.4 mg/kg to 2.5 mg/kg, or 0.6 mg/kg to 1.8 mg/kg, or 0.04 mg/kg to 2.5 mg/kg, or 0.06 mg/kg to 1.8 mg/kg, or 2.0 mg/kg to 4.0 mg/kg, or 2.0 mg/kg to 3.0 mg/kg, or 3.0 mg/kg to 4.0 mg/kg, or 0.2 mg/kg to 0.4 mg/kg, or 0.2 mg/kg to 0.3 mg/kg, or 0.3 mg/kg to 0.4 mg/kg, or 0.001 mg/kg to 5 mg/kg, or 0.001 mg/kg to 0.5 mg/kg, or 0.01 mg/kg to 0.5 mg/kg. In some embodiments, the SV2A inhibitor (e.g., levetiracetam, brivaracetam, or seletracetam), or the pharmaceutically acceptable salt, hydrate, solvate, polymorph, or isomer thereof, is administered at a daily dose of 0.1 mg to 500 mg, 0.1 mg to 350 mg, 0.7 mg to 350 mg, 3 mg to 300 mg, 3 mg to 150 mg, 3 mg to 110 mg, 7 mg to 70 mg, 70 mg to 350 mg, 100 mg to 300 mg, or 125 mg to 250 mg; or 0.0015 mg/kg to 7 mg/kg, 0.0015 mg/kg to 5 mg/kg, 0.01 mg/kg to 5 mg/kg, 0.05 mg/kg to 4 mg/kg, 0.05 mg/kg to 2 mg/kg, 0.05 mg/kg to 1.5 mg/kg, 0.1 mg/kg to 1 mg/kg, 1 mg/kg to 5 mg/kg, 1.5 mg/kg to 4 mg/kg, or 1.8 mg/kg to 3.6 mg/kg. Other doses higher than, intermediate to, or less than these doses may also be used and may be determined by one skilled in the art following the methods of this disclosure. For repeated administrations over several days or weeks or longer, depending on the condition, the treatment is sustained until a sufficient level of cognitive function is achieved.

In certain embodiments of the disclosure, the dose of the SV2A inhibitor (e.g., levetiracetam, brivaracetam, or seletracetam), or the pharmaceutically acceptable salt, hydrate, solvate, polymorph, or isomer thereof, is 0.001-5 mg/kg/day. In certain embodiments of the disclosure, the dose of the SV2A inhibitor (e.g., levetiracetam, brivaracetam, or seletracetam), or the pharmaceutically acceptable salt, hydrate, solvate, polymorph, or isomer thereof, is 0.07-350 mg/day. Doses that may be used include, but are not limited to 0.001 mg/kg/day, 0.0015 mg/kg/day, 0.002 mg/kg/day, 0.005 mg/kg/day, 0.0075 mg/kg/day, 0.01 mg/kg/day, 0.015 mg/kg/day, 0.02 mg/kg/day, 0.03 mg/kg/day, 0.04 mg/kg/day, 0.05 mg/kg/day, 0.1 mg/kg/day, 0.2 mg/kg/day, 0.3 mg/kg/day, 0.4 mg/kg/day, 0.5 mg/kg/day, 0.75 mg/kg/day, 1.0 mg/kg/day, 1.5 mg/kg/day, 2.0 mg/kg/day, 2.5 mg/kg/day, 3.0 mg/kg/day, 4.0 mg/kg/day, or 5.0 mg/kg/day. In some embodiments, the dose of the SV2A inhibitor (e.g., levetiracetam, brivaracetam, or seletracetam), or the pharmaceutically acceptable salt, hydrate, solvate, polymorph, or isomer thereof, is 0.001-0.5 mg/kg/day or 0.01-0.5 mg/kg/day. In some embodiments, the dose of the SV2A inhibitor (e.g., levetiracetam, brivaracetam, or seletracetam), or the pharmaceutically acceptable salt, hydrate, solvate, polymorph, or isomer thereof, is 0.07-35 mg/day or 0.7-35 mg/day. Other doses higher than, intermediate to, or less than these doses may also be used and may be determined by one skilled in the art following the methods of this disclosure.

In certain embodiments of the disclosure, the dose of the SV2A inhibitor (e.g., levetiracetam, brivaracetam, or seletracetam), or the pharmaceutically acceptable salt, hydrate, solvate, polymorph, or isomer thereof, is 0.1 to 5 mg/kg/day. In certain embodiments of the disclosure, the dose of the SV2A inhibitor (e.g., levetiracetam, brivaracetam, or seletracetam), or the pharmaceutically acceptable salt, hydrate, solvate, polymorph, or isomer thereof, is 7 to 350 mg/day. Doses that may be used include, but are not limited to, 0.1 mg/kg/day, 0.5 mg/kg/day, 1 mg/kg/day, 1.5 mg/kg/day, 2 mg/kg/day, 2.5 mg/kg/day, 3 mg/kg/day, 4 mg/kg/day, or 5 mg/kg/day. In certain embodiments, the dose is 1-2 mg/kg/day. In certain embodiments, the dose is 70-140 mg/day. In other embodiments of the disclosure, the dose of the SV2A inhibitor (e.g., levetiracetam, brivaracetam, or seletracetam), or the pharmaceutically acceptable salt, hydrate, solvate, polymorph, or isomer thereof, is 0.1 to 0.2 mg/kg/day. Other doses higher than, intermediate to, or less than these doses may also be used and may be determined by one skilled in the art following the methods of this disclosure.

In certain embodiments of the disclosure, the dose of the SV2A inhibitor (e.g., levetiracetam, brivaracetam, or seletracetam), or the pharmaceutically acceptable salt, hydrate, solvate, polymorph, or isomer thereof, is 0.01 to 2.5 mg/kg/day. In certain embodiments of the disclosure, the dose of the SV2A inhibitor (e.g., levetiracetam, brivaracetam, or seletracetam), or the pharmaceutically acceptable salt, hydrate, solvate, polymorph, or isomer thereof, is 0.7-180 mg/day. Doses that may be used include, but are not limited to, 0.01 mg/kg/day, 0.02 mg/kg/day, 0.03 mg/kg/day, 0.04 mg/kg/day, 0.06 mg/kg/day, 0.08 mg/kg/day, 0.12 mg/kg/day, 0.14 mg/kg/day, 0.16 mg/kg/day, 0.18 mg/kg/day, 0.2 mg/kg/day, 0.4 mg/kg/day, 0.6 mg/kg/day, 0.8 mg/kg/day, 1.0 mg/kg/day, 1.2 mg/kg/day, 1.4 mg/kg/day, 1.6 mg/kg/day, 1.8 mg/kg/day, 2.0 mg/kg/day, 2.2 mg/kg/day, 2.4 mg/kg/day, or 2.5 mg/kg/day. In some embodiments, the dose of the SV2A inhibitor (e.g., levetiracetam, brivaracetam, or seletracetam), or the pharmaceutically acceptable salt, hydrate, solvate, polymorph, or isomer thereof, is 0.1-2.5 mg/kg/day, 0.1-0.2 mg/kg/day, 0.2-0.4 mg/kg/day, 0.4-2.5 mg/kg/day, 0.6-1.8 mg/kg/day, 0.04-2.5 mg/kg/day or 0.06-1.8 mg/kg/day. In some embodiments, the dose of the SV2A inhibitor (e.g., levetiracetam, brivaracetam, or seletracetam), or the pharmaceutically acceptable salt, hydrate, solvate, polymorph, or isomer thereof, is 7-180 mg/day, 7-15 mg/day, 14-30 mg/day, 25-180 mg/day, 40-130 mg/day, 2.5-180 mg/day, or 4-130 mg/day. In some embodiments of the disclosure, the dose of the SV2A inhibitor (e.g., levetiracetam, brivaracetam, or seletracetam), or the pharmaceutically acceptable salt, hydrate, solvate, polymorph, or isomer thereof, is 40 to 130 mg, 140 to 300 mg, 200 to 300 mg or 140 to 200 mg. In some embodiments of the disclosure, the dose of the SV2A inhibitor (e.g., levetiracetam, brivaracetam, or seletracetam), or the pharmaceutically acceptable salt, hydrate, solvate, polymorph, or isomer thereof, is 190 mg to 220 mg. In some embodiments of the disclosure, the dose of the SV2A inhibitor (e.g., levetiracetam, brivaracetam, or seletracetam), or the pharmaceutically acceptable salt, hydrate, solvate, polymorph, or isomer thereof, is 190 mg to 240 mg. In some embodiments of the disclosure, the dose of the SV2A inhibitor (e.g., levetiracetam, brivaracetam, or seletracetam), or the pharmaceutically acceptable salt, hydrate, solvate, polymorph, or isomer thereof, is about 220 mg. Other doses higher than, intermediate to, or less than these doses may also be used and may be determined by one skilled in the art following the methods of this disclosure.

In certain embodiments of the disclosure, the dose of the SV2A inhibitor (e.g., levetiracetam, brivaracetam, or seletracetam), or the pharmaceutically acceptable salt, hydrate, solvate, polymorph, or isomer thereof, is 0.0015 to 7 mg/kg/day. In certain embodiments of the disclosure, the dose of the SV2A inhibitor (e.g., levetiracetam, brivaracetam, or seletracetam), or the pharmaceutically acceptable salt, hydrate, solvate, polymorph, or isomer thereof, is about 0.1-500 mg/day. Daily doses that may be used include, but are not limited to, 0.0015 mg/kg, 0.002 mg/kg, 0.0025 mg/kg, 0.005 mg/kg, 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.2 mg/kg, 1.4 mg/kg, 1.5 mg/kg, 1.6 mg/kg, 1.8 mg/kg, 2.0 mg/kg, 2.2 mg/kg, 2.4 mg/kg, 2.5 mg/kg, 2.6 mg/kg, 2.8 mg/kg, 3.0 mg/kg, 3.5 mg/kg, 4.0 mg/kg, 4.5 mg/kg, 5.0 mg/kg, 6.0 mg/kg, or 7.0 mg/kg; or 0.1 mg, 0.15 mg, 0.18 mg, 0.35 mg, 0.7 mg, 1.5 mg, 2.0 mg, 2.5 mg, 2.8 mg, 3.0 mg, 3.5 mg, 4.2 mg, 5 mg, 5.5 mg, 6.0 mg, 7 mg, 8 mg, 9 mg, 10 mg, 12 mg, 15 mg, 20 mg, 25 mg, 28 mg, 30 mg, 35 mg, 40 mg, 45 mg, 50 mg, 55 mg, 60 mg, 70 mg, 75 mg, 80 mg, 85 mg, 90 mg, 95 mg, 100 mg, 110 mg, 120 mg, 125 mg, 140 mg, 150 mg, 170 mg, 175 mg, 180 mg, 190 mg, 200 mg, 210 mg, 225 mg, 250 mg, 280 mg, 300 mg, 350 mg, 400 mg, or 500 mg. In some embodiments, the daily dose of SV2A inhibitor (e.g., levetiracetam, brivaracetam, or seletracetam), or the pharmaceutically acceptable salt, hydrate, solvate, polymorph, or isomer thereof, that can be used in the methods, uses, pharmaceutical compositions for use, or combinations for use of this disclosure include, without limitation, 0.0015-5 mg/kg, 0.05-4 mg/kg, 0.05-2.0 mg/kg, 0.05-1.5 mg/kg, 0.1-1.0 mg/kg, 1-5 mg/kg, 1.5-4.0 mg/kg, 1.8-3.6 mg/kg, 0.01-0.8 mg/kg, 0.01-1 mg/kg, 0.01-1.5 mg/kg, 0.01-2 mg/kg, 0.01-2.5 mg/kg, 0.01-3 mg/kg, 0.01-3.5 mg/kg, 0.01-4 mg/kg, 0.01-5 mg/kg, 0.025-0.8 mg/kg, 0.025-1 mg/kg, 0.025-1.5 mg/kg, 0.025-2 mg/kg, 0.025-2.5 mg/kg, 0.025-3 mg/kg, 0.025-3.5 mg/kg, 0.025-4 mg/kg, 0.05-0.8 mg/kg, 0.05-1 mg/kg, 0.05-1.5 mg/kg, 0.05-2 mg/kg, 0.05-2.5 mg/kg, 0.05-3 mg/kg, 0.05-3.5 mg/kg, 0.05-4 mg/kg, 0.075-0.8 mg/kg, 0.075-1 mg/kg, 0.075-1.5 mg/kg, 0.075-2 mg/kg, 0.075-2.5 mg/kg, 0.075-3 mg/kg, 0.075-3.5 mg/kg, 0.075-4 mg/kg, 0.1-0.8 mg/kg, 0.1-1 mg/kg, 0.1-1.5 mg/kg, 0.1-2 mg/kg, 0.1-2.5 mg/kg, 0.1-3 mg/kg, 0.1-3.5 mg/kg, 0.1-4 mg/kg, 0.2-0.8 mg/kg, 0.2-1 mg/kg, 0.2-1.5 mg/kg, 0.2-2 mg/kg, 0.2-2.5 mg/kg, 0.2-3 mg/kg, 0.2-3.5 mg/kg, 0.2-4 mg/kg, 0.5-0.8 mg/kg, 0.5-1 mg/kg, 0.5-1.5 mg/kg, 0.5-2 mg/kg, 0.5-2.5 mg/kg, 0.5-3 mg/kg, 0.5-3.5 mg/kg, or 0.5-4 mg/kg; or 0.1-350 mg, 0.7-50 mg, 0.7-75 mg, 0.7-100 mg, 0.7-150 mg, 0.7-180 mg, 0.7-225 mg, 0.7-250 mg, 0.7-280 mg, 1.8-50 mg, 1.8-75 mg, 1.8-100 mg, 1.8-150 mg, 1.8-180 mg, 1.8-225 mg, 1.8-250 mg, 1.8-280 mg, 3.5-50 mg, 3.5-75 mg, 3.5-100 mg, 3.5-150 mg, 3.5-180 mg, 3.5-225 mg, 3.5-250 mg, 3.5-280 mg, 5-50 mg, 5-75 mg, 5-100 mg, 5-150 mg, 5-180 mg, 5-225 mg, 5-250 mg, 5-280 mg, 7-50 mg, 7-75 mg, 7-100 mg, 7-150 mg, 7-180 mg, 7-225 mg, 7-250 mg, 7-280 mg, 15-50 mg, 15-75 mg, 15-100 mg, 15-150 mg, 15-180 mg, 15-225 mg, 15-250 mg, 15-280 mg, 35-50 mg, 35-75 mg, 35-100 mg, 35-150 mg, 35-180 mg, 35-225 mg, 35-250 mg, 35-280 mg, 0.1-500 mg, 3-300 mg, 3-150 mg, 3-110 mg, 7-70 mg, 70-350 mg, 100-300 mg, 190-220 mg, 190-240 mg, or 125-250 mg. In some embodiments, the daily dose of SV2A inhibitor (e.g., levetiracetam, brivaracetam, or seletracetam), or the pharmaceutically acceptable salt, hydrate, solvate, polymorph, or isomer thereof, that can be used in the methods, uses, pharmaceutical compositions for use, or combinations for use of this disclosure includes 0.1-350 mg/day. In some embodiments, the daily dose of SV2A inhibitor (e.g., levetiracetam, brivaracetam, or seletracetam), or the pharmaceutically acceptable salt, hydrate, solvate, polymorph, or isomer thereof, that can be used in the methods, uses, pharmaceutical compositions for use, or combinations for use of this disclosure includes about 220 mg/day. Other doses higher than, intermediate to, or less than these doses may also be used and may be determined by one skilled in the art following the methods of this disclosure.

In some embodiments, the total daily dose of SV2A inhibitor (e.g., levetiracetam, brivaracetam, or seletracetam), or the pharmaceutically acceptable salt, hydrate, solvate, polymorph, or isomer thereof, that can be used in the methods, uses, pharmaceutical compositions for use, or combinations for use of this disclosure includes 0.1-350 mg/day. In some embodiments, the SV2A inhibitor (e.g., levetiracetam, brivaracetam, or seletracetam), or the pharmaceutically acceptable salt, hydrate, solvate, polymorph, or isomer thereof, is administered at a total daily dose of 0.1 to 5 mg/kg (e.g., in the case of administration every 12 hours of a daily dose of 2 mg/kg, each administration is 1 mg/kg). In some embodiments, the SV2A inhibitor (e.g., levetiracetam, brivaracetam, or seletracetam), or the pharmaceutically acceptable salt, hydrate, solvate, polymorph, or isomer thereof, is administered every 24 hours at a daily dose of 1 to 2 mg/kg. In some embodiments, the SV2A inhibitor (e.g., levetiracetam, brivaracetam, or seletracetam), or the pharmaceutically acceptable salt, hydrate, solvate, polymorph, or isomer thereof, is administered every 24 hours at a daily dose of 0.1-0.2 mg/kg. In some embodiments, the SV2A inhibitor (e.g., levetiracetam, brivaracetam, or seletracetam), or the pharmaceutically acceptable salt, hydrate, solvate, polymorph, or isomer thereof, is administered at a daily dose of 0.01 to 2.5 mg/kg (e.g., in the case of administration every 12 hours of a daily dose of 0.8 mg/kg, each administration is 0.4 mg/kg). In some embodiments, the SV2A inhibitor (e.g., levetiracetam, brivaracetam, or seletracetam), or the pharmaceutically acceptable salt, hydrate, solvate, polymorph, or isomer thereof, is administered at a daily dose of 0.1 to 2.5 mg/kg. In some embodiments, the SV2A inhibitor (e.g., levetiracetam, brivaracetam, or seletracetam), or the pharmaceutically acceptable salt, hydrate, solvate, polymorph, or isomer thereof, is administered at a daily dose of 0.4 to 2.5 mg/kg. In some embodiments, the SV2A inhibitor (e.g., levetiracetam, brivaracetam, or seletracetam), or the pharmaceutically acceptable salt, hydrate, solvate, polymorph, or isomer thereof, is administered at a daily dose of 0.6 to 1.8 mg/kg. In some embodiments, the SV2A inhibitor (e.g., levetiracetam, brivaracetam, or seletracetam), or the pharmaceutically acceptable salt, hydrate, solvate, polymorph, or isomer thereof, is administered at a daily dose of 0.04-2.5 mg/kg. In some embodiments, the SV2A inhibitor (e.g., levetiracetam, brivaracetam, or seletracetam), or the pharmaceutically acceptable salt, hydrate, solvate, polymorph, or isomer thereof, is administered at a daily dose of 0.06-1.8 mg/kg. In some embodiments, the SV2A inhibitor (e.g., levetiracetam, brivaracetam, or seletracetam), or the pharmaceutically acceptable salt, hydrate, solvate, polymorph, or isomer thereof, is administered at a daily dose of 0.001-5 mg/kg. In some embodiments, the SV2A inhibitor (e.g., levetiracetam, brivaracetam, or seletracetam), or the pharmaceutically acceptable salt, hydrate, solvate, polymorph, or isomer thereof, is administered at a daily dose of 0.001-0.5 mg/kg. In some embodiments, the SV2A inhibitor (e.g., levetiracetam, brivaracetam, or seletracetam), or the pharmaceutically acceptable salt, hydrate, solvate, polymorph, or isomer thereof, is administered at a daily dose of 0.01-0.5 mg/kg. Other doses higher than, intermediate to, or less than these doses may also be used and may be determined by one skilled in the art following the methods of this disclosure.

In certain embodiments of the disclosure, the SV2A inhibitor, or the pharmaceutically acceptable salt, hydrate, solvate, polymorph, or isomer thereof, is levetiracetam, or a pharmaceutically acceptable salt, hydrate, solvate, polymorph, or isomer thereof. The levetiracetam, or the pharmaceutically acceptable salt, hydrate, solvate, polymorph, or isomer thereof, may be administered at a daily dose of 1 to 2 mg/kg, or 0.1 to 2.5 mg/kg, or 0.4 to 2.5 mg/kg, or 0.6 to 1.8 mg/kg, or 2.0 to 3.0 mg/kg, or 3.0 to 4.0 mg/kg, or 2.0 to 4.0 mg/kg, or 0.1 to 5 mg/kg, or 70 to 140 mg, or 7 to 180 mg, or 25 to 180 mg, or 40 to 130 mg, or 140 to 300 mg, or 200 to 300 mg, or 140 to 200 mg, or 7 to 350 mg. The levetiracetam, or the pharmaceutically acceptable salt, hydrate, solvate, polymorph, or isomer thereof, may be administered at a daily dose of 190 mg to 220 mg. The levetiracetam, or the pharmaceutically acceptable salt, hydrate, solvate, polymorph, or isomer thereof, may be administered at a daily dose of 190 mg to 240 mg. The levetiracetam, or the pharmaceutically acceptable salt, hydrate, solvate, polymorph, or isomer thereof, may be administered at a daily dose of 220 mg. Other doses higher than, intermediate to, or less than these doses may also be used and may be determined by one skilled in the art following the methods of this disclosure.

In some embodiments, the levetiracetam, or a pharmaceutically acceptable salt, hydrate, solvate, polymorph, or isomer thereof, is administered at a daily dose of 1 mg/kg to 2 mg/kg, or 0.1 mg/kg to 2.5 mg/kg, or 0.4 mg/kg to 2.5 mg/kg, or 0.6 mg/kg to 1.8 mg/kg, or 2.0 mg/kg to 3.0 mg/kg, or 3.0 mg/kg to 4.0 mg/kg, or 2.0 mg/kg to 4.0 mg/kg, or 0.1 mg/kg to 5 mg/kg, or 70 mg to 140 mg, or 7 mg to 180 mg, or 25 mg to 180 mg, or 40 mg to 130 mg, or 140 to 300 mg, or 200 to 300 mg, or 140 to 200 mg, or 7 mg to 350 mg, 70 mg to 350 mg, 100 mg to 300 mg, or 125 mg to 250 mg, or 0.1 mg/kg to 5 mg/kg, 1 mg/kg to 5 mg/kg, 1.5 mg/kg to 4 mg/kg, or 1.8 mg/kg to 3.6 mg/kg. In some embodiments, the levetiracetam, or a pharmaceutically acceptable salt, hydrate, solvate, polymorph, or isomer thereof, is administered at a daily dose of 190 mg to 220 mg. In some embodiments, the levetiracetam, or a pharmaceutically acceptable salt, hydrate, solvate, polymorph, or isomer thereof, is administered at a daily dose of 190 mg to 240 mg. In some embodiments, the levetiracetam, or a pharmaceutically acceptable salt, hydrate, solvate, polymorph, or isomer thereof, is administered at a daily dose of 220 mg. Other doses higher than, intermediate to, or less than these doses may also be used and may be determined by one skilled in the art following the methods of this disclosure.

In other embodiments, the levetiracetam, or the pharmaceutically acceptable salt, hydrate, solvate, polymorph, or isomer thereof, is administered according to one of the daily dose ranges indicated as “+” listed in Table 1 or Table 2.

TABLE 1
Daily Doses of Levetiracetam

| | Lower range | | | | | |
|---|---|---|---|---|---|---|
| Upper range | 0.1 mg/kg | 0.4 mg/kg | 0.6 mg/kg | 1 mg/kg | 2 mg/kg | 3 mg/kg |
| 1.8 mg/kg | + | + | + | + | | |
| 2 mg/kg | + | + | + | + | | |
| 2.5 mg/kg | + | + | + | + | + | |
| 3 mg/kg | + | + | + | + | + | |
| 4 mg/kg | + | + | + | + | + | + |
| 5 mg/kg | + | + | + | + | + | + |

TABLE 2
Daily Doses of Levetiracetam in a Human Subject

| | Lower range | | | | | |
|---|---|---|---|---|---|---|
| Upper range | 7 mg | 25 mg | 40 mg | 70 mg | 140 mg | 200 mg |
| 130 mg | + | + | + | + | | |
| 140 mg | + | + | + | + | | |
| 180 mg | + | + | + | + | + | |
| 200 mg | + | + | + | + | + | |
| 300 mg | + | + | + | + | + | + |
| 350 mg | + | + | + | + | + | + |

In certain embodiments of the disclosure, the SV2A inhibitor, or the pharmaceutically acceptable salt, hydrate, solvate, polymorph, or isomer thereof, is levetiracetam, or a pharmaceutically acceptable salt, hydrate, solvate, polymorph, or isomer thereof. In some embodiments, the levetiracetam, or the pharmaceutically acceptable salt, hydrate, solvate, polymorph, or isomer thereof, is administered at a daily dose of 0.1-5 mg/kg, 1-5 mg/kg, 1.5-4 mg/kg, 1.8-3.6 mg/kg, 7-350 mg, 70-350 mg, 100-300 mg, or 125-250 mg. In some embodiments, the levetiracetam, or a pharmaceutically acceptable salt, hydrate, solvate, polymorph, or isomer thereof, is administered at a daily dose of 190 mg to 220 mg. In some embodiments, the levetiracetam, or a pharmaceutically acceptable salt, hydrate, solvate, polymorph, or isomer thereof, is administered at a daily dose of 190 mg to 240 mg. In some embodiments, the levetiracetam, or a pharmaceutically acceptable salt, hydrate, solvate, polymorph, or isomer thereof, is administered at a daily dose of 220 mg. Other doses higher than, intermediate to, or less than these doses may also be used and may be determined by one skilled in the art following the methods of this disclosure.

In some embodiments, the levetiracetam, or the pharmaceutically acceptable salt, hydrate, solvate, polymorph, or isomer thereof, or a pharmaceutical composition comprising any of the foregoing, administered is in an extended release form (e.g., a controlled release form, a prolonged release form, a sustained release form, a delayed release form, or a slow release form). In some embodiments, the levetiracetam, or the pharmaceutically acceptable salt, hydrate, solvate, polymorph, or isomer thereof, or a pharmaceutical composition comprising any of the foregoing, is administered once or twice daily.

In certain embodiments of the disclosure, the SV2A inhibitor, or the pharmaceutically acceptable salt, hydrate, solvate, polymorph, or isomer thereof, is brivaracetam, or a pharmaceutically acceptable salt, hydrate, solvate, polymorph, or isomer thereof. In some embodiments, the brivaracetam, or the pharmaceutically acceptable salt, hydrate, solvate, polymorph, or isomer thereof, is administered at a daily dose of 0.1 to 0.2 mg/kg, or 0.01 to 2.5 mg/kg, or 0.04 to 2.5 mg/kg, or 0.06 to 1.8 mg/kg, or 0.2 to 0.4 mg/kg, or 7 to 15 mg, or 0.7 to 180 mg, or 2.5 to 180 mg, or 4.0 to 130 mg, or 14 to 30 mg. Other doses higher than, intermediate to, or less than these doses may also be used and may be determined by one skilled in the art following the methods of this disclosure.

In other embodiments, the brivaracetam, or the pharmaceutically acceptable salt, hydrate, solvate, polymorph, or isomer thereof, is administered at a daily dose of at least 0.1 mg, 0.5 mg, 0.75 mg, 1.0 mg, 1.5 mg, or 2.0 mg, but no more than a daily dose of 2.5 mg, 5 mg, 10 mg, 15 mg, 20 mg, 25 mg, 30 mg, or 35 mg. In other embodiments, the brivaracetam, or the pharmaceutically acceptable salt, hydrate, solvate, polymorph, or isomer thereof, is administered at a daily dose of at least 0.0015 mg/kg, 0.0075 mg/kg, 0.01 mg/kg, 0.015 mg/kg, 0.02 mg/kg, or 0.03 mg/kg, but no more than a daily dose of 0.5 mg/kg, 0.4 mg/kg, 0.3 mg/kg, 0.2 mg/kg, 0.15 mg/kg, 0.1 mg/kg, 0.05 mg/kg, or 0.04 mg/kg. Other doses higher than, intermediate to, or less than these doses may also be used and may be determined by one skilled in the art following the methods of this disclosure.

In some embodiments, the brivaracetam, or the pharmaceutically acceptable salt, hydrate, solvate, polymorph, or isomer thereof, is administered at a daily dose of about 0.1 mg/kg to 0.2 mg/kg, or about 0.01 mg/kg to 2.5 mg/kg, or about 0.04 mg/kg to 2.5 mg/kg, or about 0.06 mg/kg to 1.8 mg/kg, or about 0.2 mg/kg to 0.4 mg/kg, or about 7 mg to 15 mg, or about 0.7 mg to 180 mg, or about 2.5 mg to 180 mg, or about 4.0 mg to 130 mg, or about 14 mg to 30 mg. In other embodiments, the brivaracetam, or the pharmaceutically acceptable salt, hydrate, solvate, polymorph, or isomer thereof, is administered at a daily dose of about 0.1 mg-350 mg, 0.7 mg-350 mg, 3 mg-300 mg, 3 mg-150 mg, 3 mg-110 mg, or 7 mg-70 mg; or 0.0015 mg/kg-5 mg/kg, 0.01 mg/kg-5 mg/kg, 0.05 mg/kg-4.0 mg/kg, 0.05 mg/kg-2 mg/kg, 0.05 mg/kg-1.5 mg/kg, or 0.1 mg/kg-1 mg/kg. Other doses higher than, intermediate to, or less than these doses may also be used and may be determined by one skilled in the art following the methods of this disclosure.

In other embodiments, the brivaracetam, or the pharmaceutically acceptable salt, hydrate, solvate, polymorph, or isomer thereof, is administered according to one of the daily dose ranges indicated as “+” listed in Table 3 or Table 4. For example, the brivaracetam, or the pharmaceutically acceptable salt, hydrate, solvate, polymorph, or isomer thereof, may be administered at a daily dose of 0.1-35 mg, 0.5-35 mg, 0.75-35 mg, 1.0-35 mg, 1.5-35 mg, 2.0-35 mg, 0.1-30 mg, 0.1-25 mg, 0.1-20 mg, 0.1-15 mg, 0.1-10 mg, 0.1-5 mg, 0.1-2.5 mg, 0.0015-0.5 mg/kg, 0.0075-0.5 mg/kg, 0.01-0.5 mg/kg, 0.015-0.5 mg/kg, 0.02-0.5 mg/kg, 0.03-0.5 mg/kg, 0.0015-0.4 mg/kg, 0.0015-0.3 mg/kg, 0.0015-0.2 mg/kg, 0.0015-0.15 mg/kg, 0.0015-0.1 mg/kg, 0.0015-0.05 mg/kg, or 0.0015-0.04 mg/kg. Other doses higher than, intermediate to, or less than these doses may also be used and may be determined by one skilled in the art following the methods of this disclosure.

TABLE 3
Daily Doses of Brivaracetam

| | Lower range | | | | | |
|---|---|---|---|---|---|---|
| | 0.0015 | 0.0075 | 0.01 | 0.015 | 0.02 | 0.03 |
| Upper range | mg/kg | mg/kg | mg/kg | mg/kg | mg/kg | mg/kg |
| 0.04 mg/kg | + | + | + | + | + | + |
| 0.05 mg/kg | + | + | + | + | + | + |
| 0.1 mg/kg | + | + | + | + | + | + |
| 0.15 mg/kg | + | + | + | + | + | + |
| 0.2 mg/kg | + | + | + | + | + | + |
| 0.3 mg/kg | + | + | + | + | + | + |
| 0.4 mg/kg | + | + | + | + | + | + |
| 0.5 mg/kg | + | + | + | + | + | + |

TABLE 4
Daily Doses of Brivaracetam in a Human Subject

| | Lower range | | | | | |
|---|---|---|---|---|---|---|
| Upper range | 0.1 mg | 0.5 mg | 0.75 mg | 1.0 mg | 1.5 mg | 2.0 mg |
| 2.5 mg | + | + | + | + | + | + |
| 5 mg | + | + | + | + | + | + |
| 10 mg | + | + | + | + | + | + |
| 15 mg | + | + | + | + | + | + |
| 20 mg | + | + | + | + | + | + |
| 25 mg | + | + | + | + | + | + |
| 30 mg | + | + | + | + | + | + |
| 35 mg | + | + | + | + | + | + |

In other embodiments, the brivaracetam, or the pharmaceutically acceptable salt, hydrate, solvate, polymorph, or isomer thereof, is administered at a daily dose of at least 0.0015 mg/kg, 0.002 mg/kg, 0.0025 mg/kg, 0.005 mg/kg, 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.2 mg/kg, 0.3 mg/kg, 0.4 mg/kg, 0.5 mg/kg, but no more than a daily dose of 1 mg/kg, 1.2 mg/kg, 1.4 mg/kg, 1.5 mg/kg, 1.6 mg/kg, 1.8 mg/kg, 2.0 mg/kg, 2.2 mg/kg, 2.4 mg/kg, 2.5 mg/kg, 2.6 mg/kg, 2.8 mg/kg, 3.0 mg/kg, 3.5 mg/kg, 4.0 mg/kg, 4.5 mg/kg, or 5.0 mg/kg. In other embodiments, the brivaracetam, or the pharmaceutically acceptable salt, hydrate, solvate, polymorph, or isomer thereof, is administered at a daily dose of at least 0.1 mg, 0.15 mg, 0.18 mg, 0.35 mg, 0.7 mg, 1.5 mg, 2.0 mg, 2.5 mg, 2.8 mg, 3.0 mg, 3.5 mg, 4.2 mg, 5 mg, 5.5 mg, 6.0 mg, 7 mg, 10 mg, 15 mg, 20 mg, 25 mg, 28 mg, 30 mg, or 35 mg but no more than a daily dose of 70 mg, 80 mg, 85 mg, 100 mg, 110 mg, 125 mg, 140 mg, 150 mg, 170 mg, 175 mg, 180 mg, 190 mg, 200 mg, 210 mg, 225 mg, 250 mg, 280 mg, 300 mg, or 350 mg. In some embodiments, the brivaracetam, or the pharmaceutically acceptable salt, hydrate, solvate, polymorph, or isomer thereof, may be administered at a daily dose of 0.0015-5 mg/kg, 0.1-350 mg, 0.01-5 mg/kg, 0.7-350 mg, 0.05-4 mg/kg, 3-300 mg, 0.05-2.0 mg/kg, 3-150 mg, 0.05-1.5 mg, 3-110 mg, 0.1-1.0 mg/kg, 7-70 mg. Other doses higher than, intermediate to, or less than these doses may also be used and may be determined by one skilled in the art following the methods of this disclosure.

In other embodiments, the brivaracetam, or the pharmaceutically acceptable salt, hydrate, solvate, polymorph, or isomer thereof, is administered according to one of the daily dose ranges indicated as “+” listed in Table 5 or Table 6. For example, the brivaracetam, or the pharmaceutically acceptable salt, hydrate, solvate, polymorph, or isomer thereof, may be administered at a daily dose of 0.01-0.8 mg/kg, 0.01-1 mg/kg, 0.01-1.5 mg/kg, 0.01-2 mg/kg, 0.01-2.5 mg/kg, 0.01-3 mg/kg, 0.01-3.5 mg/kg, 0.01-4 mg/kg, 0.01-5 mg/kg, 0.025-0.8 mg/kg, 0.025-1 mg/kg, 0.025-1.5 mg/kg, 0.025-2 mg/kg, 0.025-2.5 mg/kg, 0.025-3 mg/kg, 0.025-3.5 mg/kg, 0.025-4 mg/kg, 0.05-0.8 mg/kg, 0.05-1 mg/kg, 0.05-1.5 mg/kg, 0.05-2 mg/kg, 0.05-2.5 mg/kg, 0.05-3 mg/kg, 0.05-3.5 mg/kg, 0.05-4 mg/kg, 0.075-0.8 mg/kg, 0.075-1 mg/kg, 0.075-1.5 mg/kg, 0.075-2 mg/kg, 0.075-2.5 mg/kg, 0.075-3 mg/kg, 0.075-3.5 mg/kg, 0.075-4 mg/kg, 0.1-0.8 mg/kg, 0.1-1 mg/kg, 0.1-1.5 mg/kg, 0.1-2 mg/kg, 0.1-2.5 mg/kg, 0.1-3 mg/kg, 0.1-3.5 mg/kg, 0.1-4 mg/kg, 0.2-0.8 mg/kg, 0.2-1 mg/kg, 0.2-1.5 mg/kg, 0.2-2 mg/kg, 0.2-2.5 mg/kg, 0.2-3 mg/kg, 0.2-3.5 mg/kg, 0.2-4 mg/kg, 0.5-0.8 mg/kg, 0.5-1 mg/kg, 0.5-1.5 mg/kg, 0.5-2 mg/kg, 0.5-2.5 mg/kg, 0.5-3 mg/kg, 0.5-3.5 mg/kg, or 0.5-4 mg/kg; or 0.7-50 mg, 0.7-75 mg, 0.7-100 mg, 0.7-150 mg, 0.7-180 mg, 0.7-225 mg, 0.7-250 mg, 0.7-280 mg, 1.8-50 mg, 1.8-75 mg, 1.8-100 mg, 1.8-150 mg, 1.8-180 mg, 1.8-225 mg, 1.8-250 mg, 1.8-280 mg, 3.5-50 mg, 3.5-75 mg, 3.5-100 mg, 3.5-150 mg, 3.5-180 mg, 3.5-225 mg, 3.5-250 mg, 3.5-280 mg, 5-50 mg, 5-75 mg, 5-100 mg, 5-150 mg, 5-180 mg, 5-225 mg, 5-250 mg, 5-280 mg, 7-50 mg, 7-75 mg, 7-100 mg, 7-150 mg, 7-180 mg, 7-225 mg, 7-250 mg, 7-280 mg, 15-50 mg, 15-75 mg, 15-100 mg, 15-150 mg, 15-180 mg, 15-225 mg, 15-250 mg, 15-280 mg, 35-50 mg, 35-75 mg, 35-100 mg, 35-150 mg, 35-180 mg, 35-225 mg, 35-250 mg, or 35-280 mg. Other doses higher than, intermediate to, or less than these doses may also be used and may be determined by one skilled in the art following the methods of this disclosure.

TABLE 5
Daily Doses of Brivaracetam (mg/kg)

| | Lower range | | | | | | | | |
|---|---|---|---|---|---|---|---|---|---|
| Upper range | 0.0015 | 0.01 | 0.025 | 0.04 | 0.05 | 0.075 | 0.1 | 0.2 | 0.5 |
| 0.8 | + | + | + | + | + | + | + | + | + |
| 1 | + | + | + | + | + | + | + | + | + |
| 1.5 | + | + | + | + | + | + | + | + | + |
| 2 | + | + | + | + | + | + | + | + | + |
| 2.5 | + | + | + | + | + | + | + | + | + |
| 3 | + | + | + | + | + | + | + | + | + |
| 3.5 | + | + | + | + | + | + | + | + | + |
| 4 | + | + | + | + | + | + | + | + | + |
| 5 | + | + | + | + | + | + | + | + | + |

TABLE 6
Daily Doses of Brivaracetam in a Human Subject (mg)

| | Lower range | | | | | | | | |
|---|---|---|---|---|---|---|---|---|---|
| Upper range | 0.1 | 0.7 | 1.8 | 3.0 | 3.5 | 5 | 7 | 15 | 35 |
| 50 | + | + | + | + | + | + | + | + | + |
| 75 | + | + | + | + | + | + | + | + | + |
| 100 | + | + | + | + | + | + | + | + | + |
| 110 | + | + | + | + | + | + | + | + | + |
| 150 | + | + | + | + | + | + | + | + | + |
| 180 | + | + | + | + | + | + | + | + | + |
| 225 | + | + | + | + | + | + | + | + | + |
| 250 | + | + | + | + | + | + | + | + | + |
| 280 | + | + | + | + | + | + | + | + | + |
| 300 | + | + | + | + | + | + | + | + | + |
| 350 | + | + | + | + | + | + | + | + | + |

In some embodiments, the brivaracetam, or the pharmaceutically acceptable salt, hydrate, solvate, polymorph, or isomer thereof, or a pharmaceutical composition comprising any of the foregoing, administered is in an extended release form (e.g., a controlled release form, a prolonged release form, a sustained release form, a delayed release form, or a slow release form). In some embodiments, the brivaracetam, or the pharmaceutically acceptable salt, hydrate, solvate, polymorph, or isomer thereof, or a pharmaceutical composition comprising any of the foregoing, administered is administered once or twice daily.

In certain embodiments of the disclosure, the SV2A inhibitor, or the pharmaceutically acceptable salt, hydrate, solvate, polymorph, or isomer thereof, is seletracetam, or a pharmaceutically acceptable salt, hydrate, solvate, polymorph, or isomer thereof. In some embodiments, the seletracetam, or the pharmaceutically acceptable salt, hydrate, solvate, polymorph, or isomer thereof, is administered at a daily dose of at least 0.1 mg, 0.5 mg, 0.75 mg, 1.0 mg, 1.5 mg, or 2.0 mg, but no more than a daily dose of 2.5 mg, 5 mg, 10 mg, 15 mg, 20 mg, 25 mg, 30 mg, or 35 mg. In other embodiments, the seletracetam, or a pharmaceutically acceptable salt, hydrate, solvate, polymorph, or isomer thereof, is administered at a daily dose of at least 0.0015 mg/kg, 0.0075 mg/kg, 0.01 mg/kg, 0.015 mg/kg, 0.02 mg/kg, or 0.03 mg/kg, but no more than a daily dose of 0.5 mg/kg, 0.4 mg/kg, 0.3 mg/kg, 0.2 mg/kg, 0.15 mg/kg, 0.1 mg/kg, 0.05 mg/kg, or 0.04 mg/kg. Other doses higher than, intermediate to, or less than these doses may also be used and may be determined by one skilled in the art following the methods of this disclosure.

In other embodiments, the seletracetam, or the pharmaceutically acceptable salt, hydrate, solvate, polymorph, or isomer thereof, is administered at a daily dose of about 0.1 mg-350 mg, 0.7 mg-350 mg, 3 mg-300 mg, 3 mg-150 mg, 3 mg-110 mg, or 7 mg-70 mg; or 0.0015 mg/kg-5 mg/kg, 0.01 mg/kg-5 mg/kg, 0.05 mg/kg-4.0 mg/kg, 0.05 mg/kg-2 mg/kg, 0.05 mg/kg-1.5 mg/kg, or 0.1 mg/kg-1 mg/kg. Other doses higher than, intermediate to, or less than these doses may also be used and may be determined by one skilled in the art following the methods of this disclosure.

In certain embodiments of the disclosure, the seletracetam, or the pharmaceutically acceptable salt, hydrate, solvate, polymorph, or isomer thereof, is administered according to one of the daily dose ranges indicated as “+” listed in Table 7 or Table 8. For example, the seletracetam, or the pharmaceutically acceptable salt, hydrate, solvate, polymorph, or isomer thereof, may be administered at a daily dose of 0.1-35 mg, 0.5-35 mg, 0.75-35 mg, 1.0-35 mg, 1.5-35 mg, 2.0-35 mg, 0.1-30 mg, 0.1-25 mg, 0.1-20 mg, 0.1-15 mg, 0.1-10 mg, 0.1-5 mg, 0.1-2.5 mg, 0.0015-0.5 mg/kg, 0.0075-0.5 mg/kg, 0.01-0.5 mg/kg, 0.015-0.5 mg/kg, 0.02-0.5 mg/kg, 0.03-0.5 mg/kg, 0.0015-0.4 mg/kg, 0.0015-0.3 mg/kg, 0.0015-0.2 mg/kg, 0.0015-0.15 mg/kg, 0.0015-0.1 mg/kg, 0.0015-0.05 mg/kg, or 0.0015-0.04 mg/kg. Other doses higher than, intermediate to, or less than these doses may also be used and may be determined by one skilled in the art following the methods of this disclosure.

TABLE 7
Daily Doses of Selectracetam

| | Lower range | | | | | |
|---|---|---|---|---|---|---|
| | 0.0015 | 0.0075 | 0.01 | 0.015 | 0.02 | 0.03 |
| Upper range | mg/kg | mg/kg | mg/kg | mg/kg | mg/kg | mg/kg |
| 0.04 mg/kg | + | + | + | + | + | + |
| 0.05 mg/kg | + | + | + | + | + | + |
| 0.1 mg/kg | + | + | + | + | + | + |
| 0.15 mg/kg | + | + | + | + | + | + |
| 0.2 mg/kg | + | + | + | + | + | + |
| 0.3 mg/kg | + | + | + | + | + | + |
| 0.4 mg/kg | + | + | + | + | + | + |
| 0.5 mg/kg | + | + | + | + | + | + |

TABLE 8
Daily Doses of Selectracetam in a Human Subject

| | Lower range | | | | | |
|---|---|---|---|---|---|---|
| Upper range | 0.1 mg | 0.5 mg | 0.75 mg | 1.0 mg | 1.5 mg | 2.0 mg |
| 2.5 mg | + | + | + | + | + | + |
| 5 mg | + | + | + | + | + | + |
| 10 mg | + | + | + | + | + | + |
| 15 mg | + | + | + | + | + | + |
| 20 mg | + | + | + | + | + | + |
| 25 mg | + | + | + | + | + | + |
| 30 mg | + | + | + | + | + | + |
| 35 mg | + | + | + | + | + | + |

In other embodiments, the seletracetam, or the pharmaceutically acceptable salt, hydrate, solvate, polymorph, or isomer thereof, is administered at a daily dose of at least 0.0015 mg/kg, 0.002 mg/kg, 0.0025 mg/kg, 0.005 mg/kg, 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.2 mg/kg, 0.3 mg/kg, 0.4 mg/kg, 0.5 mg/kg, but no more than a daily dose of 1 mg/kg, 1.2 mg/kg, 1.4 mg/kg, 1.5 mg/kg, 1.6 mg/kg, 1.8 mg/kg, 2.0 mg/kg, 2.2 mg/kg, 2.4 mg/kg, 2.5 mg/kg, 2.6 mg/kg, 2.8 mg/kg, 3.0 mg/kg, 3.5 mg/kg, 4.0 mg/kg, 4.5 mg/kg, or 5.0 mg/kg. In other embodiments, the seletracetam, or the pharmaceutically acceptable salt, hydrate, solvate, polymorph, or isomer thereof, is administered at a daily dose of at least 0.1 mg, 0.15 mg, 0.18 mg, 0.35 mg, 0.7 mg, 1.5 mg, 2.0 mg, 2.5 mg, 2.8 mg, 3.0 mg, 3.5 mg, 4.2 mg, 5 mg, 5.5 mg, 6.0 mg, 7 mg, 10 mg, 15 mg, 20 mg, 25 mg, 28 mg, 30 mg, or 35 mg but no more than a daily dose of 70 mg, 80 mg, 85 mg, 100 mg, 110 mg, 125 mg, 140 mg, 150 mg, 170 mg, 175 mg, 180 mg, 190 mg, 200 mg, 210 mg, 225 mg, 250 mg, 280 mg, 300 mg, or 350 mg. In some embodiments, the brivaracetam, or the pharmaceutically acceptable salt, hydrate, solvate, polymorph, or isomer thereof, may be administered at a daily dose of 0.0015-5 mg/kg, 0.1-350 mg, 0.01-5 mg/kg, 0.7-350 mg, 0.05-4 mg/kg, 3-300 mg, 0.05-2.0 mg/kg, 3-150 mg, 0.05-1.5 mg, 3-110 mg, 0.1-1.0 mg/kg, 7-70 mg. Other doses higher than, intermediate to, or less than these doses may also be used and may be determined by one skilled in the art following the methods of this disclosure.

In other embodiments, the seletracetam, or the pharmaceutically acceptable salt, hydrate, solvate, polymorph, or isomer thereof, is administered according to one of the daily dose ranges indicated as “+” listed in Table 9 or Table 10. For example, the seletracetam, or the pharmaceutically acceptable salt, hydrate, solvate, polymorph, or isomer thereof, may be administered at a daily dose of 0.01-0.8 mg/kg, 0.01-1 mg/kg, 0.01-1.5 mg/kg, 0.01-2 mg/kg, 0.01-2.5 mg/kg, 0.01-3 mg/kg, 0.01-3.5 mg/kg, 0.01-4 mg/kg, 0.01-5 mg/kg, 0.025-0.8 mg/kg, 0.025-1 mg/kg, 0.025-1.5 mg/kg, 0.025-2 mg/kg, 0.025-2.5 mg/kg, 0.025-3 mg/kg, 0.025-3.5 mg/kg, 0.025-4 mg/kg, 0.05-0.8 mg/kg, 0.05-1 mg/kg, 0.05-1.5 mg/kg, 0.05-2 mg/kg, 0.05-2.5 mg/kg, 0.05-3 mg/kg, 0.05-3.5 mg/kg, 0.05-4 mg/kg, 0.075-0.8 mg/kg, 0.075-1 mg/kg, 0.075-1.5 mg/kg, 0.075-2 mg/kg, 0.075-2.5 mg/kg, 0.075-3 mg/kg, 0.075-3.5 mg/kg, 0.075-4 mg/kg, 0.1-0.8 mg/kg, 0.1-1 mg/kg, 0.1-1.5 mg/kg, 0.1-2 mg/kg, 0.1-2.5 mg/kg, 0.1-3 mg/kg, 0.1-3.5 mg/kg, 0.1-4 mg/kg, 0.2-0.8 mg/kg, 0.2-1 mg/kg, 0.2-1.5 mg/kg, 0.2-2 mg/kg, 0.2-2.5 mg/kg, 0.2-3 mg/kg, 0.2-3.5 mg/kg, 0.2-4 mg/kg, 0.5-0.8 mg/kg, 0.5-1 mg/kg, 0.5-1.5 mg/kg, 0.5-2 mg/kg, 0.5-2.5 mg/kg, 0.5-3 mg/kg, 0.5-3.5 mg/kg, or 0.5-4 mg/kg; or 0.7-50 mg, 0.7-75 mg, 0.7-100 mg, 0.7-150 mg, 0.7-180 mg, 0.7-225 mg, 0.7-250 mg, 0.7-280 mg, 1.8-50 mg, 1.8-75 mg, 1.8-100 mg, 1.8-150 mg, 1.8-180 mg, 1.8-225 mg, 1.8-250 mg, 1.8-280 mg, 3.5-50 mg, 3.5-75 mg, 3.5-100 mg, 3.5-150 mg, 3.5-180 mg, 3.5-225 mg, 3.5-250 mg, 3.5-280 mg, 5-50 mg, 5-75 mg, 5-100 mg, 5-150 mg, 5-180 mg, 5-225 mg, 5-250 mg, 5-280 mg, 7-50 mg, 7-75 mg, 7-100 mg, 7-150 mg, 7-180 mg, 7-225 mg, 7-250 mg, 7-280 mg, 15-50 mg, 15-75 mg, 15-100 mg, 15-150 mg, 15-180 mg, 15-225 mg, 15-250 mg, 15-280 mg, 35-50 mg, 35-75 mg, 35-100 mg, 35-150 mg, 35-180 mg, 35-225 mg, 35-250 mg, or 35-280 mg. Other doses higher than, intermediate to, or less than these doses may also be used and may be determined by one skilled in the art following the methods of this disclosure.

TABLE 9
Daily Doses of Selectracetam (mg/kg)

| | Lower range | | | | | | | | |
|---|---|---|---|---|---|---|---|---|---|
| Upper range | 0.0015 | 0.01 | 0.025 | 0.04 | 0.05 | 0.075 | 0.1 | 0.2 | 0.5 |
| 0.8 | + | + | + | + | + | + | + | + | + |
| 1 | + | + | + | + | + | + | + | + | + |
| 1.5 | + | + | + | + | + | + | + | + | + |
| 2 | + | + | + | + | + | + | + | + | + |
| 2.5 | + | + | + | + | + | + | + | + | + |
| 3 | + | + | + | + | + | + | + | + | + |
| 3.5 | + | + | + | + | + | + | + | + | + |
| 4 | + | + | + | + | + | + | + | + | + |
| 5 | + | + | + | + | + | + | + | + | + |

TABLE 10
Daily Doses of Selectracetam in a Human Subject (mg)

| | Lower range | | | | | | | | |
|---|---|---|---|---|---|---|---|---|---|
| Upper range | 0.1 | 0.7 | 1.8 | 3.0 | 3.5 | 5 | 7 | 15 | 35 |
| 50 | + | + | + | + | + | + | + | + | + |
| 75 | + | + | + | + | + | + | + | + | + |
| 100 | + | + | + | + | + | + | + | + | + |
| 110 | + | + | + | + | + | + | + | + | + |
| 150 | + | + | + | + | + | + | + | + | + |
| 180 | + | + | + | + | + | + | + | + | + |
| 225 | + | + | + | + | + | + | + | + | + |
| 250 | + | + | + | + | + | + | + | + | + |
| 280 | + | + | + | + | + | + | + | + | + |
| 300 | + | + | + | + | + | + | + | + | + |
| 350 | + | + | + | + | + | + | + | + | + |

In some embodiments, the seletracetam, or a pharmaceutically acceptable salt, hydrate, solvate, polymorph, or isomer thereof, or a pharmaceutical composition comprising any of the foregoing, administered is in an extended release form (e.g., a controlled release form, a prolonged release form, a sustained release form, a delayed release form, or a slow release form). In some embodiments, the seletracetam, or the pharmaceutically acceptable salt, hydrate, solvate, polymorph, or isomer thereof, or a pharmaceutical composition comprising any of the foregoing, administered is administered once or twice daily.

The SV2A inhibitor (e.g., levetiracetam, brivaracetam, or seletracetam), or the pharmaceutically acceptable salt, hydrate, solvate, polymorph, or isomer thereof, may be administered at a subtherapeutic dosage level when provided in combination with the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, due to the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), the pharmaceutically acceptable salt, hydrate, solvate, polymorph, or isomer thereof, dependent increase in the therapeutic index of the SV2A inhibitor (e.g., levetiracetam, brivaracetam, or seletracetam), or the pharmaceutically acceptable salt, hydrate, solvate, polymorph, or isomer thereof. In some embodiments, the therapeutic index of the SV2A inhibitor (e.g., levetiracetam, brivaracetam, or seletracetam), or the pharmaceutically acceptable salt, hydrate, solvate, polymorph, or isomer thereof, due to the combination with the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, is greater than the therapeutic index of the SV2A inhibitor (e.g., levetiracetam, brivaracetam, or seletracetam), or the pharmaceutically acceptable salt, hydrate, solvate, polymorph, or isomer thereof, administered in the absence of the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, by at least about 1.5× or 2.0× or 2.5× or 3.0× or 3.5× or 4.0× or 4.5× or 5.0× or 5.5× or 6.0× or 6.5× or 7.0× or 7.5× or 8.0× or 8.5× or 9.0× or 9.5× or 10×, or greater than about 10×. In some embodiments, combinations of an SV2A inhibitor (e.g., levetiracetam, brivaracetam, or seletracetam), or the pharmaceutically acceptable salt, hydrate, solvate, polymorph, or isomer thereof, with the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, reduces the dosage of the SV2A inhibitor (e.g., levetiracetam, brivaracetam, or seletracetam), or the pharmaceutically acceptable salt, hydrate, solvate, polymorph, or isomer thereof, required for its therapeutic effect.

In some embodiments of the disclosure, the SV2A inhibitor (e.g., levetiracetam, brivaracetam, or seletracetam), or the pharmaceutically acceptable salt, hydrate, solvate, polymorph, or isomer thereof, that is administered in combination with the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, is administered at a daily dose of about 0.001 mg/kg to 5 mg/kg, or about 0.1 to 5 mg/kg, or about 1 to 2 mg/kg, or about 0.1 to 0.2 mg/kg, or about 0.01 to 2.5 mg/kg, or about 0.1 to 2.5 mg/kg, or about 0.4 to 2.5 mg/kg, or about 0.6 to 1.8 mg/kg, or about 0.04 to 2.5 mg/kg, or about 0.06 to 1.8 mg/kg, or about 0.01 to 1 mg/kg, or about 0.001 to 1 mg/kg, or about 0.5 mg/kg to 5 mg/kg, or about 0.05 mg/kg to 0.5 mg/kg.

In some embodiments, the amount of the SV2A inhibitor (e.g., levetiracetam, brivaracetam, or seletracetam), or the pharmaceutically acceptable salt, hydrate, solvate, polymorph, or isomer thereof, that is administered in combination with the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, is a subtherapeutic amount (as compared to the therapeutic dose of the SV2A inhibitor (e.g., levetiracetam, brivaracetam, or seletracetam), or the pharmaceutically acceptable salt, hydrate, solvate, polymorph, or isomer thereof, when administered alone). Such subtherapeutic amount, may be, for example, a daily dose, administered at a daily dose of less than 5 mg/kg, less than 2.5 mg/kg, less than 2 mg/kg, less than 1.75 mg/kg, less than 1.6 mg/kg, less than 1.5 mg/kg, less than 1 mg/kg, less than 0.8 mg/kg, less than 0.6 mg/kg, less than 0.5 mg/kg, less than 0.4 mg/kg, less than 0.3 mg/kg, less than 0.2 mg/kg, less than 0.1 mg/kg, less than 0.05 mg/kg, less than 0.04 mg/kg, less than 0.03 mg/kg, less than 0.02 mg/kg, less than 0.01 mg/kg, less than 0.005 mg/kg, or less than 0.001 mg/kg.

GABA_{A} α5 receptor agonists (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or pharmaceutically acceptable salts, hydrates, solvates, polymorphs, or isomers thereof, may be administered at a dosage level up to conventional dosage levels. Alternatively, the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, may be administered at dosage levels distinct from conventional levels when provided in combination with an SV2A inhibitor (e.g., levetiracetam, brivaracetam, or seletracetam), or the pharmaceutically acceptable salt, hydrate, solvate, polymorph, or isomer thereof, due to an SV2A inhibitor (e.g., levetiracetam, brivaracetam, or seletracetam), or a pharmaceutically acceptable salt, hydrate, solvate, polymorph, or isomer thereof, dependent increase in the therapeutic index of the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof. In some embodiments, the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, may be administered at doses as disclosed, for example, in WO 2015/095783, WO 2016/205739, WO 2018/130868, WO 2018/130869, WO 2019/246300, and U.S. 62/950,886, all of which are specifically incorporated herein by reference.

In some embodiments, the therapeutic index of the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, due to the combination with an SV2A inhibitor (e.g., levetiracetam, brivaracetam, or seletracetam), or a pharmaceutically acceptable salt, hydrate, solvate, polymorph, or isomer thereof, is greater than the therapeutic index of the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, administered in the absence of an SV2A inhibitor (e.g., levetiracetam, brivaracetam, or seletracetam), or a pharmaceutically acceptable salt, hydrate, solvate, polymorph, or isomer thereof, by at least about 1.5× or 2.0× or 2.5× or 3.0× or 3.5× or 4.0× or 4.5× or 5.0× or 5.5× or 6.0× or 6.5× or 7.0× or 7.5× or 8.0× or 8.5× or 9.0× or 9.5× or 10×, or greater than about 10×. In some embodiments, combinations of the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, with the SV2A inhibitor (e.g., levetiracetam, brivaracetam, or seletracetam), or the pharmaceutically acceptable salt, hydrate, solvate, polymorph, or isomer thereof, reduces the dosage of the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, required for its therapeutic effect. In some embodiments, the amount of the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, administered in combination with the SV2A inhibitor (e.g., levetiracetam, brivaracetam, or seletracetam), or the pharmaceutically acceptable salt, hydrate, solvate, polymorph, or isomer thereof, is a sub therapeutic amount. The doses useful for the GABA_{A} α5 receptor agonists (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salts, hydrates, solvates, polymorphs, or isomers thereof, are readily determined by those skilled in the art, using the methods of this disclosure.

In certain embodiments, wherein an SV2A inhibitor (e.g., levetiracetam, brivaracetam, or seletracetam), or a pharmaceutically acceptable salt, hydrate, solvate, polymorph, or isomer thereof, is administered in combination with a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, the dosage of both the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are each sub-therapeutic with respect to treating a CNS disorder with cognitive impairment when administered alone. In certain embodiments, wherein an SV2A inhibitor (e.g., levetiracetam, brivaracetam, or seletracetam), or a pharmaceutically acceptable salt, hydrate, solvate, polymorph, or isomer thereof, is administered in combination with a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, the dosage of both the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are each sub-therapeutic with respect to treating cognitive impairment associated with a brain cancer or for treating a brain cancer itself when administered alone. In certain embodiments, wherein an SV2A inhibitor (e.g., levetiracetam, brivaracetam, or seletracetam), or a pharmaceutically acceptable salt, hydrate, solvate, polymorph, or isomer thereof, is administered in combination with a GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or a pharmaceutically acceptable salt, hydrate, solvate, polymorph, or isomer thereof, the dosage of both the SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, are each sub-therapeutic with respect to treating Parkinson's disease psychosis when administered alone.

In some embodiments, a suitable amount of the SV2A inhibitor (e.g., levetiracetam, brivaracetam, or seletracetam), or the pharmaceutically acceptable salt, hydrate, solvate, polymorph, or isomer thereof, is administered so as to reduce the dose of the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof (e.g., a dose required to effect a degree of cognitive function improvement or treat age-associated cognitive impairment), by at least about 20%, at least about 30%, at least about 40%, or at least about 50%, at least about 60%, at least about 70%, at least about 80%, at least about 90% or more from the dose of the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, normally used when administered alone (i.e., individually and not in combination with other pharmaceutical compositions or compounds). The reduction may be reflected in terms of amount administered at a given administration and/or amount administered over a given period of time (reduced frequency).

In some embodiments, a suitable amount of the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, is administered so as to reduce the dose of the SV2A inhibitor (e.g., levetiracetam, brivaracetam, or seletracetam), or the pharmaceutically acceptable salt, hydrate, solvate, polymorph, or isomer thereof (e.g., a dose required to effect a degree of cognitive function improvement or treat age-associated cognitive impairment), by at least about 20%, at least about 30%, at least about 40%, or at least about 50%, at least about 60%, at least about 70%, at least about 80%, at least about 90% or more from the dose of the SV2A inhibitor (e.g., levetiracetam, brivaracetam, or seletracetam), or the pharmaceutically acceptable salt, hydrate, solvate, polymorph, or isomer thereof, normally used when administered alone (i.e., individually and not in combination with other pharmaceutical compositions or compounds). The reduction may be reflected in terms of amount administered at a given administration and/or amount administered over a given period of time (reduced frequency).

In certain embodiments of the disclosure, the combined administration of an SV2A inhibitor (e.g., levetiracetam, brivaracetam, or seletracetam), or the pharmaceutically acceptable salt, hydrate, solvate, polymorph, or isomer thereof, and the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, can attain a longer or improved therapeutic effect in the subject than that attained by administering only the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, or only the SV2A inhibitor (e.g., levetiracetam, brivaracetam, or seletracetam), or the pharmaceutically acceptable salt, hydrate, solvate, polymorph, or isomer thereof, by at least about 1.5×, or 2.0×, or 2.5×, or 3.0×, or 3.5×, or 4.0×, or 4.5×, or 5.0×, or 5.5×, or 6.0×, or 6.5×, or 7.0×, or 7.5×, or 8.0×, or 8.5×, or 9.0×, or 9.5×, or 10×, or greater than about 10×.

In some embodiments wherein the SV2A inhibitor (e.g., levetiracetam, brivaracetam, or seletracetam), or the pharmaceutically acceptable salt, hydrate, solvate, polymorph, or isomer thereof, is in an extended release form (e.g., a controlled release form, a prolonged release form, a sustained release form, a delayed release form, or a slow release form), administration is once daily. In some embodiments wherein the SV2A inhibitor (e.g., levetiracetam, brivaracetam, or seletracetam), or the pharmaceutically acceptable salt, hydrate, solvate, polymorph, or isomer thereof, is in an extended release form (e.g., a controlled release form, a prolonged release form, a sustained release form, a delayed release form, or a slow release form), administration is twice daily. In some embodiments wherein the SV2A inhibitor (e.g., levetiracetam, brivaracetam, or seletracetam), or the pharmaceutically acceptable salt, hydrate, solvate, polymorph, or isomer thereof, is in an extended release form (e.g., a controlled release form, a prolonged release form, a sustained release form, a delayed release form, or a slow release form), administration is more than once daily (e.g., two times, three times, or four times daily).

In some embodiments wherein the SV2A inhibitor (e.g., levetiracetam, brivaracetam, or seletracetam), or the pharmaceutically acceptable salt, hydrate, solvate, polymorph, or isomer thereof, is in an immediate release form, administration is once daily. In some embodiments wherein the SV2A inhibitor (e.g., levetiracetam, brivaracetam, or seletracetam), or the pharmaceutically acceptable salt, hydrate, solvate, polymorph, or isomer thereof, is in an immediate release form, administration is twice daily. In some embodiments wherein the SV2A inhibitor (e.g., levetiracetam, brivaracetam, or seletracetam), or the pharmaceutically acceptable salt, hydrate, solvate, polymorph, or isomer thereof, is in an immediate release form, administration is more than once daily (e.g., two times, three times, or four times daily).

In some embodiments wherein the SV2A inhibitor (e.g., levetiracetam, brivaracetam, or seletracetam), or the pharmaceutically acceptable salt, hydrate, solvate, polymorph, or isomer thereof, is in a non-extended release form, administration is once daily. In some embodiments wherein the SV2A inhibitor (e.g., levetiracetam, brivaracetam, or seletracetam), or the pharmaceutically acceptable salt, hydrate, solvate, polymorph, or isomer thereof, is in a non-extended release form, administration is twice daily. In some embodiments wherein the SV2A inhibitor (e.g., levetiracetam, brivaracetam, or seletracetam), or the pharmaceutically acceptable salt, hydrate, solvate, polymorph, or isomer thereof, is in a non-extended release form, administration is more than once daily (e.g., two times, three times, or four times daily).

In some embodiments wherein the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, is in an extended release form (e.g., a controlled release form, a prolonged release form, a sustained release form, a delayed release form, or a slow release form), administration is once daily. In some embodiments wherein the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, is in an extended release form (e.g., a controlled release form, a prolonged release form, a sustained release form, a delayed release form, or a slow release form), administration is twice daily. In some embodiments wherein the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, is in an extended release form (e.g., a controlled release form, a prolonged release form, a sustained release form, a delayed release form, or a slow release form), administration is more than once daily (e.g., two times, three times, or four times daily).

In some embodiments wherein the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, is in an immediate release form, administration is once daily. In some embodiments wherein the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, is in an immediate release form, administration is twice daily. In some embodiments wherein the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, is in an immediate release form, administration is more than once daily (e.g., two times, three times, or four times daily).

In some embodiments wherein the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, is in a non-extended release form, administration is once daily. In some embodiments wherein the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, is in a non-extended release form, administration is twice daily. In some embodiments wherein the GABA_{A} α5 receptor agonist (e.g., a compound of Formula I, Formula II, or Formula IV; Compounds 1-740 as described above, Compounds 1-114; or Compound 1, Form A; Compound 1, Form B; Compound 1, Form C; Compound 1, Form E; or Compound 1, Form F), or the pharmaceutically acceptable salt, hydrate, solvate, polymorph, or isomer thereof, is in a non-extended release form, administration is more than once daily (e.g., two times, three times, or four times daily).

It will be understood by one of ordinary skill in the art that the pharmaceutical compositions, methods, uses, combinations, pharmaceutical compositions for use, or combinations for use described herein may be adapted and modified as is appropriate for the application being addressed and that the pharmaceutical compositions, methods, uses, combinations, pharmaceutical compositions for use, or combinations for use described herein may be employed in other suitable applications, and that such other additions and modifications will not depart from the scope hereof.

This disclosure will be better understood from the Experimental Details which follow. However, one skilled in the art will readily appreciate that the specific methods and results discussed are merely illustrative of the disclosure as described more fully in the embodiments which follow thereafter.

### EXAMPLES

### Introduction and Models of Cognitive Impairment

A variety of conditions characterized by cognitive impairment, e.g., Age-Associated Memory Impairment (AAMI), Mild Cognitive Impairment (MCI) and Age-related Cognitive Decline (ARCD) are believed to be related to aging. Others are related to disease, for example, AD. Animal models serve as an important resource for developing and evaluating treatments for such age-related cognitive impairments. Features that characterize age-related cognitive impairment in animal models typically extend to age-related cognitive impairment in humans. Efficacy in such animal models is, thus, predictive of efficacy in humans.

Of available models, a Long-Evans rat model of cognitive impairment is particularly well suited for distinguishing the difference between cognitive impairment related to illness and that related to aging. Indeed, extensive behavioral characterization has identified a naturally occurring form of cognitive impairment in an outbred strain of aged Long-Evans rats (Charles River Laboratories; Gallagher et al., Behav. Neurosci. 107:618-626, (1993)). In a behavioral assessment with the Morris Water Maze (MWM), rats learn and remember the location of an escape platform guided by a configuration of spatial cues surrounding the maze. The cognitive basis of performance is tested in probe trials using measures of the animal's spatial bias in searching for the location of the escape platform. Aged rats in the study population have no difficulty swimming to a visible platform, but an age-dependent impairment is detected when the platform is camouflaged, requiring the use of spatial information. Performance for individual aged rats in the outbred Long-Evans strain varies greatly. For example, a proportion of those rats perform on a par with young adults. However, approximately 40-50% fall outside the range of young performance. This variability among aged rats reflects reliable individual differences. Thus, within the aged population some animals are cognitively impaired and designated aged-impaired (AI) and other animals are not impaired and are designated aged-unimpaired (AU). See, e.g., Colombo et al., Proc. Natl. Acad. Sci. 94: 14195-14199, (1997); Gallagher and Burwell, Neurobiol. Aging 10: 691-708, (1989); Rapp and Gallagher, Proc. Natl. Acad. Sci. 93: 9926-9930, (1996); Nicolle et al., Neuroscience 74: 741-756, (1996); and Nicolle et al., J. Neurosci. 19: 9604-9610, (1999).

We used the above-described rat model to identify individual AI and AU rats. We then conducted behavioral assessment on AI rats while administering various pharmacological treatments.

### Example 1: Effect of Levetiracetam in Aged-Impaired Rats

### Morris Water Maze Results

Six aged-impaired (AI) Long-Evans rats (as characterized above) were tested for their memory of new spatial information in the Morris water maze (MWM), under different drug/control treatment conditions (vehicle control and two different dosage levels of levetiracetam). A retention trial was performed after the training trials, as described below.

The MWM apparatus consists of a large, circular pool (diameter 1.53 m; height, 0.58 m) filled with water (27° C.) that is made opaque through the addition of non-toxic pigment or some other substance. In the typical “hidden platform” version of the test, rats are trained to find a camouflaged white escape platform (height, 34.5 cm) that is positioned in the center of one quadrant of the maze about 1.0 cm below the water surface. This platform can be retracted to the bottom of the tank or raised to its normal position from outside the maze during behavioral testing. The location of the platform remains constant from trial to trial. Because there are no local cues that mark the position of the platform, the rat's ability to locate it efficiently from any starting position at the perimeter of the pool depends on using information surrounding the maze. The maze is surrounded by black curtains to which white patterns are affixed to provide a configuration of spatial cues. A second platform (height 37.5 cm), with its surface painted black is elevated 2 cm above the water surface during cue training to control for factors unrelated to cognition. The behavior of a rat in the pool is recorded by a camera that is suspended 2.5 m above the center of the pool. The camera is connected to a video tracking system (HVS Image Advanced Tracker VP200) and a PC computer running HVS software developed by Richard Baker of HVS Image, Hampton, UK.

The MWM protocol is optimized for sensitivity to the effects of aging on cognition and for measures of reliable individual differences within the aged population of out-bred Long-Evans rats (Gallagher et al. Behav. Neurosci. 107:618-626, (1993)). Rats receive three trials per day for 8 consecutive days, using a 60 sec inter-trial interval. On each training trial, the rat is released into the maze from one of four equally spaced starting positions around the perimeter of the pool. The starting position varies from trial to trial, thus preventing the use of a response strategy (e.g., always turning left from the start location to locate the escape platform). If a rat does not locate the escape platform within 90 sec on any trial, the experimenter guides the rat to the platform, where it remains for 30 sec. Every sixth trial consists of a probe trial to assess the development of spatial bias in the maze. During these trials, the rat swims with the platform retracted to the bottom of the pool for 30 sec, at which time the platform is raised to its normal position for completion of the escape trial. At the completion of the protocol using the hidden platform, rats are assessed for cue learning using the visible platform. The location of this platform varies from trial to trial in a single session of 6 training trials.

The proximity of the animal's position with respect to the goal is used to analyze the training trial and probe trial performance. The proximity measure is obtained by sampling the position of the animal in the maze (10 times/sec) to provide a record of distance from the escape platform in 1 sec averages. For both probe trials and training trials, a correction procedure is implemented so that trial performance is relatively unbiased by differences in distance to the goal from the various start locations at the perimeter of the pool. In making this correction, the average swimming speed is calculated for each trial (path length/latency). Then, the amount of time required to swim to the goal at that speed from the start location used for the trial is removed from the record prior to computing trial performance, i.e., cumulative distance on training trials and average distance from the goal on probe trials. Thus, scores obtained using the proximity measure are designed to reflect search error, representing deviations from an optimal search, i.e. direct path to the goal and search in the immediate vicinity of that location during probe trials.

Computer records of video-tracking are compiled to provide data on each rat's performance in the maze. Measures on training trials and probe trials are analyzed by Analysis of Variance (ANOVA).

An average proximity measure on interpolated probe trials is used to calculate a spatial learning index for each individual subject as described in detail in Gallagher et al., Behav. Neurosci. 107:618-26, (1993). When a rat rapidly learns to search for the platform close to its position, its spatial learning index is low.

AI rats were given six training trials per training day with a 60-sec inter-trial interval between each training trial for two consecutive days. On each training trial, the rat was released in the maze from one of four equally spaced starting positions around the perimeter of the pool. If the rat did not locate the escape platform within 90 sec on any trial, the experimenter guided the rat to the platform, where it remained for 30 sec. 30 minutes to 1 hour prior to all the training trials on each training day, AI rats were pretreated with one of three drug conditions: 1) vehicle control (0.9% saline solution); 2) levetiracetam (5m/kg/day); and 3) levetiracetam (10 mg/kg/day); through intraperitoneal (i.p.) injection. The same six AI rats were used for the entire trials so that each treatment condition was tested on all six rats. Therefore, to counterbalance any potential bias, both the location of the escape platform and the spatial cues surrounding the water maze were different in the three treatment conditions. Therefore, using one set of locations and spatial cues, two rats were treated with saline control solution, two with levetiracetam (5m/kg/day) and two with levetiracetam (10 mg/kg/day). Using the second set of locations and spatial cues, the two rats treated with saline control solution in the first test were treated with either levetiracetam (5m/kg/day) or levetiracetam (10 mg/kg/day), and the two rats previously treated with levetiracetam (5m/kg/day) were treated with either saline control solution or levetiracetam (10 mg/kg/day), and the two rats previously treated with levetiracetam (10 mg/kg/day) were treated with either saline control solution or levetiracetam (5m/kg/day). Using the last set of locations and spatial cues, the rat groupings were again switched so that each group was treated with a different condition than they had been treated previously.

After the second training day and completion of the twelve training trials (over the two days), the rat was returned to its home cage and placed in the animal housing room. After a delay of 24 hours from the last training trial, the rat was given one testing trial (the “retention trial”), which was the same MWM task as the training trials, but with the escape platform removed.

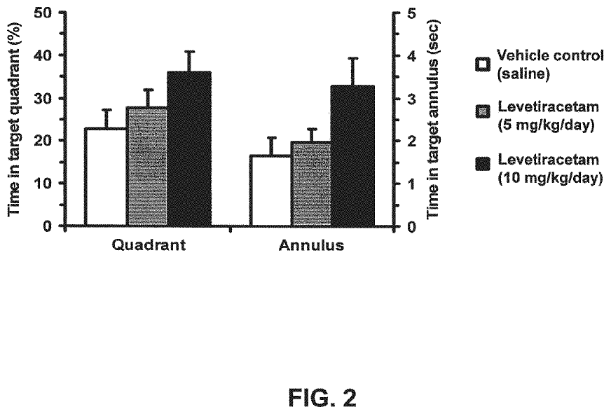

For the retention trial, the MWM circular pool was divided into 4 quadrants. The particular quadrant where the escape platform was placed in the training trials is referred as “target quadrant”. The particular region where the platform was located in the training trials is referred as “target annulus”. In the retention trial, the time the AI rats spent swimming in the target quadrant is measured and further plotted as a percentage of total swimming time. FIG. 2 displays the results of one such set of retention trials. The time the AI rats spend in the target annulus is also measured. FIG. 2 displays the results of one such set of retention trials. Time data are collected for all drug treatment conditions.

In the retention trial, whose results are depicted in FIG. 2, the time the AI rats spent in the target quadrant was approximately 25%, which is a performance equivalent to them having no memory of the platform location. This performance did not significantly improve in the group treated with levetiracetam at 5 mg/kg/day. However, the group treated with levetiracetam at 10 mg/kg/day demonstrated significantly improved memory as compared to vehicle-treated controls, as indicated by a significant increase in the time spent in the target quadrant to approximately 35% of total swimming time (see FIG. 2). That level of performance is equivalent to young and age-unimpaired rats, indicating that treatment with 10 mg/kg/day levetiracetam resulted in a significant recovery of the AI rats' ability to navigate this MWM. The effectiveness of the 10 mg/kg/day levetiracetam treatment was also seen in the time spent in the target annulus (see FIG. 2).

### Radial Arm Maze Results

The effects of levetiracetam on the spatial memory retention of aged-impaired (AI) rats were assessed in a Radial Arm Maze (RAM) behavioral task using vehicle control and five different dosage levels of levetiracetam (1.25 mg/kg/day, 2.5 mg/kg/day, 5 mg/kg/day, 10 mg/kg/day and 20 mg/kg/day). RAM behavioral tasks were performed on ten AI rats. All six treatment conditions were tested on all ten rats, as described above for the MWM test.

The RAM apparatus used consisted of eight equidistantly spaced arms. An elevated maze arm (7 cm width×75 cm length) projected from each facet of an octagonal center platform (30 cm diameter, 51.5 cm height). Clear side walls on the arms were 10 cm high and were angled at 65° to form a trough. A food well (4 cm diameter, 2 cm deep) was located at the distal end of each arm. Froot Loops™ (Kellogg Company) were used as rewards. Blocks constructed of Plexiglass™ (30 cm height×12 cm width) could be positioned to prevent entry to any arm. Numerous extra maze cues surrounding the apparatus were also provided.

The AI rats were initially subjected to a pre-training test (Chappell et al. Neuropharmacology 37: 481-487, 1998). The pre-training test consisted of a habituation phase (4 days), a training phase on the standard win-shift task (18 days) and another training phase (14 days) in which a brief delay was imposed between presentation of a subset of arms designated by the experimenter (e.g., 5 arms available and 3 arms blocked) and completion of the eight-arm win-shift task (i.e., with all eight arms available).

In the habituation phase, rats were familiarized to the maze for an 8-minute session on four consecutive days. In each of these sessions food rewards were scattered on the RAM, initially on the center platform and arms and then progressively confined to the arms. After this habituation phase, a standard training protocol was used, in which a food pellet was located at the end of each arm. Rats received one trial each day for 18 days. Each daily trial terminated when all eight food pellets had been obtained or when either 16 choices were made or 15 minutes had elapsed. After completion of this training phase, a second training phase was carried out in which the memory demand was increased by imposing a brief delay during the trial. At the beginning of each trial, three arms of the eight-arm maze were blocked. Rats were allowed to obtain food on the five arms to which access was permitted during this initial ‘information phase’ of the trial. Rats were then removed from the maze for 60 seconds, during which time the barriers on the maze were removed, thus allowing access to all eight arms. Rats were then placed back onto the center platform and allowed to obtain the remaining food rewards during this ‘retention test’ phase of the trial. The identity and configuration of the blocked arms varied across trials.

The number of “errors” the AI rats made during the retention test phase was tracked. An error occurred in the trial if the rats entered an arm from which food had already been retrieved in the pre-delay component of the trial, or if it re-visited an arm in the post-delay session that had already been visited.

After completion of the pre-training test, rats were subjected to trials with more extended delay intervals, i.e., a one-hour delay, between the information phase (presentation with some blocked arms) and the retention test (presentation of all arms). During the delay interval, rats remained off to the side of the maze in the testing room, on carts in their individual home cages. AI rats were pretreated 30-40 minutes before daily trials with a one-time shot of the following six conditions: 1) vehicle control (0.9% saline solution); 2) levetiracetam (1.25 mg/kg/day); 3) levetiracetam (2.5 mg/kg/day); 4) levetiracetam (5 mg/kg/day); 5) levetiracetam (10 mg/kg/day); 6) levetiracetam (20 mg/kg/day); through intraperitoneal (i.p.) injection. Injections were given every other day with intervening washout days. Each AI rat was treated with all six conditions within 23 days of testing. To counterbalance any potential bias, drug effect was assessed using ascending-descending dose series, i.e., the dose series was given first in an ascending order and then repeated in a descending order. Therefore, each dose had two determinations.

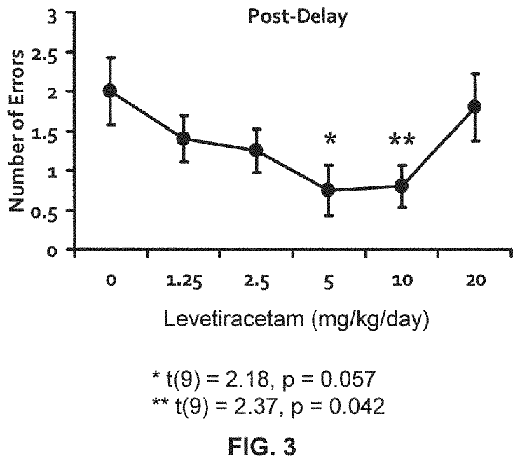

Parametric statistics (paired t-tests) was used to compare the retention test performance of the AI rats in the one-hour delay version of the RAM task in the context of different doses of levetiracetam and vehicle control (see FIG. 3). The average numbers of errors that occurred in the trials were also significantly fewer with levetiracetam treatment of 5 mg/kg/day (average no. of errors±standard error of the mean (SEM)=0.75±0.32) and 10 mg/kg/day (average no. of errors±SEM=0.80±0.27) than using vehicle control (average no. of errors±SEM=2.00±0.42). Relative to vehicle control treatment, levetiracetam significantly improved memory performance at 5 mg/kg/day (t(9)=2.18, p=0.057) and 10 mg/kg/day (t(9)=2.37, p=0.042).

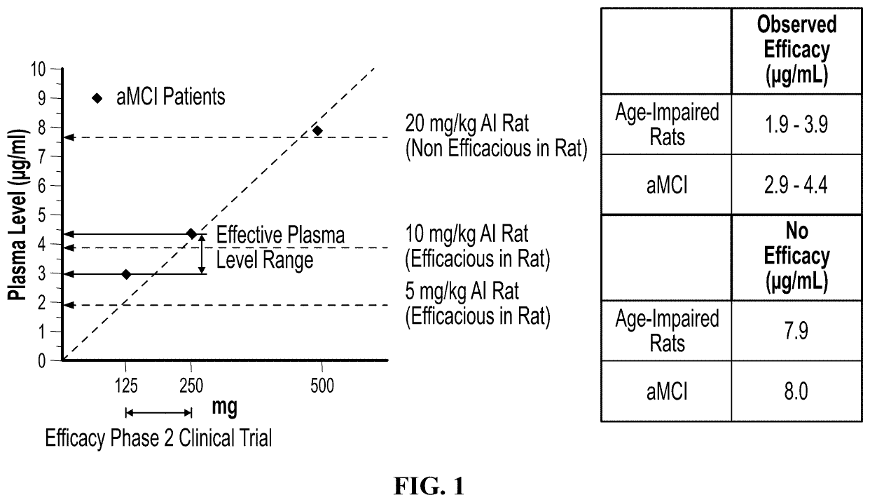

To calculate the dose of levetiracetam for treatment of age-dependent cognitive impairment in humans, we assessed the levetiracetam plasma level in the rat after treatment, determined the corresponding human plasma level, and then extrapolated to provide the levetiracetam dose. See, FIG. 1 and Example 6.

### Example 2: Effect of Levetiracetam in Human Subjects with aMCI

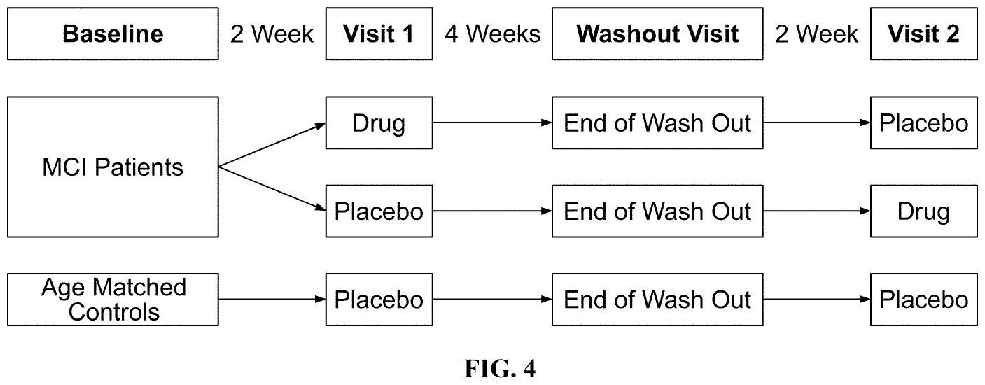

A within-subjects trial of 8 weeks duration, involving 17 amnestic MCI (aMCI) subjects and 17 age-matched controls with a low dose treatment of levetiracetam is conducted. During the course of the study, each aMCI subject receives both drug and placebo treatments separately in two periods of two weeks each, with the order of treatments among different aMCI subjects counterbalanced (see FIG. 4). Age-matched control subjects treated with placebo serve as a further control. Cognitive testing and fMRI imaging data are obtained from the subjects after each two-week period of drug/placebo treatment.

### Participants and Clinical Characterization

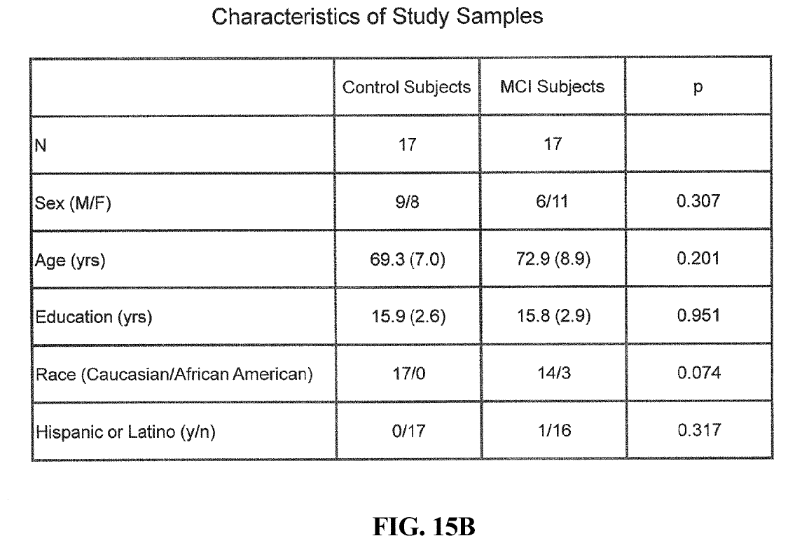

17 right-handed aMCI patients are recruited from the Alzheimer's Disease Research Center (ADRC) at the Johns Hopkins Hospital and other referrals. An additional 17 right-handed healthy volunteers are recruited from the pool of control participants in the ADRC and other referrals. All participants are administered the Telephone Interview of Cognitive Status to determine if they are likely to pass the entry criteria of the study (including criteria for MRI scanning). All participants further undergo neurological, psychiatric, and neuropsychological examination using standardized instruments and methods. The psychiatric evaluation includes administration of the Structured Clinical Interview for DSM-IV Axis I Disorders and the Clinical Dementia Rating (CDR) scale. All aMCI patients have CDR scores of 0.5. Diagnosis of aMCI is based on the criteria proposed by Petersen et al. (e.g., “Mild cognitive impairment: Aging to Alzheimer's Disease,” Oxford University Press, N.Y. (2003), which include a memory complaint (corroborated by an informant), impaired memory function on testing (1.5 standard deviations below norm), otherwise preserved cognitive functioning (within 1 standard deviation of norm), no decline in functional ability, and no dementia. Final aMCI diagnoses are reached by clinical consensus. Exclusion criteria include major neurological or psychiatric disorders, head trauma with loss of consciousness, history of drug abuse or dependency, and general contraindications to an MRI examination (e.g. cardiac pacemaker, aneurysm coils, claustrophobia). Each aMCI subject is required to have a study partner (i.e., an informant) who can provide information about the subject's daily function and assure that medications are taken appropriately. See FIGS. 15A and 15B.

Study Visits: The study consists of 4 visits over the course of 8 weeks (see FIG. 4). The Baseline Visit is for the purpose of performing medical, neurological, psychiatric, and neurocognitive assessments. Visits 1 and 2 are identical to the Baseline Visit but include a fMRI session. The Washout Visit, at the end of a 4-week washout period, is for the purpose of a brief clinical assessment and initiation of the second drug/placebo phase.

Baseline Visit: At the screening visit, informed consent is obtained from the subject (and an informant in the case of MCI subjects). The subject and the informant participate in a standardized clinical interview that is used to determine the degree of the subject's functional impairment in daily life, based on the Clinical Dementia Rating (CDR) scale. The subject's medical, neurological, and psychiatric history is obtained (including a review of current medications), as well as the family history of dementia. Brief medical, neurological and psychiatric exams are conducted (including vital signs). Blood is drawn in order to perform standard laboratory tests needed to determine if the subject meets the entry criteria. The subject is re-screened for contraindications to MRI scanning, using the standard form employed at the Kirby Imaging Center. Brief cognitive testing is performed (described in section on neuropsychological assessment below). These assessments are used to determine if the subject meets the entry criteria. All of the foregoing are completed using standardized forms. If the subject meets entry criteria for the study, the subject is given the study medication (drug or placebo, randomly selected), and instructions about how it should be taken. The subject is advised about the potential for having suicidal thoughts and advised to stop taking the medication and immediately contact the study physician if this occurs.

Visit 1: At the end of the first drug/placebo period 2 weeks after the Baseline Visit, the medical, neurological and psychiatric evaluations and cognitive testing are repeated. The subject is also clinically evaluated for suicidal ideation. Blood is drawn again to repeat the standard tests and to determine whether there are any changes related to drug treatment; the subject's blood levetiracetam level is also obtained. All medication dispensed at the Baseline Visit (drug or placebo) is collected and subject compliance with the medication regimen is assessed. The first fMRI session (with cognitive tests) is conducted on the same day, either immediately before or immediately after the clinical assessment. Subjects discontinue first period treatment at this visit.

Washout Visit: At the end of a washout period (4 weeks) following Visit 1, the subject receives a brief medical screening, including a medical and psychiatric evaluation. Blood is drawn to obtain the blood levetiracetam level (to confirm washout). The subject is provided with new medication (drug or placebo, alternated from what was assigned in the previous treatment period) for the final phase of the study with instructions about how it should be taken.

Visit 2: At approximately 2 weeks after the Washout Visit (i.e., 2 weeks after starting the second treatment period), the medical, neurological and psychiatric evaluations and the cognitive testing are repeated. The subject is clinically evaluated for suicidal ideation. Blood is drawn again to repeat the standard tests and to determine whether there were any changes related to drug treatment; the subject's blood levetiracetam level is also obtained. All medication dispensed at the Washout Visit is collected and subject compliance with the medication regimen is assessed. The second fMRI session (with cognitive tests) is repeated on the same day, either immediately before or immediately after the clinical assessment.

### Neuropsychological Assessment

All participants undergo neuropsychological evaluation at the time of assessment for treatment efficacy (Visits 1 and 2), as well as at the Baseline Visit. The evaluation occurs outside of the scanner and includes the Buschke Selective Reminding Test (Buschke and Fuld, 1974) and the Verbal Paired Associates subtest, the Logical Memory subtest, the Visual Reproduction subtest of the Wechsler Memory Scale-Revised (WMS-R) (Wechsler, 1997), and the Benton Visual Retention Test, as these tasks are particularly sensitive to medial temporal lobe function and early memory problems (Marquis et al., 2002 and Masur et al., 1994). Additionally, subjects are asked to complete tests of more general cognitive function such as tests to assess general mental status, executive function, attention and general naming ability. All neuropsychological tests are administered by a trained research assistant during a 60-minute session. As the three neuropsychological assessments in this study occur within a time period of 8 weeks, different versions of the neuropsychological tests are used to minimize test specific practice effects. Breaks are provided to the subject as needed.

### Drug Administration

As described above, the drug treatment period is the two weeks preceding Visit 1 or 2 (with the two-week period preceding the other Visit being the placebo phase). For the subjects receiving the drug treatment, half a scored 250 mg tablet of levetiracetam is used to achieve a dose of 125 mg twice a day, which is approximately 3.6 mg/kg/day (assuming an average adult human weight of 70 kg).

All drug and placebo preparations are performed on a 1:1 allocation. The pharmacy randomizes patients to drug dose and condition as they enroll and keep a list of drug assignment.

Levetiracetam is rapidly and almost completely absorbed after oral administration, and its bioavailability is not affected by food. Plasma half-life of levetiracetam is approximately 7±1 hour (expected to be 9-10 hours in elderly due to decreased renal function). Absorption is rapid, with peak plasma concentrations occurring about 1 hour following oral administration. Steady state can be achieved after 2 days of multiple twice-daily dosing.

A typical starting dose of levetiracetam in treating epilepsy in humans is 500 mg twice a day, which is approximately 14.3 mg/kg/day. The dosage is then is increased until optimal efficacy, up to 50 mg/kg/day. Thus, the dose used in this experiment is a quarter of the lowest human dose used for treating epilepsy.

To calculate the dose of levetiracetam for treatment of age-dependent cognitive impairment in humans, we assessed the levetiracetam plasma level in the rat after treatment, determined the corresponding human plasma level, and then extrapolated to provide the levetiracetam dose. See, FIG. 1 and Example 6.

### MRI Data Acquisition

Imaging data are obtained through high-resolution methods developed in the Stark laboratory. Data are collected on a Phillips 3 Tesla scanner (Eindhoven, The Netherlands) equipped with an 8-channel SENSE (Sensitivity Encoding) head coil, located at the F.M. Kirby Research Center for Functional Brain Imaging at the Kennedy Krieger Institute (Baltimore, Md.). High-resolution echo-planar images are collected using an acquisition matrix of 64×64, a repetition time of 1500 milliseconds, an echo time of 30 milliseconds, a flip angle of 70 degrees, a SENSE factor of 2, and an isotropic resolution of 1.5 mm×1.5 mm×1.5 mm with no gap. Nineteen oblique slices are acquired parallel to the principal longitudinal axis of the hippocampus and covered the entire medial temporal lobe region bilaterally. In addition to the functional runs, a whole-brain MPRAGE structural scan (parameters: 150 oblique slices, 1 mm isotropic resolution) is acquired.

### Image Analysis

Data analysis is carried out using the Analysis for Functional Neuroimages (AFNI, release 2008_07_18_1710) software. Images are first co-registered to correct for within- and across-scan head motion. Acquisitions in which a significant motion event occur (more than 3 degrees of rotation or 2 mm of translation in any direction relative to prior acquisition), plus and minus one-time repetition for 1.5 seconds, are excluded from the analyses. Structural anatomical data are registered to standard stereotaxic space (Talairach & Tournoux, 1988), and the same parameters are subsequently applied to the functional data. Behavioral vectors are produced to model different trial types.

The ROI-LDDMM (large deformation diffeomorphic metric mapping of the region of interest) method, a technique for cross-subject alignment, increases the power of multisubject regional fMRI studies by focusing the alignment power specifically on the ROIs (regions of interest) and not elsewhere in the brain. First, all subjects' anatomical and functional scans are normalized to the Talairach atlas using AFNI. Sub-regions of the medial temporal lobe and the hippocampus (bilateral entorhinal cortex, perirhinal cortex, Para hippocampal cortex, CA3/dentate region, CA1 region, and subiculum) are segmented in three dimensions on the MPRAGE scans. The labels for the CA3 region and dentate gyrus (DG) are combined. The anatomically defined ROIs are then used to calculate the ROI-LDDMM 3D vector field transformation for each subject using a customized template based on the mean of the entire sample tested as the target. The ROI-LDDMM transformations for each individual subject's ROIs are then applied to the fit coefficient maps.

Group data are analyzed using a two-way Analysis of Variance (ANOVA) with trial types and group as fixed factors, and subject as a random factor nested within group. A liberal peak threshold of p<0.05, along with a spatial extent threshold of 10 voxels are used to define functional ROIs on the overall F statistic. This approach, rather than using a direct pair-wise contrast, reduces voxel selection biases because any differences amongst the various conditions allowed for a voxel to be selected. This threshold is then combined with the anatomical segmentations to only include voxels inside the regions of interest. This serves to exclude voxels that does not change with any of the model's factors, effectively limiting the analysis to voxels showing any changes with task condition or group. Voxels within each functional ROI are collapsed for further analysis.

Cognitive Tests During fMRI Scans at Visits 1 and 2

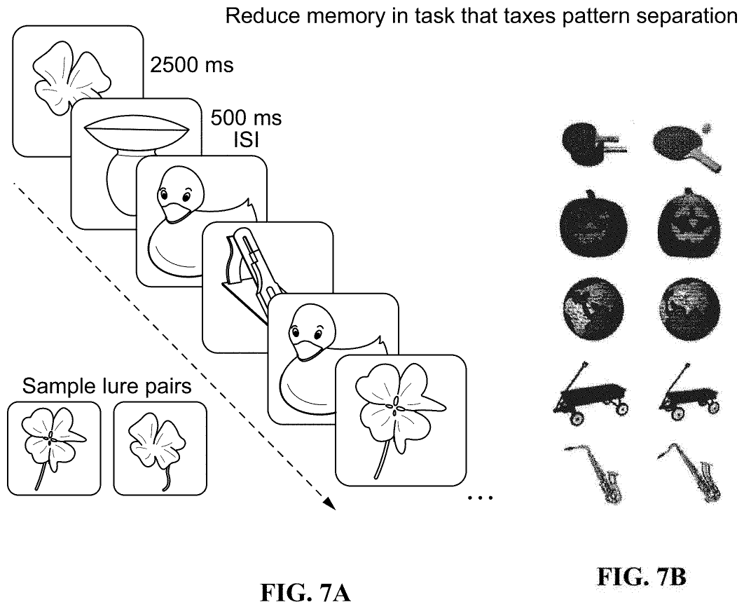

The activity of the subject's medial temporal lobe is measured by functional MRI during the subject's participation in an explicit 3-alternative forced choice task, where participants view novel, repeated and similar (“lure”) stimuli. The Psychophysics Toolbox extensions in MATLAB® 7.0 (The MathWorks, Natick, Mass.) are used for stimulus presentation and behavioral data collection. Stimuli are color photographs of common objects. Each participant undergoes a series of testing runs during the functional imaging sessions, each run consisting of a mix of three types of image pairs: similar pairs, identical pairs and unrelated foils. These image pairs are fully randomized throughout the run and presented individually as a series of images (see FIG. 7A). Participants are instructed to make a judgment as to whether each object seen is new, old or similar. Of critical interest are the participants' responses when presented with the second of the pair of similar objects (the “lure”; see FIG. 7B). The correct identification by the subject of lure stimuli as “similar,” provides behavioral evidence of pattern separation, i.e., the separation of similar experiences into distinct non-overlapping representations. However, an incorrect identification of lure stimuli as “old” or “new,” indicates a failure of pattern separation. Identification of lure stimuli as “old” indicates that the subject focused on the similarities between the lure stimulus and the earlier-shown partner image. Identification of the lure stimulus as “new” indicates that the subject failed to recall the earlier-shown partner image altogether. Each run also contains a number of baseline trials that use a challenging perceptual discrimination task known to provide a lower and more-stable estimate of baseline activity in the medial temporal lobe (Stark & Squire, 2001 PNAS; Law et al, 2005).

A survey of the activity level of various subregions in the medial temporal lobe during the cognitive test, as measured by fMRI, shows that aMCI subjects have hyperactive DG/CA3 regions and a hypoactive entorhinal cortex during the performance of memory tasks, compared to age-matched control subjects.

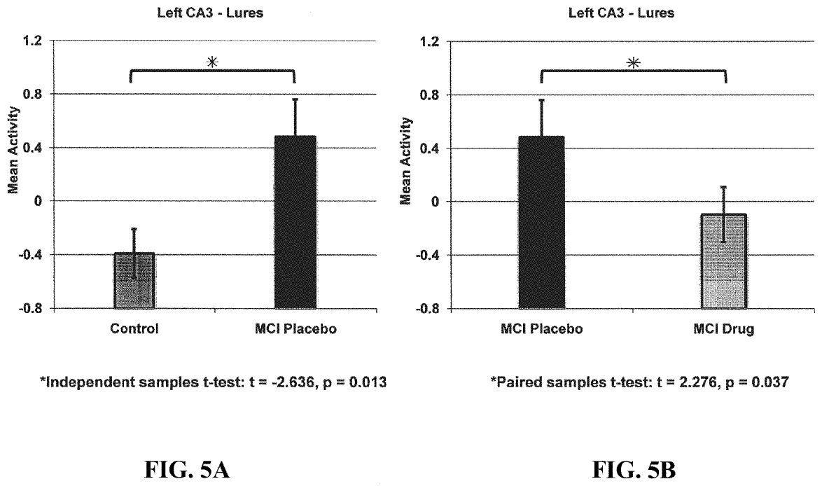

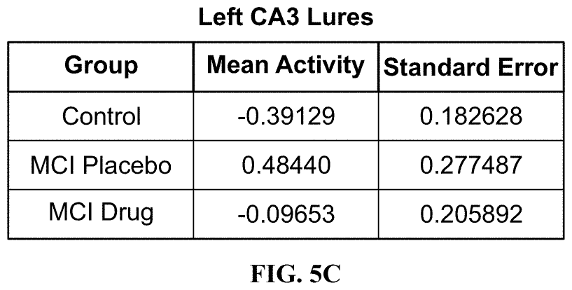

We assess the level of activity in DG/CA3 during successful memory judgments in control and aMCI subjects. The mean activity is calculated from the average activity, as measured by fMRI, during the presentation of lure stimuli correctly identified by subject as “similar” that is calibrated for baseline activity. FIG. 5A shows that aMCI patients exhibit DG/CA3 hyperactivity when making these judgments (p=0.013). FIG. 5B, however, shows that treatment with levetiracetam reduces DG/CA3 hyper-activity in aMCI subjects (p=0.037). The activity level in the aMCI subject treated with the drug, in fact, is normalized to the extent that that it is statistically indistinguishable from the activity of control subjects treated with placebo. See FIG. 5C for the mean activity values shown in FIGS. 5A and 5B.

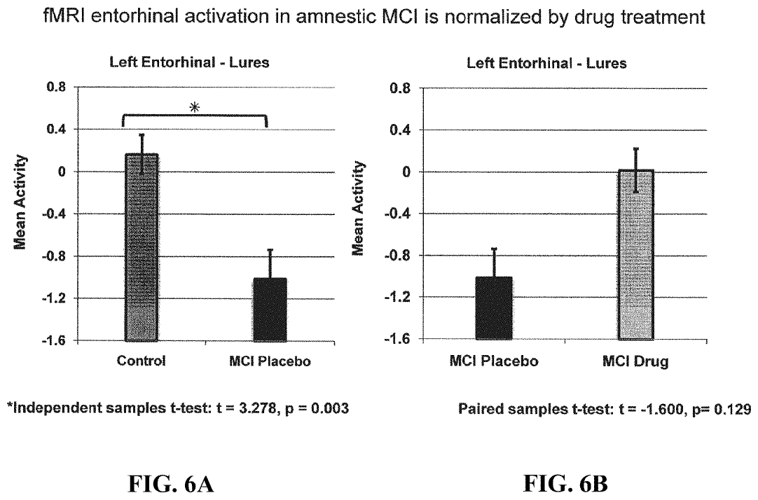

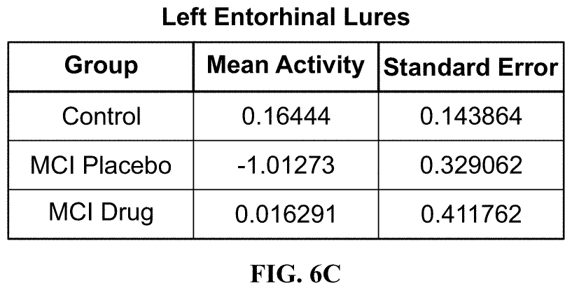

The activity level during successful memory judgments in EC is significantly lower in placebo-treated aMCI subjects compared to controls (p=0.003). See FIG. 6A. However, levetiracetam treatment normalizes activity in aMCI subjects in EC as well. See FIG. 6B. Levetiracetam treatment increases EC activity during memory judgments in aMCI subjects, such that it is statistically indistinguishable from placebo-treated control subjects. See FIG. 6B. See FIG. 6C for the mean activity values shown in FIGS. 6A and 6B.

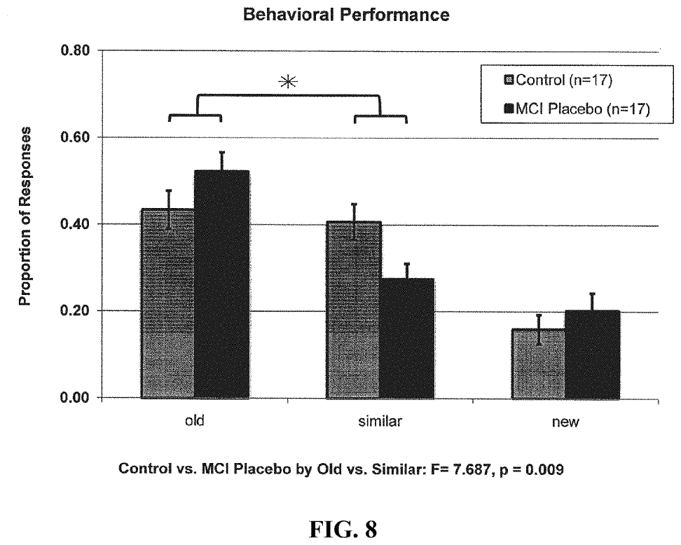

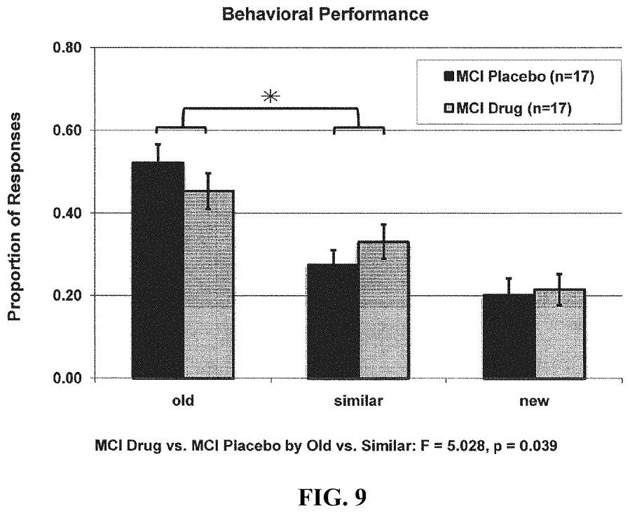

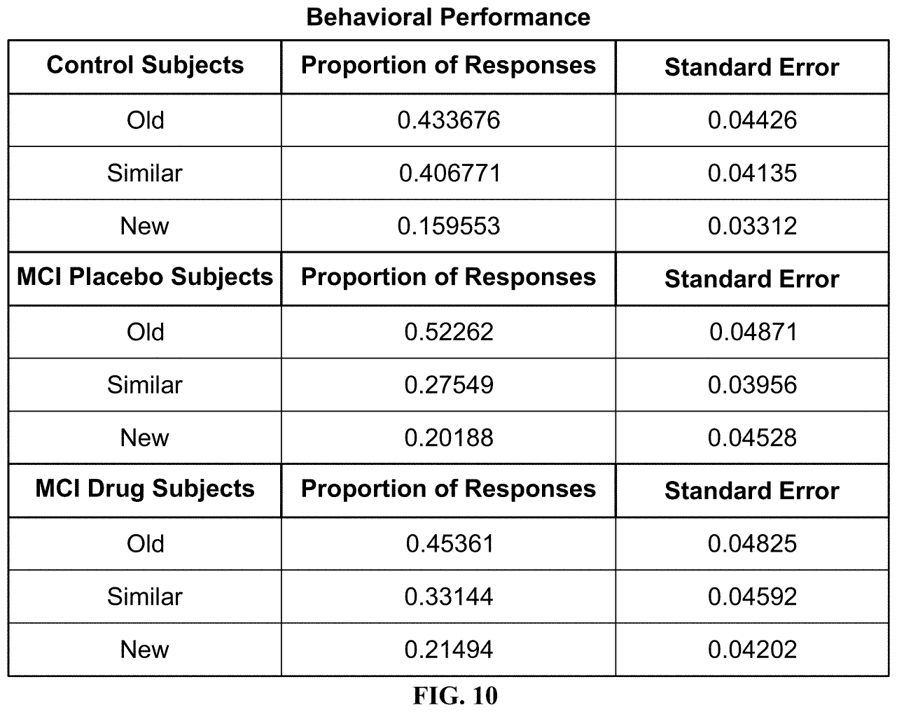

The normalization of DG/CA3 and EC activity during memory judgments by levetiracetam treatment is mirrored in the change seen in the aMCI subjects' performance in the cognitive task. With placebo treatment, aMCI patients perform worse than control subjects, correctly identify lure items as “similar” less often and incorrectly identifying them as “old” more often (p=0.009). See FIG. 8. However, the performance of aMCI subjects improves significantly under levetiracetam treatment. See FIG. 9. The interaction of more correct “similar” identifications with less incorrect “old” identifications under drug treatment results in a significant improvement in the performance of this memory task (p=0.039). See FIG. 10 for a table of the data represented in FIGS. 8 and 9.

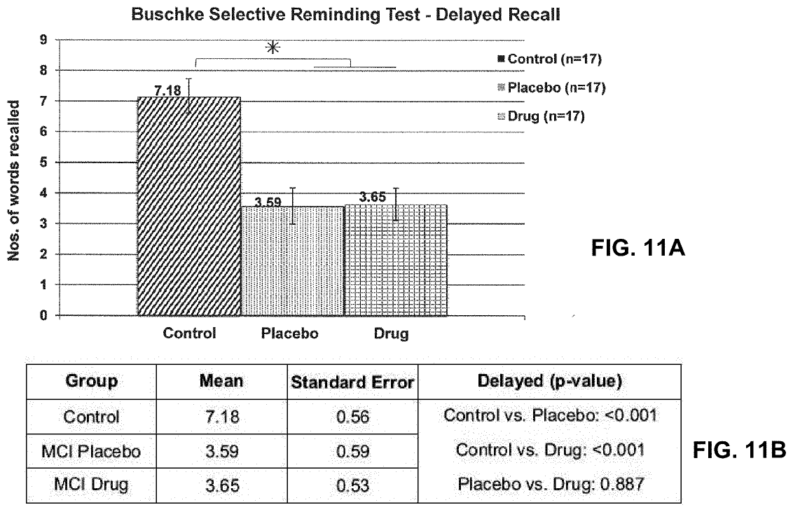

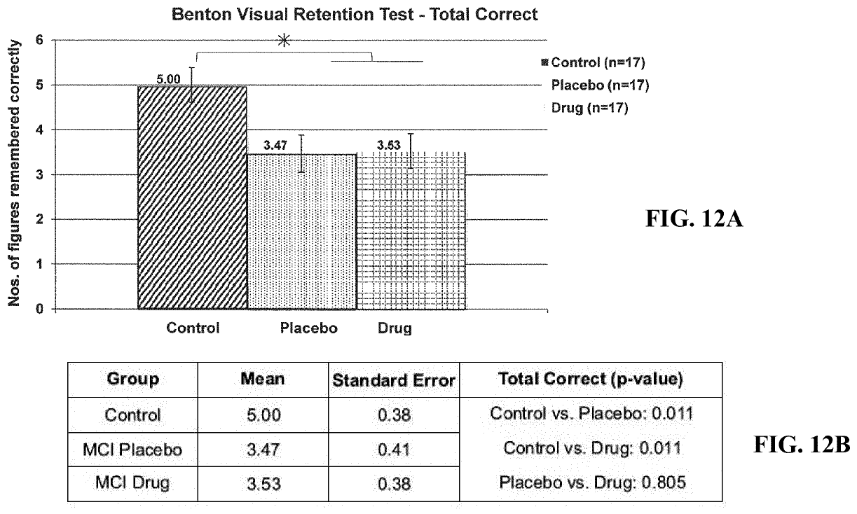

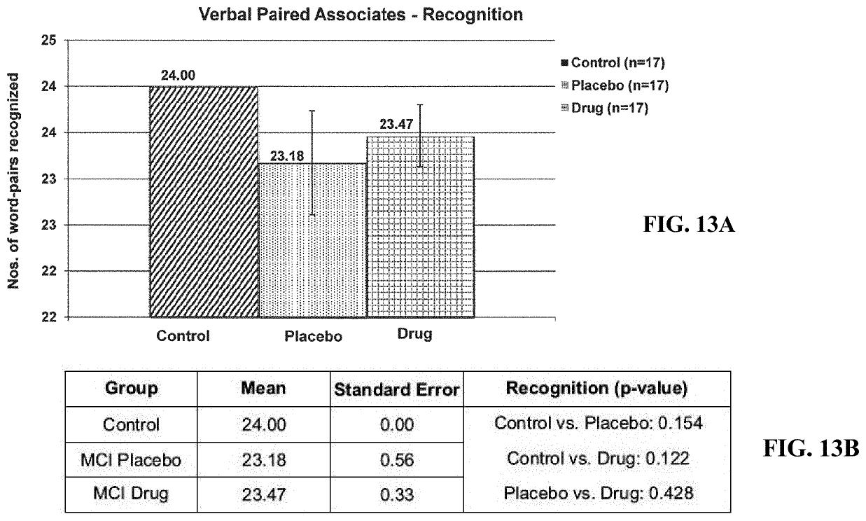

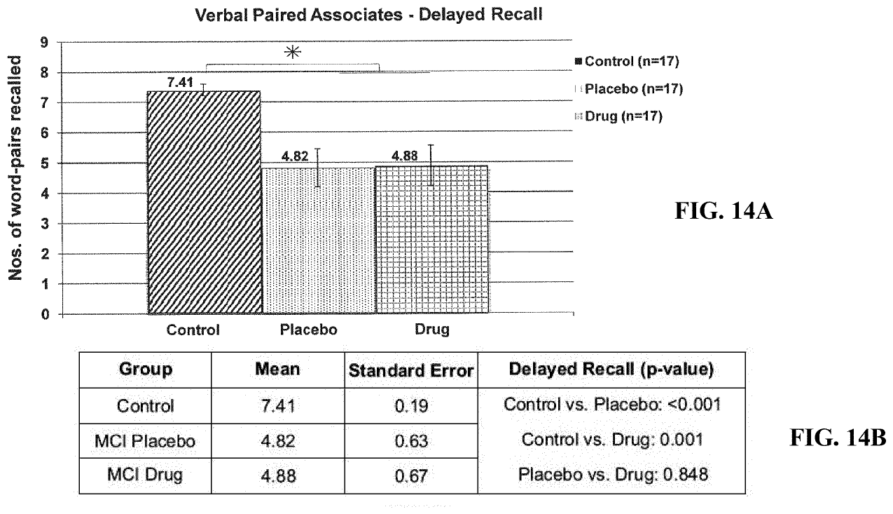

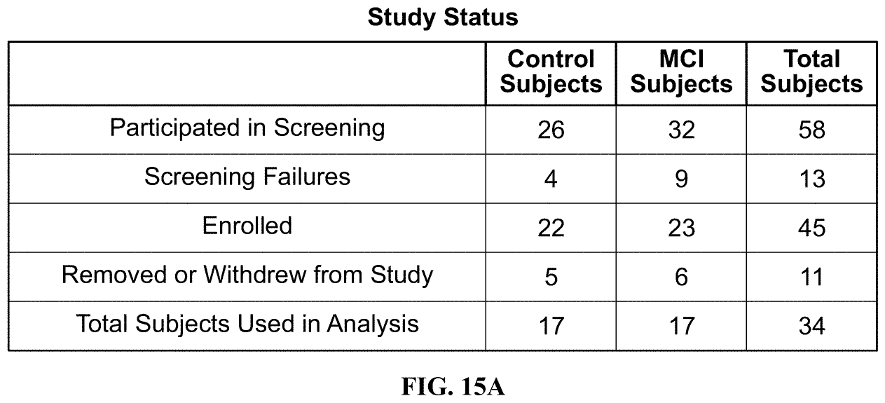

The performance of control-placebo subjects and aMCI subjects with drug or placebo treatment is also compared in other common cognitive tests, such as the Buschke Selective Reminding Test-Delayed Recall (FIGS. 11A and 11B), the Benton Visual Retention Test (FIGS. 12A and 12B), Verbal Paired Associates Test-Recognition (FIGS. 13A and 13B) and Verbal Paired Associates Test-Delayed Recall (FIGS. 14A and 14B). In all of these tests, aMCI subjects treated with placebo perform worse than placebo-treated control subjects, and levetiracetam treatment fail to rescue performance in aMCI subjects.

There are a number of possible reasons why levetiracetam treatment does not help aMCI subjects with performance in these other cognitive tests. The explicit 3-alternative forced choice task done in the fMRI study is a task that is especially sensitive to DG/CA3 function. As such, the performance of the subjects in this task may be particularly attuned to the changes in DG/CA3 activity resulting from levetiracetam treatment. Further, the aMCI subjects were treated with levetiracetam for only two weeks prior to the administration of the cognitive tests. It is contemplated that a treatment duration of longer than two weeks, e.g., 16 weeks or 8 months, for the drug treatment will result in improved efficacy. Finally, comparative animal studies indicate that an even lower dose would be more effective. The human dosage of 125 mg twice a day is equivalent to a rat dosage of 22.3 mg/kg/day. As is shown in Example 1 and FIG. 3, 20 mg/kg levetiracetam is too high a dose in rats, and it fails to improve the performance of AI rats in the radial maze task. The effective doses of levetiracetam used in the animal model are 5-10 mg/kg. To calculate the dose of levetiracetam for treatment of age-dependent cognitive impairment in humans, we assessed the levetiracetam plasma level in the rat after treatment, determined the corresponding human plasma level, and then extrapolated to provide the levetiracetam dose. See, FIG. 1 and Example 6.

### Example 3: Effect of Levetiracetam in Human Subjects with aMCI

A within-subjects trial of 8 weeks duration, involving 38 amnestic MCI (aMCI) subjects and 17 age-matched controls with a low dose treatment of levetiracetam is conducted. During the course of the study, each aMCI subject receives both drug and placebo treatments separately in two periods of two weeks each, with the order of treatments among different aMCI subjects counterbalanced (see FIG. 4). Age-matched control subjects treated with placebo serve as a further control. Cognitive testing and fMRI imaging data are obtained from the subjects after each two-week period of drug/placebo treatment.

### Participants and Clinical Characterization

38 right-handed aMCI patients are recruited from the Alzheimer's Disease Research Center (ADRC) at the Johns Hopkins Hospital and other referrals. An additional 17 right-handed healthy volunteers are recruited from the pool of control participants in the ADRC and other referrals. All participants are administered the Telephone Interview of Cognitive Status to determine if they are likely to pass the entry criteria of the study (including criteria for MRI scanning). All participants further undergo neurological, psychiatric, and neuropsychological examination using standardized instruments and methods. The psychiatric evaluation includes administration of the Structured Clinical Interview for DSM-IV Axis I Disorders and the Clinical Dementia Rating (CDR) scale. All aMCI patients have CDR scores of 0.5. Diagnosis of aMCI is based on the criteria proposed by Petersen et al. (e.g., “Mild cognitive impairment: Aging to Alzheimer's Disease,” Oxford University Press, N.Y. (2003), which include a memory complaint (corroborated by an informant), impaired memory function on testing (generally 1.5 standard deviations below the norm and at least 1 standard deviation below the norm), otherwise preserved cognitive functioning (within 1 standard deviation of norm), no decline in functional ability, and no dementia. Final aMCI diagnoses are reached by clinical consensus. Exclusion criteria include major neurological or psychiatric disorders, head trauma with loss of consciousness, history of drug abuse or dependency, and general contraindications to an MRI examination (e.g. cardiac pacemaker, aneurysm coils, claustrophobia). Each aMCI subject is required to have a study partner (i.e., an informant) who can provide information about the subject's daily function and assure that medications are taken appropriately.

Study Visits: The study consists of 4 visits over the course of 8 weeks (see FIG. 4). The Baseline Visit is for performing medical, neurological, psychiatric, and neurocognitive assessments. Visits 1 and 2 are identical to the Baseline Visit but include an fMRI session. The Washout Visit, at the end of a 4-week washout period, is for the purpose of a brief clinical assessment and initiation of the second drug/placebo phase.

Baseline Visit: At the screening visit, informed consent is obtained from the subject (and an informant in the case of MCI subjects). The subject and the informant participate in a standardized clinical interview that is used to determine the degree of the subject's functional impairment in daily life, based on the Clinical Dementia Rating (CDR) scale. The subject's medical, neurological, and psychiatric history is obtained (including a review of current medications), as well as the family history of dementia. Brief medical, neurological and psychiatric exams are conducted (including vital signs). Blood is drawn in order to perform standard laboratory tests needed to determine if the subject meets the entry criteria. The subject is re-screened for contraindications to MRI scanning, using the standard form employed at the Kirby Imaging Center. Brief cognitive testing is performed (described in section on neuropsychological assessment below). These assessments are used to determine if the subject meets the entry criteria. All of the foregoing are completed using standardized forms. If the subject meets entry criteria for the study, the subject is randomly assigned to either the 62.5 mg BID or 250 mg BID study group and given the study medication (drug or placebo, randomly selected), and instructions about how it should be taken. The subject is advised about the potential for having suicidal thoughts and advised to stop taking the medication and immediately contact the study physician if this occurs.

Visit 1: At the end of the first drug/placebo period 2 weeks after the Baseline Visit, the medical, neurological and psychiatric evaluations and cognitive testing are repeated. The subject is also clinically evaluated for suicidal ideation. Blood is drawn again to repeat the standard tests and to determine whether there are any changes related to drug treatment; the subject's blood levetiracetam level is also obtained. All medication dispensed at the Baseline Visit (drug or placebo) is collected and subject compliance with the medication regimen is assessed. The first fMRI session (with cognitive tests) is conducted on the same day, either immediately before or immediately after the clinical assessment. Subjects discontinue first period treatment at this visit.

Washout Visit: At the end of a washout period (4 weeks) following Visit 1, the subject receives a brief medical screening, including a medical and psychiatric evaluation. Blood is drawn to obtain the blood levetiracetam level (to confirm washout). The subject is provided with new medication (drug or placebo, alternated from what was assigned in the previous treatment period) for the final phase of the study with instructions about how it should be taken.

Visit 2: At approximately 2 weeks after the Washout Visit (i.e., 2 weeks after starting the second treatment period), the medical, neurological and psychiatric evaluations and the cognitive testing are repeated. The subject is clinically evaluated for suicidal ideation. Blood is drawn again to repeat the standard tests and to determine whether there were any changes related to drug treatment; the subject's blood levetiracetam level is also obtained. All medication dispensed at the Washout Visit is collected and subject compliance with the medication regimen is assessed. The second fMRI session (with cognitive tests) is repeated on the same day, either immediately before or immediately after the clinical assessment.

### Neuropsychological Assessment

All participants undergo neuropsychological evaluation at the time of assessment for treatment efficacy (Visits 1 and 2), as well as at the Baseline Visit. The evaluation occurs outside of the scanner and includes the Buschke Selective Reminding Test (Buschke and Fuld, 1974) and the Verbal Paired Associates subtest, the Logical Memory subtest, the Visual Reproduction subtest of the Wechsler Memory Scale-Revised (WMS-R) (Wechsler, 1997), and the Benton Visual Retention Test, as these tasks are particularly sensitive to medial temporal lobe function and early memory problems (Marquis et al., 2002 and Masur et al., 1994). Additionally, subjects are asked to complete tests of more general cognitive function such as tests to assess general mental status, executive function, attention and general naming ability. All neuropsychological tests are administered by a trained research assistant during a 60-minute session. As the three neuropsychological assessments in this study occur within a time period of 8 weeks, different versions of the neuropsychological tests are used to minimize test specific practice effects. Breaks are provided to the subject as needed.

### Drug Administration

As described above, the drug treatment period is the two weeks preceding Visit 1 or 2 (with the two-week period preceding the other Visit being the placebo phase). For the subjects receiving the 250 mg BID (BID stands for twice daily) drug treatment, two 250 mg tablets of levetiracetam are used to achieve a dose of 250 mg twice a day, i.e., 500 mg/day, which is approximately 7.1 mg/kg/day (assuming an average adult human weight of 70 kg). For the subjects receiving the 62.5 mg BID drug treatment, a quarter of a scored 250 mg tablet of levetiracetam is used to achieve a dose of 62.5 twice a day, i.e., 125 mg/day which is approximately 1.5 mg/kg/day.

All drug and placebo preparations are performed on a 1:1 allocation. The pharmacy randomizes patients to drug dose and condition as they enroll and keep a list of drug assignment.

Levetiracetam is rapidly and almost completely absorbed after oral administration, and its bioavailability is not affected by food. Plasma half-life of levetiracetam is approximately 7±1 hour (expected to be 9-10 hours in elderly due to decreased renal function). Absorption is rapid, with peak plasma concentrations occurring about 1 hour following oral administration. Steady state can be achieved after 2 days of multiple twice-daily dosing.

A typical starting dose of levetiracetam in treating epilepsy in humans is 500 mg twice a day, which is approximately 14.3 mg/kg/day. The dosage is then is increased until optimal efficacy, up to 50 mg/kg/day. Thus, the 250 mg BID dose (500 mg/day) used in this experiment is one-half of the lowest human dose used for treating epilepsy. The 62.5 mg BID dose (125 mg/day) is one eighth of the lowest human dose used for treating epilepsy.

### MRI Data Acquisition

Imaging data are obtained through high-resolution methods developed in the Stark laboratory. Data are collected on a Phillips 3 Tesla scanner (Eindhoven, The Netherlands) equipped with an 8-channel SENSE (Sensitivity Encoding) head coil, located at the F.M. Kirby Research Center for Functional Brain Imaging at the Kennedy Krieger Institute (Baltimore, Md.). High-resolution echo-planar images are collected using an acquisition matrix of 64×64, a repetition time of 1500 milliseconds, an echo time of 30 milliseconds, a flip angle of 70 degrees, a SENSE factor of 2, and an isotropic resolution of 1.5 mm×1.5 mm×1.5 mm with no gap. Nineteen oblique slices are acquired parallel to the principal longitudinal axis of the hippocampus and covered the entire medial temporal lobe region bilaterally. In addition to the functional runs, a whole-brain MPRAGE structural scan (parameters: 231 oblique slices, 0.65 mm isotropic resolution) is acquired.

### Image Analysis

Data analysis is carried out using the Analysis for Functional Neuroimages (AFNI, release 2010_10_19_1028) software. Images are first co-registered to correct for within- and across-scan head motion. Acquisitions in which a significant motion event occur (more than 3 degrees of rotation or 2 mm of translation in any direction relative to prior acquisition), plus and minus one-time repetition for 1.5 seconds, are excluded from the analyses. Structural anatomical data are registered to standard stereotaxic space (Talairach & Tournoux, 1988), and the same parameters are subsequently applied to the functional data. Behavioral vectors are produced to model different trial types.

The ROI-LDDMM (large deformation diffeomorphic metric mapping of the region of interest) method, a technique for cross-subject alignment, increases the power of multisubject regional fMRI studies by focusing the alignment power specifically on the ROIs (regions of interest) and not elsewhere in the brain. First, all subjects' anatomical and functional scans are normalized to the Talairach atlas using AFNI. Sub-regions of the medial temporal lobe and the hippocampus (bilateral entorhinal cortex, perirhinal cortex, Para hippocampal cortex, CA3/dentate region, CA1 region, and subiculum) are segmented in three dimensions on the MPRAGE scans. The labels for the CA3 region and dentate gyrus (DG) are combined. The anatomically defined ROIs are then used to calculate the vector field transformation for each subject using the Advanced Normalization Tools (ANTs) software package and a customized template based on the mean of the entire sample tested as the target. The resulting vector transformations for each individual subject's ROIs are then applied to the fit coefficient maps.

Group data are analyzed using a two-way Analysis of Variance (ANOVA) with trial types and group as fixed factors, and subject as a random factor nested within group. A liberal peak threshold of p<0.07, along with a spatial extent threshold of 40 voxels are used to define functional ROIs on the overall F statistic. This approach, rather than using a direct pair-wise contrast, reduces voxel selection biases because any differences amongst the various conditions allowed for a voxel to be selected. This threshold is then combined with the anatomical segmentations to only include voxels inside the regions of interest. This serves to exclude voxels that does not change with any of the model's factors, effectively limiting the analysis to voxels showing any changes with task condition or group. Voxels within each functional ROI are collapsed for further analysis.

Cognitive Tests During fMRI Scans at Visits 1 and 2

The activity of the subject's medial temporal lobe is measured by functional MRI during the subject's participation in an explicit 3-alternative forced choice task, where participants view novel, repeated and similar (“lure”) stimuli. The Psychophysics Toolbox extensions in MATLAB® 7.0 (The MathWorks, Natick, Mass.) is used for stimulus presentation and behavioral data collection. Stimuli are color photographs of common objects. Each participant undergoes a series of testing runs during the functional imaging sessions, each run consisting of a mix of three types of image pairs: similar pairs, identical pairs and unrelated foils. These image pairs are fully randomized throughout the run and presented individually as a series of images (see FIG. 7A). Participants are instructed to make a judgment as to whether each object seen is new, old or similar. Of critical interest are the participants' responses when presented with the second of the pair of similar objects (the “lure”; see FIG. 7B). The correct identification by the subject of lure stimuli as “similar,” provides behavioral evidence of pattern separation, i.e., the separation of similar experiences into distinct non-overlapping representations. However, an incorrect identification of lure stimuli as “old” or “new,” indicates a failure of pattern separation. Identification of lure stimuli as “old” indicates that the subject focused on the similarities between the lure stimulus and the earlier-shown partner image. Identification of the lure stimulus as “new” indicates that the subject failed to recall the earlier-shown partner image altogether. Each run also contains a number of baseline trials that use a challenging perceptual discrimination task known to provide a lower and more-stable estimate of baseline activity in the medial temporal lobe (Stark & Squire, 2001 PNAS; Law et al, 2005).

A survey of the activity level of various subregions in the medial temporal lobe during the cognitive test, as measured by fMRI, shows that aMCI subjects have hyperactive DG/CA3 regions and a hypoactive entorhinal cortex during the performance of memory tasks, compared to age-matched control subjects.

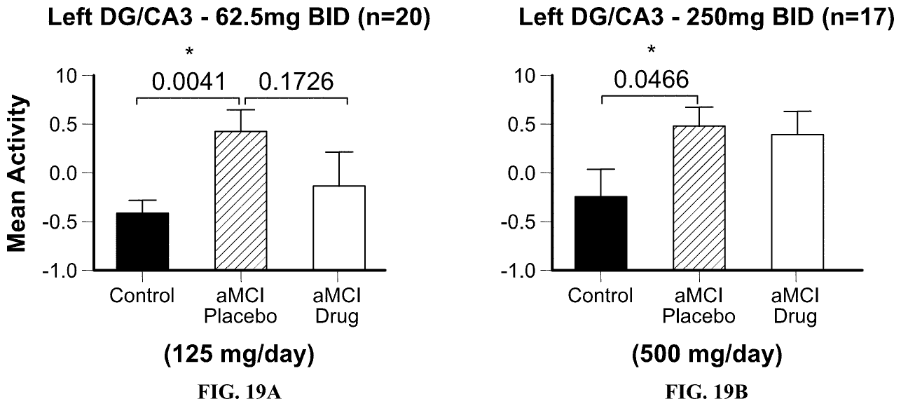

We assess the level of activity in DG/CA3 during successful memory judgments in control and aMCI subjects. The mean activity is calculated from the average activity, as measured by fMRI, during the presentation of lure stimuli correctly identified by subject as “similar” that is calibrated for baseline activity. FIGS. 19A and 19B show that aMCI patients in both the 62.5 mg BID cohort (N=20) and 250 mg BID cohort (N=17) exhibit DG/CA3 hyperactivity when making these judgments (p=0.0041 and p=0.0466 respectively). Treatment with levetiracetam does not significantly reduce the DG/CA3 hyperactivity in aMCI subjects in the 250 mg BID or the 62.5 mg BID cohort.

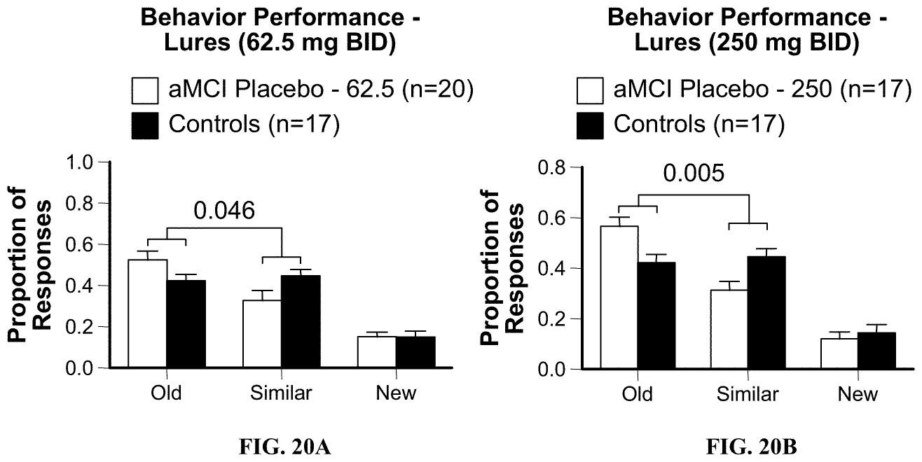

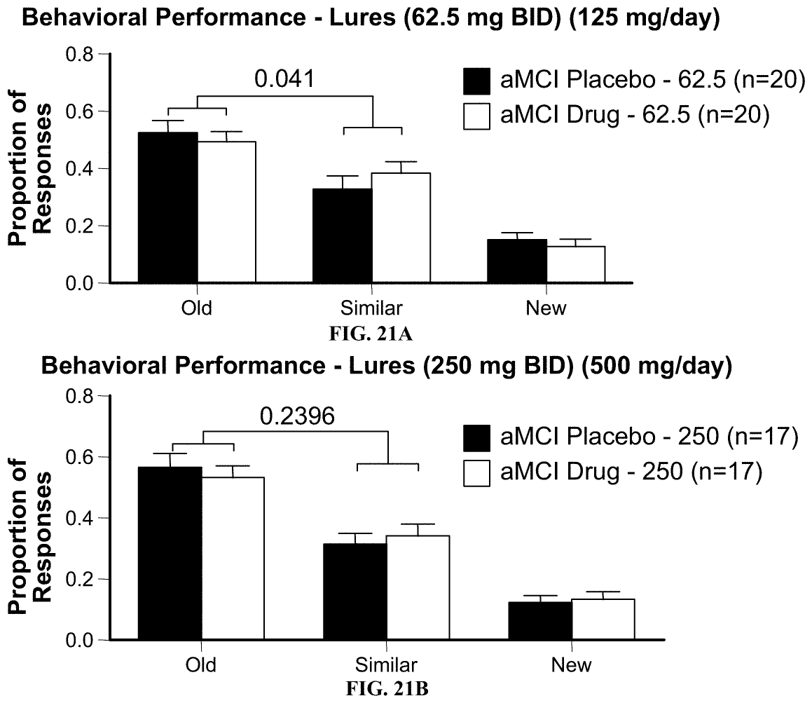

The level of DG/CA3 activity during memory judgments by levetiracetam treatment is mirrored in the change seen in the aMCI subjects' performance in the cognitive task. With placebo treatment, aMCI patients perform worse than control subjects, correctly identify lure items as “similar” less often and incorrectly identifying them as “old” more often in both the 62.5 mg BID cohort and the 250 mg BID cohort. See FIGS. 20A and 20B. However, the performance of aMCI subjects improves significantly under 62.5 mg BID levetiracetam treatment. See FIG. 21A. The interaction of more correct “similar” identifications with less incorrect “old” identifications under drug treatment results in a significant improvement in the performance of this memory task (p=0.041). The performance of aMCI subjects does not significantly improve under 250 mg BID levetiracetam treatment (p=0.2396). See FIG. 21B.

### Example 4: Effect of Brivaracetam and Seletractam in Aged-Impaired Rats

### Subjects

Aged, male Long-Evans rats were obtained at 8-9 month of age from Charles River Laboratories (Raleigh, N.C.) and housed in a vivarium at Johns Hopkins University until 24-26 month of age. Young rats obtained from the same source were housed in the same vivarium and tested at 6 month of age. All rats were individually housed at 25° C. and maintained on a 12 hr light/dark cycle. Food and water were provided ad libitum unless noted otherwise. The rats were examined for health and pathogen-free status throughout the experiments, as well as necropsies at the time of sacrifice. All procedures in the current investigations were approved by the Institutional Animal Care and Use Committee in accordance with the National Institutes of Health directive.

### Background Characterization of Cognitive Status

All rats were screened in a standardized assessment of spatial cognition prior to the studies with experimental treatments. That background assessment used a well-established Morris Water Maze protocol. The MWM protocol was substantially the same as the one described in Example 1. See, also, Gallagher et al., Behav. Neurosci. 107:618-626, (1993). Briefly, the rats were trained for eight days (three trials per day) to locate a camouflaged escape platform that remained at the same location throughout training in a water maze. Every sixth trial consisted of a probe trial (free swim with no escape platform) that served to assess the development of a spatially localized search for the escape platform. During these probe trials, a learning index was generated from the proximity of the rat to the escape platform and was used to define impairment in the aged rats. The learning index is the sum of weighted proximity scores obtained during probe trials, with low scores reflecting a search near the escape platform and high scores reflecting searches farther away from the platform (Gallagher et al, 1993). Cue training (visible escape platform) occurred on the last day of training to test for sensorimotor and motivational factors independent of spatial learning. Aged rats with impaired spatial memory performance (i.e., those with learning index scores outside the young “normative” range) but successful cued training performance were characterized as Aged-Impaired rats (i.e., AI rats). The AI rats were used for the studies as described below.

### Treatments

The radial arm maze experiments used acute administration of seletracetam (0-4 mg/kg), brivaracetam (0-4 mg/kg), or saline vehicle given by intraperitoneal injection (in a volume of 1 ml/kg) 30-40 min prior to test sessions. In the chronic treatment experiment, memory-impaired aged rats were implanted subcutaneously in the intrascapular region with osmotic mini-pumps (ALZET, Durect Corporation, Cupertino, Calif.) with brivaracetam (2 mg/kg/day) or saline vehicle starting two weeks prior to assessment in the water maze.

### Behavioral Assessment in the Radial Arm Maze

A radial arm maze (RAM) task was used to assess effects of acute drug treatment with seletracetam and brivaracetam. This protocol allowed within-subject assessment across drugs at different doses. The radial maze consisted of eight arms projecting from each side of an octagonal center platform, with a food well located at the distal end of each arm. Plexiglas blocks could be positioned to prevent entry into any arm. Extra-maze cues were provided in the room surrounding the maze and illumination was provided by an overhead light.

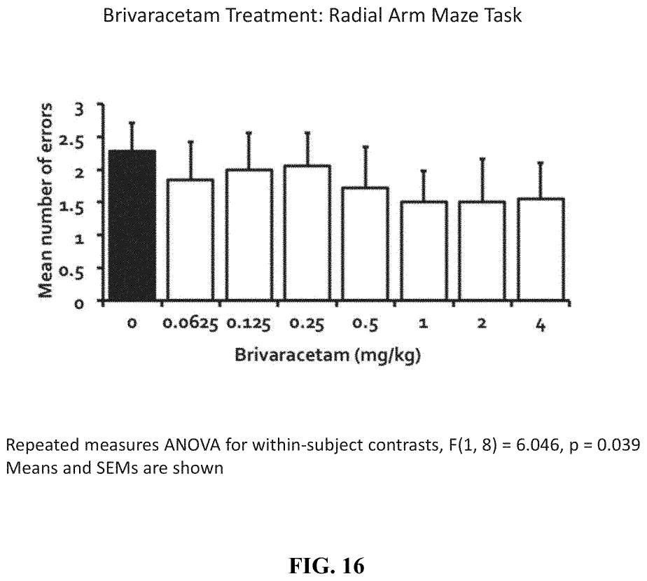

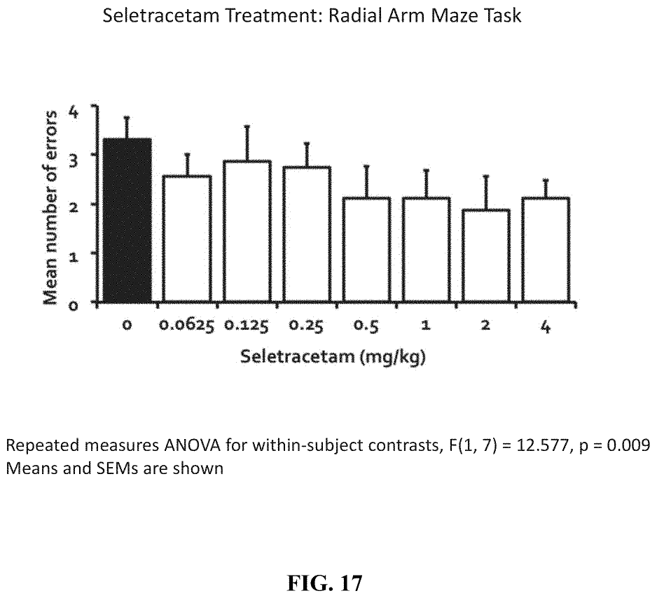

Pre-training, as described in detail in Chappell et al. Neuropharmacology 37: 481-487, (1998), consisted of habituation, standard win-shift training, and win-shift training with delays interposed between information and memory test phases. Drug treatments began two days after the completion of pre-training. Three arms were blocked at the beginning of each trial (information phase). The identity and configuration of the blocked arms were varied across trials. Food-deprived rats were allowed to retrieve food reward (Kellogg's Froot Loops™ cereal) from the five unblocked arms. The rat was then removed from the maze for 2 hr (retention interval), during which time the barriers on the blocked arms were removed allowing access to all eight arms. Rats were then placed back onto the center platform and allowed to retrieve the remaining food rewards (memory test phase). An error consisted of returning to an arm (all four paws on the arm) from which food had already been obtained. Memory-impaired aged rats (n=8 for seletracetam, and n=9 for brivaracetam) were first tested with a series of drug doses in ascending/descending order; each dose was thus tested twice, with one washout day in between each determination. The number of errors made in the retention phase after the 2-hr delay was used to assess memory performance. See FIG. 16 and FIG. 17. A series of different doses of brivaracetam was tested: 0.0625 mg/kg, 0.125 mg/kg, 0.25 mg/kg, 0.5 mg/kg, 1 mg/kg, 2 mg/kg and 4 mg/kg. A series of different doses of seletracetam was tested: 0.0625 mg/kg, 0.125 mg/kg, 0.25 mg/kg, 0.5 mg/kg, 1 mg/kg, 2 mg/kg and 4 mg/kg. As shown in FIG. 16, brivaracetam has a significant effect as a function of dose in the range tested (repeated measures ANOVA for within-subject contrasts, F(1, 8)=6.046, p=0.039). As shown in FIG. 17, seletracetam also has a significant effect as a function of dose in the range tested (repeated measures ANOVA for within-subject contrasts, F(1,7)=12.577, p=0.009).

### Behavioral Assessment in the Water Maze

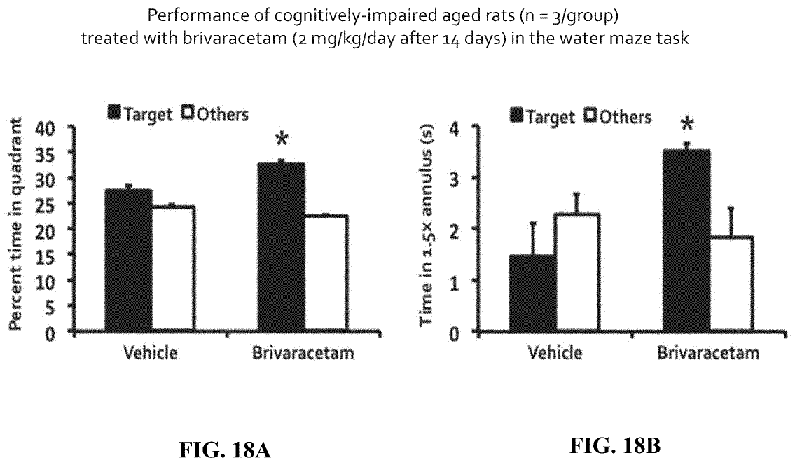

Rats were trained and tested in a novel water maze environment to assess the effect of drug treatment. The water maze used here was housed in a different building and was surrounded by curtains with a novel set of patterns relative to the maze used for initial assessment of cognitive status. The training protocol consisted of 6 trials per day for 2 days to locate a submerged escape platform. On each trial, a rat was released in the maze from one of four equally spaced starting positions around the perimeter of the pool. The starting position varied from trial to trial. If the rat did not locate the escape platform within 60 s on any trial, the experimenter guided and placed the rat on the platform, where it remained for 20 s. The rat was then removed from the platform and placed in a holding cage for another 40 s before the next trial. Approximately 24 hr after the last training trial, a probe test in the absence of the escape platform was given to assess spatial memory. Results of the behavior assessment in the Water Maze task were shown in FIG. 18A and FIG. 18B. Rats treated with brivaracetam at 2 mg/kg/day (t(2)=10.000, p=0.010) but not vehicle (t(2)=1.964, p=0.188) showed a significant spatial bias for the target quadrant compared to the other control quadrants. In addition, brivaracetam-treated rats (2 mg/kg/day) spent significantly more time in the target quadrant than the vehicle-treated rats, t(4)=3.881, p=0.018. Brivaracetam-treated rats (2 mg/kg/day) spent significantly more time in the target annulus (area surrounding the location of the escape platform) than the vehicle-treated rats, t(4)=3.109, p=0.036.

### Example 5: Chronic Treatment with Levetiracetam in Aged-Impaired Rats

### Subjects

Aged, male Long-Evans rats were obtained at 8-9 month of age from Charles River Laboratories (Raleigh, N.C.) and housed in a vivarium at Johns Hopkins University until 24-26 month of age. Young rats obtained from the same source were housed in the same vivarium and tested at 6 month of age. All rats were individually housed at 25° C. and maintained on a 12 hr light/dark cycle. Food and water were provided ad libitum unless noted otherwise. The rats were examined for health and pathogen-free status throughout the experiments, as well as necropsies at the time of sacrifice. All procedures in the current investigations were approved by the Institutional Animal Care and Use Committee in accordance with the National Institutes of Health directive.

### Background Behavioral Characterization

All rats were screened in a standardized assessment of spatial cognition prior to the studies with experimental treatments. That background assessment used a well-established Morris water maze protocol as described in Gallagher et al, 1993. Briefly, the rats were trained for eight days (three trials per day) to locate a camouflaged escape platform that remained at the same location throughout training in a water maze. Every sixth trial consisted of a probe trial (free swim with no escape platform) that served to assess the development of a spatially localized search for the escape platform. During these probe trials, a learning index was generated from the proximity of the rat to the escape platform and was used to define impairment in the aged rats. The learning index is the sum of weighted proximity scores obtained during probe trials, with low scores reflecting a search near the escape platform and high scores reflecting searches farther away from the platform (Gallagher et al, 1993). Cue training (visible escape platform) occurred on the last day of training to test for sensorimotor and motivational factors independent of spatial learning. Aged rats with impaired spatial memory performance (i.e., those with learning index scores outside the young “normative” range) but successful cued training performance were used for the studies as described below.

### Surgery and Treatments

Under isoflurane anesthesia, memory-impaired aged rats were implanted subcutaneously in the intrascapular region with osmotic mini-pumps (ALZET, Durect Corporation, Cupertino, Calif.) with levetiracetam (10 mg/kg/day) or saline vehicle for four weeks prior to perfusion. Young rats, which served as controls, received either saline vehicle in mini-pumps or no implantation.

### Perfusion and Tissue Preparation

At the end of the 4-week treatment period, rats were anesthetized with isoflurane and perfused transcardiacally with 0.1 M phosphate buffer saline, followed by 4% paraformaldehyde in phosphate buffer. Brains were removed and post-fixed in paraformaldehyde overnight. The brains were then moved into 4% paraformaldehyde in phosphate buffer containing 16% sucrose. The brains were then sectioned with a freezing microtome on the coronal plane at 40 μm and stored in either 4% paraformaldehyde at 4° C. for in situ hybridization or cryoprotectant at −20° C. for immunohistochemistry.

### Probe Synthesis

Probe templates were synthesized as described in Haberman et al. (2008). Initial primer sequences for reelin were as follows: left, agtactcagacgtgcagtgg, right, ctcatgaagcaaagtccaa; PCR products were verified by restriction endonuclease digestion. Initial PCR products were amplified further with the same PCR primers that had been modified by the addition of T7 or SP6 RNA polymerase binding sites. PCR products containing T7 and SP6 extensions were purified by SVgel and a PCR cleanup kit (Promega). 35S-UTP labeled riboprobe was then generated using the Maxiscript kit (Ambion). The probe was then phenol/chloroform extracted and precipitated in ethanol at −80° C. The final probe was resuspended in RNase-free water and the specific activity was determined by scintillation counter.

### In Situ Hybridization

In situ hybridization was carried out as described by Haberman et al., (2008). Free-floating tissue sections were washed in 0.75% glycine in 0.1M phosphate buffer two times, followed by a single wash in phosphate buffer. After that, sections were reacted in Proteinase K buffer containing 1.0 μg/ml proteinase K for 30 minutes at 37° C. Sections were then treated with acetic anhydride solution (11.3% triethanolamine, 0.25% acetic anhydride, 0.04 M acetic acid) for 10 minutes at room temperature. This was followed by two 15-minute washes in 2× sodium chloride/citrate buffer (SSC buffer; 20× concentration, 3M NaCl, 0.3M sodium citrate). Next, sections were transferred to hybridization buffer containing 20% formamide, 0.4×Denhardt's solution, 4% dextran sulfate, and 1.6×SSC) supplemented with 0.25 mg/ml tRNA, 0.33 mg/ml sheared salmon sperm DNA, 100 mM DTT, and 1×107 cpm/ml 35S-UTP-labeled probe for overnight reaction at 60° C. The following day, sections were washed at 60° C. in 4×SSC/0.01M DTT and 2×SSC/50% formamide. They were then incubated with RNase (20 μg/ml) at 37° C. for 30 min. Sections were washed with progressively decreasing concentrations of SSC before mounting on slides. Slides were dried overnight, exposed to a phosphorimager screen, and quantified by using ImageQuant (GE Healthcare). Digital images were acquired of entorhinal cortical sections from the same levels for all animals and the subregion of interest was outlined and quantified. Sections were averaged to obtain a single score for each animal.

### Immunohistochemistry

Tissue was labeled with anti-SOM antiserum (Santa Cruz Biotechnology; cat. no. SC7819-P) using an established immunoperoxidase protocol and tissue sections were processed concurrently to minimize inter-replication variability (Haberman et al., 2009). The anti-SOM antiserum can detect somatostatin. Briefly, sections were washed in 0.1M phosphate-buffered saline (PBS) to remove cryoprotectant, and endogenous peroxidases were quenched in 0.3% H202 in PBS. After additional PBS washes, sections were blocked in 5% normal horse serum in PBS with 0.3% Triton. Sections were then incubated with primary antibody at a dilution of 1:1600 in PBS containing 0.15% Triton and 3% normal serum for 72 hours at 4° C. with agitation. Following primary antibody incubation, sections were washed in PBS and reacted with horse anti-goat IgG biotinylated secondary antibody (Vector Laboratories Inc., Burlingame, Calif.) diluted in PBS with 0.15% Triton and 5% normal horse serum for 45 minutes. The secondary antibody was detected with avidin-biotin complex (ABC Elite; Vector Laboratories Inc., Burlingame, Calif.) and the avidin-biotin complex was visualized with nickel-enhanced diaminobenzadine (Vector Laboratories Inc., Burlingame, Calif.). Tissue sections were mounted onto coated slides and dried, dehydrated with increasing concentrations of ethanol, cleared with xylene, and coverslipped using DPX mounting media.

Interneuron quantification was performed using a Zeiss Axioplan 2 microscope equipped with a motorized stage. All analyses were conducted blind with regards to animal age and cognitive status. The dentate hilar region was defined using the Paxinos and Watson rat brain atlas (1998). Dorsal hilar neuron counts were derived bilaterally from four matched tissue sections per animal with a 40× objective lens (Bregma −3.80 mm to −4.16 mm). Neuron counts were analyzed as the total number of hilar interneurons per hippocampal section for each rat.

### Results

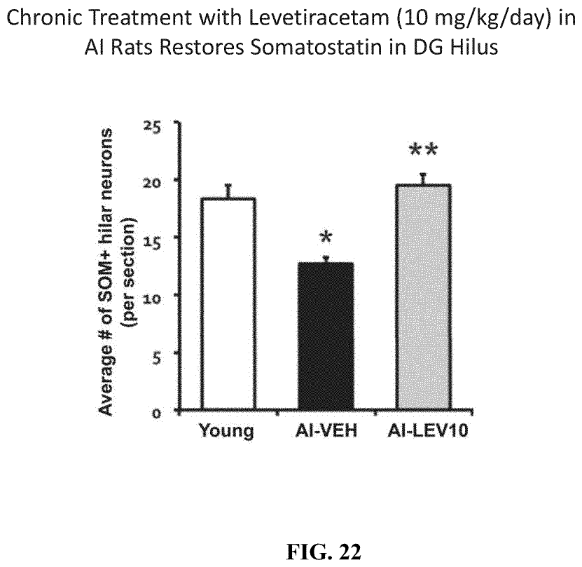

Somatostatin is a peptide hormone that regulates the endocrine system and affects neurotransmission and cell proliferation via interaction with G protein-coupled somatostatin receptors and inhibition of the release of numerous secondary hormones. Somatostatin levels in the brain have been shown to drop as low as 10-20% in association with aging and Alzheimer's disease progression. A four-week treatment with levetiracetam at a dose of 10 mg/kg/day in aged-impaired rats restores the levels of somatostatin in DG hilus. See FIG. 22. Aged-impaired rats that were administered a saline vehicle rather than drug possessed significantly lower numbers of SOM-immunoreactive hilar neurons relative to both young and levetiracetam treated aged rats (N=18; F2,20=15.739, p<0.001; AI-LEV vs Y, p=0.679; AI-LEV vs AI-VEH, p<0.01; AI-VEH vs Y, p<0.001).

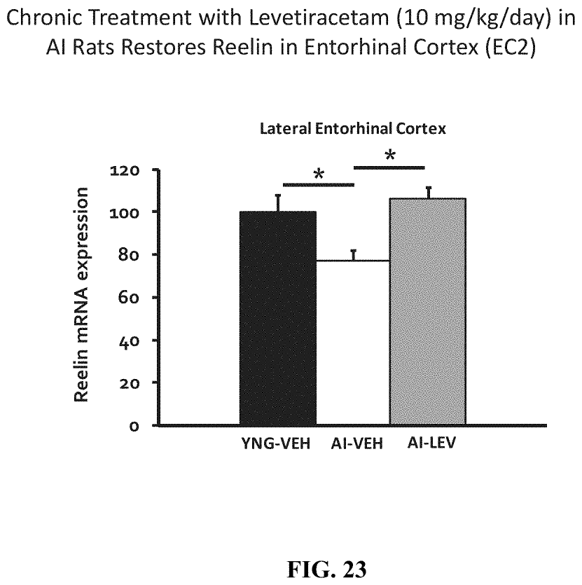

Reelin is a large secreted extracellular matrix glycoprotein that helps regulate processes of neuronal migration and positioning in the developing brain by controlling cell-cell interactions. Reduced reelin expression in EC2 neurons has been observed in aged rats with memory loss, in hAPPJ20 AD mice, as well as in human AD brains (Chin et al. 2007; Stranahan et al. 2010). A four-week treatment with levetiracetam at a dose of 10 mg/kg/day in aged-impaired rats restores the levels of reelin in Entorhinal Cortex (EC2). See FIG. 23. A one-way ANOVA shows a significant difference among the groups, F(2, 20)=5.035, p=0.017. Additional analysis shows that reelin mRNA expression in the lateral entorhinal cortex of AI rats treated with vehicle controls (AI-VEH) is significantly lower than that of young rats, t(13)=2.790, p=0.015. Treatment with levetiracetam in AI rats at a dose of 10 mg/kg/day for 28 days (AI-LEV) significantly increased the expression of reelin, t(13)=2.386, p=0.033 (compared to AI-VEH).

### Example 6: Evaluation of Levetiracetam Blood Plasma Levels

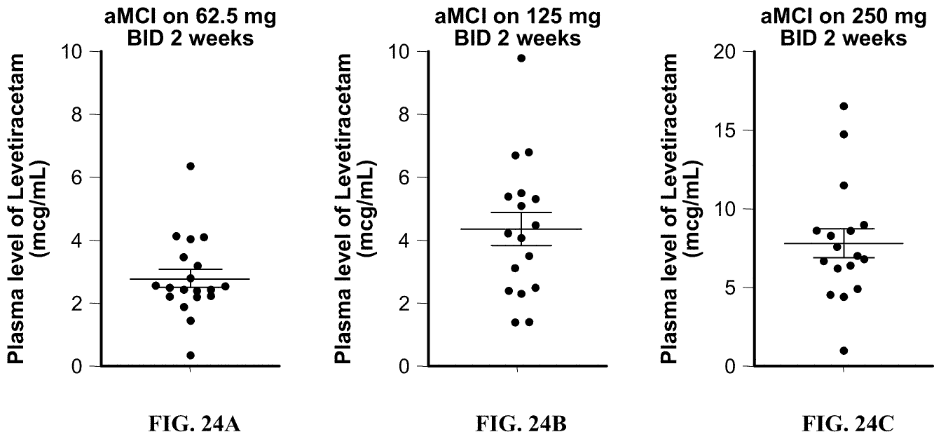

Human: in the human studies described in Examples 2 and 3, a subject's levetiracetam blood plasma level was assessed at each visit. The subject's blood was drawn by the Johns Hopkins Phlebotomy Service and analysis of levetiracetam blood plasma levels was conducted either by the Johns Hopkins Core laboratory or by MedTox Laboratories in St. Paul, Minn. for the 62.5 mg BID cohort, the 125 mg BID cohort and the 250 mg BID cohort. Upon completion of the levetiracetam treatment, subjects in the 62.5 mg BID cohort showed a mean levetiracetam blood plasma level of 2.88 mcg/ml (SEM±0.288), while levels in the 125 mg BID had 4.4 mcg/ml (SEM±0.53) and subjects in the 250 mg BID cohort showed mean levetiracetam blood plasma level of 7.9 mcg/ml (SEM±0.92). See FIGS. 24A-24C.

Rats: Blood was drawn from aged-impaired rats by cardiac puncture during perfusion after a 28-day levetiracetam treatment period and sent for analysis of levetiracetam plasma levels by MedTox Laboratories in St. Paul, Minn. Aged-impaired rats treated with 10 mg/kg/day of levetiracetam showed a mean levetiracetam blood plasma level of 3.8 mcg/ml (SEM±0.255), while those treated with 60 mg/kg/day showed a mean levetiracetam blood plasma level of 22.4 mcg/ml (SEM±3.371). See FIG. 1.

### Example 7: Synergy of Compound 1 and Levetiracetam

### Subjects

Aged, male Long-Evans rats were obtained at 9 months of age from Charles River Laboratories (Raleigh, N.C.) and housed in a vivarium at Johns Hopkins University until background behavioral assessment in a water maze at 24 months of age. Young rats obtained from the same source were housed in the same vivarium and were included in the background assessment at 6 months of age but were not used for drug testing in a radial arm maze task. All rats were individually housed at 25° C. and maintained on a 12 h light/dark cycle. Food and water were provided ad libitum unless noted otherwise. The rats were examined for health and pathogen-free status throughout the experiments, as well as necropsies at the time of killing. All procedures were in accordance with NIH guidelines using protocols approved by the Institutional Animal Care and Use Committee at Johns Hopkins University.

### Background Behavioral Assessment

All rats were screened in a standardized assessment of spatial cognition before the commencement of drug studies. The background assessment used a well-established Morris water maze protocol as described in detail elsewhere (Gallagher et al, 1993). Briefly, the rats were trained for 8 days (three trials per day) to locate a camouflaged escape platform that remained at the same location throughout training in a water maze. Every sixth trial consisted of a probe trial (free swim with no escape platform) that served to assess the development of a spatially localized search for the escape platform. During these probe trials, a learning index was generated from the proximity of the rat to the escape platform and was used to define impairment in the aged rats. The learning index is the sum of weighted proximity scores obtained during probe trials, with low scores reflecting a search near the escape platform and high scores reflecting searches farther away from the platform (Gallagher et al, 1993). Cue training (visible escape platform) occurred on the last day of training to test for sensorimotor and motivational factors independent of spatial learning. Aged rats with impaired spatial memory performance (i.e., those with learning index scores outside the young ‘normative’ range) but successful cued training performance were used for the studies as described below.

1. Acute Treatment with Compound 1 Via PO on Radial Arm Maze

Food-deprived aged rats maintained at approximately 85% free-feeding weights were tested for their hippocampal-dependent memory in a radial arm maze task under varying doses of Compound 1 (a GABA_{A} α5 receptor agonist):

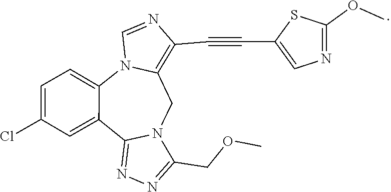 [SMILES: COCc1nnc2n1Cc1c(C#Cc3cnc(OC)s3)ncn1-c1ccc(Cl)cc1-2]

The radial arm maze apparatus used consisted of eight equidistantly spaced arms. An elevated maze arm (7 cm width×75 cm length) projected from each facet of an octagonal center platform (30 cm diameter, 51.5 cm height). Clear side walls on the arms were 10 cm high and are angled at 65° to form a trough. A food well (4 cm diameter, 2 cm deep) was located at the distal end of each arm. Froot Loops™ (Kellogg Company) were used as rewards. Blocks constructed of Plexiglass™ (30 cm height×12 cm width) could be positioned to prevent entry to any arm. Numerous extra maze cues surrounding the apparatus were also provided. The rats were initially subjected to pre-training (Chappell et al., 1998). Pre-training consisted of a habituation phase, a training phase on the standard win-shift task and another training phase in which a progressively longer delay was imposed between presentation of a subset of arms designated by the experimenter (five arms available and three arms blocked) and completion of the eight-arm win-shift task (i.e., with all eight arms available).

In the habituation phase, rats were familiarized to the maze for a 10-minute session on several days. In each of these sessions, food rewards were scattered on the maze, initially on the center platform and arms and then progressively confined to the arms. After this habituation phase, a standard training protocol was used, in which a food pellet is located at the end of each arm. Rats received one trial each day. Each daily trial terminates when all eight food pellets have been obtained or when either 16 choices are made or 10 minutes had elapsed. After completion of this training phase, a second training phase was carried out in which the memory demand was increased by imposing a brief delay during the trial. At the beginning of each trial, three arms of the eight-arm maze were blocked. Rats were allowed to obtain food on the five arms to which access was permitted during this initial ‘information phase’ of the trial. Rats were then removed from the maze for progressively longer delays over days (1 min, 30 min, 60 min, etc), during which time the barriers on the maze were removed, thus allowing access to all eight arms. Rats were then placed back onto the center platform and allowed to obtain the remaining food rewards during this ‘retention test’ phase of the trial. The identity and configuration of the blocked arms varied across trials.

The number of errors the rats made during the retention test phase was tallied. An error occurred in the trial if the rats entered an arm from which food had already been retrieved in the pre-delay component of the trial, or if it re-visited an arm in the post-delay session that had already been visited. After completion of the pre-training test, rats were tested on the task with different doses of Compound 1 using a 5-hr memory retention delay between the information and the test trial.

The efficacy of Compound 1 was tested using oral gavages (PO), in which the drug was administered 30-40 min before each information trial at a volume of 10 ml/kg. Doses tested were 0, 3, 10, and 30 mg/kg using ascending-descending dose series; that is, the dose series was given first in an ascending order and then repeated in a descending order. Therefore, each dose had two determinations; the average number of errors made from the two determinations for each dose was used for analysis. Each drug test was given every other day with intervening washout days, and the vehicle used to deliver the drug was 20% Tween-80.

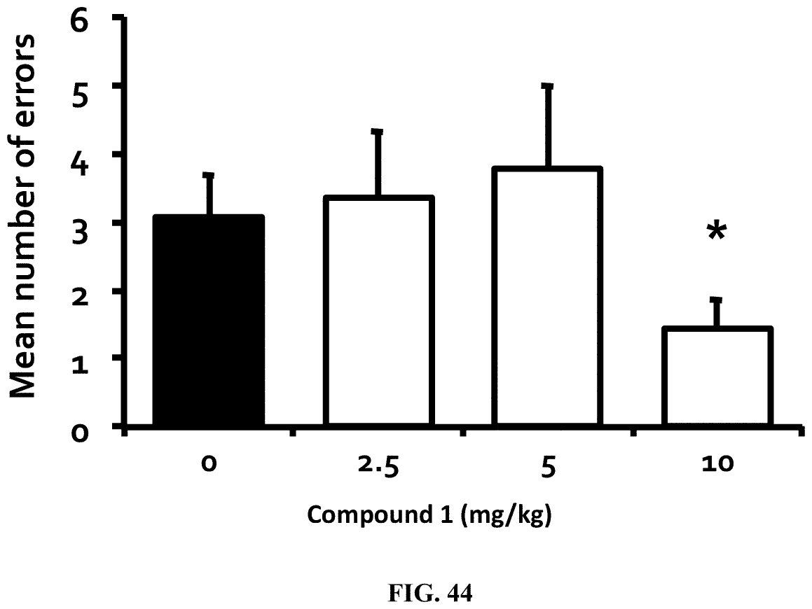

The results demonstrate that aged-impaired rats treated with Compound 1 at a dose of 10 mg/kg performed the radial arm maze with fewer errors (FIG. 44). These results indicate that Compound 1 improves the cognition of aged-impaired rats.

2. Acute and Chronic Treatments with Compound 1 on Water Maze

Rats were trained and tested in a novel water maze environment to assess the effect of the treatments. The water maze used here was housed in a different room and was surrounded by curtains with a novel set of patterns relative to the maze used for initial assessment of cognitive status. The training and testing protocol used was identical to the spatial learning-activated protocol described in Haberman et al., (2008, Proceedings of the National Academy of Sciences USA, 105, 10601-10606). The task required rats to swim to a visible escape platform at a fixed location in the presence of spatial cues for 8 training trials with an inter-trial interval of 8 min. An hour after the last training trial, rats were given a probe test in the absence of the escape platform (free swim) to assess the memory of the platform location as measured by time spent searching at the target location.

To assess the acute and chronic effects of Compound 1 treatment, rats received 15-16 days of drug injections with assessment on the water maze on the first day (acute effect) and last day (chronic effect) of treatment. Different surrounding spatial cues and escape location in the water maze were used for the initial and subsequent assessments. Compound 1 was given at 10 mg/kg using intraperitoneal injection (IP) at a volume of 1 ml/kg. On days of water maze assessment, the drug was given 30-40 min before the first training trial. The vehicle used to deliver Compound 1 consisted of 10% N-methyl-2-pyrrolidone (NMP), 45% PEG-400, 11.25% of 2-hydroxypropyl-β-cyclodextrin (HPCD) at 25% concentration, and 33.75% of distilled water.

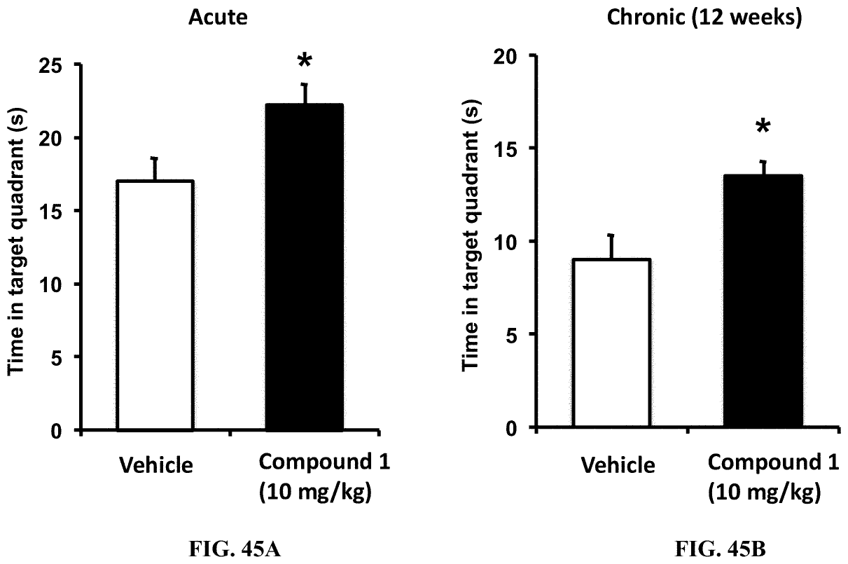

The results demonstrate that rats treated acutely or chronically with Compound 1 at a dose of 10 mg/kg spent more time in the target quadrant of the Morris Water Maze (FIGS. 45A and 45B). The results indicate that Compound 1 improves the cognition of aged-impaired rats.

### 3. Combination Treatment of Compound 1 and Levetiracetam on Radial Arm Maze

Pretraining procedures on the radial arm maze were the same as described above. Once rats were trained on the task procedures, we assessed a range of Compound 1 doses to identify subtherapeutic doses when administered on its own. We then combined each of those subtherapeutic doses of Compound 1 with levetiracetam (an SV2A inhibitor) to assess synergistic interaction between the two drugs. Following that, we reassessed those same subtherapeutic Compound 1 doses at the end to confirm that they were indeed subthreshold when given on its own.

Rats were first tested with Compound 1 in which the drug was administered 30-40 min before each information trial using intraperitoneal injection (IP) at a volume of 1 ml/kg. Doses were tested in the following order: 0, 2.5, 5, and 10 mg/kg. The results indicated that Compound 1 at 2.5 and 5 mg/kg were subtherapeutic; those two doses of Compound 1 were hence selected for combination therapy with levetiracetam (see next paragraph). Following combination therapy, Compound 1 at 2.5 and 5 mg/kg were tested again on their own to confirm that those two doses were subthreshold; the doses were tested in the following order: 5, 2.5, 0 mg/kg. The number of memory errors made at each of those subthreshold doses from the two determinations (at the beginning and at the end of the study) was averaged for analysis.

For combination therapy, both Compound 1 and levetiracetam were administered using IP injections at a volume of 1 ml/kg and were given 30-40 min before each information trial. The drug doses were tested in the following order: (1) Compound 1 at 0 mg/kg (vehicle) combined with levetiracetam at 0 mg/kg (vehicle), (2) Compound 1 at 5 mg/kg combined with levetiracetam at 1.25 mg/kg, (3) Compound 1 at 2.5 mg/kg combined with levetiracetam at 2.5 mg/kg, and (4) Compound 1 at 0 mg/kg (vehicle) combined with levetiracetam at 0 mg/kg (vehicle). Baseline assessment with vehicles was thus tested twice (once at the beginning and once at the end of the series); the average number of errors made from those two baseline determinations was used for analysis. Each combination drug test was given every other day with intervening washout days. The vehicle used to deliver Compound 1 consisted of 10% N-methyl-2-pyrrolidone (NMP), 45% PEG-400, 11.25% of 2-hydroxypropyl-β-cyclodextrin (HPCD) at 25% concentration, and 33.75% of distilled water, and the vehicle used to deliver levetiracetam was saline. Subtherapeutic doses of levetiracetam (e.g., less than 5 mg/kg) were selected based on the findings of Koh et al. (2010, Neuropsychopharmacology, 35, 1016-1025).

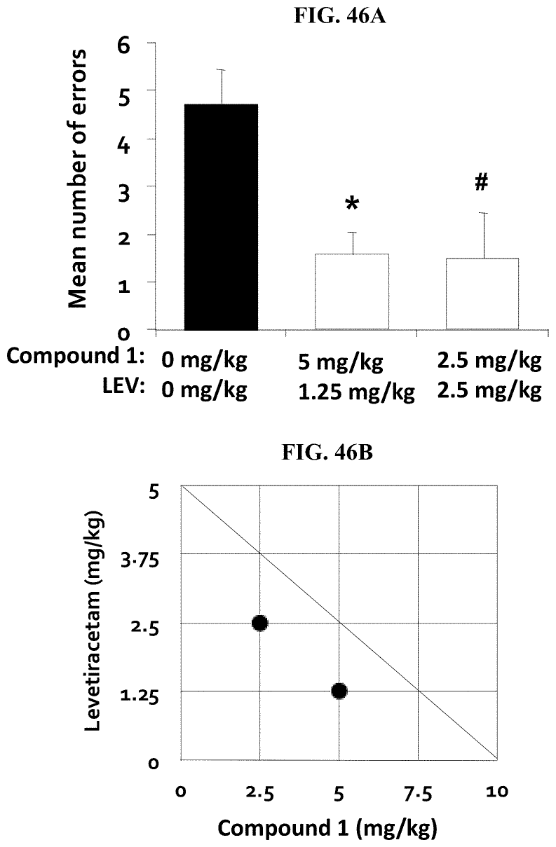

The results demonstrate that aged-impaired rats treated with a combination of Compound 1 at a dose of 2.5 mg/kg and levetiracetam at a dose of 2.5 mg/kg or a combination of Compound 1 at a dose of 5 mg/kg and levetiracetam at a dose of 2.5 mg/kg performed the radial arm maze with fewer errors (FIGS. 46A and 46B). These results indicate that the combination of Compound 1 and levetiracetam may improve the cognition of aged-impaired rats, even when given at subtherapeutic or low doses. The dose of Compound 1 in combination with levetiracetam was lower than that administered in the Morris water maze and radial arm maze studies with Compound 1 alone detailed above (2.5 mg/kg or 5 mg/kg versus 10 mg/kg above), yet an effect was still observed and it was greater than expected (FIG. 46B; isobolographic analysis). Moreover, the dose of levetiracetam was lower than what is typically effective (see Example 1, where levetiracetam, on its own, was effective in reducing the number of errors by AI rats in the radial arm maze at 5-10 mg/kg doses, but not at 1.25 mg/kg or 2.5 mg/kg), yet was still effective in combination with Compound 1. The data suggest that the combination of Compound 1 and levetiracetam had a synergistic effect on the treatment of cognitive impairment in AI rats.

### Example 8: Compound 1 Salt and Polymorph Screening

### General Procedures for Salt and Polymorph Screening

### Anti-Solvent Additions

Solutions were contacted with anti-solvents. These anti-solvent additions were added to help lower the solubility of the solvent system and induce crystallization.

### Cooling and Slow Cools

Solutions were prepared in the selected solvent or solvent/anti-solvent system. These solutions were chilled below room temperature within a refrigerator for varying lengths of time in an attempt to induce nucleation. The presence or absence of solids was noted. Upon observation of solids, in quantities sufficient for analysis, isolation of material was conduction. If insufficient quantities were present further cooling was performed in a freezer. Samples were either isolated for analysis wet or as dry powders.

### Fast Evaporation

Solutions were prepared in selected solvents and agitated between aliquot additions to assist in dissolution. Once a mixture reached complete dissolution, as judged by visual observation, the solution was filtered through a 0.2-μm nylon filter and allowed to evaporate at ambient temperature in an uncapped vial or at ambient under nitrogen. The solids that formed were isolated for evaluation.

### Slow Evaporation

Solutions were prepared in selected solvents and agitated between aliquot additions to assist in dissolution. Once a mixture reached complete dissolution, as judged by visual observation, the solution was filtered through a 0.2-μm nylon filter into a sample vial. The vial opening was covered with foil and pierced 3× to slow and allowed to evaporate at ambient. The solids that formed were isolated for evaluation.

### Slurry

Solutions were prepared by adding enough solids to a given solvent so that excess solids were present. The mixture was then agitated in a sealed vial at either ambient or an elevated temperature. After a given amount of time, the solids were isolated for analysis.

### Solubility Estimation

Aliquots of various solvents were added to measured amounts of a given material with agitation (typically sonication) at stated temperatures until complete dissolution was achieved, as judged by visual observation. If dissolution occurred after the addition of the first aliquot, values are reported as “>”. If dissolution did not occur, values are reported as “<”.

### General Instrumental Techniques for Salt and Polymorph Screening

The method(s) used to analyze this (these) compound(s) has(have) been neither validated by nor formally transferred to AMRI SSCI, LLC. The results from the analyses are therefore not suitable for use where regulations require validated methods (e.g., post-IND development or batch/lot release).

### Differential Scanning Calorimetry (DSC)

DSC was performed using a Mettler-Toledo DSC3+ differential scanning calorimeter. A tau lag adjustment is performed with indium, tin, and zinc. The temperature and enthalpy are adjusted with octane, phenyl salicylate, indium, tin and zinc. The adjustment is then verified with octane, phenyl salicylate, indium, tin, and zinc. The sample was placed into a hermetically sealed aluminum DSC pan, the weight was accurately recorded, and the sample was inserted into the DSC cell. A weighed aluminum pan configured as the sample pan was placed on the reference side of the cell. The pan lid was pierced prior to sample analysis. The samples were analyzed from −25° C. to 250° C. at 10° C./min.

### Dynamic Vapor Sorption (DVS)

Dynamic vapor sorption data was collected on a Surface Measurement System DVS Intrinsic instrument. The samples were not dried prior to analysis. Sorption and desorption data were collected over a range from 5% to 95% RH in 10% RH increments under a nitrogen purge. The equilibrium criteria used for the analyses were 0.001 dm/dt weight change in 5 minutes with a minimum step time of 30 minutes and maximum equilibration time of 180 minutes with a 3-minute data logging interval. Data were not corrected for the initial moisture content of the sample. The samples were identified as having low, limited or significant hygroscopicity based on the definitions in the below table.

| Term | Definition |
|---|---|
| Low hygroscopicity | Material exhibits < 0.5 wt % water uptake |
| | over a specified RH range. |
| Limited hygroscopicity | Material exhibits < 2.0 wt % water uptake |
| | over a specified RH range. |
| Significant hygroscopicity | Material exhibits ≥ 2.0 wt % water uptake |
| | over a specified RH range. |

### Thermogravimetry (TGA or TGA/DSC)

Thermogravimetric analyses were performed using a Mettler-Toledo TGA/DSC3+ analyzer. Temperature calibration was performed using calcium oxalate, indium, tin, and zinc. The sample was placed in an aluminum pan. The pan was hermetically sealed, the lid was pierced, and the pan was then inserted into the TG furnace. A weighed aluminum pan configured as the sample pan was placed on the reference platform. The furnace was heated under nitrogen. Samples were analyzed from 25° C. to 350° C. at 10° C./min.

Thermogravimetric analyses typically experience a period of equilibration at the start of each analysis, indicated by red parentheses on the thermograms. The starting temperature for relevant weight loss calculations is selected at a point beyond this region (typically above 35° C.) for accuracy.

DSC analysis on this instrument is less sensitive than on the DSC3+ differential scanning calorimeter. Therefore, samples with sufficient solids were analyzed by both instruments and only the TGA thermogram from this instrument is reported.

### X-ray Powder Diffraction (XRPD)

### Transmission Geometry (Most Samples)

XRPD patterns were collected with a PANalytical X'Pert PRO MPD or a PANalytical Empyrean diffractometer using an incident beam of Cu radiation produced using an Optix long, fine-focus source. An elliptically graded multilayer mirror was used to focus Cu Kα X-rays through the specimen and onto the detector. Prior to the analysis, a silicon specimen (NIST SRM 640e) was analyzed to verify the observed position of the Si 111 peak is consistent with the NIST-certified position. A specimen of the sample was sandwiched between 3-μm-thick films and analyzed in transmission geometry. A beam-stop, short antiscatter extension, and antiscatter knife edge were used to minimize the background generated by air. Sober slits for the incident and diffracted beams were used to minimize broadening from axial divergence. Diffraction patterns were collected using a scanning position-sensitive detector (X'Celerator) located 240 mm from the specimen and Data Collector software v. 5.5.

### Salt Screen of Compound 1

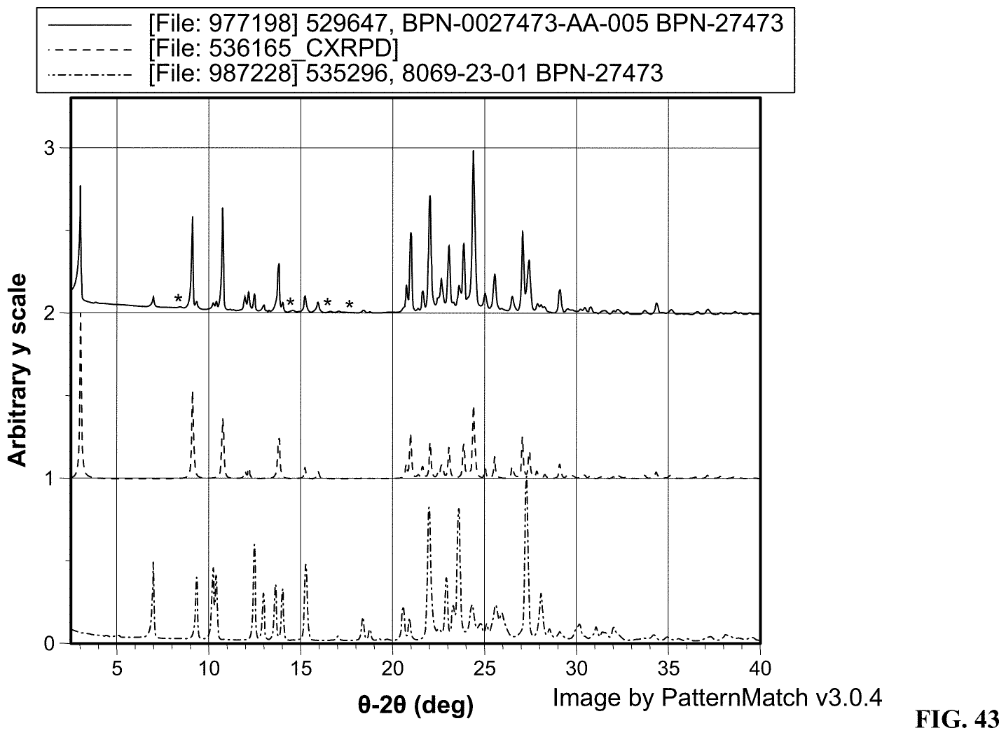

Unless otherwise indicated, reference to the use of Compound 1 in the below procedures refers to a mixture of Form A and Form B that is observable, in some circumstances, after carrying out the synthesis of Compound 1 (as detailed in WO 2019/246300; see FIG. 43), referred to herein as “crude”.

Based on the weakly basic pKₐ values of Compound 1, strong acids were selected for salt formation. The eight strong acids used include hydrochloric, sulfuric, benzenesulfonic, ethane-1,2-disulfonic, methanesulfonic, naphthalene-1,5-disulfonic, naphthalene-2-sulfonic and toluenesulfonic.

These experiments generally involved the direct addition of 0.5, 1, or 2 molar equivalents of acidic solutions to solutions or suspensions of the free base form of Compound 1. Materials were harvested immediately if precipitation of sufficient quantity occurred. If necessary, additional steps including (but not limited to) cooling, anti-solvent addition, melting/cooling, evaporation, and/or slurrying were performed in attempts to increase yield or crystallinity of the resulting material.

The products were qualitatively evaluated for crystallinity by polarized light microscopy (PLM) and/or x-ray powder diffraction (XRPD). Crystalline materials were successfully isolated with all eight strong acids used. However, the edisylate salt was found to be decomposed as determined by ¹H NMR. With the exception of the edisylate, at least one representative crystalline salt from each counterion was isolated. The data acquisition parameters for each pattern are displayed above the image in the Data section of this report. All images have the instrument labeled as X'Pert PRO MPD regardless of the instrument used.

### Compound 1 Free Base Polymorph Screen

Unless otherwise indicated, reference to the use of Compound 1 in the below procedures refers to a mixture of Form A and Form B that is observable, in some circumstances, after carrying out the synthesis of Compound 1 (as detailed in WO 2019/246300; see FIG. 43), referred to herein as “crude”.

A solvent-based screen designed to identify crystalline forms of Compound 1 is summarized in Table 11. More than 60 evaporative, slurry, crash precipitation, and cooling experiments were conducted. In some instances, solids were purposefully analyzed wet to further increase the likelihood of identifying hydrated or solvated forms. Water activity slurries were utilized to evaluate the propensity of Compound 1 to form hydrates and to help identify the stability range in which they would occur. Non-solvent based methods consisting of heat-induced transformations were also included. In addition, experiments to help determine the relative thermodynamic stability between anhydrous forms at various temperatures were conducted (see Relative Thermodynamic Stability).

TABLE 11
Polymorph Experiments of Compound 1

| Solvent | Method | Observation¹ | Result (Observed Form) |
|---|---|---|---|
| acetone | slurry, ambient, 1 d | off white, yellow solution | A + B |
| ACN | slurry, ambient, 1 d | white, yellow solution | A + B |
| DCM | 1. dissolved | 1. yellow solution | A |
| | 2. treated w/ charcoal | 2. reduction of hue | |
| | 3. fast evaporation | 3. aciculars, B | |
| | flash evap at 80 ° C. | solids formed rapidly, fine | A |
| | | aciculars, B | |
| | N₂ evaporation | off white solids, fines, oily | A |
| | | areas | |
| | solvent/anti-solvent | | E + A |
| | 1. 1 mL of DCM | 1. turbid | |
| | solution filtered into | 2.fine aciculars, B | |
| | 10 ml Et₂O | | |
| | 2. 10 ml of Et₂O added | | |
| | rotary evaporation | off white fines, B | D + A + peaks |
| | rotary evaporation | fines, B | D + A |
| Solvent | Method | Observation² | Result (Observed Form) |
| DCM | 1. seeded w/ Form E of Compound 1 | 1. — | A |
| | 2. N₂ fast evaporation | 2. — | |
| | 1. saturated solution at ambient, | 1. clear solution | A |
| | filtered | 2. seed retained | |
| | 2. seeded w/ Form E of Compound 1 | 3. fine aciculars, B | |
| | 3. Et₂O added | | |
| | 1. volume reduced w/ heat | 1. clear solution | A |
| | 2. seeded w/ Form E of Compound 1 | 2. seed retained | |
| | 3. cold Et₂O added | 3. precipitates formed | |
| | 4. filtered | 4. aciculars, B | |
| | 1. saturated solution at ambient, | 1. clear solution | A + E |
| | filtered | 2.— | |
| | 2. seeded w/ Form E of Compound 1 | 3. fine aciculars, B | |
| | in cold Et₂O | | |
| | 3. filtered | | |
| DMF | solvent/anti-solvent | | |
| | 1. 80° C. DMF solution filtered into EtOH | 1. no changes | A |
| | (RT) w/ Form A of Compound 1 | 2. nucleation, ~2 min. | |
| | 2. stirred, 1 hour 3. filtered, | 3. off-white solids | |
| | vacuum dried overnight | | |
| | solvent/anti-solvent 1. 80° C. DMF solution | 1. nucleation ~10 min. | A + E |
| | filtered into EtOH (RT) w/8010-94-03 | 2. — | |
| | (Form E) | 3. — | |
| | 2. filtered, EtOH wash | | |
| | 3. vacuum overnight | | |
| | slow cool, 80° C. treated with charcoal | white fine aciculars, B | A |
| | solvent/anti-solvent | | |
| | 1. DMF solution filtered into water | 1. gel, few fines, B | diffuse |
| | 2. sub sample isolated on slide, | 2. gel collapsed, fines | scatter |
| | smashed | 3. — | |
| | 3. bulk seeded with slide material | 4. −5. gel | |
| | 4. slurry, ambient, 1 d | | |
| | 5. centrifuged sub sample | | |
| | 5. slurry Compound 1 centrifuged, | would not filter | A |
| | decanted, dried under N₂ | | |
| Solvent | Method | Observation³ | Result (Observed Form) |
| DMF | solvent/anti-solvent | | A⁴ |
| | 1. DMF solution | 1. clear solution | |
| | filtered into ethanol | 2. blades | |
| | 2. refrigerated, 3 d | | |
| | solvent/anti-solvent 1. 80° C. | 1. clear solution | A |
| | DMF solution filtered into | 2. nucleation after ~2 min. | |
| | EtOH (RT) | 3. off-white solids | |
| | 2. stirring | | |
| | 3. filtered, vacuum | | |
| | dried overnight | | |
| | 1. heated to reflux to | 1. clear | A |
| | reduce EtOH | 2. no changes | |
| | 2. water added | 3. aciculars after 2 days | |
| | 3. slurry, 6 days | 4. light brown aciculars, B | |
| | 4. filtered, rinsed w/water, | | |
| | N₂ dried | | |
| Et₂O | slurry, ambient, 1 d | off white, yellow solution | A + B |
| EtOH | slow cool, 50° C. | aciculars, wispy, B | A |
| EtOAc | slurry, ambient, 1 d | off white, yellow solution | A + B |
| MeOH | slurry, ambient, 1 d | white, yellow solution | C |
| | 1. iterative wash 2. | 1. white solids | C + peaks |
| | filter cake rinsed | 2. — | |
| | with DCM | | |
| | slurry, ambient, ½ hr, N₂ | — | C |
| | dried at 60° C. | | |
| | 1. Form F MeOH, sonicated | 1. gel then broke | C + peaks |
| | 2. centrifuged, decanted | 2. — | |
| THF | fast evaporation | yellow dendritic, B | A |
| | slow cool | white fines, B | E |
| toluene | slurry, ambient, 1 d | off-white, yellow solution | A + B |
| Water | slurry, ambient, 1 d | tan solids, yellow solution | A + B |
| | Napsylate salt of Compound 1 | gel and fines, B | disordered E |
| | and Form E of Compound 1 | | |
| | slurry, ambient, 8 d | | |
| | Disordered E from above sub | gel | diffuse |
| | sampled after 2 days, | | scatter |
| | centrifuge, left wet | | |
| | HCl salt of Compound 1 slurry, | tan solids | disordered F |
| | ambient, 6 days | | |
| water/DMF | slurry, ambient, 19 d | — | A + B |
| 90:10 v/v | | | |
| (0.97 a_{w}) | | | |
| Solvent | Method | Observation⁵ | Result (Observed Form) |
| water/DMF | A mixture of Form | — | A |
| 90:10 v/v | A and B of | | |
| (0.97 a_{w}) | Compound 1 slurry, | | |
| | ambient, 6 d | | |
| | 1. 2 ml DMF | 1. irregular mass, limited | diffuse scatter + F |
| | solution (12 mg/mL) | amount of fines, B | |
| | slowly added to 18 mL | 2. filtrate pH 4.5 | |
| | H₂O seeded w/ 13.8 mg | | |
| | of Form F of Compound 1 | | |
| | 2. subdivided | | |
| | Form F of | — | diffuse scatter + F |
| | Compound 1 left | | |
| | at ambient, 5 d | | |
| | Form F of | — | diffuse scatter + F |
| | Compound 1 left at | | |
| | ambient, 14 d | | |
| | Form F of Compound 1 | | |
| | 1. 55° C. slurry, 1 d | 1. solids settled, blades | Form E |
| | 2. continued 5 d total | 2. no changes | |
| | 3. filtered, N₂ dried | 3. low recovery | |
| | Form F of Compound 1 | | |
| | 1. dilute HCl added | 1. pH: 1.5 | diffuse scatter + F |
| | 2. subdivided | 2. — | |
| | 3. ambient, 5 d | 3. — | |
| | 2. Form F of Compound 1 | 2. — | diffuse scatter + F |
| | 3. ambient, 9 d | 3. — | |
| water/DMF | slurry, ambient, 19 d | — | A +B |
| 50:50 v/v | A mixture of Form | — | A + B |
| (0.70 a_{w}) | A and B of | | |
| | Compound 1 slurry, | | |
| | ambient, 6 d | | |
| | 0.5 mL DMF solution (12 | aciculars, B | A |
| | mg/mL) slowly added to | | |
| | 0.5 mL H₂O seeded w/ | | |
| | 6.7 mg Form F | | |
| water/DMF | 1 mL water added slowly | aciculars after 5 min., B | A |
| 25:75 v/v | to 3 mL DMF solution (12 | | |
| (0.45 a_{w}) | mg/mL) | | |
| | 1 mL water seeded w/ | aciculars, B | A |
| | 8010-85-03 (Form F) added | | |
| | to 3 mL DMF solution (12 | | |
| | mg/mL) | | |

¹B = birefringent.
²B = birefringent.
³B = birefringent.
⁴Single crystal isolated
⁵B = birefringent.

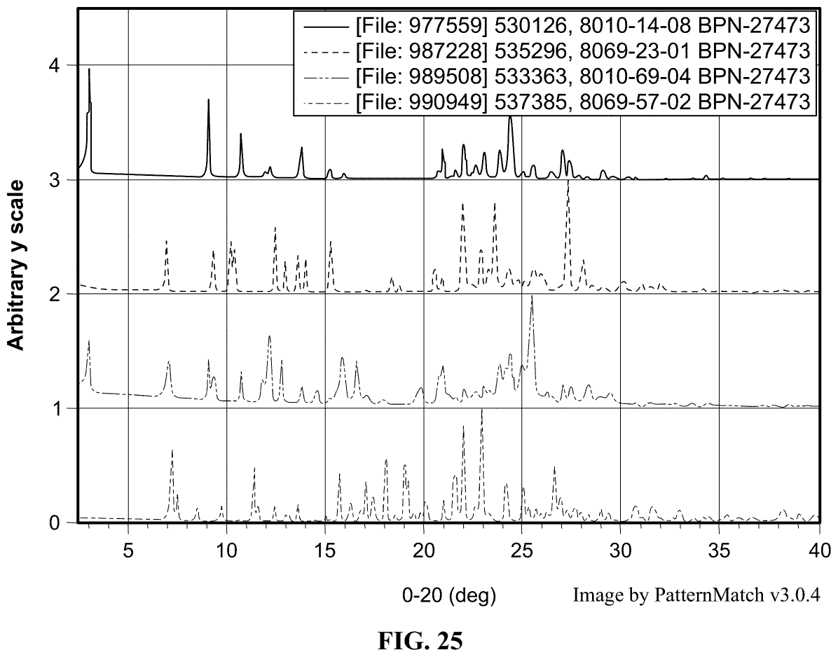

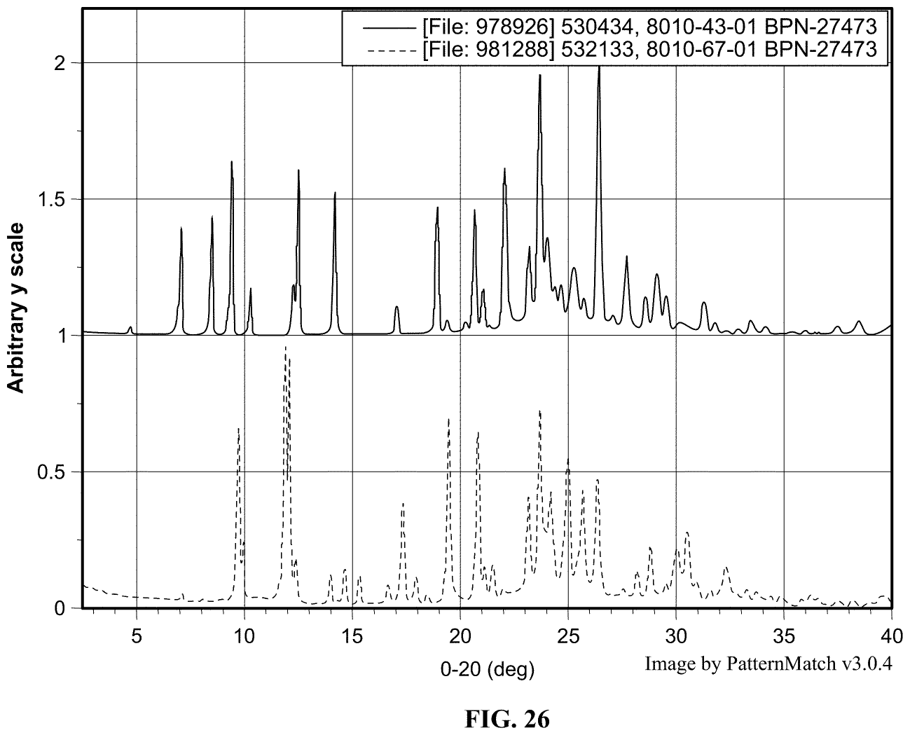

Forms A, B, Material D, and Form E are anhydrous forms of Compound 1; Form F is a hydrate; and Form C is a methanolate. The X-ray powder patterns of these forms are compared in FIGS. 25 and 26. Crystalline Form A Anhydrate of compound 2 exhibits limited hygroscopicity, a decomposition onset of 207° C., and was identified as the most thermodynamically stable, relative to the other anhydrous forms of Compound 1. Crystalline Form B of Compound 1 is a metastable desolvate, and is obtained through the desolvation of crystalline Form C Methanolate upon overnight exposure to 80° C. Crystalline Form E of Compound 1 is a metastable anhydrate and was most frequently observed through the disproportionation of various salts of Compound 1 in water. Crystalline Form F of Compound 1 is a hydrate and was generated by slurrying the HCl salt of Compound 1, in water. It is probable that the hydrate results from the displacement of CF from the crystal structure, which is unlikely to occur without the HCl salt as an intermediate. The hydrate was shown to remain unchanged for 5 days under vacuum at ambient temperature but does dehydrate with concomitant decomposition upon exposure to 100° C. Characterization data is discussed in more detail in subsequent sections below.

### Anhydrous Forms

### Form A, Stable Anhydrate

Crystalline Form A is an anhydrate of Compound 1 with a decomposition onset of 207° C. (FIGS. 27A and 27B). Form A is the most thermodynamically stable, relative to the other anhydrous forms, at ambient temperature (see Relative Thermodynamic Stability).

Form A was routinely observed from various solvents and can be generated through slurries in solvents with adequate solubility, evaporations, cooling of saturated solutions, and solvent/anti-solvent additions (see Table 11). For example, dissolving Compound 1 in dichloromethane (DCM) followed by either flash evaporation at 80° C. or under N₂ led to the isolation of Form A of Compound 1. Further seeding the DCM solution with Form E of Compound 1, followed by fast evaporation under N₂ also resulted in the isolation of pure Form A of Compound 1. Various other experiments in dimethylformamide (DMF), tetrahydrofuran (THF), Ethanol (EtOH), and water DMF mixtures also lead to the isolation of pure Form A of Compound 1.

The XRPD pattern and the peak list for Form A of compound 1 is illustrated in FIG. 29 (experimental, top) and Table 12, respectively.

TABLE 12
Observed peaks for Form A of Compound 1.

| ° 2θ | d space (Å) | Intensity (%) |
|---|---|---|
| 3.02 ± 0.20 | 29.232 ± 1.935 | 100 |
| 9.10 ± 0.20 | 9.710 ± 0.213 | 71 |
| 10.74 ± 0.20 | 8.231 ± 0.153 | 43 |
| 11.95 ± 0.20 | 7.400 ± 0.123 | 10 |
| 12.16 ± 0.20 | 7.273 ± 0.119 | 15 |
| 13.79 ± 0.20 | 6.416 ± 0.093 | 32 |
| 15.22 ± 0.20 | 5.817 ± 0.076 | 12 |
| 15.93 ± 0.20 | 5.559 ± 0.069 | 8 |
| 18.46 ± 0.20 | 4.802 ± 0.052 | 4 |
| 20.74 ± 0.20 | 4.279 ± 0.041 | 11 |
| 20.97 ± 0.20 | 4.233 ± 0.040 | 30 |
| 21.36 ± 0.20 | 4.156 ± 0.038 | 6 |
| 21.62 ± 0.20 | 4.107 ± 0.038 | 11 |
| 22.04 ± 0.20 | 4.030 ± 0.036 | 33 |
| 22.66 ± 0.20 | 3.921 ± 0.034 | 16 |
| 23.06 ± 0.20 | 3.854 ± 0.033 | 27 |
| 23.86 ± 0.20 | 3.726 ± 0.031 | 29 |
| 24.40 ± 0.20 | 3.645 ± 0.029 | 59 |
| 25.03 ± 0.20 | 3.555 ± 0.028 | 10 |
| 25.55 ± 0.20 | 3.484 ± 0.027 | 16 |
| 26.51 ± 0.20 | 3.360 ± 0.025 | 11 |
| 27.07 ± 0.20 | 3.291 ± 0.024 | 28 |
| 27.43 ± 0.20 | 3.249 ± 0.023 | 19 |
| 27.85 ± 0.20 | 3.201 ± 0.023 | 7 |
| 28.29 ± 0.20 | 3.152 ± 0.022 | 5 |
| 29.09 ± 0.20 | 3.067 ± 0.021 | 11 |
| 29.52 ± 0.20 | 3.023 ± 0.020 | 6 |
| 30.44 ± 0.20 | 2.934 ± 0.019 | 5 |
| 30.76 ± 0.20 | 2.904 ± 0.018 | 4 |

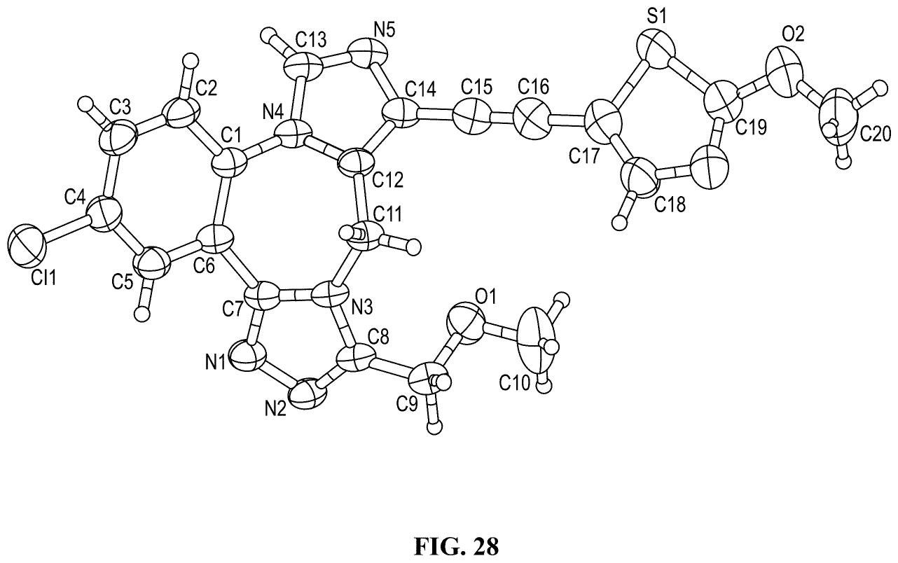

The single-crystal structure of Form A was determined successfully (FIG. 28). Single crystals suitable for X-ray diffraction of Form A were obtained by dissolving Compound 1 in dimethylformamide, filtering the solution into ethanol, and cooling the mixture in a refrigerator (4° C.) for a period of 3 days to induce crystallization of Form A. The crystal system is monoclinic and the space group is Cite. The cell parameters and calculated volume are: a=58.1415(14) Å, b=4.03974(8) Å, c=17.1204(3) Å, α=90°, β=90.261(2°), γ=90°, V=4021.15(14) Å³. The molecular weight is 438.89 g mol⁻¹ with Z=8, resulting in a calculated density of 1.450 g cm⁻³. Further details of the crystal data and crystallographic data collection parameters are summarized in Table 13. The asymmetric unit contains one Compound 1 molecule. The thiazole and ether are rotated by 180°, refining to 88% occupancy in the predominant orientation. An atomic displacement ellipsoid drawing of Compound 1 Form A in the predominant orientation is shown in FIG. 28. The calculated XRPD pattern, from the single crystal data, is compared to the experimental pattern in FIG. 29.

TABLE 13
Crystal Data and Data Collection Parameters for Form A.

| Empirical formula | C₂₀ H₁₅ClN₆O₂S |
|---|---|
| Formula weight (g mol⁻¹) | 438.89 |
| Temperature (K) | 293(9) |
| Wavelength (Å) | 1.54184 |
| Crystal system | monoclinic |
| Space group | C2/c |
| Unit cell parameters | |
| a = 58.1415(14) Å | α = 90° |
| b = 4.03974(8) Å | β = 90.261(2)° |
| c = 17.1204(3) Å | γ = 90° |
| Unit cell volume (Å⁻³) | 4021.15(14) |
| Cell formula units, Z | 8 |
| Calculated density (g cm⁻³) | 1.450 |
| Absorption coefficient (mm⁻¹) | 2.917 |
| F(000) | 1808 |
| Crystal size (mm³) | 0.38 × 0.04 × 0.02 |
| Reflections used for cell measurement | 6912 |
| ϑ range for cell measurement | 5.1510°-76.6050° |
| Total reflections collected | 9098 |
| Index ranges | −53 ≤ h ≤ 71; −4 ≤ k ≤ 4; −21 ≤/≤ 21 |
| ϑ range for data collection | ϑₘᵢₙ = 4.563°, ϑₘₐₓ = 77.412° |
| Completeness to ϑₘₐₓ | 95% |
| Completeness to ϑ_{full} = 67.684° | 99.8% |
| Absorption correction | multi-scan |
| Transmission coefficient range | 0.681-1.000 |
| Refinement method | full matrix least-squares on F² |
| Independen reflections | 4047 [Rᵢₙₜ = 0.0225, R_{σ} = 0.0298] |
| Reflections [ I > 2σ(I) ] | 3294 |
| Reflections/restraints/parameters | 4047/7/299 |
| Goodness-of-fit on F² | S = 1.03 |
| Final residuals [ I > 2σ(I) ] | R = 0.0381, R_{w} = 0.1057 |
| Final residuals [ all reflections ] | R = 0.0471, R_{w} = 0.1125 |
| Largest diff. peak and hole (e Å⁻³) | 0.247, −0.281 |
| Max/mean shift/standard uncertainty | 0.001/0.000 |

Thermograms of Form A are shown in FIGS. 27A and 27B. The TGA does not show weight loss up to 207° C., consistent with an anhydrous form. The DSC curve exhibits an exotherm, due to decomposition, with an onset at about 207° C.

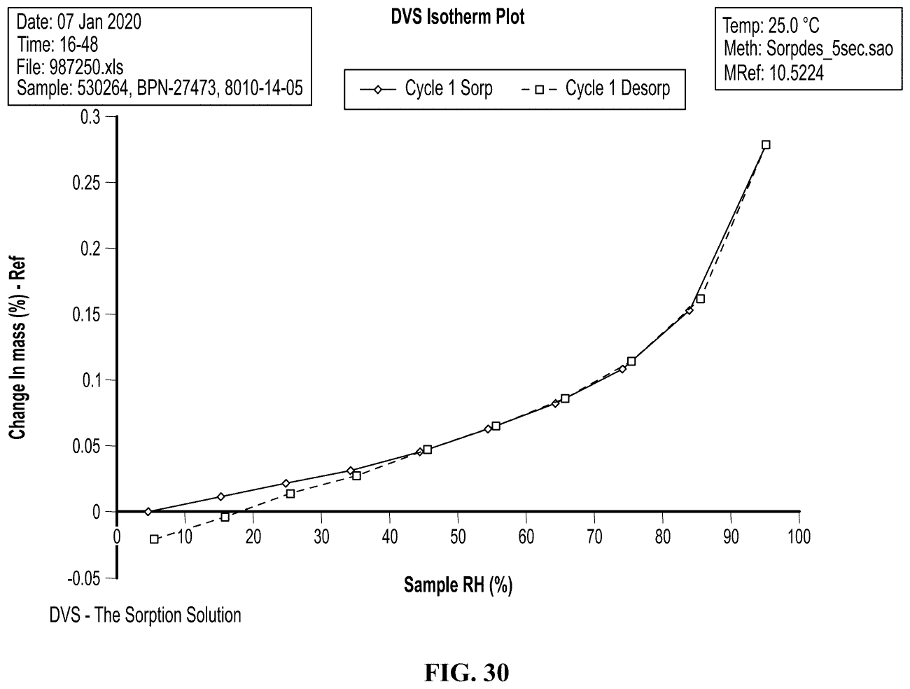

The dynamic vapor sorption (DVS) isotherm suggests that Form A exhibits low hygroscopicity (FIG. 30). Hygroscopicity can be described as low, limited, or significant in part on concepts presented in reference (see Dynamic Vapor Absorption Experimental). The weight change through the sorption/desorption cycle was negligible at ˜0.3% with no hysteresis. The material recovered from the DVS experiment was identified as the same as the starting material by XRPD.

### Form B, Metastable Desolvate

Form B is a metastable anhydrate of Compound 1 obtained through the desolvation of polymorphic Form C Methanolate of Compound 1 upon overnight exposure to 80° C. Based on the thermograms for Form C, the desolvated form (Form B) exhibits a decomposition onset at about 190° C. Form B was shown to convert to Form A in solvent-mediated experiments at ambient temperature (see Relative Thermodynamic Stability), confirming that Form B is metastable relative to Form A at that condition.

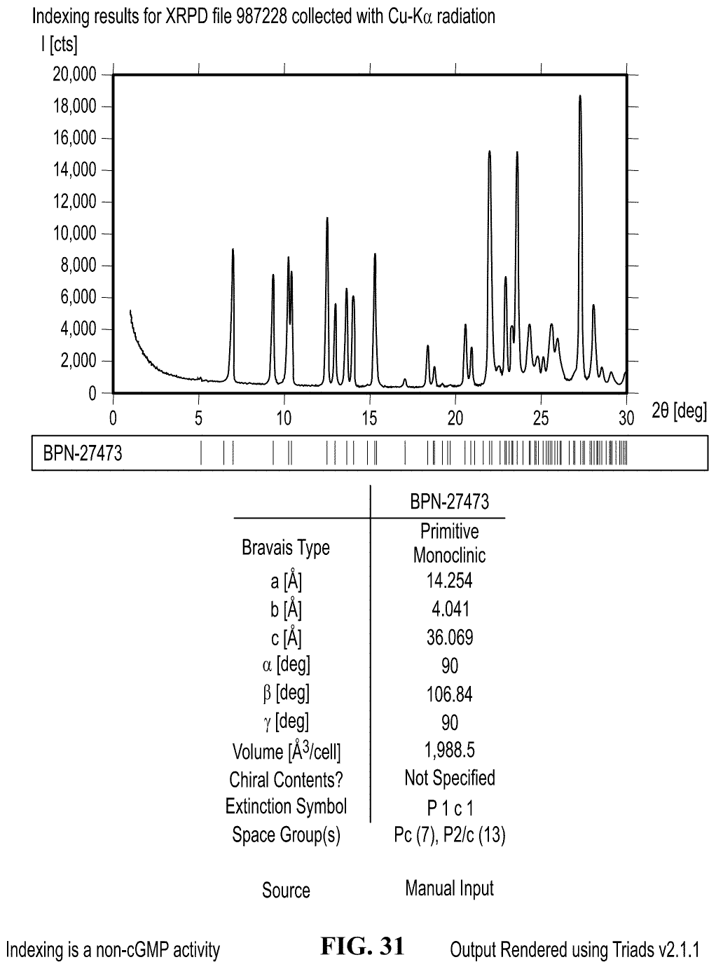

The XRPD pattern and its peak list for Form B of compound 1 are illustrated in FIG. 34 and Table 14, respectively The XRPD pattern of Form B was successfully indexed and provides a robust description of the crystalline form through tentative crystallographic unit cell parameters and strong evidence that the pattern is representative of a single crystalline phase (FIG. 31). The form has a monoclinic unit cell likely containing four Compound 1 molecules. Consequently, the formula unit volume of 497 Å³ calculated from the indexing results would be consistent with an anhydrous form.

TABLE 14
Observed peaks for Form B of compound 1.

| ° 2θ | d space (Å) | Intensity (%) |
|---|---|---|
| 5.09 ± 0.20 | 17.347 ± 0.681 | 5 |
| 6.99 ± 0.20 | 12.636 ± 0.361 | 47 |
| 9.34 ± 0.20 | 9.461 ± 0.202 | 39 |
| 10.23 ± 0.20 | 8.640 ± 0.168 | 46 |
| 10.40 ± 0.20 | 8.499 ± 0.163 | 41 |
| 12.48 ± 0.20 | 7.087 ± 0.113 | 58 |
| 12.97 ± 0.20 | 6.820 ± 0.105 | 30 |
| 13.62 ± 0.20 | 6.496 ± 0.095 | 35 |
| 14.01 ± 0.20 | 6.316 ± 0.090 | 32 |
| 15.28 ± 0.20 | 5.794 ± 0.075 | 47 |
| 17.03 ± 0.20 | 5.202 ± 0.061 | 5 |
| 18.38 ± 0.20 | 4.823 ± 0.052 | 16 |
| 18.76 ± 0.20 | 4.726 ± 0.050 | 9 |
| 19.23 ± 0.20 | 4.612 ± 0.048 | 3 |
| 19.68 ± 0.20 | 4.507 ± 0.045 | 3 |
| 20.57 ± 0.20 | 4.314 ± 0.041 | 23 |
| 20.92 ± 0.20 | 4.243 ± 0.040 | 15 |
| 21.98 ± 0.20 | 4.041 ± 0.036 | 81 |
| 22.54 ± 0.20 | 3.941 ± 0.035 | 9 |
| 22.92 ± 0.20 | 3.877 ± 0.033 | 39 |
| 23.29 ± 0.20 | 3.816 ± 0.032 | 22 |
| 23.60 ± 0.20 | 3.767 ± 0.031 | 81 |
| 24.31 ± 0.20 | 3.658 ± 0.030 | 23 |
| 24.78 ± 0.20 | 3.590 ± 0.029 | 12 |
| 25.11 ± 0.20 | 3.544 ± 0.028 | 12 |
| 25.60 ± 0.20 | 3.477 ± 0.027 | 23 |
| 25.94 ± 0.20 | 3.432 ± 0.026 | 18 |
| 27.28 ± 0.20 | 3.266 ± 0.023 | 100 |
| 28.07 ± 0.20 | 3.176 ± 0.022 | 30 |
| 28.52 ± 0.20 | 3.127 ± 0.021 | 9 |
| 29.09 ± 0.20 | 3.067 ± 0.021 | 7 |
| 30.17 ± 0.20 | 2.960 ± 0.019 | 12 |
| 31.07 ± 0.20 | 2.876 ± 0.018 | 10 |

### Material D, Metastable Anhydrate

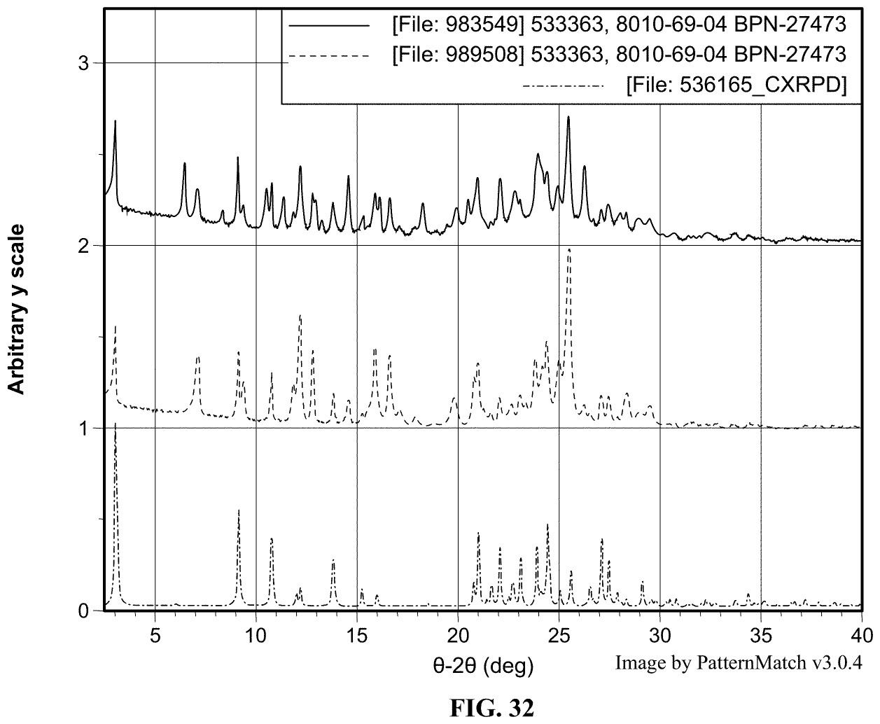

Material D of Compound 1 is tentatively identified as an anhydrate. Material D was only obtained as a mixture with Form A (and additional unidentified peaks) from failed attempts to isolate amorphous Compound 1 through rotary evaporations out of DCM. Although the additional unidentified peaks in the XRPD diffractogram were no longer evident after 7 weeks of ambient storage, Material D still remained (FIG. 32). This implies that Material D exhibits some kinetic stability at ambient temperature. Regardless, Material D was shown to convert to Form A in solvent-mediated experiments at ambient temperature (see Relative Thermodynamic Stability), confirming that Material D is metastable relative to Form A at that condition.

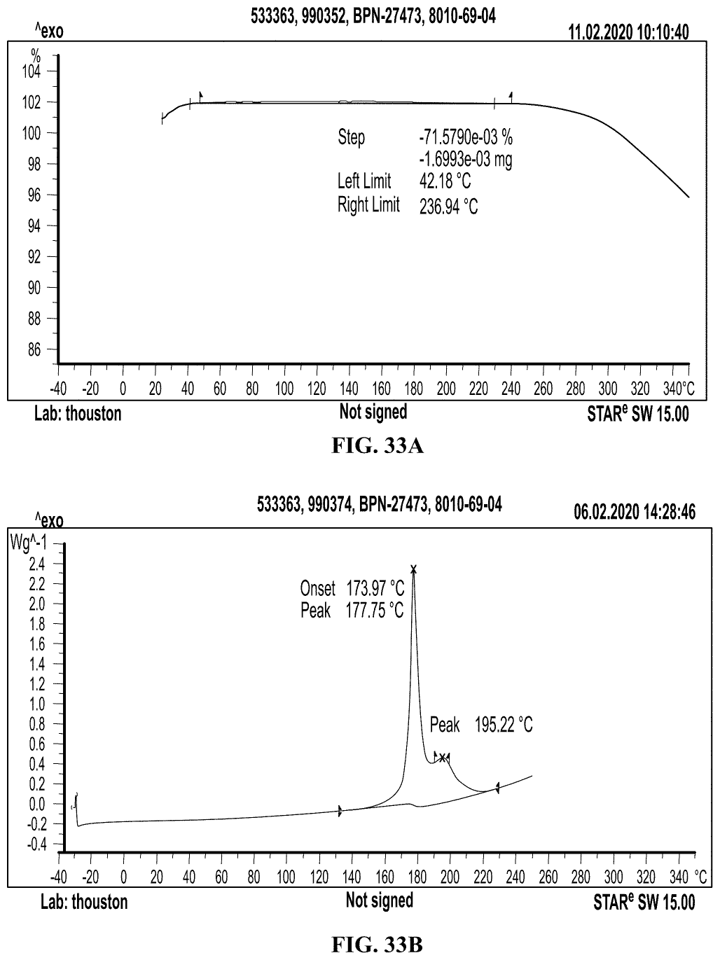

Thermograms of Material D (as a mixture with Form A) are shown in FIGS. 33A and 33B. The TGA does not show weight loss up to 237° C., consistent with a mixture of anhydrous forms. The DSC exhibits exotherms, due to decomposition, with an onset near 174° C.

### Form E, Metastable Anhydrate

Form E is an anhydrate of Compound 1 with a decomposition onset of 201° C. (FIGS. 36A and 36B). Form E is metastable relative to Form A; the relative thermodynamic relationship was confirmed with interconversion slurry experiments performed at ambient temperature, 55° C., and 77° C. (see Relative Thermodynamic Stability). Form E was most frequently observed through the disproportionation of various salts of Compound 1 in water. A crystal suitable for single crystal x-ray diffraction was obtained by slowly cooling a THF solution saturated with amorphous Compound 1.

The XRPD pattern and the peak list for Form E of compound 1 is illustrated in FIG. 35 (experimental, top) and Table 15, respectively.

TABLE 15
Observed peaks for Form E of compound 1.

| ° 2θ | d space (Å) | Intensity (%) |
|---|---|---|
| 7.24 ± 0.20 | 12.198 ± 0.336 | 65 |
| 7.49 ± 0.20 | 11.788 ± 0.314 | 27 |
| 8.48 ± 0.20 | 10.413 ± 0.245 | 14 |
| 9.73 ± 0.20 | 9.079 ± 0.186 | 17 |
| 10.71 ± 0.20 | 8.255 ± 0.154 | 5 |
| 11.40 ± 0.20 | 7.757 ± 0.136 | 50 |
| 11.57 ± 0.20 | 7.640 ± 0.132 | 16 |
| 12.43 ± 0.20 | 7.116 ± 0.114 | 16 |
| 13.00 ± 0.20 | 6.806 ± 0.104 | 8 |
| 13.13 ± 0.20 | 6.738 ± 0.102 | 9 |
| 13.61 ± 0.20 | 6.500 ± 0.095 | 18 |
| 14.32 ± 0.20 | 6.181 ± 0.086 | 3 |
| 14.53 ± 0.20 | 6.092 ± 0.083 | 4 |
| 15.04 ± 0.20 | 5.885 ± 0.078 | 10 |
| 15.27 ± 0.20 | 5.797 ± 0.075 | 4 |
| 15.74 ± 0.20 | 5.624 ± 0.071 | 45 |
| 16.30 ± 0.20 | 5.435 ± 0.066 | 20 |
| 16.82 ± 0.20 | 5.268 ± 0.062 | 13 |
| 17.05 ± 0.20 | 5.197 ± 0.061 | 38 |
| 17.44 ± 0.20 | 5.080 ± 0.058 | 23 |
| 18.07 ± 0.20 | 4.906 ± 0.054 | 58 |
| 18.53 ± 0.20 | 4.784 ± 0.051 | 9 |
| 19.03 ± 0.20 | 4.660 ± 0.049 | 51 |
| 19.19 ± 0.20 | 4.621 ± 0.048 | 39 |
| 19.51 ± 0.20 | 4.546 ± 0.046 | 10 |
| 19.85 ± 0.20 | 4.469 ± 0.045 | 11 |
| 20.09 ± 0.20 | 4.416 ± 0.044 | 21 |
| 21.01 ± 0.20 | 4.225 ± 0.040 | 22 |
| 21.54 ± 0.20 | 4.122 ± 0.038 | 42 |
| 21.62 ± 0.20 | 4.106 ± 0.038 | 44 |
| 22.01 ± 0.20 | 4.035 ± 0.036 | 86 |
| 22.64 ± 0.20 | 3.924 ± 0.034 | 16 |
| 22.92 ± 0.20 | 3.877 ± 0.033 | 100 |
| 23.29 ± 0.20 | 3.816 ± 0.032 | 10 |
| 24.18 ± 0.20 | 3.678 ± 0.030 | 36 |
| 25.04 ± 0.20 | 3.554 ± 0.028 | 33 |
| 25.33 ± 0.20 | 3.513 ± 0.027 | 17 |
| 25.69 ± 0.20 | 3.465 ± 0.027 | 14 |
| 25.93 ± 0.20 | 3.434 ± 0.026 | 9 |
| 26.21 ± 0.20 | 3.397 ± 0.025 | 12 |
| 26.64 ± 0.20 | 3.344 ± 0.025 | 51 |
| 26.93 ± 0.20 | 3.309 ± 0.024 | 24 |
| 27.24 ± 0.20 | 3.271 ± 0.024 | 12 |
| 27.64 ± 0.20 | 3.225 ± 0.023 | 15 |
| 27.97 ± 0.20 | 3.187 ± 0.022 | 10 |
| 28.35 ± 0.20 | 3.145 ± 0.022 | 9 |
| 28.99 ± 0.20 | 3.078 ± 0.021 | 14 |
| 29.37 ± 0.20 | 3.038 ± 0.020 | 11 |
| 30.72 ± 0.20 | 2.908 ± 0.018 | 16 |
| 31.07 ± 0.20 | 2.876 ± 0.018 | 7 |
| 31.57 ± 0.20 | 2.831 ± 0.017 | 17 |

The single-crystal structure of Form E was determined successfully (FIG. 34). The crystal system is monoclinic and the space group is P2₁/n. The cell parameters and calculated volume are: a=11.83974(13) Å, b=23.5195(2) Å, c=14.48807(17) Å, α=90°, β=101.5333(11°), γ=90°, V=3952.96(7) Å³. The molecular weight is 438.89 g mol⁻¹ with Z=8, resulting in a calculated density of 1.475 g cm⁻³. Further details of the crystal data and crystallographic data collection parameters are summarized in Table 16. An atomic displacement ellipsoid drawing of Compound 1 Form E is shown in FIG. 34. The asymmetric unit shown contains two Compound 1 molecules. The calculated powder pattern is compared to the experimental pattern in FIG. 35.

TABLE 16
Crystal Data and Data Collection Parameters for Form E

| Empirical formula | C₂₀ H₁₅ClN₆O₂S |
|---|---|
| Formula weight (g mol⁻¹) | 438.89 |
| Temperature (K) | 299.8(9) |
| Wavelength (Å) | 1.54184 |
| Crystal system | monoclinic |
| Space group | P2₁/n |
| Unit cell parameters | |
| a = 11.83974(13) Å | α = 90° |
| b = 23.5195(2) Å | β = 101.5333(11)° |
| c = 14.48807(17) Å | γ = 90° |
| Unit cell volume (Å⁻³) | 3952.96(7) |
| Cell formula units, Z | 8 |
| Calculated density (g cm⁻³) | 1.475 |
| Absorption coefficient (mm⁻¹) | 2.968 |
| F(000) | 1808 |
| Crystal size (mm³) | 0.19 × 0.1 × 0.04 |
| Reflections used for cell measurement | 9088 |
| ϑ range for cell measurement | 3.6260°-76.9860° |
| Total reflections collected | 21119 |
| Index ranges | −14 ≤ h ≤ 12; −21 ≤ k ≤ 29; |
| | −18 ≤/≤ 18 |
| ϑ range for data collection | ϑₘᵢₙ = 3.637°, ϑₘₐₓ = 77.701° |
| Completeness to ϑₘₐₓ | 97.4% |
| Completeness to ϑ_{full} = 67.684° | 100% |
| Absorption correction | multi-scan |
| Transmission coefficient range | 0.888-1.000 |
| Refinement method | full matrix least-squares on F² |
| Independen reflections | 8184 [Rᵢₙₜ = 0.0266, R_{σ} = 0.0339] |
| Reflections [ I > 2σ(I) ] | 6319 |
| Reflections/restraints/parameters | 8184/0/545 |
| Goodness-of-fit on F² | S = 1.04 |
| Final residuals [ I > 2σ(I) ] | R = 0.0427, R_{w} = 0.1155 |
| Final residuals [ all reflections ] | R = 0.0572, R_{w} = 0.1243 |
| Largest diff. peak and hole (e Å⁻³) | 0.315, −0.321 |

Thermograms for Form E are shown in FIGS. 36A and 36B. The TGA does not show weight loss up to ˜200° C., consistent with an anhydrous form. The DSC curve exhibits an exotherm, due to decomposition, with an onset near 201° C.

### Hydrated Forms

### Form F Hydrate

Form F is a likely hydrate of Compound 1. Form F was generated by slurrying the HCl salt of Compound 1 in water (see Table 11). The hydrate was shown to remain unchanged for 5 days under vacuum at ambient temperature but does dehydrate upon exposure to 100° C. Thermal characterization suggests that decomposition occurs immediately upon dehydration at elevated temperatures.

The XRPD patterns of the HCl salt of Compound 1 and Free Base Form F hydrate are similar (FIG. 37), suggesting that the crystal structures are also similar. It is probable that the hydrate results from the displacement of CF from the structure. Multiple attempts to crystallize a hydrated form directly from the free base were unsuccessful-even with seeding with up to 50 wt %. Instead, gels of the free base would remain in aqueous solvent systems at high water activity or would eventually crystallize to Form A at water activities of 0.7 and below. Therefore, it is unlikely that hydrate formation of the free base will occur without the HCl salt as an intermediate.

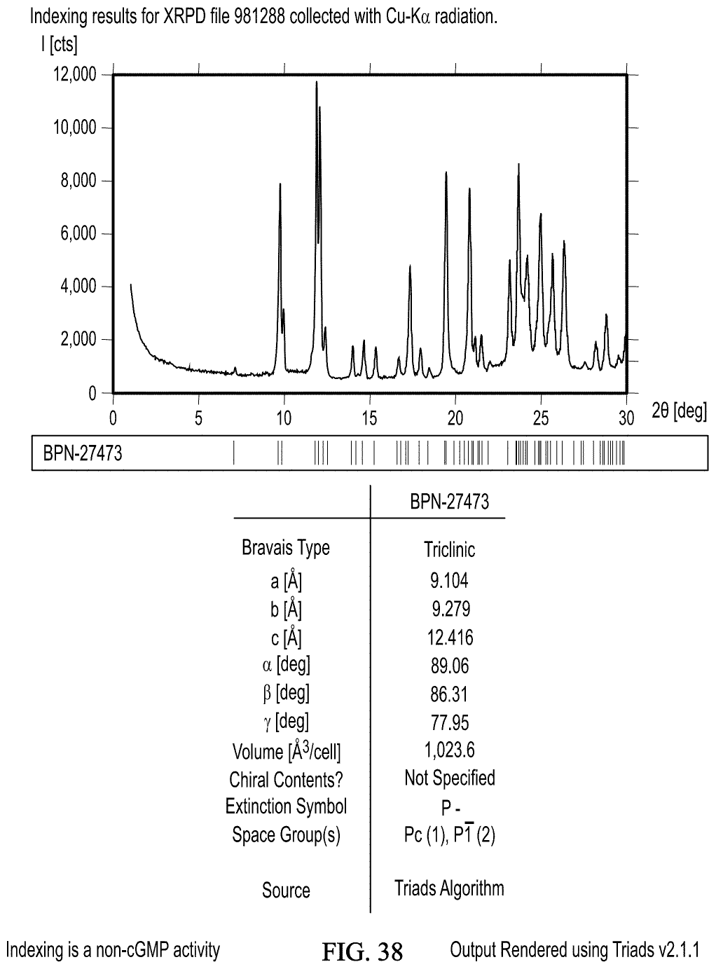

The XRPD pattern and its peak list for Form F of compound 1 are illustrated in FIG. 37 and Table 17, respectively The XRPD pattern was successfully indexed and provides strong evidence that the pattern is representative of a single crystalline phase (FIG. 38). The form has a triclinic unit cell likely containing two Compound 1 molecules. Consequently, the formula unit volume of 511 Å³ calculated from the indexing results would be consistent with a hydrate that can theoretically accommodate up to one mol/mol of water.

TABLE 17
Observed peaks for Form F of compound 1.

| ° 2θ | d space (Å) | Intensity (%) |
|---|---|---|
| 7.11 ± 0.20 | 12.423 ± 0.349 | 8 |
| 9.73 ± 0.20 | 9.083 ± 0.186 | 67 |
| 9.93 ± 0.20 | 8.900 ± 0.179 | 27 |
| 11.88 ± 0.20 | 7.443 ± 0.125 | 100 |
| 12.07 ± 0.20 | 7.327 ± 0.121 | 91 |
| 12.37 ± 0.20 | 7.150 ± 0.115 | 21 |
| 13.98 ± 0.20 | 6.330 ± 0.090 | 15 |
| 14.63 ± 0.20 | 6.050 ± 0.082 | 16 |
| 15.33 ± 0.20 | 5.775 ± 0.075 | 15 |
| 16.67 ± 0.20 | 5.314 ± 0.063 | 11 |
| 17.33 ± 0.20 | 5.113 ± 0.059 | 41 |
| 17.94 ± 0.20 | 4.940 ± 0.055 | 14 |
| 18.46 ± 0.20 | 4.802 ± 0.052 | 8 |
| 19.44 ± 0.20 | 4.562 ± 0.046 | 71 |
| 20.81 ± 0.20 | 4.265 ± 0.041 | 66 |
| 21.13 ± 0.20 | 4.201 ± 0.039 | 18 |
| 21.50 ± 0.20 | 4.130 ± 0.038 | 19 |
| 22.00 ± 0.20 | 4.037 ± 0.036 | 10 |
| 23.16 ± 0.20 | 3.837 ± 0.033 | 43 |
| 23.68 ± 0.20 | 3.754 ± 0.031 | 74 |
| 24.18 ± 0.20 | 3.678 ± 0.030 | 44 |
| 24.97 ± 0.20 | 3.563 ± 0.028 | 58 |
| 25.67 ± 0.20 | 3.468 ± 0.027 | 45 |
| 26.35 ± 0.20 | 3.380 ± 0.025 | 49 |
| 27.55 ± 0.20 | 3.235 ± 0.023 | 10 |
| 28.19 ± 0.20 | 3.163 ± 0.022 | 17 |
| 28.81 ± 0.20 | 3.096 ± 0.021 | 25 |
| 29.53 ± 0.20 | 3.022 ± 0.020 | 12 |
| 30.07 ± 0.20 | 2.969 ± 0.019 | 25 |
| 30.50 ± 0.20 | 2.929 ± 0.019 | 30 |
| 30.93 ± 0.20 | 2.889 ± 0.018 | 13 |
| 31.59 ± 0.20 | 2.830 ± 0.017 | 9 |
| 32.29 ± 0.20 | 2.770 ± 0.017 | 18 |

The solution ¹H NMR spectrum is consistent with the chemical structure of Compound 1. Peaks that could be attributed to residual organic solvent are not evident. Although derived from the HCl salt, ion chromatography quantitates a negligible amount of Cl⁻, confirming that Form F is a crystalline form of the free base.

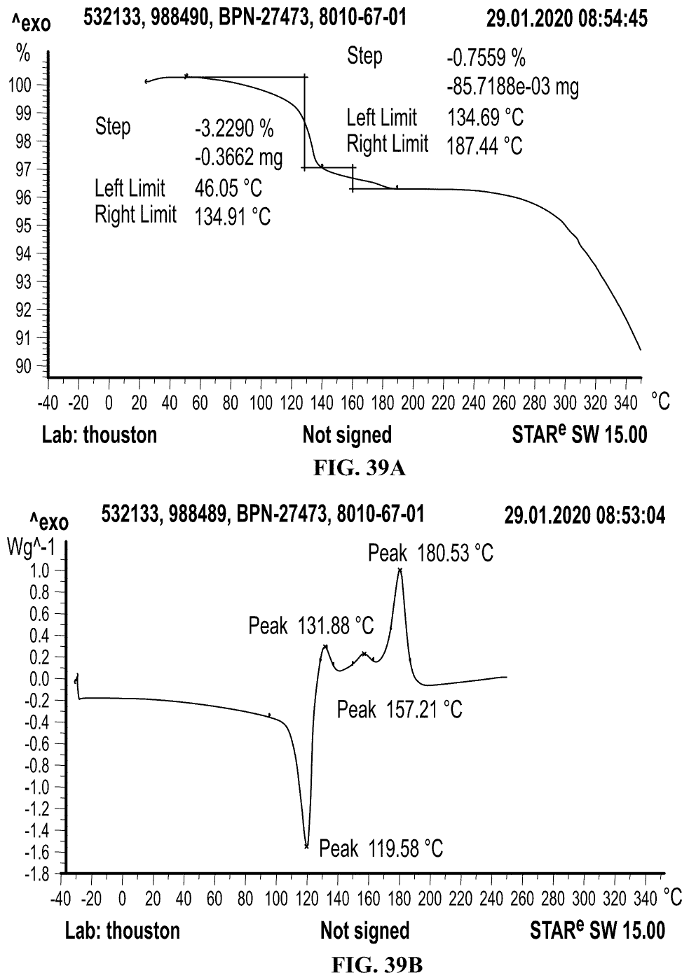

Thermograms for Form F are provided in FIGS. 39A and 39B. The TGA shows an initial 3.2% weight loss up to 135° C. and an additional 0.8% loss from 135 to 187° C. Assuming water is the only volatile (residual organic solvent was not evident in the NMR spectrum, discussed above), the weight loss in the initial step is equivalent to ˜0.8 moles of water per mole of Compound 1. The DSC curve exhibits a broad dehydration endotherm that immediately leads into exotherms above 120° C. The DSC exotherms suggest that decomposition occurs immediately upon dehydration. Accordingly, exposing the sample to 100° C. for several minutes resulted in loss of crystallinity by XRPD.

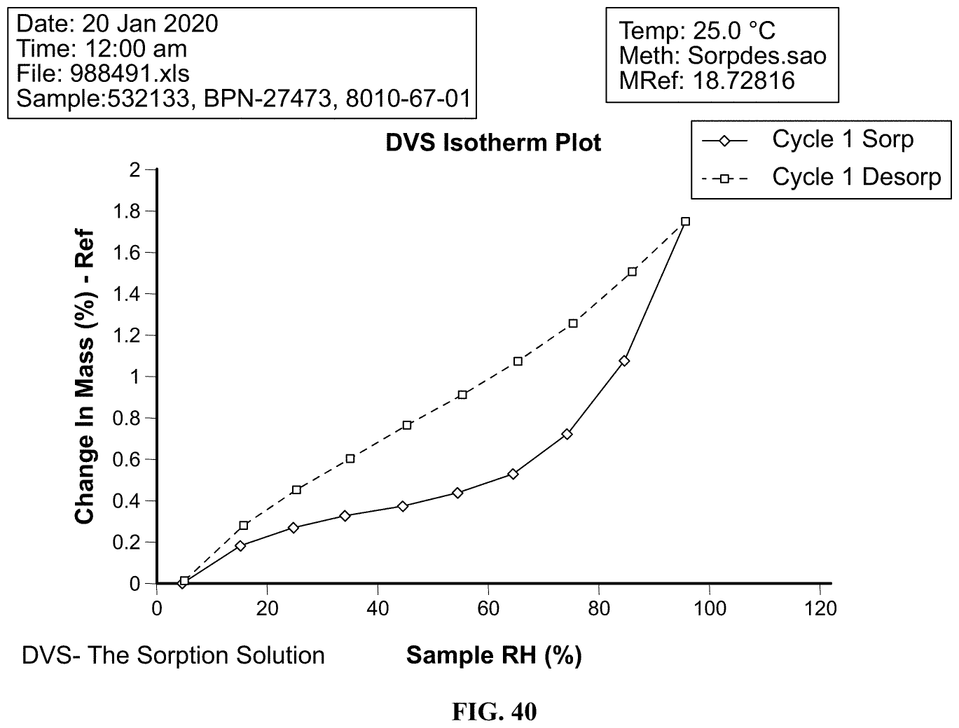

The DVS isotherm indicates Form F exhibits limited hygroscopicity (FIG. 40). A 1.8% weight gain from 5-95% RH and a 1.5% weight loss with significant hysteresis upon desorption is observed. The recovered post DVS sample was still Form F by XRPD.

### Solvated Forms

### Form C Methanolate

Form C is a methanolate observed from experiments involving methanol. In particular, amorphous Compound 1 was slurried in a methanol solution at ambient temperature for 30 minutes under N₂. The subsequent removal of the solvent at 60° C. resulted in isolation of Form C (Table 11). The solvate is kinetically stable and was shown to remain unchanged for 9 weeks under ambient conditions. However, the methanolate will desolvate to Form B (see Form B) upon overnight exposure to 80° C.

The XRPD pattern and its peak list for Form F of compound 1 are illustrated in FIG. 41 and Table 18, respectively. The XRPD pattern was successfully indexed and provides strong evidence that the pattern is representative of a single crystalline phase (FIG. 41). The form has a monoclinic unit cell likely containing four Compound 1 molecules. Consequently, the formula unit volume of 544 Å³ calculated from the indexing results would be consistent with a solvate that can theoretically accommodate up to one mol/mol of methanol.

TABLE 18
Observed peaks for Form C of compound 1.

| ° 2θ | d space (Å) | Intensity (%) |
|---|---|---|
| 4.71 ± 0.20 | 18.746 ± 0.796 | 6 |
| 7.06 ± 0.20 | 12.511 ± 0.354 | 41 |
| 8.50 ± 0.20 | 10.394 ± 0.244 | 44 |
| 9.41 ± 0.20 | 9.391 ± 0.199 | 65 |
| 10.27 ± 0.20 | 8.606 ± 0.167 | 20 |
| 12.26 ± 0.20 | 7.214 ± 0.117 | 21 |
| 12.49 ± 0.20 | 7.081 ± 0.113 | 62 |
| 14.17 ± 0.20 | 6.245 ± 0.088 | 54 |
| 17.05 ± 0.20 | 5.196 ± 0.061 | 13 |
| 18.92 ± 0.20 | 4.687 ± 0.049 | 49 |
| 19.39 ± 0.20 | 4.574 ± 0.047 | 8 |
| 20.26 ± 0.20 | 4.380 ± 0.043 | 8 |
| 20.65 ± 0.20 | 4.298 ± 0.041 | 47 |
| 21.05 ± 0.20 | 4.217 ± 0.040 | 19 |
| 22.06 ± 0.20 | 4.026 ± 0.036 | 63 |
| 23.19 ± 0.20 | 3.832 ± 0.033 | 35 |
| 23.67 ± 0.20 | 3.756 ± 0.031 | 96 |
| 24.02 ± 0.20 | 3.702 ± 0.030 | 38 |
| 24.35 ± 0.20 | 3.652 ± 0.030 | 20 |
| 24.68 ± 0.20 | 3.604 ± 0.029 | 21 |
| 25.25 ± 0.20 | 3.524 ± 0.027 | 27 |
| 25.73 ± 0.20 | 3.460 ± 0.026 | 16 |
| 26.42 ± 0.20 | 3.371 ± 0.025 | 100 |
| 27.09 ± 0.20 | 3.289 ± 0.024 | 10 |
| 27.70 ± 0.20 | 3.218 ± 0.023 | 31 |
| 28.58 ± 0.20 | 3.121 ± 0.021 | 17 |
| 29.12 ± 0.20 | 3.064 ± 0.021 | 25 |
| 29.52 ± 0.20 | 3.023 ± 0.020 | 17 |
| 30.14 ± 0.20 | 2.963 ± 0.019 | 8 |
| 31.31 ± 0.20 | 2.855 ± 0.018 | 15 |
| 31.80 ± 0.20 | 2.812 ± 0.017 | 7 |

Thermograms for Form C are provided in FIGS. 42A and 42B. The TGA shows 3.2% weight loss up to 196° C. Assuming MeOH is the only volatile, the weight loss is equivalent to 0.5 moles of MeOH per mole of Compound 1. The broad endotherms prior to 60° C. in the DSC are due to desolvation and form conversion to Form B. The exotherm, due to decomposition of the desolvated form, exhibits an onset of 190° C.

### Relative Thermodynamic Stability

Interconversion experiments were performed to identify the most thermodynamically stable anhydrous form of Compound 1 (Table 19). Interconversion or competitive slurry experiments are a solution mediated process that provides a pathway for the less soluble (more stable) crystal to grow at the expense of the more soluble crystal form. Outside the formation of a solvate or degradation, the resulting more stable polymorph from an interconversion experiment is independent of the solvent used because the more thermodynamically stable polymorph has a lower energy and therefore lower solubility. The choice of solvent affects the kinetics of polymorph conversion and not the thermodynamic relationship between polymorphic forms.

TABLE 19
Competitive Interconversion Slurry Experiments
between Crystalline Forms

| | | Solvent | | |
|---|---|---|---|---|
| Forms | Temp | (v/v) [11] | Time | Result |
| A + B | RT | DCM | 2 d | A + B |
| | | DCM | 8 d | A+ |
| | | | | B(minor) |
| | | 90:10 H₂O/DMF | 6 d | A |
| | | (0.97 a_{w}) | | |
| A + D | RT | DCM | 11 d | A |
| | | EtOH | <1 min | A |
| A + E | RT | DCM | 11 d | A |
| | 55° C. | THF | 1 d | A |
| | | THF | 1 d | A |
| | 77° C. | DMF | 1 d | A |
| | | | | disordered |
| | | DMF | 1 d | A |
| A + B + F | RT | 60:40 H₂O/DMF | 9 d | A |
| | | (0.78 a_{w}) | | |
| | | 07:93 H₂O/THF | 11 d | A |
| | | (0.91 a_{w}) | | |

Various combinations of Forms B, E, F, and Material D were slurried with Form A at ambient and elevated temperatures (for experiments involving Form E). Different solvent systems were used and included a variety of water activities. Saturated solutions were generated and then added to the mixtures composed of approximately equivalent quantities of the forms. The mixtures were slurried for a particular duration of time and the solids harvested and analyzed by XRPD.

Regardless of the mixtures used, Form A prevailed for each experiment. This suggests that Form A is more thermodynamically stable than Form B and Material D at ambient temperature and Form E at ambient temperature, 55° C., and 77° C.

### CONCLUSIONS

Based on the weakly basic pKₐ values of Compound 1, stronger acids were selected for salt formation. Crystalline materials were successfully isolated with all eight strong acids used and at least one representative crystalline sample from purported besylate, HCl, mesylate, napadisylate, napsylate, sulfate, and tosylate salts were isolated.

Free Base forms of Compound 1, Forms A, B, Material D, and Form E, are anhydrous forms; Form F is a hydrate; and Form C is a methanolate. Form A Anhydrate exhibits limited hygroscopicity, a decomposition onset of 207° C., and is identified as the most thermodynamically stable, relative to the other anhydrous forms. Form B Metastable Desolvate is obtained through the desolvation of Form C Methanolate upon overnight exposure to 80° C. Form E Metastable Anhydrate was most frequently observed through the disproportionation of various salts of Compound 1 in water. Form F Hydrate was generated by slurrying the HCl salt, HCl Form A, in water. It is probable that the hydrate results from the displacement of Cl⁻ from the crystal structure, which is unlikely to occur without the HCl salt as an intermediate. The hydrate was shown to remain unchanged for 5 days under vacuum at ambient temperature but does dehydrate with concomitant decomposition upon exposure to 100° C. From these experiments, it was determined that Form A of Compound 1 has superior stability as compared to the other polymorphs studied.

1.-26. (canceled)

27. A combination comprising:
Component A: a SV2A inhibitor, or a pharmaceutically acceptable salt, hydrate, solvate, isomer, or polymorph thereof; or a first pharmaceutical composition comprising a SV2A inhibitor, or a pharmaceutically acceptable salt, hydrate, solvate, polymorph or isomer thereof;
Component B: a GABA_{A} α5 receptor agonist, or a pharmaceutically acceptable salt, hydrate, solvate, isomer, or polymorph thereof; or a second pharmaceutical composition comprising a GABA_{A} α5 receptor agonist, or a pharmaceutically acceptable salt, hydrate, solvate, polymorph or isomer thereof; and
wherein the first pharmaceutical composition and the second pharmaceutical composition comprise a pharmaceutically acceptable carrier.

28. The combination of claim 27, wherein the GABA_{A} α5 receptor agonist, or the pharmaceutically acceptable salt, hydrate, solvate, isomer, or polymorph thereof, is
a compound of formula II: 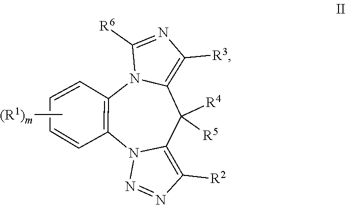
or a pharmaceutically acceptable salt, hydrate, solvate, polymorph, or isomer thereof, wherein:
m is 0-3;
each R¹ is independently selected from the group consisting of: halogen, —H, —(C1-C6)alkyl, —OH, —O((C1-C6)alkyl), —NO₂, —CN, —CF₃, —OCF₃, —OCHF₂, -OMe, —C≡C—R⁸, —CHF₂, —CH₂CF₃, —(C6-C10) aryl, —(C1-C6) alkyl-(C6-C10) aryl, -5-10 membered heteroaryl, —(C1-C6) alkyl-5-10 membered heteroaryl, and —(C3-C6) cycloalkyl; wherein R¹ is independently substituted with 0-5 R′;
R² is selected from the group consisting of:
—H, halogen, —OH, —(C1-C6)aliphatic, —O((C1-C6)alkyl), —C(O)O((C1-C6)alkyl), —C(O)NR₂, —(CR₂)₁₋₃—OR, —(CR₂)₁₋₃—O(CR₂)₁₋₃—R, —OR⁹, —C(O)R⁸, —CH₂R⁸, —CH₃, —CH₂—OR⁸, (C6-C10)-aryl-,
(C6-C10)-aryl-(C1-C12)aliphatic-,
(C6-C10)-aryl-O—(C1-C12)aliphatic-,
(C6-C10)-aryl-N(R″)—(C1-C12)aliphatic-,
(5- to 10-membered heteroaryl)-(C1-C12)aliphatic-,
(5- to 10-membered heteroaryl)-O—(C1-C12)aliphatic-,
(5- to 10-membered heteroaryl)-N(R″)—(C1-C12)aliphatic-,
(3- to 10-membered heterocyclyl)-(C1-C12)aliphatic-,
(3- to 10-membered heterocyclyl)-O—(C1-C12)aliphatic-, and
(3- to 10-membered heterocyclyl)-N(R″)—(C1-C12)aliphatic-,
wherein R² is independently substituted with 0-5 R′;
R³ is selected from the group consisting of:
—(C1-C6)alkyl, —(C2-C6)alkenyl, —C≡CH, —C≡CR⁹, —CN, halogen, —SO₂((C6-C10)-aryl), —SO₂((C1-C6)alkyl), —C(O)N((C1-C6)alkyl)₂, —C(O)NH₂, —C(O)O((C1-C6)alkyl), —C(O)((C1-C6)alkyl), —(C6-C10)aryl, 5- to 10-membered heteroaryl, 5- to 10-membered heterocyclyl, —(C1-C6)alkyl-C≡C—R¹⁰, —CH₂—O—R¹⁰, —CH₂—O—CH₂—R¹⁰ 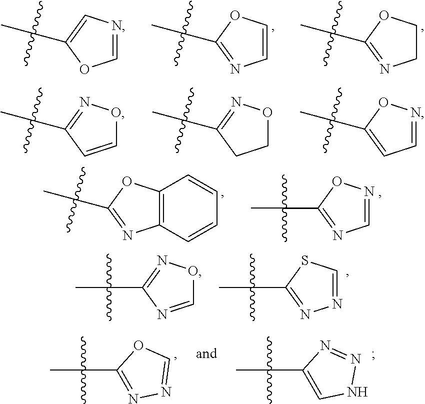
wherein each 5-member heterocycle or heteroaryl is substituted with 0-4 R₇;
wherein R³ is independently substituted with 0-5 R′;
R⁴ and R⁵ are each independently selected from the group consisting of —H, halogen, —(C1-C6)alkyl, or —(C1-C6) alkyl-(C6-C10) aryl; the (C6-C10)aryl being independently substituted with 0-5 halogen;
R⁶ is selected from the group consisting of —H and —(C1-C6)alkyl;
wherein R₇ is selected from the group consisting of —(C1-C6)alkyl, —(C3-C6)cycloalkyl, -5 to 10 membered heteroaryl, —(C6-C10) aryl, (C6-C10)aryl-(C1-C6)alkyl-, -5 to 10 membered heteroaryl-(C1-C6)alkyl, and -5-10 membered heteroaryl; wherein each R₇ is independently substituted with 0-5 R′;
wherein each R⁸ is independently selected from the group consisting of —H, —(C1-C6) alkyl, —(C3-C6) cycloalkyl, —(C1-C6)alkyl-(C3-C6)cycloalkyl, —(C1-C6)alkyl-(C6-C10)aryl, —(C6-C10) aryl, -5-10 membered heteroaryl, and —(C1-C6)alkyl-5-10 membered heteroaryl;
wherein each R⁸ excluding —H and —(C1-C6) alkyl is independently substituted by 0-5 of-halogen, —(C1-C6) alkyl, —CF₃, —OCF₃, or O—(C1-C6) alkyl;
wherein R⁹ is selected from the group consisting of —H, —(C1-C6) alkyl, —(C6-C10)aryl, -5-10 membered heteroaryl, —(C1-C6)alkyl-(C6-C10) aryl, —(C1-C6) alkyl-5-10 membered heteroaryl, —(C3-C6) cycloalkyl, —(C1-C6) alkyl-(C3-C6) cycloalkyl, —C(O)—(C6-C10)aryl, 5-10 membered heterocycle, 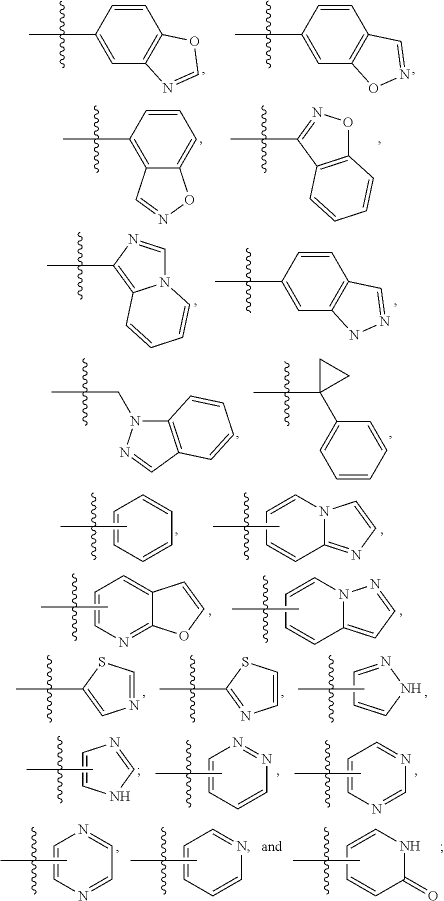
wherein each R⁹ is independently substituted with 0-5 R¹¹;
wherein R¹⁰ is selected from the group consisting of —H, halogen, —(C1-C6) alkyl, —(C6-C10) aryl, -5-10 membered heteroaryl, —(C3-C6) cycloalkyl, —CH₂—(C3-C6) cycloalkyl, —CH₂—(C6-C10) aryl, and —CH₂-5-10-membered heteroaryl,
wherein each R¹⁰ is substituted with 0-5 R′;
wherein each occurrence of R¹¹ is independently selected from the group consisting of -halogen, —CN, SCH₃, —CF₃, —OH, —OCF₃, OCHF₂, —O(C1-C6)alkyl, —(C6-C10) aryl, —(C1-C6)alkyl, and -5 to 10 membered heteroaryl;
each R is independently selected from the group consisting of:
H—,
(C1-C12)-aliphatic-,
(C3-C10)-cycloalkyl-,
(C3-C10)-cycloalkenyl-,
[(C3-C10)-cycloalkyl]—(C1-C12)-aliphatic-,
[(C3-C10)-cycloalkenyl]—(C1-C12)-aliphatic-,
[(C3-C10)-cycloalkyl]-O—(C1-C12)-aliphatic-,
[(C3-C10)-cycloalkenyl]-O—(C1-C12)-aliphatic-,
(C6-C10)-aryl-,
(C6-C10)-aryl-(C1-C12)aliphatic-,
(C6-C10)-aryl-O—(C1-C12)aliphatic-,
(C6-C10)-aryl-N(R″)—(C1-C12)aliphatic-,
3- to 10-membered heterocyclyl-,
(3- to 10-membered heterocyclyl)-(C1-C12)aliphatic-,
(3- to 10-membered heterocyclyl)-O—(C1-C12)aliphatic-,
(3- to 10-membered heterocyclyl)-N(R″)—(C1-C12)aliphatic-,
5- to 10-membered heteroaryl-,
(5- to 10-membered heteroaryl)-(C1-C12)-aliphatic-,
(5- to 10-membered heteroaryl)-O—(C1-C12)-aliphatic-; and
(5- to 10-membered heteroaryl)-N(R″)—(C1-C12)-aliphatic-;
wherein said heterocyclyl has 1-4 heteroatoms independently selected from the group consisting of N, NH, O, S, SO, and SO₂, and said heteroaryl has 1-4 heteroatoms independently selected from the group consisting of N, NH, O, and S;
wherein each occurrence of R is independently substituted with 0-5 R′;
or when two R groups bound to the same atom, the two R groups may be taken together with the atom to which they are bound to form a 3- to 10-membered aromatic or non-aromatic ring having 0-4 heteroatoms independently selected from the group consisting of N, NH, O, S, SO, and SO₂, wherein said ring is optionally substituted with 0-5 R′, and wherein said ring is optionally fused to a (C6-C10)aryl, 5- to 10-membered heteroaryl, (C3-C10)cycloalkyl, or a 3- to 10-membered heterocyclyl;
wherein each occurrence of R′ is independently selected from the group consisting of halogen, —R″, —OR″, oxo, —CH₂OR″, —CH₂NR″₂, —C(O)N(R″)₂, —C(O)OR″, —NO₂, —NCS, —CN, —CF₃, —OCF₃ and —N(R″)₂;
wherein each occurrence of R″ is independently selected from the group consisting of H, —(C1-C6)-alkyl, —(C1-C6)-aliphatic, (C3-C6)-cycloalkyl, 3- to 6-membered heterocyclyl, 5- to 10-membered heteroaryl-, (C6-C10)-aryl-, (5- to 10-membered heteroaryl)-(C1-C6)-alkyl-, (C6-C10)-aryl-(C1-C6)-alkyl-, (5- to 10-membered heteroaryl)-O—(C1-C6)-alkyl-, (C6-C10)-aryl-O—(C1-C6)-alkyl-, and (C6-C10)-aryl-O—(C1-C6)-alkyl-,
wherein each occurrence of R″ is independently substituted with 0-5 substituents selected from the group consisting of: halogen, —R^{o}, —OR^{o}, oxo, —CH₂OR^{o}, —CH₂N(R^{o})₂, —C(O)N(R^{o})₂, —C(O)OR^{o}, —NO₂, —NCS, —CN, —CF₃, —OCF₃ and —N(R^{o})₂, wherein each occurrence of R^{o} is independently selected from the group consisting of: —(C1-C6)-aliphatic, (C3-C6)-cycloalkyl, 3- to 6-membered heterocyclyl, 5- to 10-membered heteroaryl-, and (C6-C10)-aryl
or a compound of formula IV: 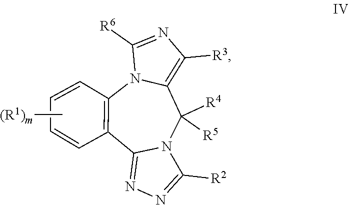
or a pharmaceutically acceptable salt, hydrate, solvate, polymorph, or isomer thereof, wherein:
m is 0-3;
each R¹ is independently selected from the group consisting of: halogen, —H, —(C1-C6)alkyl, —C≡C—R⁹, —OH, —O((C1-C6)alkyl), —NO₂, —CN, —CF₃, —OCF₃, —CHF₂, —CH₂CF₃, —(C6-C10) aryl, —(C1-C6) alkyl-(C6-C10) aryl, -5-10 membered heteroaryl, —(C1-C6) alkyl-5-10 membered heteroaryl, and —(C3-C6) cycloalkyl;
wherein R¹ is independently substituted with 0-5 R′;
R² is selected from the group consisting of —OR⁸, —SR⁸, —(CH₂)ₙOR⁸, —(CH₂)ₙO(CH₂)ₙR⁸, —(CH₂)_{P}R⁸ and —(CH₂)ₙN(R″)R¹⁰, wherein n is an integer selected from 0-4; p is an integer selected from 2-4;
wherein R² is independently substituted with 0-5 R′;
each R³ is independently selected from the group consisting of:
—H, —CN, halogen, —(C1-C6)aliphatic, —CH═CR⁹, —C≡CR⁹, —SO₂((C1-C6)alkyl), —C(O)N((C1-C6)alkyl)₂), —C(O)NH((C1-C6)aliphatic), (C6-C10)-aryl-(C1-C12)aliphatic-, —C(O)((C1-C6)alkyl), —C(O)O((C1-C6)alkyl), 5- or 6-membered heterocyclyl, 5- or 6-membered heteroaryl, —CH₂—O—R¹⁰, —CH₂—O—CH₂—R¹⁰ 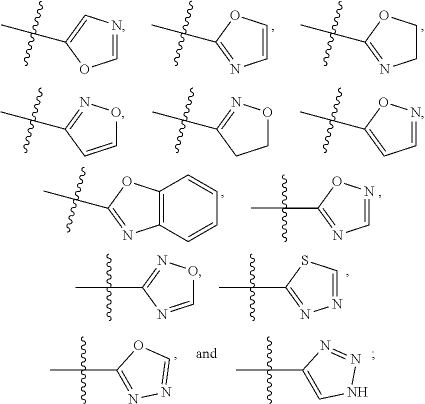
wherein each 5-10-membered heterocycle or heteroaryl are substituted with 0-3 R₇;
wherein R³ is independently substituted with 0-5 R′;
R⁴ and R⁵ are each independently selected from the group consisting of —H, halogen and —(C1-C6)alkyl;
R⁶ is selected from the group consisting of —H and —(C1-C6)alkyl;
R₇ is selected from the group consisting of —(C1-C6)alkyl, —(C3-C6)cycloalkyl, -5 to 10 membered heteroaryl, —(C6-C10) aryl, —(C6-C10)aryl-(C1-C6)alkyl, and -5 to 10 membered heteroaryl-(C1-C6)alkyl, and -5-10 membered heteroaryl;
wherein each R₇ is independently substituted with 0-5 R′;
R⁸ is independently selected from the group consisting of —H, —(C1-C6)alkyl, —(C3-C10)-cycloalkyl, (C6-C10)-aryl, or 5- to 10-membered heteroaryl, 5-10 membered heteroaryl-(C1-C6) alkyl-, —(C1-C6) alkyl-(C6-C10) aryl, and —(C1-C6) alkyl-(C3-C6) cycloalkyl;
wherein each occurrence of R⁸ is independently substituted with 0-5 R′;
wherein R⁹ is selected from the group consisting of —H, —(C1-C6) alkyl, —(C3-C6) cycloalkyl, —(C1-C6) alkyl-(C3-C6) cycloalkyl, —(C1-C6)alkyl-(C6-C10) aryl, —(C6-C10)aryl, -5-10 membered heteroaryl, —(C1-C6)alkyl-5-10 membered heteroaryl, 5-10 membered heterocycle, —C(O)—(C6-C10) aryl, 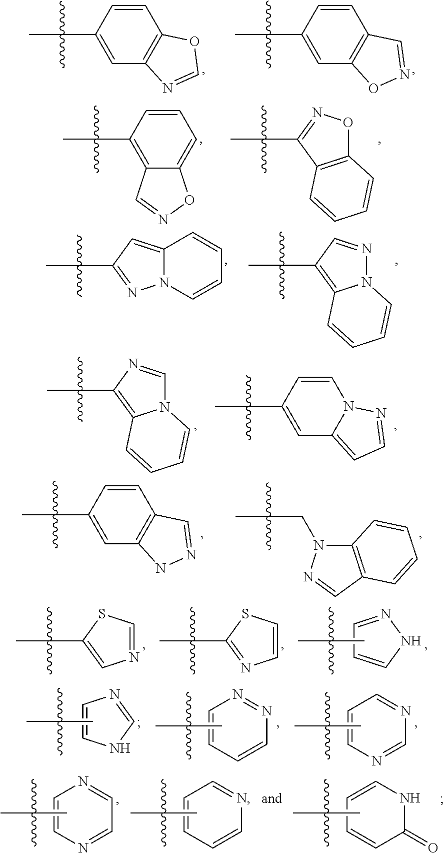
wherein each wherein each R⁹ is independently substituted with 0-5 R¹¹;
R¹⁰ is selected from the group consisting of —H, —(C1-C6) alkyl, —(C3-C10)-cycloalkyl, 3- to 10-membered heterocyclyl-, (C6-C10)-aryl, 5- to 10-membered heteroaryl, —CH₂—(C3-C6) cycloalkyl, —CH₂—(C6-C10) aryl, and —CH₂-5-10-membered heteroaryl,
wherein each occurrence of R¹⁰ is independently substituted with 0-5 R′;
wherein each occurrence of R¹¹ is independently selected from the group consisting of -halogen, —CF₃, —OCF₃, OCF₂H, —O—(C1-C6)alkyl, —(C6-C10) aryl, —(C1-C6)alkyl, —O—CH₂—(C3-C6)cycloalkyl, and -5 to 10 membered heteroaryl;
wherein each occurrence of R′ is independently selected from the group consisting of halogen, —R″, —OR″, oxo, —CH₂OR″, —CH₂NR″₂, —C(O)N(R″)₂, —C(O)OR″, —NO₂, —NCS, —CN, —CF₃, —OCF₃ and —N(R″)₂;
wherein each occurrence of R″ is independently selected from the group consisting of H, —(C1-C6)-aliphatic, —(C1-C6)-alkyl, (C3-C6)-cycloalkyl, 3- to 6-membered heterocyclyl, 5- to 10-membered heteroaryl-, (C6-C10)-aryl-, (5- to 10-membered heteroaryl)-(C1-C6)-alkyl-, (C6-C10)-aryl-(C1-C6)-alkyl-, (5- to 10-membered heteroaryl)-O—(C1-C6)-alkyl-, and (C6-C10)-aryl-O—(C1-C6)-alkyl-;
wherein each occurrence of R″ is independently substituted with 0-5 R^{t} independently selected from the group consisting of: halogen, —R^{o}, —OR^{o}, oxo, —CH₂OR^{o}, —CH₂N(R^{o})₂, —C(O)N(R^{o})₂, —C(O)OR^{o}, —NO₂, —NCS, —CN, —CF₃, —OCF₃ and —N(R^{o})₂, wherein each occurrence of R^{o} is independently selected from: —(C1-C6)-aliphatic, (C3-C6)-cycloalkyl, 3- to 6-membered heterocyclyl, 5- to 10-membered heteroaryl-, and (C6-C10)-aryl-.

29. The combination according to claim 27, wherein the SV2A inhibitor, or the pharmaceutically acceptable salt, hydrate, solvate, polymorph, or isomer thereof, is levetiracetam, seletracetam, brivaracetam, or a pharmaceutically acceptable salt, hydrate, solvate, polymorph, or isomer of any of the foregoing.

30.-35. (canceled)

36. The combination according to claim 27, wherein the GABA_{A} α5 receptor agonist is selected from the group consisting of: 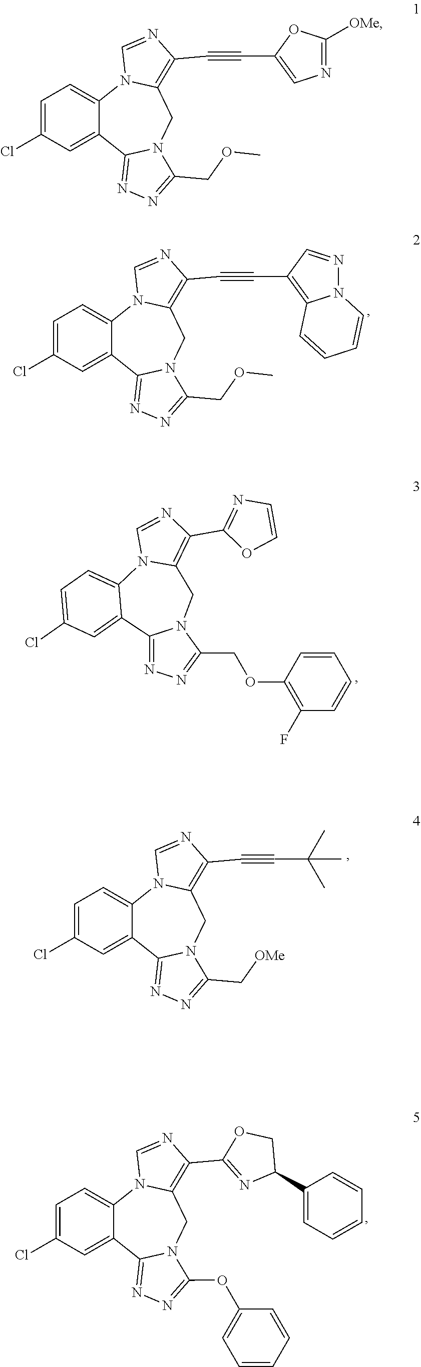 [SMILES: COCc1nnc2n1Cc1c(C#Cc3cnc(OC)o3)ncn1-c1ccc(Cl)cc1-2 ; COCc1nnc2n1Cc1c(C#Cc3cnn4ccccc34)ncn1-c1ccc(Cl)cc1-2 ; Fc1ccccc1OCc1nnc2n1Cc1c(-c3ncco3)ncn1-c1ccc(Cl)cc1-2 ; COCc1nnc2n1Cc1c(C#CC(C)(C)C)ncn1-c1ccc(Cl)cc1-2 ; Clc1ccc2c(c1)-c1nnc(Oc3ccccc3)n1Cc1c(C3=N[C@H](c4ccccc4)CO3)ncn1-2] 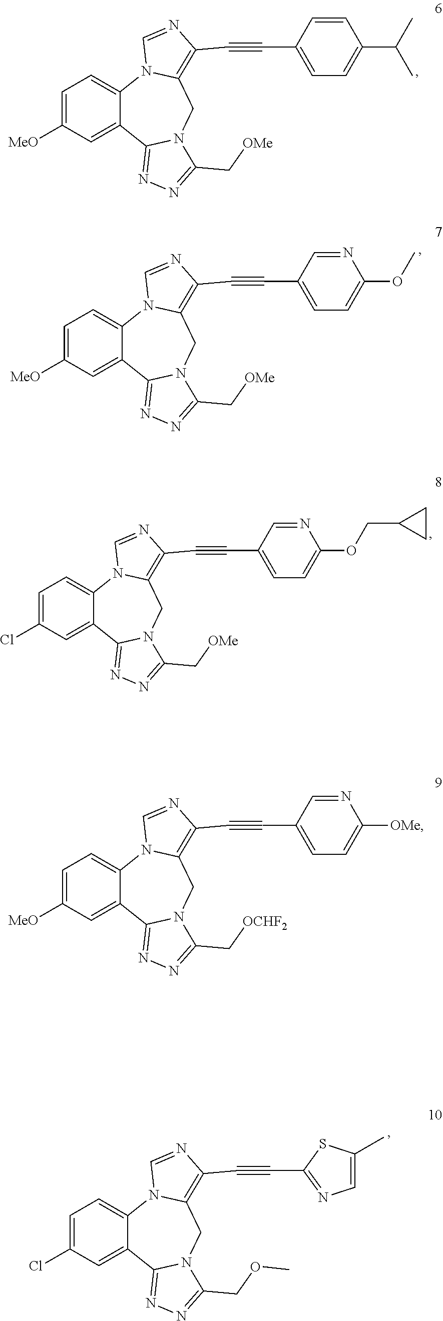 [SMILES: COCc1nnc2n1Cc1c(C#Cc3ccc(C(C)C)cc3)ncn1-c1ccc(OC)cc1-2 ; COCc1nnc2n1Cc1c(C#Cc3ccc(OC)nc3)ncn1-c1ccc(OC)cc1-2 ; COCc1nnc2n1Cc1c(C#Cc3ccc(OCC4CC4)nc3)ncn1-c1ccc(Cl)cc1-2 ; COc1ccc2c(c1)-c1nnc(COC(F)F)n1Cc1c(C#Cc3ccc(OC)nc3)ncn1-2 ; COCc1nnc2n1Cc1c(C#Cc3ncc(C)s3)ncn1-c1ccc(Cl)cc1-2] 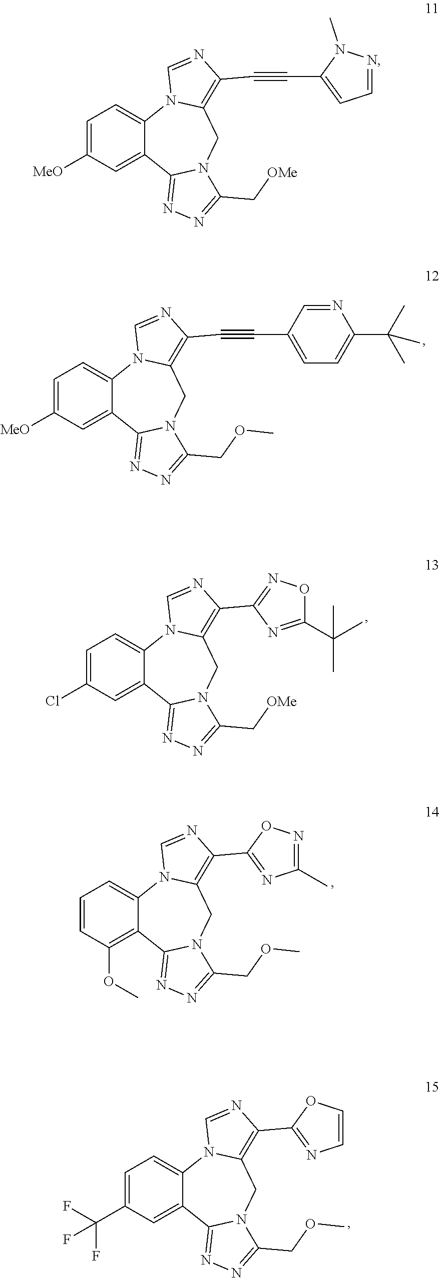 [SMILES: COCc1nnc2n1Cc1c(C#Cc3ccnn3C)ncn1-c1ccc(OC)cc1-2 ; COCc1nnc2n1Cc1c(C#Cc3ccc(C(C)(C)C)nc3)ncn1-c1ccc(OC)cc1-2 ; COCc1nnc2n1Cc1c(-c3noc(C(C)(C)C)n3)ncn1-c1ccc(Cl)cc1-2 ; COCc1nnc2n1Cc1c(-c3nc(C)no3)ncn1-c1cccc(OC)c1-2 ; COCc1nnc2n1Cc1c(-c3ncco3)ncn1-c1ccc(C(F)(F)F)cc1-2] 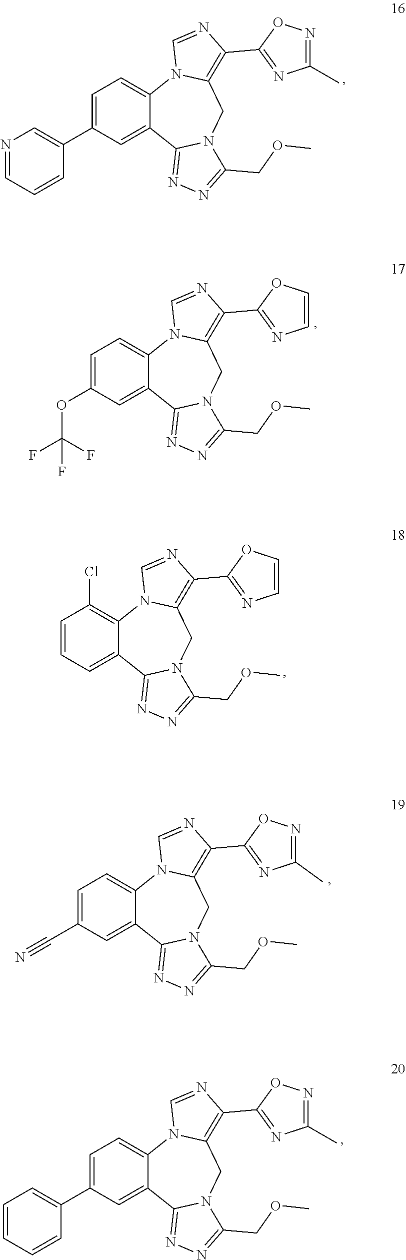 [SMILES: COCc1nnc2n1Cc1c(-c3nc(C)no3)ncn1-c1ccc(-c3cccnc3)cc1-2 ; COCc1nnc2n1Cc1c(-c3ncco3)ncn1-c1ccc(OC(F)(F)F)cc1-2 ; COCc1nnc2n1Cc1c(-c3ncco3)ncn1-c1c(Cl)cccc1-2 ; COCc1nnc2n1Cc1c(-c3nc(C)no3)ncn1-c1ccc(C#N)cc1-2 ; COCc1nnc2n1Cc1c(-c3nc(C)no3)ncn1-c1ccc(-c3ccccc3)cc1-2] 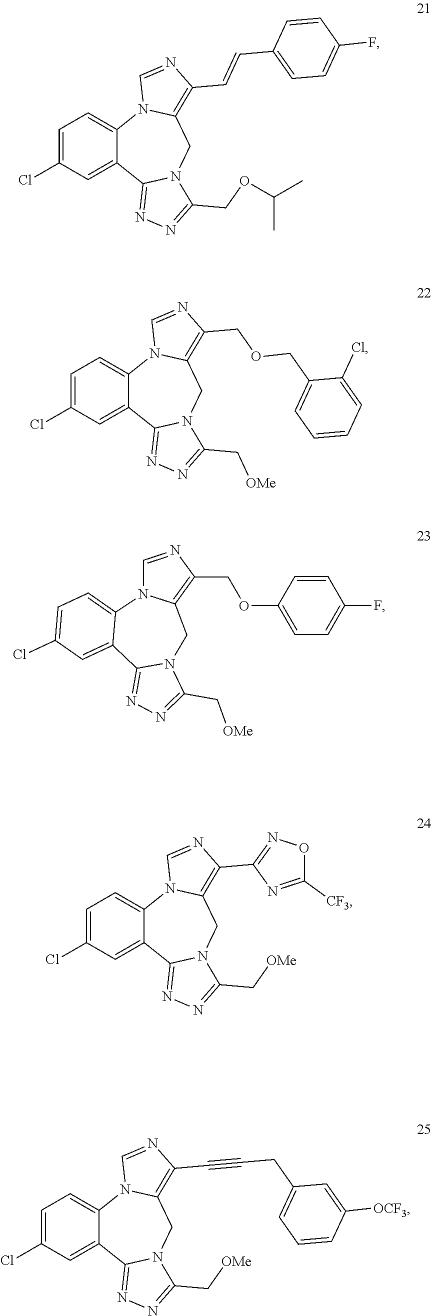 [SMILES: CC(C)OCc1nnc2n1Cc1c(/C=C/c3ccc(F)cc3)ncn1-c1ccc(Cl)cc1-2 ; COCc1nnc2n1Cc1c(COCc3ccccc3Cl)ncn1-c1ccc(Cl)cc1-2 ; COCc1nnc2n1Cc1c(COc3ccc(F)cc3)ncn1-c1ccc(Cl)cc1-2 ; COCc1nnc2n1Cc1c(-c3noc(C(F)(F)F)n3)ncn1-c1ccc(Cl)cc1-2 ; COCc1nnc2n1Cc1c(C#CCc3cccc(OC(F)(F)F)c3)ncn1-c1ccc(Cl)cc1-2] 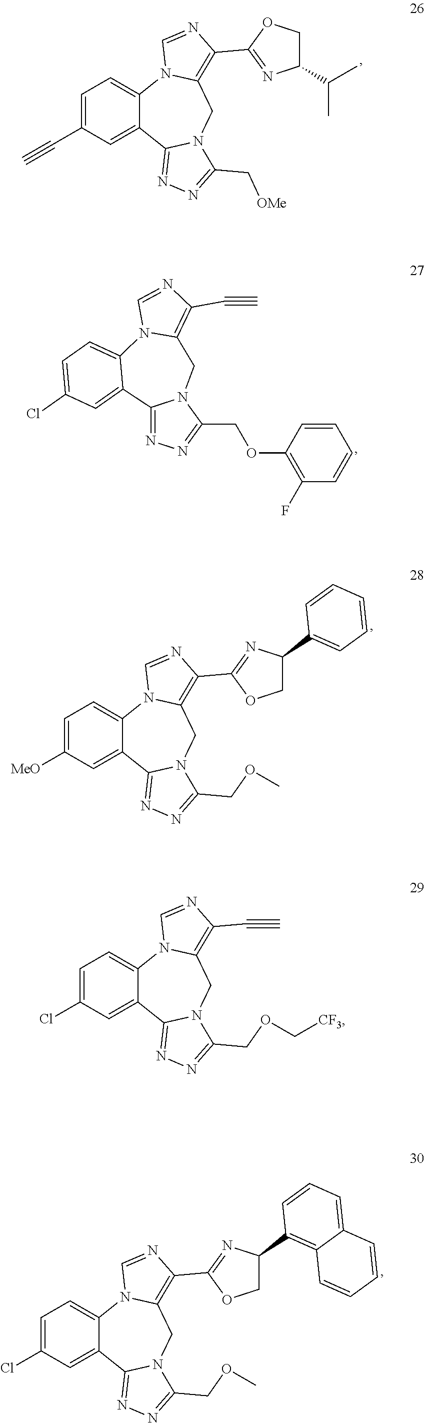 [SMILES: C#Cc1ccc2c(c1)-c1nnc(COC)n1Cc1c(C3=N[C@@H](C(C)C)CO3)ncn1-2 ; C#Cc1ncn2c1Cn1c(COc3ccccc3F)nnc1-c1cc(Cl)ccc1-2 ; COCc1nnc2n1Cc1c(C3=N[C@@H](c4ccccc4)CO3)ncn1-c1ccc(OC)cc1-2 ; C#Cc1ncn2c1Cn1c(COCC(F)(F)F)nnc1-c1cc(Cl)ccc1-2 ; COCc1nnc2n1Cc1c(C3=N[C@@H](c4cccc5ccccc45)CO3)ncn1-c1ccc(Cl)cc1-2] 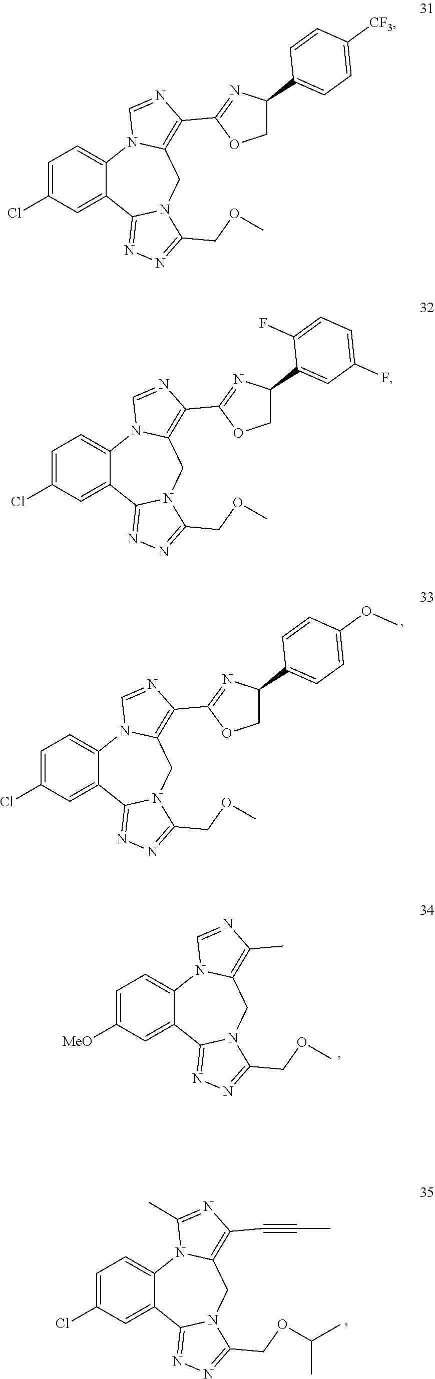 [SMILES: COCc1nnc2n1Cc1c(C3=N[C@@H](c4ccc(C(F)(F)F)cc4)CO3)ncn1-c1ccc(Cl)cc1-2 ; COCc1nnc2n1Cc1c(C3=N[C@@H](c4cc(F)ccc4F)CO3)ncn1-c1ccc(Cl)cc1-2 ; COCc1nnc2n1Cc1c(C3=N[C@@H](c4ccc(OC)cc4)CO3)ncn1-c1ccc(Cl)cc1-2 ; COCc1nnc2n1Cc1c(C)ncn1-c1ccc(OC)cc1-2 ; CC#Cc1nc(C)n2c1Cn1c(COC(C)C)nnc1-c1cc(Cl)ccc1-2] 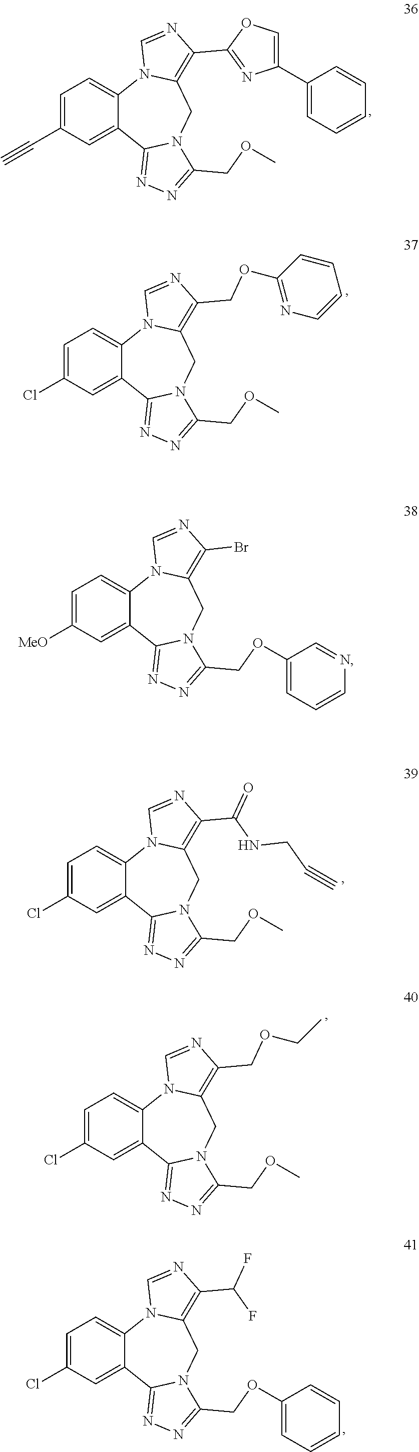 [SMILES: C#Cc1ccc2c(c1)-c1nnc(COC)n1Cc1c(-c3nc(-c4ccccc4)co3)ncn1-2 ; COCc1nnc2n1Cc1c(COc3ccccn3)ncn1-c1ccc(Cl)cc1-2 ; COc1ccc2c(c1)-c1nnc(COc3cccnc3)n1Cc1c(Br)ncn1-2 ; C#CCNC(=O)c1ncn2c1Cn1c(COC)nnc1-c1cc(Cl)ccc1-2 ; CCOCc1ncn2c1Cn1c(COC)nnc1-c1cc(Cl)ccc1-2 ; FC(F)c1ncn2c1Cn1c(COc3ccccc3)nnc1-c1cc(Cl)ccc1-2] 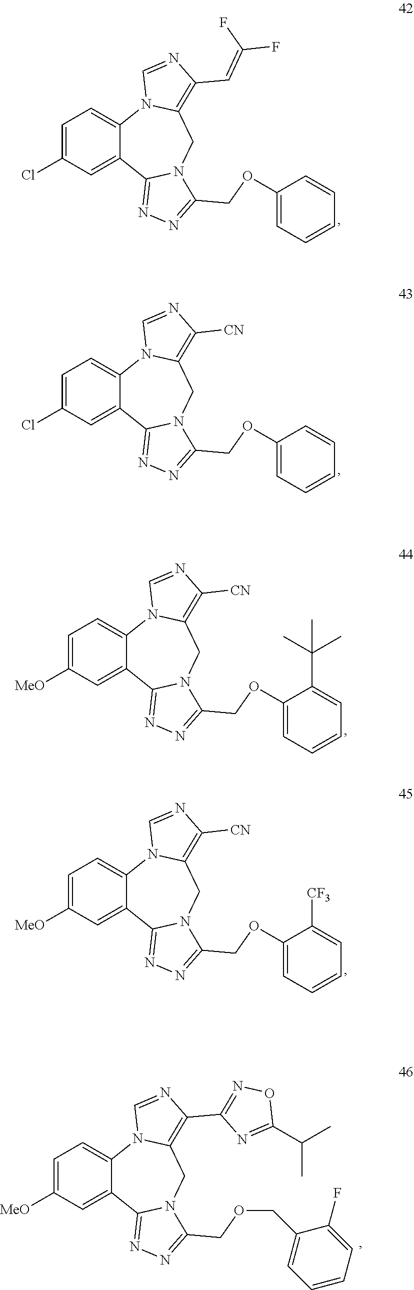 [SMILES: FC(F)=Cc1ncn2c1Cn1c(COc3ccccc3)nnc1-c1cc(Cl)ccc1-2 ; N#Cc1ncn2c1Cn1c(COc3ccccc3)nnc1-c1cc(Cl)ccc1-2 ; COc1ccc2c(c1)-c1nnc(COc3ccccc3C(C)(C)C)n1Cc1c(C#N)ncn1-2 ; COc1ccc2c(c1)-c1nnc(COc3ccccc3C(F)(F)F)n1Cc1c(C#N)ncn1-2 ; COc1ccc2c(c1)-c1nnc(COCc3ccccc3F)n1Cc1c(-c3noc(C(C)C)n3)ncn1-2] 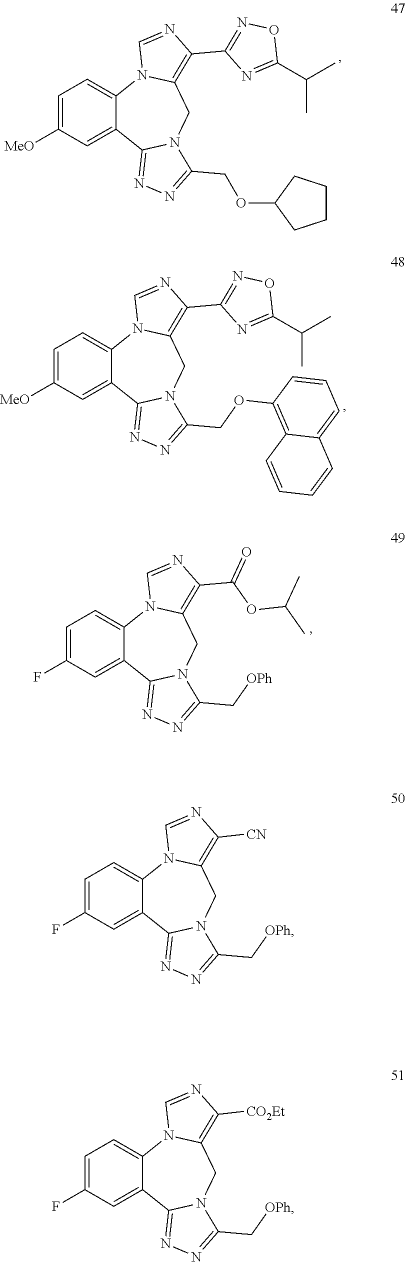 [SMILES: COc1ccc2c(c1)-c1nnc(COC3CCCC3)n1Cc1c(-c3noc(C(C)C)n3)ncn1-2 ; COc1ccc2c(c1)-c1nnc(COc3cccc4ccccc34)n1Cc1c(-c3noc(C(C)C)n3)ncn1-2 ; CC(C)OC(=O)c1ncn2c1Cn1c(COc3ccccc3)nnc1-c1cc(F)ccc1-2 ; N#Cc1ncn2c1Cn1c(COc3ccccc3)nnc1-c1cc(F)ccc1-2 ; CCOC(=O)c1ncn2c1Cn1c(COc3ccccc3)nnc1-c1cc(F)ccc1-2] 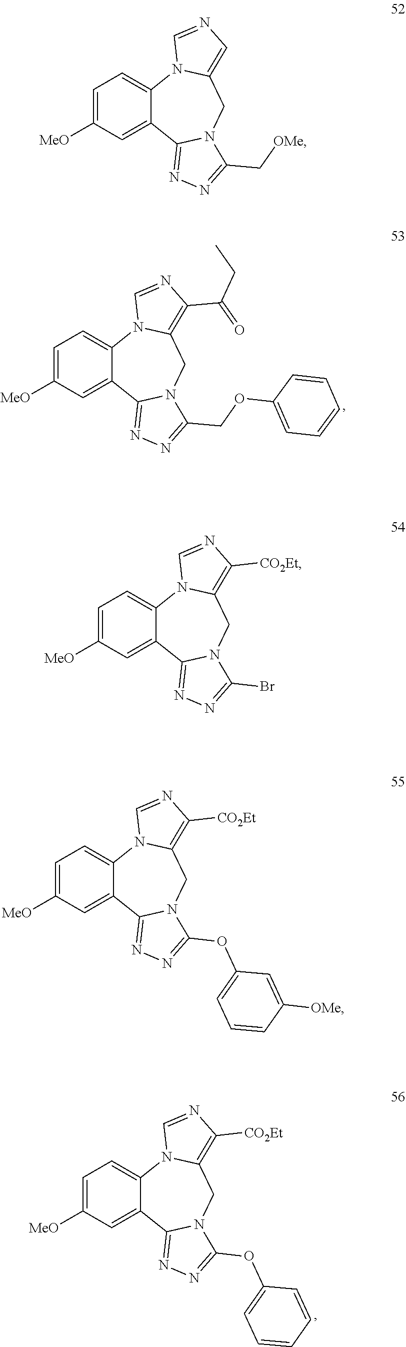 [SMILES: COCc1nnc2n1Cc1cncn1-c1ccc(OC)cc1-2 ; CCC(=O)c1ncn2c1Cn1c(COc3ccccc3)nnc1-c1cc(OC)ccc1-2 ; CCOC(=O)c1ncn2c1Cn1c(Br)nnc1-c1cc(OC)ccc1-2 ; CCOC(=O)c1ncn2c1Cn1c(Oc3cccc(OC)c3)nnc1-c1cc(OC)ccc1-2 ; CCOC(=O)c1ncn2c1Cn1c(Oc3ccccc3)nnc1-c1cc(OC)ccc1-2] 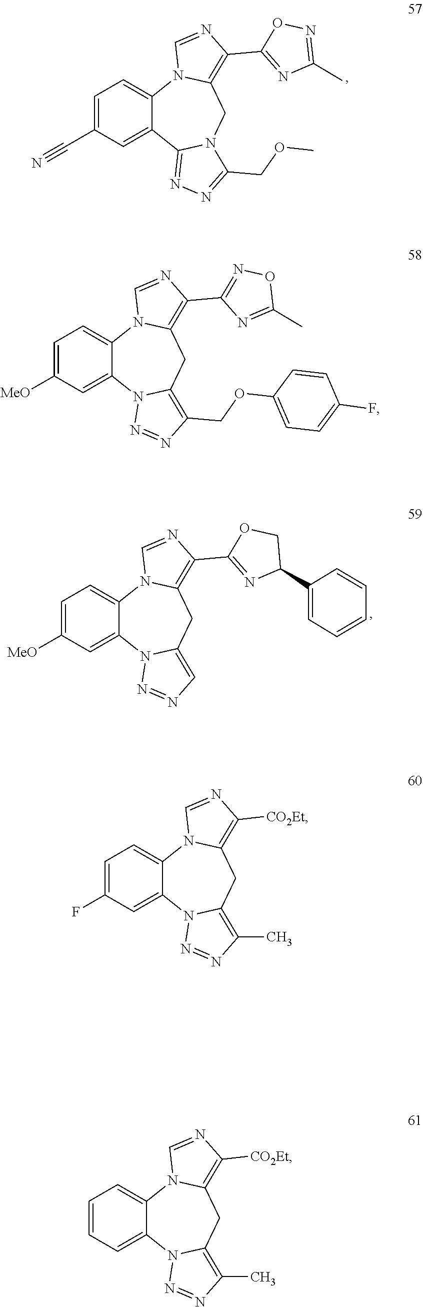 [SMILES: COCc1nnc2n1Cc1c(-c3nc(C)no3)ncn1-c1ccc(C#N)cc1-2 ; COc1ccc2c(c1)-n1nnc(COc3ccc(F)cc3)c1Cc1c(-c3noc(C)n3)ncn1-2 ; COc1ccc2c(c1)-n1nncc1Cc1c(C3=N[C@H](c4ccccc4)CO3)ncn1-2 ; CCOC(=O)c1ncn2c1Cc1c(C)nnn1-c1cc(F)ccc1-2 ; CCOC(=O)c1ncn2c1Cc1c(C)nnn1-c1ccccc1-2] 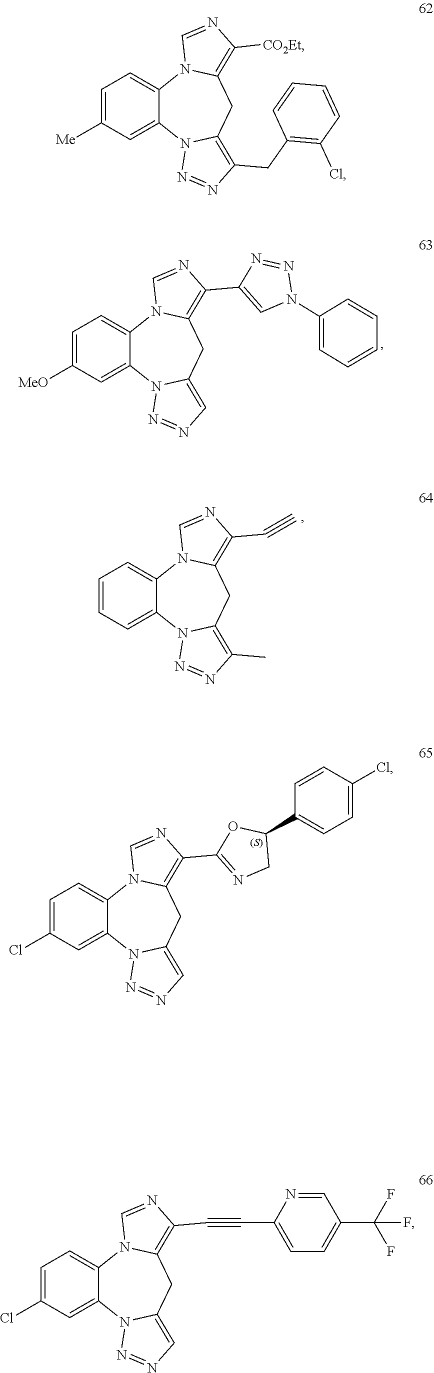 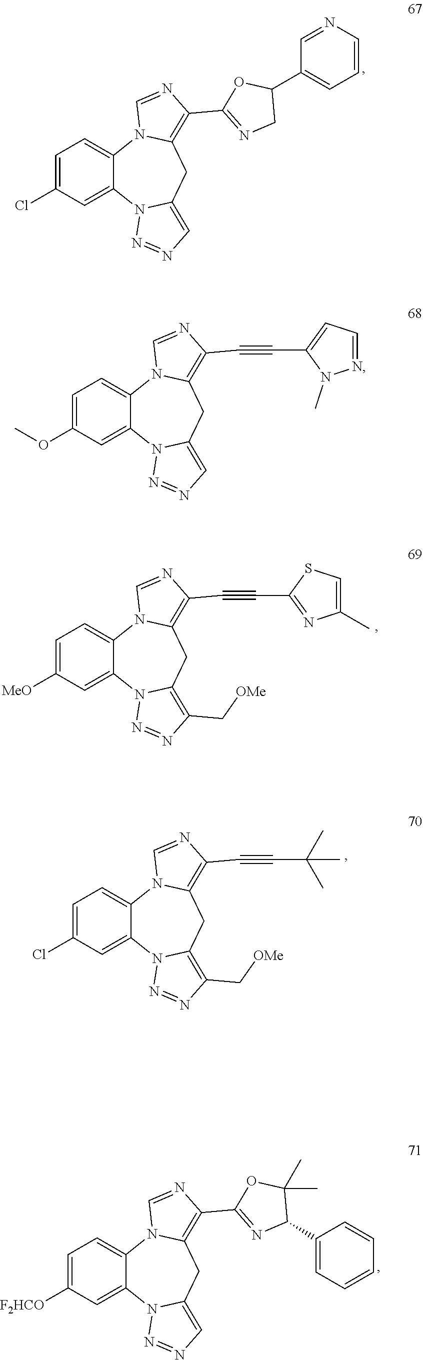 [SMILES: Clc1ccc2c(c1)-n1nncc1Cc1c(C3=NCC(c4cccnc4)O3)ncn1-2 ; COc1ccc2c(c1)-n1nncc1Cc1c(C#Cc3ccnn3C)ncn1-2 ; COCc1nnn2c1Cc1c(C#Cc3nc(C)cs3)ncn1-c1ccc(OC)cc1-2 ; COCc1nnn2c1Cc1c(C#CC(C)(C)C)ncn1-c1ccc(Cl)cc1-2 ; CC1(C)OC(c2ncn3c2Cc2cnnn2-c2cc(OC(F)F)ccc2-3)=N[C@H]1c1ccccc1] 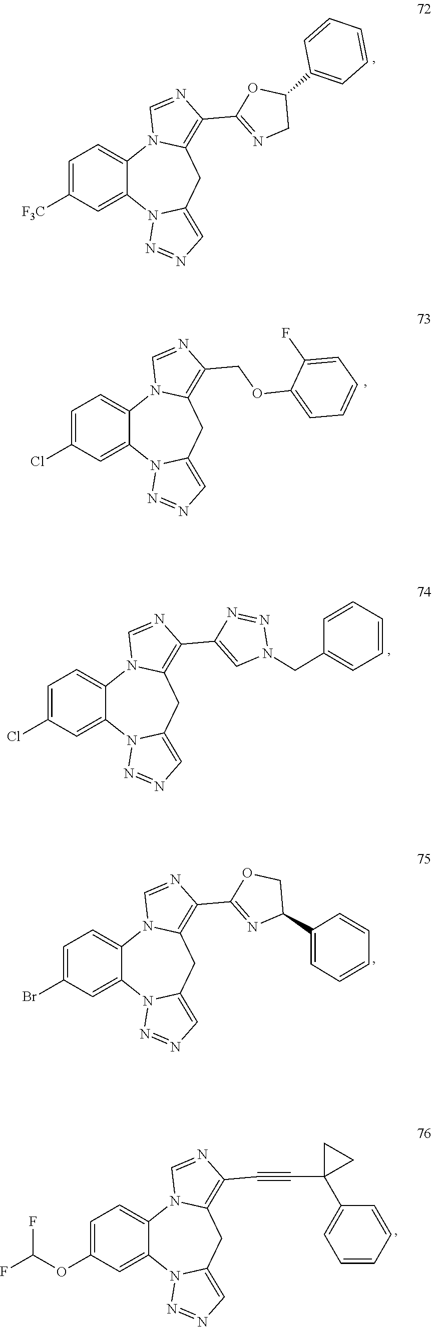 [SMILES: FC(F)(F)c1ccc2c(c1)-n1nncc1Cc1c(C3=NC[C@@H](c4ccccc4)O3)ncn1-2 ; Fc1ccccc1OCc1ncn2c1Cc1cnnn1-c1cc(Cl)ccc1-2 ; Clc1ccc2c(c1)-n1nncc1Cc1c(-c3cn(Cc4ccccc4)nn3)ncn1-2 ; Brc1ccc2c(c1)-n1nncc1Cc1c(C3=N[C@H](c4ccccc4)CO3)ncn1-2 ; FC(F)Oc1ccc2c(c1)-n1nncc1Cc1c(C#CC3(c4ccccc4)CC3)ncn1-2] 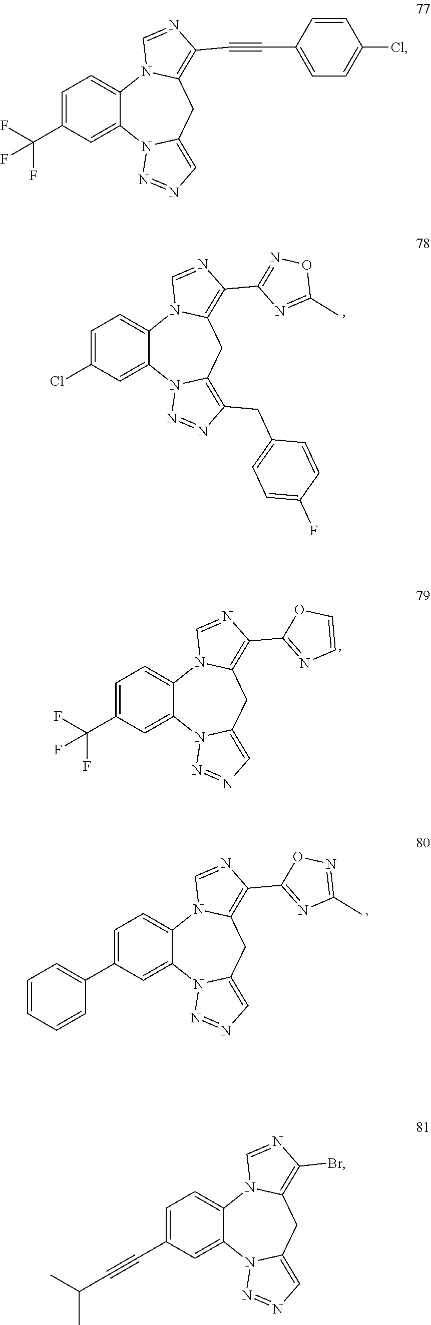 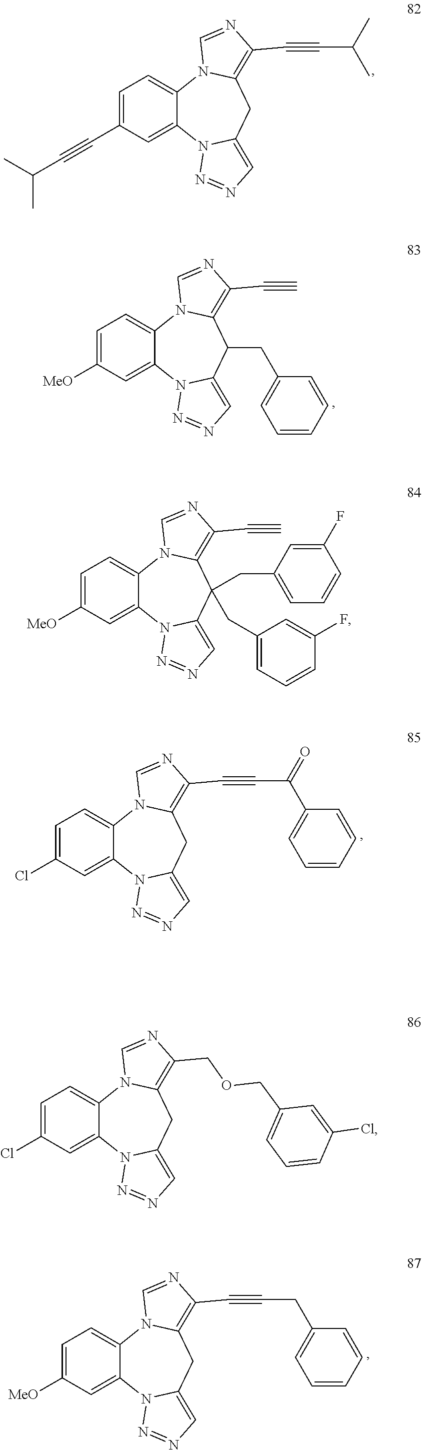 [SMILES: CC(C)C#Cc1ccc2c(c1)-n1nncc1Cc1c(C#CC(C)C)ncn1-2 ; C#Cc1ncn2c1C(Cc1ccccc1)c1cnnn1-c1cc(OC)ccc1-2 ; C#Cc1ncn2c1C(Cc1cccc(F)c1)(Cc1cccc(F)c1)c1cnnn1-c1cc(OC)ccc1-2 ; O=C(C#Cc1ncn2c1Cc1cnnn1-c1cc(Cl)ccc1-2)c1ccccc1 ; Clc1cccc(COCc2ncn3c2Cc2cnnn2-c2cc(Cl)ccc2-3)c1 ; COc1ccc2c(c1)-n1nncc1Cc1c(C#CCc3ccccc3)ncn1-2] 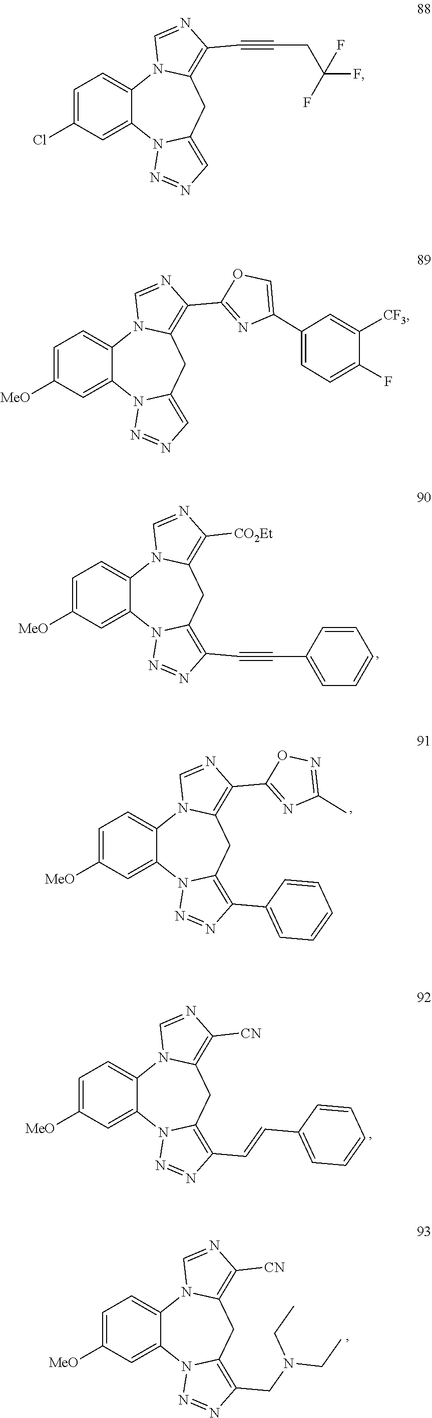 [SMILES: FC(F)(F)CC#Cc1ncn2c1Cc1cnnn1-c1cc(Cl)ccc1-2 ; COc1ccc2c(c1)-n1nncc1Cc1c(-c3nc(-c4ccc(F)c(C(F)(F)F)c4)co3)ncn1-2 ; CCOC(=O)c1ncn2c1Cc1c(C#Cc3ccccc3)nnn1-c1cc(OC)ccc1-2 ; COc1ccc2c(c1)-n1nnc(-c3ccccc3)c1Cc1c(-c3nc(C)no3)ncn1-2 ; COc1ccc2c(c1)-n1nnc(/C=C/c3ccccc3)c1Cc1c(C#N)ncn1-2 ; CCN(CC)Cc1nnn2c1Cc1c(C#N)ncn1-c1ccc(OC)cc1-2] 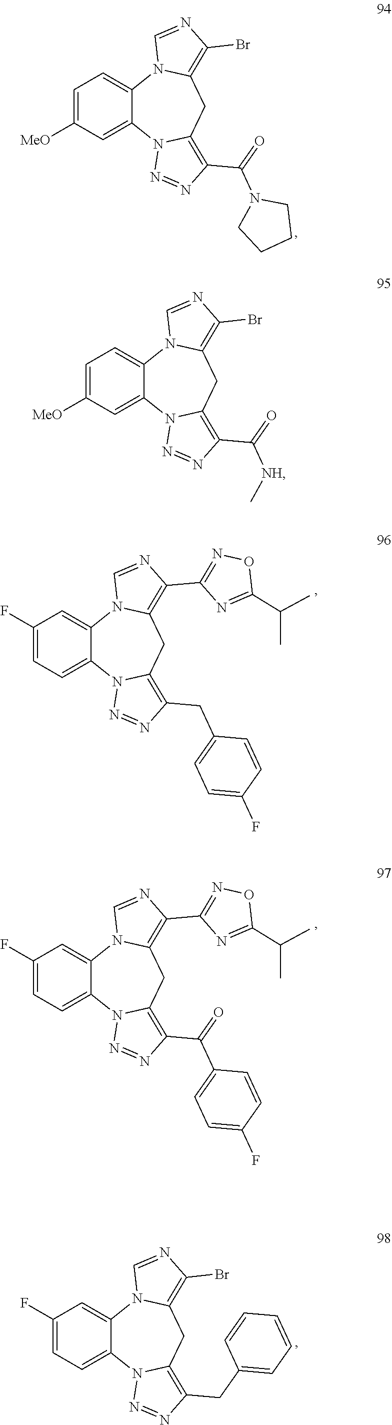 [SMILES: COc1ccc2c(c1)-n1nnc(C(=O)N3CCCC3)c1Cc1c(Br)ncn1-2 ; CNC(=O)c1nnn2c1Cc1c(Br)ncn1-c1ccc(OC)cc1-2 ; CC(C)c1nc(-c2ncn3c2Cc2c(Cc4ccc(F)cc4)nnn2-c2ccc(F)cc2-3)no1 ; CC(C)c1nc(-c2ncn3c2Cc2c(C(=O)c4ccc(F)cc4)nnn2-c2ccc(F)cc2-3)no1 ; Fc1ccc2c(c1)-n1cnc(Br)c1Cc1c(Cc3ccccc3)nnn1-2] 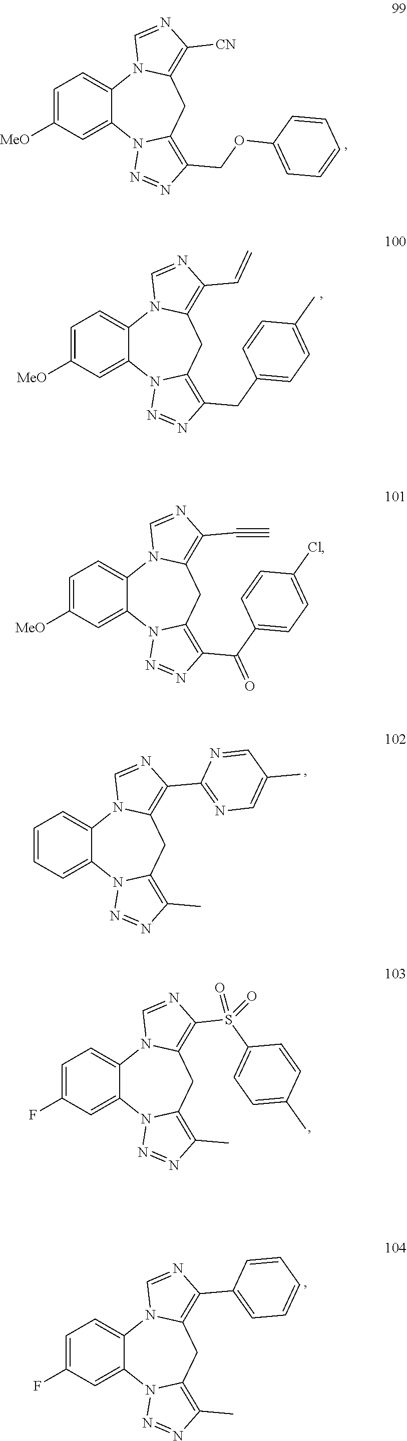 [SMILES: COc1ccc2c(c1)-n1nnc(COc3ccccc3)c1Cc1c(C#N)ncn1-2 ; C=Cc1ncn2c1Cc1c(Cc3ccc(C)cc3)nnn1-c1cc(OC)ccc1-2 ; C#Cc1ncn2c1Cc1c(C(=O)c3ccc(Cl)cc3)nnn1-c1cc(OC)ccc1-2 ; Cc1cnc(-c2ncn3c2Cc2c(C)nnn2-c2ccccc2-3)nc1 ; Cc1ccc(S(=O)(=O)c2ncn3c2Cc2c(C)nnn2-c2cc(F)ccc2-3)cc1 ; Cc1nnn2c1Cc1c(-c3ccccc3)ncn1-c1ccc(F)cc1-2] 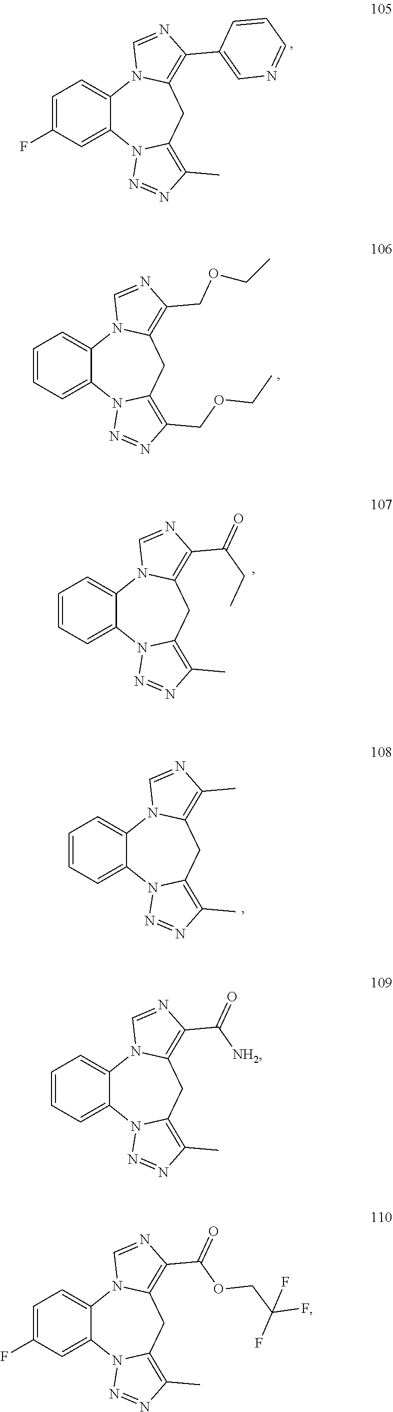 [SMILES: Cc1nnn2c1Cc1c(-c3cccnc3)ncn1-c1ccc(F)cc1-2 ; CCOCc1ncn2c1Cc1c(COCC)nnn1-c1ccccc1-2 ; CCC(=O)c1ncn2c1Cc1c(C)nnn1-c1ccccc1-2 ; Cc1ncn2c1Cc1c(C)nnn1-c1ccccc1-2 ; Cc1nnn2c1Cc1c(C(N)=O)ncn1-c1ccccc1-2 ; Cc1nnn2c1Cc1c(C(=O)OCC(F)(F)F)ncn1-c1ccc(F)cc1-2] 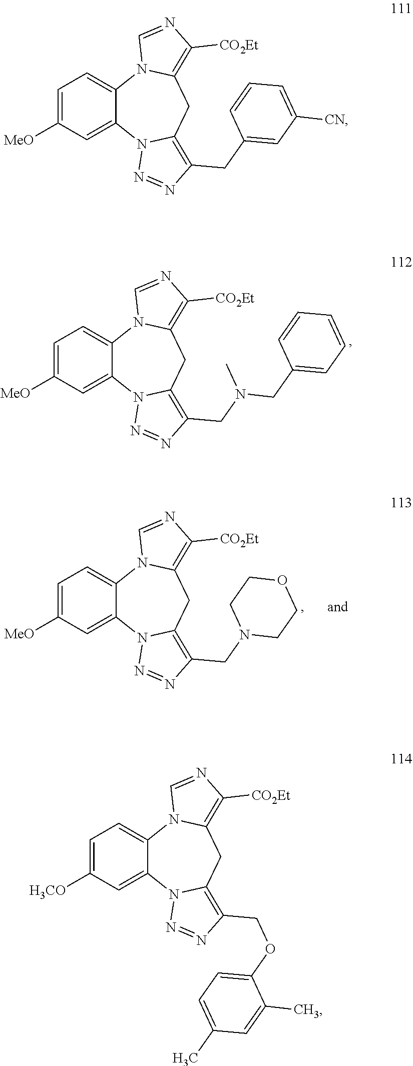 [SMILES: CCOC(=O)c1ncn2c1Cc1c(Cc3cccc(C#N)c3)nnn1-c1cc(OC)ccc1-2 ; CCOC(=O)c1ncn2c1Cc1c(CN(C)Cc3ccccc3)nnn1-c1cc(OC)ccc1-2 ; CCOC(=O)c1ncn2c1Cc1c(CN3CCOCC3)nnn1-c1cc(OC)ccc1-2 ; CCOC(=O)c1ncn2c1Cc1c(COc3ccc(C)cc3C)nnn1-c1cc(OC)ccc1-2]
or a pharmaceutically acceptable salt, hydrate, solvate, polymorph, or isomer of any of the foregoing.

37. The combination according to claim 36, wherein the GABA_{A} α5 receptor agonist is 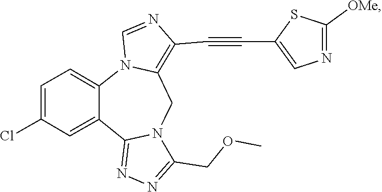 [SMILES: COCc1nnc2n1Cc1c(C#Cc3cnc(OC)s3)ncn1-c1ccc(Cl)cc1-2]
or a pharmaceutically acceptable salt, hydrate, solvate, polymorph, or isomer thereof.

38. The combination according to claim 37, wherein the compound is a polymorph crystalline form of the GABA_{A} α5 receptor agonist of 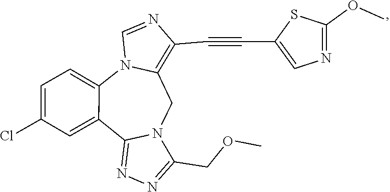 [SMILES: COCc1nnc2n1Cc1c(C#Cc3cnc(OC)s3)ncn1-c1ccc(Cl)cc1-2]
wherein the polymorph crystalline form is Form A and exhibits an XRPD comprising:
a. at least one peak selected from 3.0 and 21.0 degrees 2θ±0.2 degrees 2θ; and
b. at least one additional peak selected from the group consisting of 9.1, 10.7, 13.8, 22.0, 23.1, 23.9, 24.4, and 27.1 degrees 2θ±0.2 degrees 2θ.

39. The combination according to claim 37, wherein the GABA_{A} α5 receptor agonist is a polymorph crystalline form of 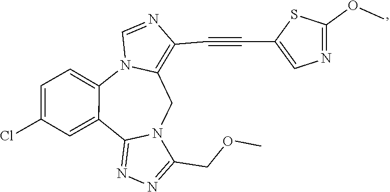 [SMILES: COCc1nnc2n1Cc1c(C#Cc3cnc(OC)s3)ncn1-c1ccc(Cl)cc1-2]
wherein the polymorph crystalline form is Form B and exhibits an XRPD comprising:
a. at least one peak selected from 13.0 and 15.3 degrees 2θ±0.2 degrees 2θ; and
b. at least one additional peak selected from the group consisting of 7.0, 9.3, 10.2, 10.4, 12.5, 13.6, 14.0, 22.0, 23.0, 23.6, and 27.3 degrees 2θ±0.2 degrees 2θ.

40. The combination according to claim 37, wherein the GABA_{A} α5 receptor agonist is a solvate crystalline form of 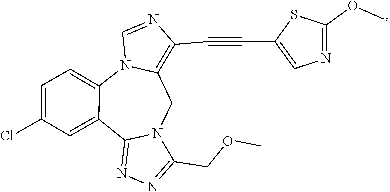 [SMILES: COCc1nnc2n1Cc1c(C#Cc3cnc(OC)s3)ncn1-c1ccc(Cl)cc1-2]
wherein the solvate crystalline form is Form C and exhibits an XRPD comprising:
a. at least one peak selected from 8.5 and 18.9 degrees 2θ±0.2 degrees 2θ; and
b. at least one additional peak selected from the group consisting of 7.1, 9.4, 10.3, 12.3, 12.5, 14.2, 20.7, 22.1, 23.2, 23.7, 24.0, and 26.4 degrees 2θ±0.2 degrees 2θ.

41. The combination according to claim 37, wherein the GABA_{A} α5 receptor agonist is a polymorph crystalline form of 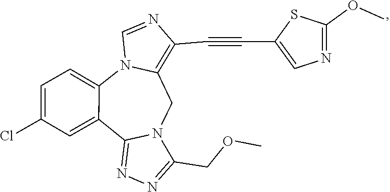 [SMILES: COCc1nnc2n1Cc1c(C#Cc3cnc(OC)s3)ncn1-c1ccc(Cl)cc1-2]
wherein the polymorph crystalline form is Form E and exhibits an XRPD comprising:
a. at least one peak selected from the group consisting of 11.4, 18.1, and 21.6 degrees 2θ±0.2 degrees 2θ; and
b. at least one additional peak selected from the group consisting of 7.2, 22.0, 23.0, 24.2, 25.0, and 26.6 degrees 2θ±0.2 degrees 2θ.

42. The combination according to claim 37, wherein the GABA_{A} α5 receptor agonist is a hydrate crystalline form of 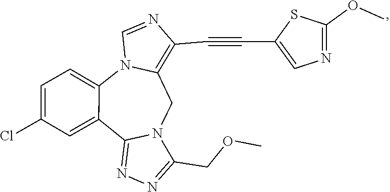 [SMILES: COCc1nnc2n1Cc1c(C#Cc3cnc(OC)s3)ncn1-c1ccc(Cl)cc1-2]
wherein the polymorph crystalline form is Form F and exhibits an XRPD comprising:
a. at least one peak selected from the group consisting of 9.9, 11.9, 17.3, 19.4, and 25.7 degrees 2θ±0.2 degrees 2θ; and
b. at least one additional peak selected from the group consisting of 9.7, 12.1, 20.8, 23.2, 23.7, 24.2, 25.0, and 26.4 degrees 2θ±0.2 degrees 2θ.

43. The combination according to claim 27, wherein the GABA_{A} α5 receptor agonist is selected form the group consisting of Compounds 1-740, or a pharmaceutically acceptable salt, hydrate, solvate, isomer, or polymorph thereof.

44. The combination according to claim 27, wherein the GABA_{A} α5 receptor agonist, or the pharmaceutically acceptable salt, hydrate, solvate, isomer, or polymorph thereof, is present in an amount between 5 mg and 1000 mg.

45. The combination according to claim 27, wherein the SV2A inhibitor, or the pharmaceutically acceptable salt, hydrate, solvate, isomer, or polymorph thereof, is present in an amount between 0.07 mg to 350 mg.

46. The combination according to claim 27, wherein one or more of the SV2A inhibitors or the GABA_{A} α5 receptor agonists, or a pharmaceutically acceptable salt, hydrate, solvate, isomer, or polymorph thereof, or the pharmaceutical compositions thereof, is in an extended release form, a non-extended release form, or an immediate release form.

47. The combination according to claim 27, wherein the GABA_{A} α5 receptor agonist, or a pharmaceutically acceptable salt, hydrate, solvate, isomer, or polymorph thereof, or the pharmaceutical composition thereof, and the SV2A inhibitor, or a pharmaceutically acceptable salt, hydrate, solvate, isomer, or polymorph thereof, or the pharmaceutical compositions thereof are in separate dosage forms packaged together, in a unit dosage form, or in separate packages for simultaneous or sequential administration.

48.-56. (canceled)

57. A method of treating cognitive impairment associated with a central nervous system (CNS) disorder in a subject in need thereof or at risk thereof, the method comprising administering to the subject the combination according to claim 27.

58.-62. (canceled)

63. The method according to claim 57, wherein the CNS disorder is age-related cognitive impairment, mild cognitive impairment (MCI), amnestic mild cognitive impairment (aMCI), dementia, Alzheimer's disease, schizophrenia, amyotrophic lateral sclerosis (ALS), post-traumatic stress disorder (PTSD), mental retardation, Parkinson's disease (PD), autism, compulsive behavior, substance addiction, bipolar disorder, or cancer-therapy-related cognitive impairment.

64. (canceled)

65. A method of treating a brain cancer in a subject in need thereof, the method comprising administering to the subject the combination according to claim 27.

66. (canceled)

67. The method according to claim 57, wherein the combination is administered subcutaneously, intravenously, orally, sublingually, buccally, transdermally, arterially, intradermally, intramuscularly, intraperitoneally, ocularly, intranasally, intraspinally or intracerebrally.

68. (canceled)

69. (canceled)

70. The method according to claim 57, wherein the combination is administered once or twice daily.

71.-110. (canceled)
